SEQUENCE LISTING (26 sequences)

SEQ ID NO: 1 - protein, 746 aa, Homo sapiens
MGQTGKKSEKGPVCWRKRVKSEYMRLRQLKRFRRADEVKSMFSSNRQKILERTEILNQEWKQRRIQPVHILTSVSSLRGTRECSVTSDLDFPTQVIPLKTLNAVASVPIMYSWSPLQQNFMVEDETVLHNIPYMGDEVLDQDGTFIEELIKNYDGKVHGDRECGFINDEIFVELVNALGQYNDDDDDDDGDDPEEREEKQKDLEDHRDDKESRPPRKFPSDKIFEAISSMFPDKGTAEELKEKYKELTEQQLPGALPPECTPNIDGPNAKSVQREQSLHSFHTLFCRRCFKYDCFLHPFHATPNTYKRKNTETALDNKPCGPQCYQHLEGAKEFAAALTAERIKTPPKRPGGRRRGRLPNNSSRPSTPTINVLESKDTDSDREAGTETGGENNDKEEEEKKDETSSSSEANSRCQTPIKMKPNIEPPENVEWSGAEASMFRVLIGTYYDNFCAIARLIGTKTCRQVYEFRVKESSIIAPAPAEDVDTPPRKKKRKHRLWAAHCRKIQLKKDGSSNHVYNYQPCDHPRQPCDSSCPCVIAQNFCEKFCQCSSECQNRFPGCRCKAQCNTKQCPCYLAVRECDPDLCLTCGAADHWDSKNVSCKNCSIQRGSKKHLLLAPSDVAGWGIFIKDPVQKNEFISEYCGEIISQDEADRRGKVYDKYMCSFLFNLNNDFVVDATRKGNKIRFANHSVNPNCYAKVMMVNGDHRIGIFAKRAIQTGEELFFDYRYSQADALKYVGIEREMEIP

SEQ ID NO: 2 - DNA, 2723 bp, Homo sapiens
ggcggcgcttgattgggctgggggggccaaataaaagcgatggcgattgggctgccgcgtttggcgctcggtccggtcgcgtccgacacccggtgggactcagaaggcagtggagccccggcggcggcggcggcggcgcgcgggggcgacgcgcgggaacaacgcgagtcggcgcgcgggacgaagaataatcatgggccagactgggaagaaatctgagaagggaccagtttgttggcggaagcgtgtaaaatcagagtacatgcgactgagacagctcaagaggttcagacgagctgatgaagtaaagagtatgtttagttccaatcgtcagaaaattttggaaagaacggaaatcttaaaccaagaatggaaacagcgaaggatacagcctgtgcacatcctgacttctgtgagctcattgcgcgggactagggagtgttcggtgaccagtgacttggattttccaacacaagtcatcccattaaagactctgaatgcagttgcttcagtacccataatgtattcttggtctcccctacagcagaattttatggtggaagatgaaactgttttacataacattccttatatgggagatgaagttttagatcaggatggtactttcattgaagaactaataaaaaattatgatgggaaagtacacggggatagagaatgtgggtttataaatgatgaaatttttgtggagttggtgaatgcccttggtcaatataatgatgatgacgatgatgatgatggagacgatcctgaagaaagagaagaaaagcagaaagatctggaggatcaccgagatgataaagaaagccgcccacctcggaaatttccttctgataaaatttttgaagccatttcctcaatgtttccagataagggcacagcagaagaactaaaggaaaaatataaagaactcaccgaacagcagctcccaggcgcacttcctcctgaatgtacccccaacatagatggaccaaatgctaaatctgttcagagagagcaaagcttacactcctttcatacgcttttctgtaggcgatgttttaaatatgactgcttcctacatcgtaagtgcaattattcttttcatgcaacacccaacacttataagcggaagaacacagaaacagctctagacaacaaaccttgtggaccacagtgttaccagcatttggagggagcaaaggagtttgctgctgctctcaccgctgagcggataaagaccccaccaaaacgtccaggaggccgcagaagaggacggcttcccaataacagtagcaggcccagcacccccaccattaatgtgctggaatcaaaggatacagacagtgatagggaagcagggactgaaacggggggagagaacaatgataaagaagaagaagagaagaaagatgaaacttcgagctcctctgaagcaaattctcggtgtcaaacaccaataaagatgaagccaaatattgaacctcctgagaatgtggagtggagtggtgctgaagcctcaatgtttagagtcctcattggcacttactatgacaatttctgtgccattgctaggttaattgggaccaaaacatgtagacaggtgtatgagtttagagtcaaagaatctagcatcatagctccagctcccgctgaggatgtggatactcctccaaggaaaaagaagaggaaacaccggttgtgggctgcacactgcagaaagatacagctgaaaaaggacggctcctctaaccatgtttacaactatcaaccctgtgatcatccacggcagccttgtgacagttcgtgcccttgtgtgatagcacaaaatttttgtgaaaagttttgtcaatgtagttcagagtgtcaaaaccgctttccgggatgccgctgcaaagcacagtgcaacaccaagcagtgcccgtgctacctggctgtccgagagtgtgaccctgacctctgtcttacttgtggagccgctgaccattgggacagtaaaaatgtgtcctgcaagaactgcagtattcagcggggctccaaaaagcatctattgctggcaccatctgacgtggcaggctgggggatttttatcaaagatcctgtgcagaaaaatgaattcatctcagaatactgtggagagattatttctcaagatgaagctgacagaagagggaaagtgtatgataaatacatgtgcagctttctgttcaacttgaacaatgattttgtggtggatgcaacccgcaagggtaacaaaattcgttttgcaaatcattcggtaaatccaaactgctatgcaaaagttatgatggttaacggtgatcacaggataggtatttttgccaagagagccatccagactggcgaagagctgttttttgattacagatacagccaggctgatgccctgaagtatgtcggcatcgaaagagaaatggaaatcccttgacatctgctacctcctcccccctcctctgaaacagctgccttagcttcaggaacctcgagtactgtgggcaatttagaaaaagaacatgcagtttgaaattctgaatttgcaaagtactgtaagaataatttatagtaatgagtttaaaaatcaactttttattgccttctcaccagctgcaaagtgttttgtaccagtgaatttttgcaataatgcagtatggtacatttttcaactttgaataaagaatacttgaacttgtccttgttgaatc

SEQ ID NO: 3 - protein, 751 aa, Homo sapiens
MGQTGKKSEKGPVCWRKRVKSEYMRLRQLKRFRRADEVKSMFSSNRQKILERTEILNQEWKQRRIQPVHILTSVSSLRGTRECSVTSDLDFPTQVIPLKTLNAVASVPIMYSWSPLQQNFMVEDETVLHNIPYMGDEVLDQDGTFIEELIKNYDGKVHGDRECGFINDEIFVELVNALGQYNDDDDDDDGDDPEEREEKQKDLEDHRDDKESRPPRKFPSDKIFEAISSMFPDKGTAEELKEKYKELTEQQLPGALPPECTPNIDGPNAKSVQREQSLHSFHTLFCRRCFKYDCFLHRKCNYSFHATPNTYKRKNTETALDNKPCGPQCYQHLEGAKEFAAALTAERIKTPPKRPGGRRRGRLPNNSSRPSTPTINVLESKDTDSDREAGTETGGENNDKEEEEKKDETSSSSEANSRCQTPIKMKPNIEPPENVEWSGAEASMFRVLIGTYYDNFCAIARLIGTKTCRQVYEFRVKESSIIAPAPAEDVDTPPRKKKRKHRLWAAHCRKIQLKKDGSSNHVYNYQPCDHPRQPCDSSCPCVIAQNFCEKFCQCSSECQNRFPGCRCKAQCNTKQCPCYLAVRECDPDLCLTCGAADHWDSKNVSCKNCSIQRGSKKHLLLAPSDVAGWGIFIKDPVQKNEFISEYCGEIISQDEADRRGKVYDKYMCSFLFNLNNDFVVDATRKGNKIRFANHSVNPNCYAKVMMVNGDHRIGIFAKRAIQTGEELFFDYRYSQADALKYVGIEREMEIP

SEQ ID NO: 4 - DNA, 2591 bp, Homo sapiens
ggcggcgcttgattgggctgggggggccaaataaaagcgatggcgattgggctgccgcgtttggcgctcggtccggtcgcgtccgacacccggtgggactcagaaggcagtggagccccggcggcggcggcggcggcgcgcgggggcgacgcgcgggaacaacgcgagtcggcgcgcgggacgaagaataatcatgggccagactgggaagaaatctgagaagggaccagtttgttggcggaagcgtgtaaaatcagagtacatgcgactgagacagctcaagaggttcagacgagctgatgaagtaaagagtatgtttagttccaatcgtcagaaaattttggaaagaacggaaatcttaaaccaagaatggaaacagcgaaggatacagcctgtgcacatcctgacttctgtgagctcattgcgcgggactagggaggtggaagatgaaactgttttacataacattccttatatgggagatgaagttttagatcaggatggtactttcattgaagaactaataaaaaattatgatgggaaagtacacggggatagagaatgtgggtttataaatgatgaaatttttgtggagttggtgaatgcccttggtcaatataatgatgatgacgatgatgatgatggagacgatcctgaagaaagagaagaaaagcagaaagatctggaggatcaccgagatgataaagaaagccgcccacctcggaaatttccttctgataaaatttttgaagccatttcctcaatgtttccagataagggcacagcagaagaactaaaggaaaaatataaagaactcaccgaacagcagctcccaggcgcacttcctcctgaatgtacccccaacatagatggaccaaatgctaaatctgttcagagagagcaaagcttacactcctttcatacgcttttctgtaggcgatgttttaaatatgactgcttcctacatccttttcatgcaacacccaacacttataagcggaagaacacagaaacagctctagacaacaaaccttgtggaccacagtgttaccagcatttggagggagcaaaggagtttgctgctgctctcaccgctgagcggataaagaccccaccaaaacgtccaggaggccgcagaagaggacggcttcccaataacagtagcaggcccagcacccccaccattaatgtgctggaatcaaaggatacagacagtgatagggaagcagggactgaaacggggggagagaacaatgataaagaagaagaagagaagaaagatgaaacttcgagctcctctgaagcaaattctcggtgtcaaacaccaataaagatgaagccaaatattgaacctcctgagaatgtggagtggagtggtgctgaagcctcaatgtttagagtcctcattggcacttactatgacaatttctgtgccattgctaggttaattgggaccaaaacatgtagacaggtgtatgagtttagagtcaaagaatctagcatcatagctccagctcccgctgaggatgtggatactcctccaaggaaaaagaagaggaaacaccggttgtgggctgcacactgcagaaagatacagctgaaaaaggacggctcctctaaccatgtttacaactatcaaccctgtgatcatccacggcagccttgtgacagttcgtgcccttgtgtgatagcacaaaatttttgtgaaaagttttgtcaatgtagttcagagtgtcaaaaccgctttccgggatgccgctgcaaagcacagtgcaacaccaagcagtgcccgtgctacctggctgtccgagagtgtgaccctgacctctgtcttacttgtggagccgctgaccattgggacagtaaaaatgtgtcctgcaagaactgcagtattcagcggggctccaaaaagcatctattgctggcaccatctgacgtggcaggctgggggatttttatcaaagatcctgtgcagaaaaatgaattcatctcagaatactgtggagagattatttctcaagatgaagctgacagaagagggaaagtgtatgataaatacatgtgcagctttctgttcaacttgaacaatgattttgtggtggatgcaacccgcaagggtaacaaaattcgttttgcaaatcattcggtaaatccaaactgctatgcaaaagttatgatggttaacggtgatcacaggataggtatttttgccaagagagccatccagactggcgaagagctgttttttgattacagatacagccaggctgatgccctgaagtatgtcggcatcgaaagagaaatggaaatcccttgacatctgctacctcctcccccctcctctgaaacagctgccttagcttcaggaacctcgagtactgtgggcaatttagaaaaagaacatgcagtttgaaattctgaatttgcaaagtactgtaagaataatttatagtaatgagtttaaaaatcaactttttattgccttctcaccagctgcaaagtgttttgtaccagtgaatttttgcaataatgcagtatggtacatttttcaactttgaataaagaatacttgaacttgtccttgttgaatc

SEQ ID NO: 5 - protein, 707 aa, Homo sapiens
MGQTGKKSEKGPVCWRKRVKSEYMRLRQLKRFRRADEVKSMFSSNRQKILERTEILNQEWKQRRIQPVHILTSVSSLRGTREVEDETVLHNIPYMGDEVLDQDGTFIEELIKNYDGKVHGDRECGFINDEIFVELVNALGQYNDDDDDDDGDDPEEREEKQKDLEDHRDDKESRPPRKFPSDKIFEAISSMFPDKGTAEELKEKYKELTEQQLPGALPPECTPNIDGPNAKSVQREQSLHSFHTLFCRRCFKYDCFLHPFHATPNTYKRKNTETALDNKPCGPQCYQHLEGAKEFAAALTAERIKTPPKRPGGRRRGRLPNNSSRPSTPTINVLESKDTDSDREAGTETGGENNDKEEEEKKDETSSSSEANSRCQTPIKMKPNIEPPENVEWSGAEASMFRVLIGTYYDNFCAIARLIGTKTCRQVYEFRVKESSIIAPAPAEDVDTPPRKKKRKHRLWAAHCRKIQLKKDGSSNHVYNYQPCDHPRQPCDSSCPCVIAQNFCEKFCQCSSECQNRFPGCRCKAQCNTKQCPCYLAVRECDPDLCLTCGAADHWDSKNVSCKNCSIQRGSKKHLLLAPSDVAGWGIFIKDPVQKNEFISEYCGEIISQDEADRRGKVYDKYMCSFLFNLNNDFVVDATRKGNKIRFANHSVNPNCYAKVMMVNGDHRIGIFAKRAIQTGEELFFDYRYSQADALKYVGIEREMEIP

SEQ ID NO: 6 - protein, 200 aa, Homo sapiens
SCPCVIAQNFCEKFCQCSSECQNRFPGCRCKAQCNTKQCPCYLAVRECDPDLCLTCGAADHWDSKNVSCKNCSIQRGSKKHLLLAPSDVAGWGIFIKDPVQKNEFISEYCGEIISQDEADRRGKVYDKYMCSFLFNLNNDFVVDATRKGNKIRFANHSVNPNCYAKVMMVNGDHRIGIFAKRAIQTGEELFFDYRYSQAD

SEQ ID NO: 7 - protein, 114 aa, Homo sapiens
HLLLAPSDVAGWGIFIKDPVQKNEFISEYCGEIISQDEADRRGKVYDKYMCSFLFNLNNDFVVDATRKGNKIRFANHSVNPNCYAKVMMVNGDHRIGIFAKRAIQTGEELFFDY

SEQ ID NO: 8 - DNA, 342 bp, Homo sapiens
catctattgctggcaccatctgacgtggcaggctgggggatttttatcaaagatcctgtgcagaaaaatgaattcatctcagaatactgtggagagattatttctcaagatgaagctgacagaagagggaaagtgtatgataaatacatgtgcagctttctgttcaacttgaacaatgattttgtggtggatgcaacccgcaagggtaacaaaattcgttttgcaaatcattcggtaaatccaaactgctatgcaaaagttatgatggttaacggtgatcacaggataggtatttttgccaagagagccatccagactggcgaagagctgttttttgattac

SEQ ID NO: 9 - protein, 746 aa, Homo sapiens
  misc_feature (641)..(641): Xaa can be any naturally occurring amino acid
MGQTGKKSEKGPVCWRKRVKSEYMRLRQLKRFRRADEVKSMFSSNRQKILERTEILNQEWKQRRIQPVHILTSVSSLRGTRECSVTSDLDFPTQVIPLKTLNAVASVPIMYSWSPLQQNFMVEDETVLHNIPYMGDEVLDQDGTFIEELIKNYDGKVHGDRECGFINDEIFVELVNALGQYNDDDDDDDGDDPEEREEKQKDLEDHRDDKESRPPRKFPSDKIFEAISSMFPDKGTAEELKEKYKELTEQQLPGALPPECTPNIDGPNAKSVQREQSLHSFHTLFCRRCFKYDCFLHPFHATPNTYKRKNTETALDNKPCGPQCYQHLEGAKEFAAALTAERIKTPPKRPGGRRRGRLPNNSSRPSTPTINVLESKDTDSDREAGTETGGENNDKEEEEKKDETSSSSEANSRCQTPIKMKPNIEPPENVEWSGAEASMFRVLIGTYYDNFCAIARLIGTKTCRQVYEFRVKESSIIAPAPAEDVDTPPRKKKRKHRLWAAHCRKIQLKKDGSSNHVYNYQPCDHPRQPCDSSCPCVIAQNFCEKFCQCSSECQNRFPGCRCKAQCNTKQCPCYLAVRECDPDLCLTCGAADHWDSKNVSCKNCSIQRGSKKHLLLAPSDVAGWGIFIKDPVQKNEFISEXCGEIISQDEADRRGKVYDKYMCSFLFNLNNDFVVDATRKGNKIRFANHSVNPNCYAKVMMVNGDHRIGIFAKRAIQTGEELFFDYRYSQADALKYVGIEREMEIP

SEQ ID NO: 10 - protein, 746 aa, Homo sapiens
MGQTGKKSEKGPVCWRKRVKSEYMRLRQLKRFRRADEVKSMFSSNRQKILERTEILNQEWKQRRIQPVHILTSVSSLRGTRECSVTSDLDFPTQVIPLKTLNAVASVPIMYSWSPLQQNFMVEDETVLHNIPYMGDEVLDQDGTFIEELIKNYDGKVHGDRECGFINDEIFVELVNALGQYNDDDDDDDGDDPEEREEKQKDLEDHRDDKESRPPRKFPSDKIFEAISSMFPDKGTAEELKEKYKELTEQQLPGALPPECTPNIDGPNAKSVQREQSLHSFHTLFCRRCFKYDCFLHPFHATPNTYKRKNTETALDNKPCGPQCYQHLEGAKEFAAALTAERIKTPPKRPGGRRRGRLPNNSSRPSTPTINVLESKDTDSDREAGTETGGENNDKEEEEKKDETSSSSEANSRCQTPIKMKPNIEPPENVEWSGAEASMFRVLIGTYYDNFCAIARLIGTKTCRQVYEFRVKESSIIAPAPAEDVDTPPRKKKRKHRLWAAHCRKIQLKKDGSSNHVYNYQPCDHPRQPCDSSCPCVIAQNFCEKFCQCSSECQNRFPGCRCKAQCNTKQCPCYLAVRECDPDLCLTCGAADHWDSKNVSCKNCSIQRGSKKHLLLAPSDVAGWGIFIKDPVQKNEFISEFCGEIISQDEADRRGKVYDKYMCSFLFNLNNDFVVDATRKGNKIRFANHSVNPNCYAKVMMVNGDHRIGIFAKRAIQTGEELFFDYRYSQADALKYVGIEREMEIP

SEQ ID NO: 11 - protein, 746 aa, Artificial Sequence
  Synthetic Polynucleotide
MGQTGKKSEKGPVCWRKRVKSEYMRLRQLKRFRRADEVKSMFSSNRQKILERTEILNQEWKQRRIQPVHILTSVSSLRGTRECSVTSDLDFPTQVIPLKTLNAVASVPIMYSWSPLQQNFMVEDETVLHNIPYMGDEVLDQDGTFIEELIKNYDGKVHGDRECGFINDEIFVELVNALGQYNDDDDDDDGDDPEEREEKQKDLEDHRDDKESRPPRKFPSDKIFEAISSMFPDKGTAEELKEKYKELTEQQLPGALPPECTPNIDGPNAKSVQREQSLHSFHTLFCRRCFKYDCFLHPFHATPNTYKRKNTETALDNKPCGPQCYQHLEGAKEFAAALTAERIKTPPKRPGGRRRGRLPNNSSRPSTPTINVLESKDTDSDREAGTETGGENNDKEEEEKKDETSSSSEANSRCQTPIKMKPNIEPPENVEWSGAEASMFRVLIGTYYDNFCAIARLIGTKTCRQVYEFRVKESSIIAPAPAEDVDTPPRKKKRKHRLWAAHCRKIQLKKDGSSNHVYNYQPCDHPRQPCDSSCPCVIAQNFCEKFCQCSSECQNRFPGCRCKAQCNTKQCPCYLAVRECDPDLCLTCGAADHWDSKNVSCKNCSIQRGSKKHLLLAPSDVAGWGIFIKDPVQKNEFISEHCGEIISQDEADRRGKVYDKYMCSFLFNLNNDFVVDATRKGNKIRFANHSVNPNCYAKVMMVNGDHRIGIFAKRAIQTGEELFFDYRYSQADALKYVGIEREMEIP

SEQ ID NO: 12 - protein, 746 aa, Artificial Sequence
  Synthetic Polynucleotide
MGQTGKKSEKGPVCWRKRVKSEYMRLRQLKRFRRADEVKSMFSSNRQKILERTEILNQEWKQRRIQPVHILTSVSSLRGTRECSVTSDLDFPTQVIPLKTLNAVASVPIMYSWSPLQQNFMVEDETVLHNIPYMGDEVLDQDGTFIEELIKNYDGKVHGDRECGFINDEIFVELVNALGQYNDDDDDDDGDDPEEREEKQKDLEDHRDDKESRPPRKFPSDKIFEAISSMFPDKGTAEELKEKYKELTEQQLPGALPPECTPNIDGPNAKSVQREQSLHSFHTLFCRRCFKYDCFLHPFHATPNTYKRKNTETALDNKPCGPQCYQHLEGAKEFAAALTAERIKTPPKRPGGRRRGRLPNNSSRPSTPTINVLESKDTDSDREAGTETGGENNDKEEEEKKDETSSSSEANSRCQTPIKMKPNIEPPENVEWSGAEASMFRVLIGTYYDNFCAIARLIGTKTCRQVYEFRVKESSIIAPAPAEDVDTPPRKKKRKHRLWAAHCRKIQLKKDGSSNHVYNYQPCDHPRQPCDSSCPCVIAQNFCEKFCQCSSECQNRFPGCRCKAQCNTKQCPCYLAVRECDPDLCLTCGAADHWDSKNVSCKNCSIQRGSKKHLLLAPSDVAGWGIFIKDPVQKNEFISENCGEIISQDEADRRGKVYDKYMCSFLFNLNNDFVVDATRKGNKIRFANHSVNPNCYAKVMMVNGDHRIGIFAKRAIQTGEELFFDYRYSQADALKYVGIEREMEIP

SEQ ID NO: 13 - protein, 746 aa, Artificial Sequence
  Synthetic Polynucleotide
MGQTGKKSEKGPVCWRKRVKSEYMRLRQLKRFRRADEVKSMFSSNRQKILERTEILNQEWKQRRIQPVHILTSVSSLRGTRECSVTSDLDFPTQVIPLKTLNAVASVPIMYSWSPLQQNFMVEDETVLHNIPYMGDEVLDQDGTFIEELIKNYDGKVHGDRECGFINDEIFVELVNALGQYNDDDDDDDGDDPEEREEKQKDLEDHRDDKESRPPRKFPSDKIFEAISSMFPDKGTAEELKEKYKELTEQQLPGALPPECTPNIDGPNAKSVQREQSLHSFHTLFCRRCFKYDCFLHPFHATPNTYKRKNTETALDNKPCGPQCYQHLEGAKEFAAALTAERIKTPPKRPGGRRRGRLPNNSSRPSTPTINVLESKDTDSDREAGTETGGENNDKEEEEKKDETSSSSEANSRCQTPIKMKPNIEPPENVEWSGAEASMFRVLIGTYYDNFCAIARLIGTKTCRQVYEFRVKESSIIAPAPAEDVDTPPRKKKRKHRLWAAHCRKIQLKKDGSSNHVYNYQPCDHPRQPCDSSCPCVIAQNFCEKFCQCSSECQNRFPGCRCKAQCNTKQCPCYLAVRECDPDLCLTCGAADHWDSKNVSCKNCSIQRGSKKHLLLAPSDVAGWGIFIKDPVQKNEFISESCGEIISQDEADRRGKVYDKYMCSFLFNLNNDFVVDATRKGNKIRFANHSVNPNCYAKVMMVNGDHRIGIFAKRAIQTGEELFFDYRYSQADALKYVGIEREMEIP

SEQ ID NO: 14 - protein, 746 aa, Artificial Sequence
  Synthetic Polynucleotide
MGQTGKKSEKGPVCWRKRVKSEYMRLRQLKRFRRADEVKSMFSSNRQKILERTEILNQEWKQRRIQPVHILTSVSSLRGTRECSVTSDLDFPTQVIPLKTLNAVASVPIMYSWSPLQQNFMVEDETVLHNIPYMGDEVLDQDGTFIEELIKNYDGKVHGDRECGFINDEIFVELVNALGQYNDDDDDDDGDDPEEREEKQKDLEDHRDDKESRPPRKFPSDKIFEAISSMFPDKGTAEELKEKYKELTEQQLPGALPPECTPNIDGPNAKSVQREQSLHSFHTLFCRRCFKYDCFLHPFHATPNTYKRKNTETALDNKPCGPQCYQHLEGAKEFAAALTAERIKTPPKRPGGRRRGRLPNNSSRPSTPTINVLESKDTDSDREAGTETGGENNDKEEEEKKDETSSSSEANSRCQTPIKMKPNIEPPENVEWSGAEASMFRVLIGTYYDNFCAIARLIGTKTCRQVYEFRVKESSIIAPAPAEDVDTPPRKKKRKHRLWAAHCRKIQLKKDGSSNHVYNYQPCDHPRQPCDSSCPCVIAQNFCEKFCQCSSECQNRFPGCRCKAQCNTKQCPCYLAVRECDPDLCLTCGAADHWDSKNVSCKNCSIQRGSKKHLLLAPSDVAGWGIFIKDPVQKNEFISECCGEIISQDEADRRGKVYDKYMCSFLFNLNNDFVVDATRKGNKIRFANHSVNPNCYAKVMMVNGDHRIGIFAKRAIQTGEELFFDYRYSQADALKYVGIEREMEIP

SEQ ID NO: 15 - protein, 746 aa, Artificial Sequence
  Synthetic Polynucleotide
  misc_feature (677)..(677): Xaa can be any naturally occurring amino acid
MGQTGKKSEKGPVCWRKRVKSEYMRLRQLKRFRRADEVKSMFSSNRQKILERTEILNQEWKQRRIQPVHILTSVSSLRGTRECSVTSDLDFPTQVIPLKTLNAVASVPIMYSWSPLQQNFMVEDETVLHNIPYMGDEVLDQDGTFIEELIKNYDGKVHGDRECGFINDEIFVELVNALGQYNDDDDDDDGDDPEEREEKQKDLEDHRDDKESRPPRKFPSDKIFEAISSMFPDKGTAEELKEKYKELTEQQLPGALPPECTPNIDGPNAKSVQREQSLHSFHTLFCRRCFKYDCFLHPFHATPNTYKRKNTETALDNKPCGPQCYQHLEGAKEFAAALTAERIKTPPKRPGGRRRGRLPNNSSRPSTPTINVLESKDTDSDREAGTETGGENNDKEEEEKKDETSSSSEANSRCQTPIKMKPNIEPPENVEWSGAEASMFRVLIGTYYDNFCAIARLIGTKTCRQVYEFRVKESSIIAPAPAEDVDTPPRKKKRKHRLWAAHCRKIQLKKDGSSNHVYNYQPCDHPRQPCDSSCPCVIAQNFCEKFCQCSSECQNRFPGCRCKAQCNTKQCPCYLAVRECDPDLCLTCGAADHWDSKNVSCKNCSIQRGSKKHLLLAPSDVAGWGIFIKDPVQKNEFISEYCGEIISQDEADRRGKVYDKYMCSFLFNLNNDFVVDXTRKGNKIRFANHSVNPNCYAKVMMVNGDHRIGIFAKRAIQTGEELFFDYRYSQADALKYVGIEREMEIP

SEQ ID NO: 16 - protein, 746 aa, Artificial Sequence
  Synthetic Ploynucleotide
  misc_feature (687)..(687): Xaa can be any naturally occurring amino acid
MGQTGKKSEKGPVCWRKRVKSEYMRLRQLKRFRRADEVKSMFSSNRQKILERTEILNQEWKQRRIQPVHILTSVSSLRGTRECSVTSDLDFPTQVIPLKTLNAVASVPIMYSWSPLQQNFMVEDETVLHNIPYMGDEVLDQDGTFIEELIKNYDGKVHGDRECGFINDEIFVELVNALGQYNDDDDDDDGDDPEEREEKQKDLEDHRDDKESRPPRKFPSDKIFEAISSMFPDKGTAEELKEKYKELTEQQLPGALPPECTPNIDGPNAKSVQREQSLHSFHTLFCRRCFKYDCFLHPFHATPNTYKRKNTETALDNKPCGPQCYQHLEGAKEFAAALTAERIKTPPKRPGGRRRGRLPNNSSRPSTPTINVLESKDTDSDREAGTETGGENNDKEEEEKKDETSSSSEANSRCQTPIKMKPNIEPPENVEWSGAEASMFRVLIGTYYDNFCAIARLIGTKTCRQVYEFRVKESSIIAPAPAEDVDTPPRKKKRKHRLWAAHCRKIQLKKDGSSNHVYNYQPCDHPRQPCDSSCPCVIAQNFCEKFCQCSSECQNRFPGCRCKAQCNTKQCPCYLAVRECDPDLCLTCGAADHWDSKNVSCKNCSIQRGSKKHLLLAPSDVAGWGIFIKDPVQKNEFISEYCGEIISQDEADRRGKVYDKYMCSFLFNLNNDFVVDATRKGNKIRFXNHSVNPNCYAKVMMVNGDHRIGIFAKRAIQTGEELFFDYRYSQADALKYVGIEREMEIP

SEQ ID NO: 17 - protein, 746 aa, Artificial Sequence
  Synthetic Polynucleotide
  misc_feature (685)..(685): Xaa can be any naturally occurring amino acid
MGQTGKKSEKGPVCWRKRVKSEYMRLRQLKRFRRADEVKSMFSSNRQKILERTEILNQEWKQRRIQPVHILTSVSSLRGTRECSVTSDLDFPTQVIPLKTLNAVASVPIMYSWSPLQQNFMVEDETVLHNIPYMGDEVLDQDGTFIEELIKNYDGKVHGDRECGFINDEIFVELVNALGQYNDDDDDDDGDDPEEREEKQKDLEDHRDDKESRPPRKFPSDKIFEAISSMFPDKGTAEELKEKYKELTEQQLPGALPPECTPNIDGPNAKSVQREQSLHSFHTLFCRRCFKYDCFLHPFHATPNTYKRKNTETALDNKPCGPQCYQHLEGAKEFAAALTAERIKTPPKRPGGRRRGRLPNNSSRPSTPTINVLESKDTDSDREAGTETGGENNDKEEEEKKDETSSSSEANSRCQTPIKMKPNIEPPENVEWSGAEASMFRVLIGTYYDNFCAIARLIGTKTCRQVYEFRVKESSIIAPAPAEDVDTPPRKKKRKHRLWAAHCRKIQLKKDGSSNHVYNYQPCDHPRQPCDSSCPCVIAQNFCEKFCQCSSECQNRFPGCRCKAQCNTKQCPCYLAVRECDPDLCLTCGAADHWDSKNVSCKNCSIQRGSKKHLLLAPSDVAGWGIFIKDPVQKNEFISEYCGEIISQDEADRRGKVYDKYMCSFLFNLNNDFVVDATRKGNKIXFANHSVNPNCYAKVMMVNGDHRIGIFAKRAIQTGEELFFDYRYSQADALKYVGIEREMEIP

SEQ ID NO: 18 - protein, 745 aa, Artificial Sequence
  Synthetic Polynucleotide
  misc_feature (641)..(641): Xaa can be any naturally occurring amino acid
  misc_feature (663)..(665): Xaa can be any naturally occurring amino acid
  misc_feature (667)..(667): Xaa can be any naturally occurring amino acid
  misc_feature (674)..(675): Xaa can be any naturally occurring amino acid
  misc_feature (677)..(677): Xaa can be any naturally occurring amino acid
  misc_feature (684)..(687): Xaa can be any naturally occurring amino acid
  misc_feature (707)..(707): Xaa can be any naturally occurring amino acid
  misc_feature (723)..(723): Xaa can be any naturally occurring amino acid
  misc_feature (725)..(725): Xaa can be any naturally occurring amino acid
  misc_feature (729)..(729): Xaa can be any naturally occurring amino acid
MGQTGKKSEKGPVCWRKRVKSEYMRLRQLKRFRRADEVKSMFSSNRQKILERTEILNQEWKQRRIQPVHILTSVSSLRGTRECSVTSDLDFPTQVIPLKTLNAVASVPIMYSWSPLQQNFMVEDETVLHNIPYMGDEVLDQDGTFIEELIKNYDGKVHGDRECGFINDEIFVELVNALGQYNDDDDDDDGDDPEEREEKQKDLEDHRDDKESRPPRKFPSDKIFEAISSMFPDKGTAEELKEKYKELTEQQLPGALPPECTPNIDGPNAKSVQREQSLHSFHTLFCRRCFKYDCFLHPFHATPNTYKRKNTETALDNKPCGPQCYQHLEGAKEFAAALTAERIKTPPKRPGGRRRGRLPNNSSRPSTPTINVLESKDTDSDREAGTETGGENNDKEEEEKKDETSSSSEANSRCQTPIKMKPNIEPPENVEWSGAEASMFRVLIGTYYDNFCAIARLIGTKTCRQVYEFRVKESSIIAPAPAEDVDTPPRKKKRKHRLWAAHCRKIQLKKDGSSNHVYNYQPCDHPRQPCDSSCPCVIAQNFCEKFCQCSSECQNRFPGCRCKAQCNTKQCPCYLAVRECDPDLCLTCGAADHWDSKNVSCKNCSIQRGSKKHLLLAPSDVAGWGIFIKDPVQKNEFISEXCGEIISQDEADRRGKVYDKYMXXXLXNLNNDFXXDXTRKGNKXXXXHSVNPNCYAKVMMVNGDHRXGIFAKRAIQTGEELFXDXRYSXADALKYVGIEREMEIP

SEQ ID NO: 19 - DNA, 8761 bp, Homo sapiens
gccgaggaggaagaggttgatggcggcggcggagctccgagagacctcggctgggcaggggccggccgtggcgggccggggactgcgcctctagagccgcgagttctcgggaattcgccgcagcggacgcgctcggcgaatttgtgctcttgtgccctcctccgggcttgggcccaggcccggcccctcgcacttgcccttaccttttctatcgagtccgcatccctctccagccactgcgacccggcgaagagaaaaaggaacttcccccaccccctcgggtgccgtcggagccccccagcccacccctgggtgcggcgcggggaccccgggccgaagaagagatttcctgaggattctggttttcctcgcttgtatctccgaaagaattaaaaatggccgagaatgtggtggaaccggggccgccttcagccaagcggcctaaactctcatctccggccctctcggcgtccgccagcgatggcacagattttggctctctatttgacttggagcacgacttaccagatgaattaatcaactctacagaattgggactaaccaatggtggtgatattaatcagcttcagacaagtcttggcatggtacaagatgcagcttctaaacataaacagctgtcagaattgctgcgatctggtagttcccctaacctcaatatgggagttggtggcccaggtcaagtcatggccagccaggcccaacagagcagtcctggattaggtttgataaatagcatggtcaaaagcccaatgacacaggcaggcttgacttctcccaacatggggatgggcactagtggaccaaatcagggtcctacgcagtcaacaggtatgatgaacagtccagtaaatcagcctgccatgggaatgaacacagggatgaatgcgggcatgaatcctggaatgttggctgcaggcaatggacaagggataatgcctaatcaagtcatgaacggttcaattggagcaggccgagggcgacagaatatgcagtacccaaacccaggcatgggaagtgctggcaacttactgactgagcctcttcagcagggctctccccagatgggaggacaaacaggattgagaggcccccagcctcttaagatgggaatgatgaacaaccccaatccttatggttcaccatatactcagaatcctggacagcagattggagccagtggccttggtctccagattcagacaaaaactgtactatcaaataacttatctccatttgctatggacaaaaaggcagttcctggtggaggaatgcccaacatgggtcaacagccagccccgcaggtccagcagccaggcctggtgactccagttgcccaagggatgggttctggagcacatacagctgatccagagaagcgcaagctcatccagcagcagcttgttctccttttgcatgctcacaagtgccagcgccgggaacaggccaatggggaagtgaggcagtgcaaccttccccactgtcgcacaatgaagaatgtcctaaaccacatgacacactgccagtcaggcaagtcttgccaagtggcacactgtgcatcttctcgacaaatcatttcacactggaagaattgtacaagacatgattgtcctgtgtgtctccccctcaaaaatgctggtgataagagaaatcaacagccaattttgactggagcacccgttggacttggaaatcctagctctctaggggtgggtcaacagtctgcccccaacctaagcactgttagtcagattgatcccagctccatagaaagagcctatgcagctcttggactaccctatcaagtaaatcagatgccgacacaaccccaggtgcaagcaaagaaccagcagaatcagcagcctgggcagtctccccaaggcatgcggcccatgagcaacatgagtgctagtcctatgggagtaaatggaggtgtaggagttcaaacgccgagtcttctttctgactcaatgttgcattcagccataaattctcaaaacccaatgatgagtgaaaatgccagtgtgccctccctgggtcctatgccaacagcagctcaaccatccactactggaattcggaaacagtggcacgaagatattactcaggatcttcgaaatcatcttgttcacaaactcgtccaagccatatttcctacgccggatcctgctgctttaaaagacagacggatggaaaacctagttgcatatgctcggaaagttgaaggggacatgtatgaatctgcaaacaatcgagcggaatactaccaccttctagctgagaaaatctataagatccagaaagaactagaagaaaaacgaaggaccagactacagaagcagaacatgctaccaaatgctgcaggcatggttccagtttccatgaatccagggcctaacatgggacagccgcaaccaggaatgacttctaatggccctctacctgacccaagtatgatccgtggcagtgtgccaaaccagatgatgcctcgaataactccacaatctggtttgaatcaatttggccagatgagcatggcccagccccctattgtaccccggcaaacccctcctcttcagcaccatggacagttggctcaacctggagctctcaacccgcctatgggctatgggcctcgtatgcaacagccttccaaccagggccagttccttcctcagactcagttcccatcacagggaatgaatgtaacaaatatccctttggctccgtccagcggtcaagctccagtgtctcaagcacaaatgtctagttcttcctgcccggtgaactctcctataatgcctccagggtctcaggggagccacattcactgtccccagcttcctcaaccagctcttcatcagaattcaccctcgcctgtacctagtcgtacccccacccctcaccatactcccccaagcataggggctcagcagccaccagcaacaacaattccagcccctgttcctacacctcctgccatgccacctgggccacagtcccaggctctacatccccctccaaggcagacacctacaccaccaacaacacaacttccccaacaagtgcagccttcacttcctgctgcaccttctgctgaccagccccagcagcagcctcgctcacagcagagcacagcagcgtctgttcctaccccaacagcaccgctgcttcctccgcagcctgcaactccactttcccagccagctgtaagcattgaaggacaggtatcaaatcctccatctactagtagcacagaagtgaattctcaggccattgctgagaagcagccttcccaggaagtgaagatggaggccaaaatggaagtggatcaaccagaaccagcagatactcagccggaggatatttcagagtctaaagtggaagactgtaaaatggaatctaccgaaacagaagagagaagcactgagttaaaaactgaaataaaagaggaggaagaccagccaagtacttcagctacccagtcatctccggctccaggacagtcaaagaaaaagattttcaaaccagaagaactacgacaggcactgatgccaactttggaggcactttaccgtcaggatccagaatcccttccctttcgtcaacctgtggaccctcagcttttaggaatccctgattactttgatattgtgaagagccccatggatctttctaccattaagaggaagttagacactggacagtatcaggagccctggcagtatgtcgatgatatttggcttatgttcaataatgcctggttatataaccggaaaacatcacgggtatacaaatactgctccaagctctctgaggtctttgaacaagaaattgacccagtgatgcaaagccttggatactgttgtggcagaaagttggagttctctccacagacactgtgttgctacggcaaacagttgtgcacaatacctcgtgatgccacttattacagttaccagaacaggtatcatttctgtgagaagtgtttcaatgagatccaaggggagagcgtttctttgggggatgacccttcccagcctcaaactacaataaataaagaacaattttccaagagaaaaaatgacacactggatcctgaactgtttgttgaatgtacagagtgcggaagaaagatgcatcagatctgtgtccttcaccatgagatcatctggcctgctggattcgtctgtgatggctgtttaaagaaaagtgcacgaactaggaaagaaaataagttttctgctaaaaggttgccatctaccagacttggcacctttctagagaatcgtgtgaatgactttctgaggcgacagaatcaccctgagtcaggagaggtcactgttagagtagttcatgcttctgacaaaaccgtggaagtaaaaccaggcatgaaagcaaggtttgtggacagtggagagatggcagaatcctttccataccgaaccaaagccctctttgcctttgaagaaattgatggtgttgacctgtgcttctttggcatgcatgttcaagagtatggctctgactgccctccacccaaccagaggagagtatacatatcttacctcgatagtgttcatttcttccgtcctaaatgcttgaggactgcagtctatcatgaaatcctaattggatatttagaatatgtcaagaaattaggttacacaacagggcatatttgggcatgtccaccaagtgagggagatgattatatcttccattgccatcctcctgaccagaagatacccaagcccaagcgactgcaggaatggtacaaaaaaatgcttgacaaggctgtatcagagcgtattgtccatgactacaaggatatttttaaacaagctactgaagatagattaacaagtgcaaaggaattgccttatttcgagggtgatttctggcccaatgttctggaagaaagcattaaggaactggaacaggaggaagaagagagaaaacgagaggaaaacaccagcaatgaaagcacagatgtgaccaagggagacagcaaaaatgctaaaaagaagaataataagaaaaccagcaaaaataagagcagcctgagtaggggcaacaagaagaaacccgggatgcccaatgtatctaacgacctctcacagaaactatatgccaccatggagaagcataaagaggtcttctttgtgatccgcctcattgctggccctgctgccaactccctgcctcccattgttgatcctgatcctctcatcccctgcgatctgatggatggtcgggatgcgtttctcacgctggcaagggacaagcacctggagttctcttcactccgaagagcccagtggtccaccatgtgcatgctggtggagctgcacacgcagagccaggaccgctttgtctacacctgcaatgaatgcaagcaccatgtggagacacgctggcactgtactgtctgtgaggattatgacttgtgtatcacctgctataacactaaaaaccatgaccacaaaatggagaaactaggccttggcttagatgatgagagcaacaaccagcaggctgcagccacccagagcccaggcgattctcgccgcctgagtatccagcgctgcatccagtctctggtccatgcttgccagtgtcggaatgccaattgctcactgccatcctgccagaagatgaagcgggttgtgcagcataccaagggttgcaaacggaaaaccaatggcgggtgccccatctgcaagcagctcattgccctctgctgctaccatgccaagcactgccaggagaacaaatgcccggtgccgttctgcctaaacatcaagcagaagctccggcagcaacagctgcagcaccgactacagcaggcccaaatgcttcgcaggaggatggccagcatgcagcggactggtgtggttgggcagcaacagggcctcccttcccccactcctgccactccaacgacaccaactggccaacagccaaccaccccgcagacgccccagcccacttctcagcctcagcctacccctcccaatagcatgccaccctacttgcccaggactcaagctgctggccctgtgtcccagggtaaggcagcaggccaggtgacccctccaacccctcctcagactgctcagccaccccttccagggcccccacctgcagcagtggaaatggcaatgcagattcagagagcagcggagacgcagcgccagatggcccacgtgcaaatttttcaaaggccaatccaacaccagatgcccccgatgactcccatggcccccatgggtatgaacccacctcccatgaccagaggtcccagtgggcatttggagccagggatgggaccgacagggatgcagcaacagccaccctggagccaaggaggattgcctcagccccagcaactacagtctgggatgccaaggccagccatgatgtcagtggcccagcatggtcaacctttgaacatggctccacaaccaggattgggccaggtaggtatcagcccactcaaaccaggcactgtgtctcaacaagccttacaaaaccttttgcggactctcaggtctcccagctctcccctgcagcagcaacaggtgcttagtatccttcacgccaacccccagctgttggctgcattcatcaagcagcgggctgccaagtatgccaactctaatccacaacccatccctgggcagcctggcatgccccaggggcagccagggctacagccacctaccatgccaggtcagcagggggtccactccaatccagccatgcagaacatgaatccaatgcaggcgggcgttcagagggctggcctgccccagcagcaaccacagcagcaactccagccacccatgggagggatgagcccccaggctcagcagatgaacatgaaccacaacaccatgccttcacaattccgagacatcttgagacgacagcaaatgatgcaacagcagcagcaacagggagcagggccaggaataggccctggaatggccaaccataaccagttccagcaaccccaaggagttggctacccaccacagcagcagcagcggatgcagcatcacatgcaacagatgcaacaaggaaatatgggacagataggccagcttccccaggccttgggagcagaggcaggtgccagtctacaggcctatcagcagcgactccttcagcaacagatggggtcccctgttcagcccaaccccatgagcccccagcagcatatgctcccaaatcaggcccagtccccacacctacaaggccagcagatccctaattctctctccaatcaagtgcgctctccccagcctgtcccttctccacggccacagtcccagcccccccactccagtccttccccaaggatgcagcctcagccttctccacaccacgtttccccacagacaagttccccacatcctggactggtagctgcccaggccaaccccatggaacaagggcattttgccagcccggaccagaattcaatgctttctcagcttgctagcaatccaggcatggcaaacctccatggtgcaagcgccacggacctgggactcagcaccgataactcagacttgaattcaaacctctcacagagtacactagacatacactagagacaccttgtagtattttgggagcaaaaaaattattttctcttaacaagactttttgtactgaaaacaatttttttgaatctttcgtagcctaaaagacaattttccttggaacacataagaactgtgcagtagccgtttgtggtttaaagcaaacatgcaagatgaacctgagggatgatagaatacaaagaatatatttttgttatggctggttaccaccagcctttcttcccctttgtgtgtgtggttcaagtgtgcactgggaggaggctgaggcctgtgaagccaaacaatatgctcctgccttgcacctccaataggttttattattttttttaaattaatgaacatatgtaatattaatagttattatttactggtgcagatggttgacatttttccctattttcctcactttatggaagagttaaaacatttctaaaccagaggacaaaaggggttaatgttactttaaaattacattctatatatatataaatatatataaatatatattaaaataccagttttttttctctgggtgcaaagatgttcattcttttaaaaaatgtttaaaaaaaaaaaaaaactgcctttcttcccctcaagtcaacttttgtgctccagaaaattttctattctgtaagtctgagcgtaaaacttcaagtattaaaataatttgtacatgtagagagaaaaatgactttttcaaaaatatacaggggcagctgccaaattgatgtattatatattgtggtttctgtttcttgaaagaatttttttcgttatttttacatctaacaaagtaaaaaaattaaaaagagggtaagaaacgattccggtgggatgattttaacatgcaaaatgtccctgggggtttcttctttgcttgctttcttcctccttaccctaccccccactcacacacacacacacacacacacacacacacacacacacacactttctataaaacttgaaaatagcaaaaaccctcaactgttgtaaatcatgcaattaaagttgattacttataaatatgaactttggatcactgtatagactgttaaatttgatttcttattacctattgttaaataaactgtgtgagacagaca

SEQ ID NO: 20 - protein, 2414 aa, Homo sapiens
MAENVVEPGPPSAKRPKLSSPALSASASDGTDFGSLFDLEHDLPDELINSTELGLTNGGDINQLQTSLGMVQDAASKHKQLSELLRSGSSPNLNMGVGGPGQVMASQAQQSSPGLGLINSMVKSPMTQAGLTSPNMGMGTSGPNQGPTQSTGMMNSPVNQPAMGMNTGMNAGMNPGMLAAGNGQGIMPNQVMNGSIGAGRGRQNMQYPNPGMGSAGNLLTEPLQQGSPQMGGQTGLRGPQPLKMGMMNNPNPYGSPYTQNPGQQIGASGLGLQIQTKTVLSNNLSPFAMDKKAVPGGGMPNMGQQPAPQVQQPGLVTPVAQGMGSGAHTADPEKRKLIQQQLVLLLHAHKCQRREQANGEVRQCNLPHCRTMKNVLNHMTHCQSGKSCQVAHCASSRQIISHWKNCTRHDCPVCLPLKNAGDKRNQQPILTGAPVGLGNPSSLGVGQQSAPNLSTVSQIDPSSIERAYAALGLPYQVNQMPTQPQVQAKNQQNQQPGQSPQGMRPMSNMSASPMGVNGGVGVQTPSLLSDSMLHSAINSQNPMMSENASVPSLGPMPTAAQPSTTGIRKQWHEDITQDLRNHLVHKLVQAIFPTPDPAALKDRRMENLVAYARKVEGDMYESANNRAEYYHLLAEKIYKIQKELEEKRRTRLQKQNMLPNAAGMVPVSMNPGPNMGQPQPGMTSNGPLPDPSMIRGSVPNQMMPRITPQSGLNQFGQMSMAQPPIVPRQTPPLQHHGQLAQPGALNPPMGYGPRMQQPSNQGQFLPQTQFPSQGMNVTNIPLAPSSGQAPVSQAQMSSSSCPVNSPIMPPGSQGSHIHCPQLPQPALHQNSPSPVPSRTPTPHHTPPSIGAQQPPATTIPAPVPTPPAMPPGPQSQALHPPPRQTPTPPTTQLPQQVQPSLPAAPSADQPQQQPRSQQSTAASVPTPTAPLLPPQPATPLSQPAVSIEGQVSNPPSTSSTEVNSQAIAEKQPSQEVKMEAKMEVDQPEPADTQPEDISESKVEDCKMESTETEERSTELKTEIKEEEDQPSTSATQSSPAPGQSKKKIFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQDRFVYTCNECKHHVETRWHCTVCEDYDLCITCYNTKNHDHKMEKLGLGLDDESNNQQAAATQSPGDSRRLSIQRCIQSLVHACQCRNANCSLPSCQKMKRVVQHTKGCKRKTNGGCPICKQLIALCCYHAKHCQENKCPVPFCLNIKQKLRQQQLQHRLQQAQMLRRRMASMQRTGVVGQQQGLPSPTPATPTTPTGQQPTTPQTPQPTSQPQPTPPNSMPPYLPRTQAAGPVSQGKAAGQVTPPTPPQTAQPPLPGPPPAAVEMAMQIQRAAETQRQMAHVQIFQRPIQHQMPPMTPMAPMGMNPPPMTRGPSGHLEPGMGPTGMQQQPPWSQGGLPQPQQLQSGMPRPAMMSVAQHGQPLNMAPQPGLGQVGISPLKPGTVSQQALQNLLRTLRSPSSPLQQQQVLSILHANPQLLAAFIKQRAAKYANSNPQPIPGQPGMPQGQPGLQPPTMPGQQGVHSNPAMQNMNPMQAGVQRAGLPQQQPQQQLQPPMGGMSPQAQQMNMNHNTMPSQFRDILRRQQMMQQQQQQGAGPGIGPGMANHNQFQQPQGVGYPPQQQQRMQHHMQQMQQGNMGQIGQLPQALGAEAGASLQAYQQRLLQQQMGSPVQPNPMSPQQHMLPNQAQSPHLQGQQIPNSLSNQVRSPQPVPSPRPQSQPPHSSPSPRMQPQPSPHHVSPQTSSPHPGLVAAQANPMEQGHFASPDQNSMLSQLASNPGMANLHGASATDLGLSTDNSDLNSNLSQSTLDIH

SEQ ID NO: 21 - protein, 695 aa, Homo sapiens
MGQTGKKSEKGPVCWRKRVKSEYMRLRQLKRFRRADEVKSMFSSNRQKILERTEILNQEWKQRRIQPVHILTSCSVTSDLDFPTQVIPLKTLNAVASVPIMYSWSPLQQNFMVEDETVLHNIPYMGDEVLDQDGTFIEELIKNYDGKVHGDRECGFINDEIFVELVNALGQYNDDDDDDDGDDPEEREEKQKDLEDHRDDKESRPPRKFPSDKIFEAISSMFPDKGTAEELKEKYKELTEQQLPGALPPECTPNIDGPNAKSVQREQSLHSFHTLFCRRCFKYDCFLHPFHATPNTYKRKNTETALDNKPCGPQCYQHLEGAKEFAAALTAERIKTPPKRPGGRRRGRLPNNSSRPSTPTINVLESKDTDSDREAGTETGGENNDKEEEEKKDETSSSSEANSRCQTPIKMKPNIEPPENVEWSGAEASMFRVLIGTYYDNFCAIARLIGTKTCRQVYEFRVKESSIIAPAPAEDVDTPPRKKKRKHRLWAAHCRKIQLKKGQNRFPGCRCKAQCNTKQCPCYLAVRECDPDLCLTCGAADHWDSKNVSCKNCSIQRGSKKHLLLAPSDVAGWGIFIKDPVQKNEFISEYCGEIISQDEADRRGKVYDKYMCSFLFNLNNDFVVDATRKGNKIRFANHSVNPNCYAKVMMVNGDHRIGIFAKRAIQTGEELFFDYRYSQADALKYVGIEREMEIP

SEQ ID NO: 22 - DNA, 2682 bp, Homo sapiens
gacgacgttcgcggcggggaactcggagtagcttcgcctctgacgtttccccacgacgcaccccgaaatccccctgagctccggcggtcgcgggctgccctcgccgcctggtctggctttatgctaagtttgagggaagagtcgagctgctctgctctctattgattgtgtttctggagggcgtcctgttgaattcccacttcattgtgtacatccccttccgttccccccaaaaatctgtgccacagggttactttttgaaagcgggaggaatcgagaagcacgatcttttggaaaacttggtgaacgcctaaataatcatgggccagactgggaagaaatctgagaagggaccagtttgttggcggaagcgtgtaaaatcagagtacatgcgactgagacagctcaagaggttcagacgagctgatgaagtaaagagtatgtttagttccaatcgtcagaaaattttggaaagaacggaaatcttaaaccaagaatggaaacagcgaaggatacagcctgtgcacatcctgacttcttgttcggtgaccagtgacttggattttccaacacaagtcatcccattaaagactctgaatgcagttgcttcagtacccataatgtattcttggtctcccctacagcagaattttatggtggaagatgaaactgttttacataacattccttatatgggagatgaagttttagatcaggatggtactttcattgaagaactaataaaaaattatgatgggaaagtacacggggatagagaatgtgggtttataaatgatgaaatttttgtggagttggtgaatgcccttggtcaatataatgatgatgacgatgatgatgatggagacgatcctgaagaaagagaagaaaagcagaaagatctggaggatcaccgagatgataaagaaagccgcccacctcggaaatttccttctgataaaatttttgaagccatttcctcaatgtttccagataagggcacagcagaagaactaaaggaaaaatataaagaactcaccgaacagcagctcccaggcgcacttcctcctgaatgtacccccaacatagatggaccaaatgctaaatctgttcagagagagcaaagcttacactcctttcatacgcttttctgtaggcgatgttttaaatatgactgcttcctacatccttttcatgcaacacccaacacttataagcggaagaacacagaaacagctctagacaacaaaccttgtggaccacagtgttaccagcatttggagggagcaaaggagtttgctgctgctctcaccgctgagcggataaagaccccaccaaaacgtccaggaggccgcagaagaggacggcttcccaataacagtagcaggcccagcacccccaccattaatgtgctggaatcaaaggatacagacagtgatagggaagcagggactgaaacggggggagagaacaatgataaagaagaagaagagaagaaagatgaaacttcgagctcctctgaagcaaattctcggtgtcaaacaccaataaagatgaagccaaatattgaacctcctgagaatgtggagtggagtggtgctgaagcctcaatgtttagagtcctcattggcacttactatgacaatttctgtgccattgctaggttaattgggaccaaaacatgtagacaggtgtatgagtttagagtcaaagaatctagcatcatagctccagctcccgctgaggatgtggatactcctccaaggaaaaagaagaggaaacaccggttgtgggctgcacactgcagaaagatacagctgaaaaagggtcaaaaccgctttccgggatgccgctgcaaagcacagtgcaacaccaagcagtgcccgtgctacctggctgtccgagagtgtgaccctgacctctgtcttacttgtggagccgctgaccattgggacagtaaaaatgtgtcctgcaagaactgcagtattcagcggggctccaaaaagcatctattgctggcaccatctgacgtggcaggctgggggatttttatcaaagatcctgtgcagaaaaatgaattcatctcagaatactgtggagagattatttctcaagatgaagctgacagaagagggaaagtgtatgataaatacatgtgcagctttctgttcaacttgaacaatgattttgtggtggatgcaacccgcaagggtaacaaaattcgttttgcaaatcattcggtaaatccaaactgctatgcaaaagttatgatggttaacggtgatcacaggataggtatttttgccaagagagccatccagactggcgaagagctgttttttgattacagatacagccaggctgatgccctgaagtatgtcggcatcgaaagagaaatggaaatcccttgacatctgctacctcctcccccctcctctgaaacagctgccttagcttcaggaacctcgagtactgtgggcaatttagaaaaagaacatgcagtttgaaattctgaatttgcaaagtactgtaagaataatttatagtaatgagtttaaaaatcaactttttattgccttctcaccagctgcaaagtgttttgtaccagtgaatttttgcaataatgcagtatggtacatttttcaactttgaataaagaatacttgaacttgtccttgttgaatc

SEQ ID NO: 23 - DNA, 10197 bp, Homo sapiens
ctgcggggcgctgttgctgtggctgagatttggccgccgcctcccccacccggcctgcgccctccctctccctcggcgcccgcccgcccgctcgcggcccgcgctcgctcctctccctcgcagccggcagggcccccgacccccgtccgggccctcgccggcccggccgcccgtgcccggggctgttttcgcgagcaggtgaaaatggctgagaacttgctggacggaccgcccaaccccaaaagagccaaactcagctcgcccggtttctcggcgaatgacagcacagattttggatcattgtttgacttggaaaatgatcttcctgatgagctgatacccaatggaggagaattaggccttttaaacagtgggaaccttgttccagatgctgcttccaaacataaacaactgtcggagcttctacgaggaggcagcggctctagtatcaacccaggaataggaaatgtgagcgccagcagccccgtgcagcagggcctgggtggccaggctcaagggcagccgaacagtgctaacatggccagcctcagtgccatgggcaagagccctctgagccagggagattcttcagcccccagcctgcctaaacaggcagccagcacctctgggcccacccccgctgcctcccaagcactgaatccgcaagcacaaaagcaagtggggctggcgactagcagccctgccacgtcacagactggacctggtatctgcatgaatgctaactttaaccagacccacccaggcctcctcaatagtaactctggccatagcttaattaatcaggcttcacaagggcaggcgcaagtcatgaatggatctcttggggctgctggcagaggaaggggagctggaatgccgtaccctactccagccatgcagggcgcctcgagcagcgtgctggctgagaccctaacgcaggtttccccgcaaatgactggtcacgcgggactgaacaccgcacaggcaggaggcatggccaagatgggaataactgggaacacaagtccatttggacagccctttagtcaagctggagggcagccaatgggagccactggagtgaacccccagttagccagcaaacagagcatggtcaacagtttgcccaccttccctacagatatcaagaatacttcagtcaccaacgtgccaaatatgtctcagatgcaaacatcagtgggaattgtacccacacaagcaattgcaacaggccccactgcagatcctgaaaaacgcaaactgatacagcagcagctggttctactgcttcatgctcataagtgtcagagacgagagcaagcaaacggagaggttcgggcctgctcgctcccgcattgtcgaaccatgaaaaacgttttgaatcacatgacgcattgtcaggctgggaaagcctgccaagttgcccattgtgcatcttcacgacaaatcatctctcattggaagaactgcacacgacatgactgtcctgtttgcctccctttgaaaaatgccagtgacaagcgaaaccaacaaaccatcctggggtctccagctagtggaattcaaaacacaattggttctgttggcacagggcaacagaatgccacttctttaagtaacccaaatcccatagaccccagctccatgcagcgagcctatgctgctctcggactcccctacatgaaccagccccagacgcagctgcagcctcaggttcctggccagcaaccagcacagcctcaaacccaccagcagatgaggactctcaaccccctgggaaataatccaatgaacattccagcaggaggaataacaacagatcagcagcccccaaacttgatttcagaatcagctcttccgacttccctgggggccacaaacccactgatgaacgatggctccaactctggtaacattggaaccctcagcactataccaacagcagctcctccttctagcaccggtgtaaggaaaggctggcacgaacatgtcactcaggacctgcggagccatctagtgcataaactcgtccaagccatcttcccaacacctgatcccgcagctctaaaggatcgccgcatggaaaacctggtagcctatgctaagaaagtggaaggggacatgtacgagtctgccaacagcagggatgaatattatcacttattagcagagaaaatctacaagatacaaaaagaactagaagaaaaacggaggtcgcgtttacataaacaaggcatcttggggaaccagccagccttaccagccccgggggctcagccccctgtgattccacaggcacaacctgtgagacctccaaatggacccctgtccctgccagtgaatcgcatgcaagtttctcaagggatgaattcatttaaccccatgtccttggggaacgtccagttgccacaagcacccatgggacctcgtgcagcctccccaatgaaccactctgtccagatgaacagcatgggctcagtgccagggatggccatttctccttcccgaatgcctcagcctccgaacatgatgggtgcacacaccaacaacatgatggcccaggcgcccgctcagagccagtttctgccacagaaccagttcccgtcatccagcggggcgatgagtgtgggcatggggcagccgccagcccaaacaggcgtgtcacagggacaggtgcctggtgctgctcttcctaaccctctcaacatgctggggcctcaggccagccagctaccttgccctccagtgacacagtcaccactgcacccaacaccgcctcctgcttccacggctgctggcatgccatctctccagcacacgacaccacctgggatgactcctccccagccagcagctcccactcagccatcaactcctgtgtcgtcttccgggcagactcccaccccgactcctggctcagtgcccagtgctacccaaacccagagcacccctacagtccaggcagcagcccaggcccaggtgaccccgcagcctcaaaccccagttcagcccccgtctgtggctacccctcagtcatcgcagcaacagccgacgcctgtgcacgcccagcctcctggcacaccgctttcccaggcagcagccagcattgataacagagtccctaccccctcctcggtggccagcgcagaaaccaattcccagcagccaggacctgacgtacctgtgctggaaatgaagacggagacccaagcagaggacactgagcccgatcctggtgaatccaaaggggagcccaggtctgagatgatggaggaggatttgcaaggagcttcccaagttaaagaagaaacagacatagcagagcagaaatcagaaccaatggaagtggatgaaaagaaacctgaagtgaaagtagaagttaaagaggaagaagagagtagcagtaacggcacagcctctcagtcaacatctccttcgcagccgcgcaaaaaaatctttaaaccagaggagttacgccaggccctcatgccaaccctagaagcactgtatcgacaggacccagagtcattacctttccggcagcctgtagatccccagctcctcggaattccagactattttgacatcgtaaagaatcccatggacctctccaccatcaagcggaagctggacacagggcaataccaagagccctggcagtacgtggacgacgtctggctcatgttcaacaatgcctggctctataatcgcaagacatcccgagtctataagttttgcagtaagcttgcagaggtctttgagcaggaaattgaccctgtcatgcagtcccttggatattgctgtggacgcaagtatgagttttccccacagactttgtgctgctatgggaagcagctgtgtaccattcctcgcgatgctgcctactacagctatcagaataggtatcatttctgtgagaagtgtttcacagagatccagggcgagaatgtgaccctgggtgacgacccttcacagccccagacgacaatttcaaaggatcagtttgaaaagaagaaaaatgataccttagaccccgaacctttcgttgattgcaaggagtgtggccggaagatgcatcagatttgcgttctgcactatgacatcatttggccttcaggttttgtgtgcgacaactgcttgaagaaaactggcagacctcgaaaagaaaacaaattcagtgctaagaggctgcagaccacaagactgggaaaccacttggaagaccgagtgaacaaatttttgcggcgccagaatcaccctgaagccggggaggtttttgtccgagtggtggccagctcagacaagacggtggaggtcaagcccgggatgaagtcacggtttgtggattctggggaaatgtctgaatctttcccatatcgaaccaaagctctgtttgcttttgaggaaattgacggcgtggatgtctgcttttttggaatgcacgtccaagaatacggctctgattgcccccctccaaacacgaggcgtgtgtacatttcttatctggatagtattcatttcttccggccacgttgcctccgcacagccgtttaccatgagatccttattggatatttagagtatgtgaagaaattagggtatgtgacagggcacatctgggcctgtcctccaagtgaaggagatgattacatcttccattgccacccacctgatcaaaaaatacccaagccaaaacgactgcaggagtggtacaaaaagatgctggacaaggcgtttgcagagcggatcatccatgactacaaggatattttcaaacaagcaactgaagacaggctcaccagtgccaaggaactgccctattttgaaggtgatttctggcccaatgtgttagaagagagcattaaggaactagaacaagaagaagaggagaggaaaaaggaagagagcactgcagccagtgaaaccactgagggcagtcagggcgacagcaagaatgccaagaagaagaacaacaagaaaaccaacaagaacaaaagcagcatcagccgcgccaacaagaagaagcccagcatgcccaacgtgtccaatgacctgtcccagaagctgtatgccaccatggagaagcacaaggaggtcttcttcgtgatccacctgcacgctgggcctgtcatcaacaccctgccccccatcgtcgaccccgaccccctgctcagctgtgacctcatggatgggcgcgacgccttcctcaccctcgccagagacaagcactgggagttctcctccttgcgccgctccaagtggtccacgctctgcatgctggtggagctgcacacccagggccaggaccgctttgtctacacctgcaacgagtgcaagcaccacgtggagacgcgctggcactgcactgtgtgcgaggactacgacctctgcatcaactgctataacacgaagagccatgcccataagatggtgaagtgggggctgggcctggatgacgagggcagcagccagggcgagccacagtcaaagagcccccaggagtcacgccggctgagcatccagcgctgcatccagtcgctggtgcacgcgtgccagtgccgcaacgccaactgctcgctgccatcctgccagaagatgaagcgggtggtgcagcacaccaagggctgcaaacgcaagaccaacgggggctgcccggtgtgcaagcagctcatcgccctctgctgctaccacgccaagcactgccaagaaaacaaatgccccgtgcccttctgcctcaacatcaaacacaagctccgccagcagcagatccagcaccgcctgcagcaggcccagctcatgcgccggcggatggccaccatgaacacccgcaacgtgcctcagcagagtctgccttctcctacctcagcaccgcccgggacccccacacagcagcccagcacaccccagacgccgcagccccctgcccagccccaaccctcacccgtgagcatgtcaccagctggcttccccagcgtggcccggactcagccccccaccacggtgtccacagggaagcctaccagccaggtgccggcccccccacccccggcccagccccctcctgcagcggtggaagcggctcggcagatcgagcgtgaggcccagcagcagcagcacctgtaccgggtgaacatcaacaacagcatgcccccaggacgcacgggcatggggaccccggggagccagatggcccccgtgagcctgaatgtgccccgacccaaccaggtgagcgggcccgtcatgcccagcatgcctcccgggcagtggcagcaggcgccccttccccagcagcagcccatgccaggcttgcccaggcctgtgatatccatgcaggcccaggcggccgtggctgggccccggatgcccagcgtgcagccacccaggagcatctcacccagcgctctgcaagacctgctgcggaccctgaagtcgcccagctcccctcagcagcaacagcaggtgctgaacattctcaaatcaaacccgcagctaatggcagctttcatcaaacagcgcacagccaagtacgtggccaatcagcccggcatgcagccccagcctggcctccagtcccagcccggcatgcaaccccagcctggcatgcaccagcagcccagcctgcagaacctgaatgccatgcaggctggcgtgccgcggcccggtgtgcctccacagcagcaggcgatgggaggcctgaacccccagggccaggccttgaacatcatgaacccaggacacaaccccaacatggcgagtatgaatccacagtaccgagaaatgttacggaggcagctgctgcagcagcagcagcaacagcagcagcaacaacagcagcaacagcagcagcagcaagggagtgccggcatggctgggggcatggcggggcacggccagttccagcagcctcaaggacccggaggctacccaccggccatgcagcagcagcagcgcatgcagcagcatctccccctccagggcagctccatgggccagatggcggctcagatgggacagcttggccagatggggcagccggggctgggggcagacagcacccccaacatccagcaagccctgcagcagcggattctgcagcaacagcagatgaagcagcagattgggtccccaggccagccgaaccccatgagcccccagcaacacatgctctcaggacagccacaggcctcgcatctccctggccagcagatcgccacgtcccttagtaaccaggtgcggtctccagcccctgtccagtctccacggccccagtcccagcctccacattccagcccgtcaccacggatacagccccagccttcgccacaccacgtctcaccccagactggttccccccaccccggactcgcagtcaccatggccagctccatagatcagggacacttggggaaccccgaacagagtgcaatgctcccccagctgaacacccccagcaggagtgcgctgtccagcgaactgtccctggtcggggacaccacgggggacacgctagagaagtttgtggagggcttgtagcattgtgagagcatcaccttttccctttcatgttcttggaccttttgtactgaaaatccaggcatctaggttctttttattcctagatggaactgcgacttccgagccatggaagggtggattgatgtttaaagaaacaatacaaagaatatatttttttgttaaaaaccagttgatttaaatatctggtctctctctttggtttttttttggcgggggggtggggggggttcttttttttccgttttgtttttgtttggggggaggggggttttgtttggattctttttgtcgtcattgctggtgactcatgcctttttttaacgggaaaaacaagttcattatattcatattttttatttgtattttcaagactttaaacatttatgtttaaaagtaagaagaaaaataatattcagaactgattcctgaaataatgcaagcttataatgtatcccgataactttgtgatgtttcgggaagatttttttctatagtgaactctgtgggcgtctcccagtattaccctggatgataggaattgactccggcgtgcacacacgtacacacccacacacatctatctatacataatggctgaagccaaacttgtcttgcagatgtagaaattgttgctttgtttctctgataaaactggttttagacaaaaaatagggatgatcactcttagaccatgctaatgttactagagaagaagccttcttttctttcttctatgtgaaacttgaaatgaggaaaagcaattctagtgtaaatcatgcaagcgctctaattcctataaatacgaaactcgagaagattcaatcactgtatagaatggtaaaataccaactcatttcttatatcatattgttaaataaactgtgtgcaacagacaaaaagggtggtccttcttgaattcatgtacatggtattaacacttagtgttcggggttttttgttatgaaaatgctgttttcaacattgtatttggactatgcatgtgttttttccccattgtatataaagtaccgcttaaaattgatataaattactgaggtttttaacatgtattctgttctttaagatccctgtaagaatgtttaaggtttttatttatttatatatattttttgagtctgttctttgtaagacatggttctggttgttcgctcatagcggagaggctggggctgcggttgtggttgtggcggcgtgggtggtggctgggaactgtggcccaggcttagcggccgcccggaggcttttcttcccggagactgaggtgggcgactgaggtgggcggctcagcgttggccccacacattcgaggctcacaggtgattgtcgctcacacagttagggtcgtcagttggtctgaaactgcatttggcccactcctccatcctccctgtccgtcgtagctgccacccccagaggcggcgcttcttcccgtgttcaggcggctccccccccccgtacacgactcccagaatctgaggcagagagtgctccaggctcgcgaggtgctttctgacttccccccaaatcctgccgctgccgcgcagcatgtcccgtgtggcgtttgaggaaatgctgagggacagacaccttggagcaccagctccggtccctgttacagtgagaaaggtcccccacttcgggggatacttgcacttagccacatggtcctgcctcccttggagtccagttccaggctcccttactgagtgggtgagacaagttcacaaaaaccgtaaaactgagaggaggaccatgggcaggggagctgaagttcatcccctaagtctaccacccccagcacccagagaacccactttatccctagtcccccaacaaaggctggtctaggtgggggtgatggtaattttagaaatcacgccccaaatagcttccgtttgggcccttacattcacagataggttttaaatagctgaatacttggtttgggaatctgaattcgaggaacctttctaagaagttggaaaggtccgatctagttttagcacagagctttgaaccttgagttataaaatgcagaataattcaagtaaaaataagaccaccatctggcacccctgaccagcccccattcaccccatcccaggaggggaagcacaggccgggcctccggtggagattgctgccactgctcggcctgctgggttcttaacctccagtgtcctcttcatcttttccacccgtagggaaaccttgagccatgtgttcaaacaagaagtggggctagagcccgagagcagcagctctaagcccacactcagaaagtggcgccctcctggttgtgcagccttttaatgtgggcagtggaggggcctctgtttcaggttatcctggaattcaaaacgttatgtaccaacctcatcctctttggagtctgcatcctgtgcaaccgtcttgggcaatccagatgtcgaaggatgtgaccgagagcatggtctgtggatgctaaccctaagtttgtcgtaaggaaatttctgtaagaaacctggaaagccccaacgctgtgtctcatgctgtatacttaagaggagaagaaaaagtcctatatttgtgatcaaaaagaggaaacttgaaatgtgatggtgtttataataaaagatggtaaaactacttggattcaaa

SEQ ID NO: 24 - protein, 2442 aa, Homo sapiens
MAENLLDGPPNPKRAKLSSPGFSANDSTDFGSLFDLENDLPDELIPNGGELGLLNSGNLVPDAASKHKQLSELLRGGSGSSINPGIGNVSASSPVQQGLGGQAQGQPNSANMASLSAMGKSPLSQGDSSAPSLPKQAASTSGPTPAASQALNPQAQKQVGLATSSPATSQTGPGICMNANFNQTHPGLLNSNSGHSLINQASQGQAQVMNGSLGAAGRGRGAGMPYPTPAMQGASSSVLAETLTQVSPQMTGHAGLNTAQAGGMAKMGITGNTSPFGQPFSQAGGQPMGATGVNPQLASKQSMVNSLPTFPTDIKNTSVTNVPNMSQMQTSVGIVPTQAIATGPTADPEKRKLIQQQLVLLLHAHKCQRREQANGEVRACSLPHCRTMKNVLNHMTHCQAGKACQVAHCASSRQIISHWKNCTRHDCPVCLPLKNASDKRNQQTILGSPASGIQNTIGSVGTGQQNATSLSNPNPIDPSSMQRAYAALGLPYMNQPQTQLQPQVPGQQPAQPQTHQQMRTLNPLGNNPMNIPAGGITTDQQPPNLISESALPTSLGATNPLMNDGSNSGNIGTLSTIPTAAPPSSTGVRKGWHEHVTQDLRSHLVHKLVQAIFPTPDPAALKDRRMENLVAYAKKVEGDMYESANSRDEYYHLLAEKIYKIQKELEEKRRSRLHKQGILGNQPALPAPGAQPPVIPQAQPVRPPNGPLSLPVNRMQVSQGMNSFNPMSLGNVQLPQAPMGPRAASPMNHSVQMNSMGSVPGMAISPSRMPQPPNMMGAHTNNMMAQAPAQSQFLPQNQFPSSSGAMSVGMGQPPAQTGVSQGQVPGAALPNPLNMLGPQASQLPCPPVTQSPLHPTPPPASTAAGMPSLQHTTPPGMTPPQPAAPTQPSTPVSSSGQTPTPTPGSVPSATQTQSTPTVQAAAQAQVTPQPQTPVQPPSVATPQSSQQQPTPVHAQPPGTPLSQAAASIDNRVPTPSSVASAETNSQQPGPDVPVLEMKTETQAEDTEPDPGESKGEPRSEMMEEDLQGASQVKEETDIAEQKSEPMEVDEKKPEVKVEVKEEEESSSNGTASQSTSPSQPRKKIFKPEELRQALMPTLEALYRQDPESLPFRQPVDPQLLGIPDYFDIVKNPMDLSTIKRKLDTGQYQEPWQYVDDVWLMFNNAWLYNRKTSRVYKFCSKLAEVFEQEIDPVMQSLGYCCGRKYEFSPQTLCCYGKQLCTIPRDAAYYSYQNRYHFCEKCFTEIQGENVTLGDDPSQPQTTISKDQFEKKKNDTLDPEPFVDCKECGRKMHQICVLHYDIIWPSGFVCDNCLKKTGRPRKENKFSAKRLQTTRLGNHLEDRVNKFLRRQNHPEAGEVFVRVVASSDKTVEVKPGMKSRFVDSGEMSESFPYRTKALFAFEEIDGVDVCFFGMHVQEYGSDCPPPNTRRVYISYLDSIHFFRPRCLRTAVYHEILIGYLEYVKKLGYVTGHIWACPPSEGDDYIFHCHPPDQKIPKPKRLQEWYKKMLDKAFAERIIHDYKDIFKQATEDRLTSAKELPYFEGDFWPNVLEESIKELEQEEEERKKEESTAASETTEGSQGDSKNAKKKNNKKTNKNKSSISRANKKKPSMPNVSNDLSQKLYATMEKHKEVFFVIHLHAGPVINTLPPIVDPDPLLSCDLMDGRDAFLTLARDKHWEFSSLRRSKWSTLCMLVELHTQGQDRFVYTCNECKHHVETRWHCTVCEDYDLCINCYNTKSHAHKMVKWGLGLDDEGSSQGEPQSKSPQESRRLSIQRCIQSLVHACQCRNANCSLPSCQKMKRVVQHTKGCKRKTNGGCPVCKQLIALCCYHAKHCQENKCPVPFCLNIKHKLRQQQIQHRLQQAQLMRRRMATMNTRNVPQQSLPSPTSAPPGTPTQQPSTPQTPQPPAQPQPSPVSMSPAGFPSVARTQPPTTVSTGKPTSQVPAPPPPAQPPPAAVEAARQIEREAQQQQHLYRVNINNSMPPGRTGMGTPGSQMAPVSLNVPRPNQVSGPVMPSMPPGQWQQAPLPQQQPMPGLPRPVISMQAQAAVAGPRMPSVQPPRSISPSALQDLLRTLKSPSSPQQQQQVLNILKSNPQLMAAFIKQRTAKYVANQPGMQPQPGLQSQPGMQPQPGMHQQPSLQNLNAMQAGVPRPGVPPQQQAMGGLNPQGQALNIMNPGHNPNMASMNPQYREMLRRQLLQQQQQQQQQQQQQQQQQQGSAGMAGGMAGHGQFQQPQGPGGYPPAMQQQQRMQQHLPLQGSSMGQMAAQMGQLGQMGQPGLGADSTPNIQQALQQRILQQQQMKQQIGSPGQPNPMSPQQHMLSGQPQASHLPGQQIATSLSNQVRSPAPVQSPRPQSQPPHSSPSPRIQPQPSPHHVSPQTGSPHPGLAVTMASSIDQGHLGNPEQSAMLPQLNTPSRSALSSELSLVGDTTGDTLEKFVEGL

SEQ ID NO: 25 - DNA, 10083 bp, Homo sapiens
ctgcggggcgctgttgctgtggctgagatttggccgccgcctcccccacccggcctgcgccctccctctccctcggcgcccgcccgcccgctcgcggcccgcgctcgctcctctccctcgcagccggcagggcccccgacccccgtccgggccctcgccggcccggccgcccgtgcccggggctgttttcgcgagcaggtgaaaatggctgagaacttgctggacggaccgcccaaccccaaaagagccaaactcagctcgcccggtttctcggcgaatgacagcacagattttggatcattgtttgacttggaaaatgatcttcctgatgagctgatacccaatggaggagaattaggccttttaaacagtgggaaccttgttccagatgctgcttccaaacataaacaactgtcggagcttctacgaggaggcagcggctctagtatcaacccaggaataggaaatgtgagcgccagcagccccgtgcagcagggcctgggtggccaggctcaagggcagccgaacagtgctaacatggccagcctcagtgccatgggcaagagccctctgagccagggagattcttcagcccccagcctgcctaaacaggcagccagcacctctgggcccacccccgctgcctcccaagcactgaatccgcaagcacaaaagcaagtggggctggcgactagcagccctgccacgtcacagactggacctggtatctgcatgaatgctaactttaaccagacccacccaggcctcctcaatagtaactctggccatagcttaattaatcaggcttcacaagggcaggcgcaagtcatgaatggatctcttggggctgctggcagaggaaggggagctggaatgccgtaccctactccagccatgcagggcgcctcgagcagcgtgctggctgagaccctaacgcaggtttccccgcaaatgactggtcacgcgggactgaacaccgcacaggcaggaggcatggccaagatgggaataactgggaacacaagtccatttggacagccctttagtcaagctggagggcagccaatgggagccactggagtgaacccccagttagccagcaaacagagcatggtcaacagtttgcccaccttccctacagatatcaagaatacttcagtcaccaacgtgccaaatatgtctcagatgcaaacatcagtgggaattgtacccacacaagcaattgcaacaggccccactgcagatcctgaaaaacgcaaactgatacagcagcagctggttctactgcttcatgctcataagtgtcagagacgagagcaagcaaacggagaggttcgggcctgctcgctcccgcattgtcgaaccatgaaaaacgttttgaatcacatgacgcattgtcaggctgggaaagcctgccaagccatcctggggtctccagctagtggaattcaaaacacaattggttctgttggcacagggcaacagaatgccacttctttaagtaacccaaatcccatagaccccagctccatgcagcgagcctatgctgctctcggactcccctacatgaaccagccccagacgcagctgcagcctcaggttcctggccagcaaccagcacagcctcaaacccaccagcagatgaggactctcaaccccctgggaaataatccaatgaacattccagcaggaggaataacaacagatcagcagcccccaaacttgatttcagaatcagctcttccgacttccctgggggccacaaacccactgatgaacgatggctccaactctggtaacattggaaccctcagcactataccaacagcagctcctccttctagcaccggtgtaaggaaaggctggcacgaacatgtcactcaggacctgcggagccatctagtgcataaactcgtccaagccatcttcccaacacctgatcccgcagctctaaaggatcgccgcatggaaaacctggtagcctatgctaagaaagtggaaggggacatgtacgagtctgccaacagcagggatgaatattatcacttattagcagagaaaatctacaagatacaaaaagaactagaagaaaaacggaggtcgcgtttacataaacaaggcatcttggggaaccagccagccttaccagccccgggggctcagccccctgtgattccacaggcacaacctgtgagacctccaaatggacccctgtccctgccagtgaatcgcatgcaagtttctcaagggatgaattcatttaaccccatgtccttggggaacgtccagttgccacaagcacccatgggacctcgtgcagcctccccaatgaaccactctgtccagatgaacagcatgggctcagtgccagggatggccatttctccttcccgaatgcctcagcctccgaacatgatgggtgcacacaccaacaacatgatggcccaggcgcccgctcagagccagtttctgccacagaaccagttcccgtcatccagcggggcgatgagtgtgggcatggggcagccgccagcccaaacaggcgtgtcacagggacaggtgcctggtgctgctcttcctaaccctctcaacatgctggggcctcaggccagccagctaccttgccctccagtgacacagtcaccactgcacccaacaccgcctcctgcttccacggctgctggcatgccatctctccagcacacgacaccacctgggatgactcctccccagccagcagctcccactcagccatcaactcctgtgtcgtcttccgggcagactcccaccccgactcctggctcagtgcccagtgctacccaaacccagagcacccctacagtccaggcagcagcccaggcccaggtgaccccgcagcctcaaaccccagttcagcccccgtctgtggctacccctcagtcatcgcagcaacagccgacgcctgtgcacgcccagcctcctggcacaccgctttcccaggcagcagccagcattgataacagagtccctaccccctcctcggtggccagcgcagaaaccaattcccagcagccaggacctgacgtacctgtgctggaaatgaagacggagacccaagcagaggacactgagcccgatcctggtgaatccaaaggggagcccaggtctgagatgatggaggaggatttgcaaggagcttcccaagttaaagaagaaacagacatagcagagcagaaatcagaaccaatggaagtggatgaaaagaaacctgaagtgaaagtagaagttaaagaggaagaagagagtagcagtaacggcacagcctctcagtcaacatctccttcgcagccgcgcaaaaaaatctttaaaccagaggagttacgccaggccctcatgccaaccctagaagcactgtatcgacaggacccagagtcattacctttccggcagcctgtagatccccagctcctcggaattccagactattttgacatcgtaaagaatcccatggacctctccaccatcaagcggaagctggacacagggcaataccaagagccctggcagtacgtggacgacgtctggctcatgttcaacaatgcctggctctataatcgcaagacatcccgagtctataagttttgcagtaagcttgcagaggtctttgagcaggaaattgaccctgtcatgcagtcccttggatattgctgtggacgcaagtatgagttttccccacagactttgtgctgctatgggaagcagctgtgtaccattcctcgcgatgctgcctactacagctatcagaataggtatcatttctgtgagaagtgtttcacagagatccagggcgagaatgtgaccctgggtgacgacccttcacagccccagacgacaatttcaaaggatcagtttgaaaagaagaaaaatgataccttagaccccgaacctttcgttgattgcaaggagtgtggccggaagatgcatcagatttgcgttctgcactatgacatcatttggccttcaggttttgtgtgcgacaactgcttgaagaaaactggcagacctcgaaaagaaaacaaattcagtgctaagaggctgcagaccacaagactgggaaaccacttggaagaccgagtgaacaaatttttgcggcgccagaatcaccctgaagccggggaggtttttgtccgagtggtggccagctcagacaagacggtggaggtcaagcccgggatgaagtcacggtttgtggattctggggaaatgtctgaatctttcccatatcgaaccaaagctctgtttgcttttgaggaaattgacggcgtggatgtctgcttttttggaatgcacgtccaagaatacggctctgattgcccccctccaaacacgaggcgtgtgtacatttcttatctggatagtattcatttcttccggccacgttgcctccgcacagccgtttaccatgagatccttattggatatttagagtatgtgaagaaattagggtatgtgacagggcacatctgggcctgtcctccaagtgaaggagatgattacatcttccattgccacccacctgatcaaaaaatacccaagccaaaacgactgcaggagtggtacaaaaagatgctggacaaggcgtttgcagagcggatcatccatgactacaaggatattttcaaacaagcaactgaagacaggctcaccagtgccaaggaactgccctattttgaaggtgatttctggcccaatgtgttagaagagagcattaaggaactagaacaagaagaagaggagaggaaaaaggaagagagcactgcagccagtgaaaccactgagggcagtcagggcgacagcaagaatgccaagaagaagaacaacaagaaaaccaacaagaacaaaagcagcatcagccgcgccaacaagaagaagcccagcatgcccaacgtgtccaatgacctgtcccagaagctgtatgccaccatggagaagcacaaggaggtcttcttcgtgatccacctgcacgctgggcctgtcatcaacaccctgccccccatcgtcgaccccgaccccctgctcagctgtgacctcatggatgggcgcgacgccttcctcaccctcgccagagacaagcactgggagttctcctccttgcgccgctccaagtggtccacgctctgcatgctggtggagctgcacacccagggccaggaccgctttgtctacacctgcaacgagtgcaagcaccacgtggagacgcgctggcactgcactgtgtgcgaggactacgacctctgcatcaactgctataacacgaagagccatgcccataagatggtgaagtgggggctgggcctggatgacgagggcagcagccagggcgagccacagtcaaagagcccccaggagtcacgccggctgagcatccagcgctgcatccagtcgctggtgcacgcgtgccagtgccgcaacgccaactgctcgctgccatcctgccagaagatgaagcgggtggtgcagcacaccaagggctgcaaacgcaagaccaacgggggctgcccggtgtgcaagcagctcatcgccctctgctgctaccacgccaagcactgccaagaaaacaaatgccccgtgcccttctgcctcaacatcaaacacaagctccgccagcagcagatccagcaccgcctgcagcaggcccagctcatgcgccggcggatggccaccatgaacacccgcaacgtgcctcagcagagtctgccttctcctacctcagcaccgcccgggacccccacacagcagcccagcacaccccagacgccgcagccccctgcccagccccaaccctcacccgtgagcatgtcaccagctggcttccccagcgtggcccggactcagccccccaccacggtgtccacagggaagcctaccagccaggtgccggcccccccacccccggcccagccccctcctgcagcggtggaagcggctcggcagatcgagcgtgaggcccagcagcagcagcacctgtaccgggtgaacatcaacaacagcatgcccccaggacgcacgggcatggggaccccggggagccagatggcccccgtgagcctgaatgtgccccgacccaaccaggtgagcgggcccgtcatgcccagcatgcctcccgggcagtggcagcaggcgccccttccccagcagcagcccatgccaggcttgcccaggcctgtgatatccatgcaggcccaggcggccgtggctgggccccggatgcccagcgtgcagccacccaggagcatctcacccagcgctctgcaagacctgctgcggaccctgaagtcgcccagctcccctcagcagcaacagcaggtgctgaacattctcaaatcaaacccgcagctaatggcagctttcatcaaacagcgcacagccaagtacgtggccaatcagcccggcatgcagccccagcctggcctccagtcccagcccggcatgcaaccccagcctggcatgcaccagcagcccagcctgcagaacctgaatgccatgcaggctggcgtgccgcggcccggtgtgcctccacagcagcaggcgatgggaggcctgaacccccagggccaggccttgaacatcatgaacccaggacacaaccccaacatggcgagtatgaatccacagtaccgagaaatgttacggaggcagctgctgcagcagcagcagcaacagcagcagcaacaacagcagcaacagcagcagcagcaagggagtgccggcatggctgggggcatggcggggcacggccagttccagcagcctcaaggacccggaggctacccaccggccatgcagcagcagcagcgcatgcagcagcatctccccctccagggcagctccatgggccagatggcggctcagatgggacagcttggccagatggggcagccggggctgggggcagacagcacccccaacatccagcaagccctgcagcagcggattctgcagcaacagcagatgaagcagcagattgggtccccaggccagccgaaccccatgagcccccagcaacacatgctctcaggacagccacaggcctcgcatctccctggccagcagatcgccacgtcccttagtaaccaggtgcggtctccagcccctgtccagtctccacggccccagtcccagcctccacattccagcccgtcaccacggatacagccccagccttcgccacaccacgtctcaccccagactggttccccccaccccggactcgcagtcaccatggccagctccatagatcagggacacttggggaaccccgaacagagtgcaatgctcccccagctgaacacccccagcaggagtgcgctgtccagcgaactgtccctggtcggggacaccacgggggacacgctagagaagtttgtggagggcttgtagcattgtgagagcatcaccttttccctttcatgttcttggaccttttgtactgaaaatccaggcatctaggttctttttattcctagatggaactgcgacttccgagccatggaagggtggattgatgtttaaagaaacaatacaaagaatatatttttttgttaaaaaccagttgatttaaatatctggtctctctctttggtttttttttggcgggggggtggggggggttcttttttttccgttttgtttttgtttggggggaggggggttttgtttggattctttttgtcgtcattgctggtgactcatgcctttttttaacgggaaaaacaagttcattatattcatattttttatttgtattttcaagactttaaacatttatgtttaaaagtaagaagaaaaataatattcagaactgattcctgaaataatgcaagcttataatgtatcccgataactttgtgatgtttcgggaagatttttttctatagtgaactctgtgggcgtctcccagtattaccctggatgataggaattgactccggcgtgcacacacgtacacacccacacacatctatctatacataatggctgaagccaaacttgtcttgcagatgtagaaattgttgctttgtttctctgataaaactggttttagacaaaaaatagggatgatcactcttagaccatgctaatgttactagagaagaagccttcttttctttcttctatgtgaaacttgaaatgaggaaaagcaattctagtgtaaatcatgcaagcgctctaattcctataaatacgaaactcgagaagattcaatcactgtatagaatggtaaaataccaactcatttcttatatcatattgttaaataaactgtgtgcaacagacaaaaagggtggtccttcttgaattcatgtacatggtattaacacttagtgttcggggttttttgttatgaaaatgctgttttcaacattgtatttggactatgcatgtgttttttccccattgtatataaagtaccgcttaaaattgatataaattactgaggtttttaacatgtattctgttctttaagatccctgtaagaatgtttaaggtttttatttatttatatatattttttgagtctgttctttgtaagacatggttctggttgttcgctcatagcggagaggctggggctgcggttgtggttgtggcggcgtgggtggtggctgggaactgtggcccaggcttagcggccgcccggaggcttttcttcccggagactgaggtgggcgactgaggtgggcggctcagcgttggccccacacattcgaggctcacaggtgattgtcgctcacacagttagggtcgtcagttggtctgaaactgcatttggcccactcctccatcctccctgtccgtcgtagctgccacccccagaggcggcgcttcttcccgtgttcaggcggctccccccccccgtacacgactcccagaatctgaggcagagagtgctccaggctcgcgaggtgctttctgacttccccccaaatcctgccgctgccgcgcagcatgtcccgtgtggcgtttgaggaaatgctgagggacagacaccttggagcaccagctccggtccctgttacagtgagaaaggtcccccacttcgggggatacttgcacttagccacatggtcctgcctcccttggagtccagttccaggctcccttactgagtgggtgagacaagttcacaaaaaccgtaaaactgagaggaggaccatgggcaggggagctgaagttcatcccctaagtctaccacccccagcacccagagaacccactttatccctagtcccccaacaaaggctggtctaggtgggggtgatggtaattttagaaatcacgccccaaatagcttccgtttgggcccttacattcacagataggttttaaatagctgaatacttggtttgggaatctgaattcgaggaacctttctaagaagttggaaaggtccgatctagttttagcacagagctttgaaccttgagttataaaatgcagaataattcaagtaaaaataagaccaccatctggcacccctgaccagcccccattcaccccatcccaggaggggaagcacaggccgggcctccggtggagattgctgccactgctcggcctgctgggttcttaacctccagtgtcctcttcatcttttccacccgtagggaaaccttgagccatgtgttcaaacaagaagtggggctagagcccgagagcagcagctctaagcccacactcagaaagtggcgccctcctggttgtgcagccttttaatgtgggcagtggaggggcctctgtttcaggttatcctggaattcaaaacgttatgtaccaacctcatcctctttggagtctgcatcctgtgcaaccgtcttgggcaatccagatgtcgaaggatgtgaccgagagcatggtctgtggatgctaaccctaagtttgtcgtaaggaaatttctgtaagaaacctggaaagccccaacgctgtgtctcatgctgtatacttaagaggagaagaaaaagtcctatatttgtgatcaaaaagaggaaacttgaaatgtgatggtgtttataataaaagatggtaaaactacttggattcaaa

SEQ ID NO: 26 - protein, 2404 aa, Homo sapiens
MAENLLDGPPNPKRAKLSSPGFSANDSTDFGSLFDLENDLPDELIPNGGELGLLNSGNLVPDAASKHKQLSELLRGGSGSSINPGIGNVSASSPVQQGLGGQAQGQPNSANMASLSAMGKSPLSQGDSSAPSLPKQAASTSGPTPAASQALNPQAQKQVGLATSSPATSQTGPGICMNANFNQTHPGLLNSNSGHSLINQASQGQAQVMNGSLGAAGRGRGAGMPYPTPAMQGASSSVLAETLTQVSPQMTGHAGLNTAQAGGMAKMGITGNTSPFGQPFSQAGGQPMGATGVNPQLASKQSMVNSLPTFPTDIKNTSVTNVPNMSQMQTSVGIVPTQAIATGPTADPEKRKLIQQQLVLLLHAHKCQRREQANGEVRACSLPHCRTMKNVLNHMTHCQAGKACQAILGSPASGIQNTIGSVGTGQQNATSLSNPNPIDPSSMQRAYAALGLPYMNQPQTQLQPQVPGQQPAQPQTHQQMRTLNPLGNNPMNIPAGGITTDQQPPNLISESALPTSLGATNPLMNDGSNSGNIGTLSTIPTAAPPSSTGVRKGWHEHVTQDLRSHLVHKLVQAIFPTPDPAALKDRRMENLVAYAKKVEGDMYESANSRDEYYHLLAEKIYKIQKELEEKRRSRLHKQGILGNQPALPAPGAQPPVIPQAQPVRPPNGPLSLPVNRMQVSQGMNSFNPMSLGNVQLPQAPMGPRAASPMNHSVQMNSMGSVPGMAISPSRMPQPPNMMGAHTNNMMAQAPAQSQFLPQNQFPSSSGAMSVGMGQPPAQTGVSQGQVPGAALPNPLNMLGPQASQLPCPPVTQSPLHPTPPPASTAAGMPSLQHTTPPGMTPPQPAAPTQPSTPVSSSGQTPTPTPGSVPSATQTQSTPTVQAAAQAQVTPQPQTPVQPPSVATPQSSQQQPTPVHAQPPGTPLSQAAASIDNRVPTPSSVASAETNSQQPGPDVPVLEMKTETQAEDTEPDPGESKGEPRSEMMEEDLQGASQVKEETDIAEQKSEPMEVDEKKPEVKVEVKEEEESSSNGTASQSTSPSQPRKKIFKPEELRQALMPTLEALYRQDPESLPFRQPVDPQLLGIPDYFDIVKNPMDLSTIKRKLDTGQYQEPWQYVDDVWLMFNNAWLYNRKTSRVYKFCSKLAEVFEQEIDPVMQSLGYCCGRKYEFSPQTLCCYGKQLCTIPRDAAYYSYQNRYHFCEKCFTEIQGENVTLGDDPSQPQTTISKDQFEKKKNDTLDPEPFVDCKECGRKMHQICVLHYDIIWPSGFVCDNCLKKTGRPRKENKFSAKRLQTTRLGNHLEDRVNKFLRRQNHPEAGEVFVRVVASSDKTVEVKPGMKSRFVDSGEMSESFPYRTKALFAFEEIDGVDVCFFGMHVQEYGSDCPPPNTRRVYISYLDSIHFFRPRCLRTAVYHEILIGYLEYVKKLGYVTGHIWACPPSEGDDYIFHCHPPDQKIPKPKRLQEWYKKMLDKAFAERIIHDYKDIFKQATEDRLTSAKELPYFEGDFWPNVLEESIKELEQEEEERKKEESTAASETTEGSQGDSKNAKKKNNKKTNKNKSSISRANKKKPSMPNVSNDLSQKLYATMEKHKEVFFVIHLHAGPVINTLPPIVDPDPLLSCDLMDGRDAFLTLARDKHWEFSSLRRSKWSTLCMLVELHTQGQDRFVYTCNECKHHVETRWHCTVCEDYDLCINCYNTKSHAHKMVKWGLGLDDEGSSQGEPQSKSPQESRRLSIQRCIQSLVHACQCRNANCSLPSCQKMKRVVQHTKGCKRKTNGGCPVCKQLIALCCYHAKHCQENKCPVPFCLNIKHKLRQQQIQHRLQQAQLMRRRMATMNTRNVPQQSLPSPTSAPPGTPTQQPSTPQTPQPPAQPQPSPVSMSPAGFPSVARTQPPTTVSTGKPTSQVPAPPPPAQPPPAAVEAARQIEREAQQQQHLYRVNINNSMPPGRTGMGTPGSQMAPVSLNVPRPNQVSGPVMPSMPPGQWQQAPLPQQQPMPGLPRPVISMQAQAAVAGPRMPSVQPPRSISPSALQDLLRTLKSPSSPQQQQQVLNILKSNPQLMAAFIKQRTAKYVANQPGMQPQPGLQSQPGMQPQPGMHQQPSLQNLNAMQAGVPRPGVPPQQQAMGGLNPQGQALNIMNPGHNPNMASMNPQYREMLRRQLLQQQQQQQQQQQQQQQQQQGSAGMAGGMAGHGQFQQPQGPGGYPPAMQQQQRMQQHLPLQGSSMGQMAAQMGQLGQMGQPGLGADSTPNIQQALQQRILQQQQMKQQIGSPGQPNPMSPQQHMLSGQPQASHLPGQQIATSLSNQVRSPAPVQSPRPQSQPPHSSPSPRIQPQPSPHHVSPQTGSPHPGLAVTMASSIDQGHLGNPEQSAMLPQLNTPSRSALSSELSLVGDTTGDTLEKFVEGL

(19) United States
(12) Patent Application Publication
(10) Pub. No.: US 2022/0193084 A1
(43) Pub. Date: Jun. 23, 2022

(54) INHIBITORS OF EZH2 AND METHODS OF USE THEREOF

(71) Applicant: Epizyme, Inc., Cambridge, MA (US)

(72) Inventors: Stephen BLAKEMORE, Littleton, MA (US); Scott Richard DAIGLE, Newburyport, MA (US)

(21) Appl. No.: 17/546,843

(22) Filed: Dec. 9, 2021

Related U.S. Application Data
(63) Continuation of application No. 16/060,164, filed on Jun. 7, 2018, now abandoned.
(60) Provisional application No. 62/409,320, filed on Oct. 17, 2016.
(60) Provisional application No. 62/264,169, filed on Dec. 7, 2015.

(51) Int. Cl. A61K 31/5377; C12Q 1/6886

(52) U.S. Cl. CPC A61K 31/5377; A61K 9/0053; C12Q 1/6886

(57) ABSTRACT

The disclosure provides a method of treating cancer in a subject in need thereof including administering to the subject a therapeutically-effective amount of an enhancer of a zeste homolog 2 (EZH2) inhibitor. In certain embodiments of this method, the subject has one or more mutations in one or more sequences encoding a gene listed in Tables 1-9, Tables 17-19, and/or FIGS. 19-22.

### RELATED APPLICATIONS

This application is a continuation of U.S. application Ser. No. 16/060,164, filed on Jun. 7, 2018, which is a U.S. National Phase application, filed under 35 U.S.C. § 371, of International Application No. PCT/US2016/065447, filed on Dec. 7, 2016, which claims priority to, and the benefit of, U.S. Provisional Application Nos. 62/264,169, filed Dec. 7, 2015, and 62/409,320 filed Oct. 17, 2016, the contents of each of which are incorporated herein by reference in their entireties.

### INCORPORTATION-BY-REFERENCE OF SEQUENCE LISTING

The Sequence Listing is provided as a file entitled “EPIZ-059_N01US Sequence Listing ST25.txt” created on Dec. 9, 2021, which is 202 kilobytes in size. The information in the electronic format of the sequence listing is incorporated herein by reference in its entirety.

### BACKGROUND

There is a long-felt yet unmet need for effective treatments for certain cancers caused by genetic alterations that result in EZH2-dependent oncogenesis.

### SUMMARY

In some aspects, the disclosure provides a method of treating cancer comprising administering a therapeutically effective amount of an inhibitor of Enhancer to Zeste Homolog 2 (EZH2) to a subject in need thereof, wherein the subject has at least one mutation in one or more sequences encoding a gene or gene product listed in Tables 1-9, Tables 17-19, and/or FIGS. 19A-22C.

In some aspects, the disclosure provides an inhibitor of Enhancer to Zeste Homolog 2 (EZH2) for use in treating cancer, wherein the inhibitor is for administration in a therapeutically effective amount of to a subject in need thereof, and wherein the subject has at least one mutation in one or more sequences encoding a gene or gene product listed in Tables 1-9, Tables 17-19, and/or FIGS. 19A-22C.

In some aspects, the disclosure provides a method, which comprises selecting a subject having cancer for treatment with an EZH2 inhibitor based on the presence of at least one mutation associated with a positive response (e.g., a positive mutation) to such treatment in the subject and/or based on the absence of at least one mutation associated with no response or with a negative response (e.g., a negative mutation) to such treatment in the subject.

The disclosure also provides a method, comprising selecting a subject having cancer for treatment with an EZH2 inhibitor based on the presence of a mutation profile in the subject that matches a mutation profile of a patient exhibiting a complete or partial response or stable disease in any of FIGS. 19A-22C.

The disclosure further provides a method of treating cancer comprising administering a therapeutically effective amount of an inhibitor of Enhancer to Zeste Homolog 2 (EZH2) to a subject; wherein the subject has a mutation in a sequence encoding a human histone acetyltransferase (HAT), wherein the mutation decreases a function of the HAT.

The methods and EZH2 inhibitors for use disclosed herein may have one or more of the following features.

In some embodiments, the subject has at least one mutation in one or more sequences encoding: MYD88 (e.g., GenBank Accession No. NM_001172567.1, NM_002468.4, NM_001172568.1, NM_001172569.1, and NM_001172566.1), STAT6A (e.g., GenBank Accession No. NM_001178078.1, NM_003153.4, NM_001178079.1, NM_001178080.1, or NM_001178081.1), SOCS1 (e.g., GenBank Accession No. NM_003745.1), MYC (e.g., GenBank Accession No. NM_002467.4), HIST1H1E (e.g., GenBank Accession No. NM_005321.2), ABL1 (e.g., GenBank Accession No. NM_005157), ACVR1 (e.g., GenBank Accession No. NM_001105.4), AKT1 (e.g., GenBank Accession No. NM_001014431.1), AKT2 (e.g., GenBank Accession No. NM_001243027.2), ALK (e.g., GenBank Accession No. NM_004304.4), APC (e.g., GenBank Accession No. NM_000038.5), AR (e.g., GenBank Accession No. NM_000044.3), ARID1A (e.g., GenBank Accession No. NM_006015.4), ARID1B (e.g., GenBank Accession No. NM_020732.3), ASXL1 (e.g., GenBank Accession No. NM_015338.5), ATM (e.g., GenBank Accession No. NM_000051.3), ATRX (e.g., GenBank Accession No. NM_000489.4), AURKA (e.g., GenBank Accession No. NM_003600.3), AXIN2 (e.g., GenBank Accession No. NM_004655.3), BAP1 (e.g., GenBank Accession No. NM_004656.3), BCL2 (e.g., GenBank Accession No. NM_000633.2), BCR (e.g., GenBank Accession No. X02596.1), BLM (e.g., GenBank Accession No. NM_000057.3), BMPR1A (e.g., GenBank Accession No. NM_004329.2), BRAF (e.g., GenBank Accession No. NM_004333.4), BRCA1 (e.g., GenBank Accession No. NM_007294.3), BRCA2 (e.g., GenBank Accession No. NM_000059.3), BRIP1 (e.g., GenBank Accession No. NM_032043.21), BTK (e.g., GenBank Accession No. NM_001287344.1), BUB1B (e.g., GenBank Accession No. NM_001211.5), CALR (e.g., GenBank Accession No. NM_004343.3), CBL (e.g., GenBank Accession No. NM_005188.3), CCND1 (e.g., GenBank Accession No. NM_053056.2), CCNE1 (e.g., GenBank Accession No. NM_001322262.1), CDC73 (e.g., GenBank Accession No. NM_024529.4), CDH1 (Accession No. NM_001317186.1), CDK4 (e.g., GenBank Accession No. NM_000075.3), CDK6 (e.g., GenBank Accession No. NM_001145306.1), CDKN1B (e.g., GenBank Accession No. NM_004064.4), CDKN2A (e.g., GenBank Accession No. NM_001195132.1), CDKN2B (e.g., GenBank Accession No. NM_078487.2), CDKN2C (e.g., GenBank Accession No. NM_078626.2), CEBPA (e.g., GenBank Accession No. NM_001285829.1), CHEK2 (e.g., GenBank Accession No. NM_145862.2), CIC (e.g., GenBank Accession No. NM_015125.4), CREBBP (e.g., GenBank Accession No. NM_001079846.1), CSF1R (e.g., GenBank Accession No. NM_001288705.2), CTNNB1 (e.g., GenBank Accession No. NM_001098209.1), CYLD (e.g., GenBank Accession No. NM_001042355.1), DAXX (Accession No. NM_001141969.1), DDB2 (e.g., GenBank Accession No. NM_001300734.1), DDR2 (e.g., GenBank Accession No. NM_001014796.1), DICER1 (e.g., GenBank Accession No. NM_001291628.1), DNMT3A (e.g., GenBank Accession No. NM_001320893.1), EGFR (e.g., GenBank Accession No. NM_001346900.1), EP300 (e.g., GenBank Accession No. NM_001429.3), ERBB2 (e.g., GenBank Accession No. NM_001289936.1), ERBB3 (e.g., GenBank Accession No. NM_001982.3), ERBB4 (e.g., GenBank Accession No. NM_005235.2), ERCC1 (e.g., GenBank Accession No. NM_001166049.1), ERCC2 (e.g., GenBank Accession No. NM_001130867.1), ERCC3 (e.g., GenBank Accession No. NM_001303418.1), ERCC4 (Accession No. NM_005236.2), ERCCS (e.g., GenBank Accession No. NM_000123.3), ESR1 (e.g., GenBank Accession No. NM_001291241.1), ETV1 (e.g., GenBank Accession No. NM_001163147.1), ETVS (Accession No. NM_004454.2), EWSR1 (e.g., GenBank Accession No. NM_001163287.1), EXT1 (e.g., GenBank Accession No. NM_000127.2), EXT2 (Accession No. NM_001178083.1), FANCA (e.g., GenBank Accession No. NM_001286167.1), FANCB (Accession No. NM_001324162.1), FANCC (e.g., GenBank Accession No. NM_001243744.1), FANCD2 (e.g., GenBank Accession No. NM_001319984.1), FANCE (e.g., GenBank Accession No. NM_021922.2), FANCF (e.g., GenBank Accession No NM_022725.3.), FANCG (e.g., GenBank Accession No. NM_004629.1), FANCI (e.g., GenBank Accession No. NM_018193.2), FANCL (Accession No. NM_001114636.1), FANCM (e.g., GenBank Accession No. NM_001308133.1), FBXW7 (e.g., GenBank Accession No. NM_018315.4), FGFR1 (Accession No.) NM_001174065.1, FGFR2 (e.g., GenBank Accession No. NM_000141.4), FGFR3 (e.g., GenBank Accession No. NM_001163213.1), FGFR4 (e.g., GenBank Accession No. NM_213647.2), FH (e.g., GenBank Accession No. NM_000143.3), FLCN (e.g., GenBank Accession No. NM_144606.5), FLT3 (e.g., GenBank Accession No. NM_004119.2), FLT4 (e.g., GenBank Accession No. NM_002020.4), FOXL2 (e.g., GenBank Accession No. NM_023067.3), GATA1 (e.g., GenBank No. NM_002049.3), GATA2 (e.g., GenBank Accession No. NM_001145662.1), GNA11 (e.g., GenBank Accession No. NM_002067.4), GNAQ (e.g., GenBank Accession No. NM_002072.4), GNAS (e.g., GenBank Accession No. NM_080425.3), GPC3 (e.g., GenBank Accession No. NM_001164619.1), H3F3A (e.g., GenBank Accession No. NM_002107.4), H3F3B (e.g., GenBank Accession No. NM_005324.4), HNF1A (e.g., GenBank Accession No. NM_000545.6), HRAS (e.g., GenBank Accession No. NM_001130442.2), IDH1 (e.g., GenBank Accession No. NM_001282387.1), IDH2 (e.g., GenBankAccession No. NM_001290114.1), IGF1R (e.g., GenBank Accession No. NM_001291858.1), IGF2R (e.g., GenBank Accession No. NM_000876.3), IKZF1 (e.g., GenBank Accession No. NM_001291847.1), JAK1 (e.g., GenBank Accession No. NM_001321857.1), JAK2 (e.g., GenBank Accession No. NM_001322195.1), JAK3 (e.g., GenBank Accession No. NM_000215.3), KDR (e.g., GenBank Accession No. NM_002253.2), KIT (e.g., GenBank Accession No. NM_001093772.1), KRAS (e.g., GenBank Accession No. NM_033360.3), MAML1 (e.g., GenBank Accession No. NM_014757.4), MAP2K1 (e.g., GenBank Accession No. NM_002755.3), MAP2K4 (e.g., GenBank Accession No. NM_001281435.1), MDM2 (e.g., GenBank Accession No. NM_001145337.2), MDM4 (e.g., GenBank Accession No. NM_001278519.1), MED12 (e.g., GenBank Accession No. NM_005120.2), MEN1 (e.g., GenBank Accession No. NM_130804.2), MET (e.g., GenBank Accession No NM_000245.3), MLH1 (e.g., GenBank Accession No. NM_000249.3), MLL (e.g., GenBank Accession No. AF232001.1), MPL (e.g., GenBank Accession No. NM_005373.2), MSH2 (e.g., GenBank Accession No. NM_000251.2), MSH6 (e.g., GenBank Accession No. NM_000179.2), MTOR (Accession No. NM_004958.3), MUTYH (e.g., GenBank Accession No. NM_001048171.1), MYC (e.g., GenBank Accession No. NM_002467.4), MYCL1 (e.g., GenBank Accession No NM_001033081.2), MYCN (e.g., GenBank Accession No. NM_001293231.1), NBN (e.g., GenBank Accession No. NM_001024688.2), NCOA3 (e.g., GenBank Accession No. NM_001174087.1), NF1 (e.g., GenBank Accession No. NM_001042492.2), NF2 (e.g., GenBank Accession No. NM_181831.2), NKX2-1(e.g., GenBank Accession No. NM_001079668.2), NOTCH1 (e.g., GenBank Accession No. NM_017617.4), NOTCH2 (e.g., GenBank Accession No NM_001200001.1), NOTCH3 (e.g., GenBank Accession No. NM_000435.2), NOTCH4 (Accession No. NR 134950.1), NPM1 (e.g., GenBank Accession No. NM_002520.6), NRAS (Accession No. NM_002524.4), NTRK1 (e.g., GenBank Accession No. NM_001007792.1), PALB2 (e.g., GenBank Accession No. NM_024675.3), PAXS (e.g., GenBank Accession No. NM_001280552.1), PBRM1 (e.g., GenBank Accession No. NM_181042.4), PDGFRA (e.g., GenBank Accession No. NM_006206.4), PHOX2B (e.g., GenBank Accession No. NM_003924.3), PIK3CA (e.g., GenBank Accession No. NM_006218.3), PIK3R1 (Accession No. NM_001242466.1), PMS1 (e.g., GenBank Accession No. NM_001321051.1), PMS2 (e.g., GenBank Accession No. NM_000535.6), POLD1 (e.g., GenBank Accession No. NM_001308632.1), POLE (e.g., GenBank Accession No. NM_006231.3), POLH (e.g., GenBank Accession No. NM_001291970.1), POT1 (e.g., GenBank Accession No. NM_001042594.1), PRKAR1A (e.g., GenBank Accession No. NM_001278433.1), PRSS1 (e.g., GenBank Accession No. NM_002769.4), PTCH1 (e.g., GenBank Accession No. NM_000264.3), PTEN (e.g., GenBank Accession No. NM_000314.6), PTPN11 (e.g., GenBank Accession No. NM_001330437.1), RAD51C (e.g., GenBank Accession No. NR 103873.1), RAF1 (e.g., GenBank Accession No. NM_002880.3), RB1 (e.g., GenBank Accession No. NM_000321.2), RECQL4 (e.g., GenBank Accession No. NM_004260.3), RET (e.g., GenBank Accession No.), RNF43(e.g., GenBank Accession No. NM_017763.5), ROS1 (e.g., GenBank Accession No. NM_002944.2), RUNX1 (e.g., GenBank Accession No. NM_001122607.1), SBDS (e.g., GenBank Accession No. NM_016038.2), SDHAF2 (e.g., GenBank Accession No. NM_017841.2), SDHB (e.g., GenBank Accession No.), SDHC (e.g., GenBank Accession No.), SDHD (e.g., GenBank Accession No. NM_001276503.1), SF3B1 (e.g., GenBank Accession No. NM_001308824.1), SMAD2 (e.g., GenBank Accession No. NM_001135937.2), SMAD3 (e.g., GenBank Accession No. NM_001145104.1), SMAD4 (e.g., GenBank Accession No. NM_005359.5), SMARCB1 (e.g., GenBank Accession No. NM_001007468.2), SMO (e.g., GenBank Accession No. NM_005631.4), SRC (e.g., GenBank Accession No. NM_005417.4), STAG2 (e.g., GenBank Accession No. NM_001282418.1), STK11 (e.g., GenBank Accession No. NM_000455.4), SUFU (e.g., GenBank Accession No. NM_001178133.1), TERT (e.g., GenBank Accession No. NM_001193376.1), TET2 (e.g., GenBank Accession No. NM_017628.4), TGFBR2 (e.g., GenBank Accession No. NM_001024847.2), TNFAIP3 (e.g., GenBank Accession No. NM_001270508.1), TOP1 (e.g., GenBank Accession No. NM_003286.3), TP53 (e.g., GenBank Accession No. NM_000546.5), TSC1 (e.g., GenBank Accession No. NM_001162427.1), TSC2 (e.g., GenBank Accession No. NM_001318832.1), TSHR (e.g., GenBank Accession No. NM_000369.2), VHL (e.g., GenBank Accession No. NM_000551.3), WAS (e.g., GenBank Accession No. NM_000377.2), WRN (e.g., GenBank Accession No. NM_000553.4), WT1 (e.g., GenBank Accession No. NM_000378.4), XPA (e.g., GenBank Accession No. NM_000380.3), XPC (e.g., GenBank Accession No. NM_004628.4), and/or XRCC1 (e.g., GenBank Accession No. NM_006297.2). It will be understood that the sequences provided above and elsewhere herein are exemplary, and serve to illustrate sequences suitable for some embodiments of the present disclosure. It will also be understood that, in some embodiments, the sequence encoding the gene product referred to herein is a genomic DNA sequence. The skilled artisan will be aware of additional suitable sequences beyond the exemplary, non-limiting RNA sequences provided above, for each gene or gene product (e.g., transcript, mRNA, or protein) referred to herein, or will be able to ascertain such suitable sequences without more than routine effort based on the present disclosure and the knowledge in the art.

In some embodiments, the subject has at least one mutation in one or more sequences encoding: ABL1, ACVR1, AKT1, AKT2, ALK, APC, AR, ARID1A, ARID1B, ASXL1, ATM, ATRX, AURKA, AXIN2, BAP1, BCL2, BCR, BLM, BMPR1A, BRAF, BRCA1, BRCA2, BRIP1, BTK, BUB1B, CALR, CBL, CCND1, CCNE1, CDC73, CDH1, CDK4, CDK6, CDKN1B, CDKN2A, CDKN2B, CDKN2C, CEBPA, CHEK2, CIC, CREBBP, CSF1R, CTNNB1, CYLD, DAXX, DDB2, DDR2, DICER1, DNMT3A, EGFR, EP300, ERBB2, ERBB3, ERBB4, ERCC1, ERCC2, ERCC3, ERCC4, ERCC5, ESR1, ETV1, ETV5, EWSR1, EXT1, EXT2, EZH2, FANCA, FANCB, FANCC, FANCD2, FANCE, FANCF, FANCG, FANCI, FANCL, FANCM, FBXW7, FGFR1, FGFR2, FGFR3, FGFR4, FH, FLCN, FLT3, FLT4, FOXL2, GATA1, GATA2, GNA11, GNAQ, GNAS, GPC3, H3F3A, H3F3B, HNF1A, HRAS, IDH1, IDH2, IGF1R, IGF2R, IKZF1, JAK1, JAK2, JAK3, KDR, KIT, KRAS, MAML1, MAP2K1, MAP2K4, MDM2, MDM4, MED12, MEN1, MET, MLH1, MLL, MPL, MSH2, MSH6, MTOR, MUTYH, MYC, MYCL1, MYCN, MYD88, NBN, NCOA3, NF1, NF2, NKX2-1, NOTCH1, NOTCH2, NOTCH3, NOTCH4, NPM1, NRAS, NTRK1, PALB2, PAX5, PBRM1, PDGFRA, PHOX2B, PIK3CA, PIK3R1, PMS1, PMS2, POLD1, POLE, POLH, POT1, PRKAR1A, PRSS1, PTCH1, PTEN, PTPN11, RAD51C, RAF1, RB1, RECQL4, RET, RNF43, ROS1, RUNX1, SBDS, SDHAF2, SDHB, SDHC, SDHD, SF3B1, SMAD2, SMAD3, SMAD4, SMARCB1, SMO, SRC, STAG2, STK11, SUFU, TERT, TET2, TGFBR2, TNFAIP3, TOP1, TP53, TSC1, TSC2, TSHR, VHL, WAS, WRN, WT1, XPA, XPC, and/or XRCC1.

In some embodiments, the subject has at least one mutation in one or more sequences encoding: ARID1A, ATM, B2M, BCL2, BCL6, BCL7A, BRAF, BTG1, CARD11, CCND3, CD58, CD79B, CDKN2A, CREBBP, EP300, EZH2, FOXO1, GNA13, HIST1H1B, HIST1H1C, HIST1H1E, IKZF3, IRF4, ITPKB, KDM6A, KIT, KMT2D, KRAS, MEF2B, MYC, MYD88, NOTCH1, NOTCH2, NRAS, PIK3CA, PIM1, POU2F2, PRDM1, PTEN, PTPN1, PTPN11, PTPN6, PTPRD, RB1, S1PR2, SGK1, SMARCB1, SOCS1, STAT6, TBL1XR1, TNFAIP3, TNFRSF14, TP53, and/or XPO1.

In some embodiments, the subject has at least one mutation in one or more sequences encoding: AKT1, ALK, ARID1A, ATM, B2M, BCL2, BCL6, BCL7A, BTG2, CARD11, CCND3, CD79B, CDKN2A, CREBBP, EP300, EZH2, FBXW7, FOXO1, HLA-C, HRAS, IKZF3, IRF4, KDM6A, KRAS, MEF2B, MYD88, NOTCH1, NPM1, NRAS, PIK3CA, PIM1, PRDM1, PTEN, RB1, RBBP4, SMARCB1, SUZ12, TNFRSF14, and/or TP53.

In some embodiments, the subject has at least one mutation in one or more sequences encoding: ALK, EWSR1, ROS1, BCL2, MLL, TMPRSS2, BCR, MYC, FGFR3, BRAF, NTRK1, TACC3, DNAJB1, PDGFRA, EGFR, PDGFRB, ETV1, PRKACA, ETV4, RAF1, ETV5, RARA, ETV6, and/or RET.

In some embodiments, the subject has at least one mutation in one or more sequences encoding: ALK (Intron 19), BCL2 (MBR breakpoint region), BCL2 (MCR breakpoint region), BCL6, CD274, CIITA, MYC (entire Gene +40 kbp upstream), and/or PDCD1LG2.

In some embodiments, the subject has at least one mutation in one or more sequences encoding: BCL2, CD274 (PDL1), FOXP1, JAK2, KDM4C, PDCD1LG2 (PDL2), and/or REL.

In some embodiments, the subject has at least one mutation in one or more sequences encoding: ARID1A, ATM, B2M, BCL2, BCL6, BCL7A, BRAF, CARD11, CCND3, CD274 (PDL1), CD58, CD79B, CDKN2A, CIITA, CREBBP, EZH2 (non-Y646), EZH2 (Y646), EP300, FOXO1, FOXP1, GNA13, HIST1H1B, HIST1H1C, HIST1H1E, IRF4, IZKF3, JAK2, KDM4C, KDM6A, KIT, KMT2D, KRAS, MEF2B, MYC, MYD88, NOTCH1, NOTCH2, NRAS, PDCD1LG2 (PDL2), PIK3CA, PIM1, POU2F2, PRDM1, PTEN, PTPN11, PTPN6, PTPRD, REL, SOCS1, STAT6, TNFAIP3, TNFRSF14, and/or TP53.

In some embodiments, the subject has at least one mutation in one or more sequences encoding: ARID1A, B2M, BCL2, BCL6, CARD11, CCND3, CD274 (PDL1), CD58, CD79B, CDKN2A, CREBBP, EZH2, EP300, FOXO1, GNA13, HIST1H1B, HIST1H1C, HIST1H1E, KMT2D, KRAS, MEF2B, MYC, MYD88 (273P), PDCD1LG2 (PDL2), PIM1, POU2F2, PRDM1, SOCS1, STAT6, TNFAIP3, and/or TNFRSF14.

In some embodiments, the at least one mutation decreases the function of a protein encoded by the mutated sequence as compared to the function of the protein encoded by the wild-type sequence. In some embodiments, the at least one mutation is a loss-of-function mutation.

In some embodiments, the method further comprises detecting the at least one mutation in the subject.

In some embodiments, the detecting comprises subjecting a sample obtained from the subject to a sequence analysis assay.

In some embodiments, the inhibitor of EZH2 is

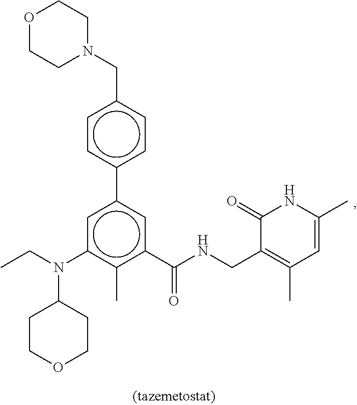

or a pharmaceutically-acceptable salt thereof

In some embodiments, the inhibitor of EZH2 is administered orally.

In some embodiments, the inhibitor of EZH2 is formulated as a tablet.

In some embodiments, the therapeutically effective amount of the inhibitor of EZH2 is between 100 mg and 3200 mg per day. -In some embodiments, the therapeutically effective amount of the inhibitor of EZH2 is 100 mg, 200 mg, 400 mg, 600 mg, 800 mg, 1000 mg, 1200 mg, 1400 mg, 1600 mg or 3200 mg per day. In some embodiments, the therapeutically effective amount is 1600 mg per day. In some embodiments, the therapeutically effective amount of the inhibitor of is administered at 800 mg twice per day (BID).

In some embodiments, the at least one mutation decreases a level of acetylation of a lysine (K) on histone (3) compared to a level of acetylation of the same lysine by a wild type HAT.

In some embodiments, the lysine (K) on histone (3) is at position 27 (H3K27).

In some embodiments, the at least one mutation occurs in a sequence of an EP300 gene or in a sequence encoding histone acetyltransferase p300.

In some embodiments, the at least one mutation results in a substitution of serine (S) for phenylalanine (F) at position 1289 of histone acetylransferase p300.

In some embodiments, the mutation may occur in a sequence of an EP300 gene or protein encoding Histone acetyltransferase p300. The mutation may occur in a sequence of the EP300 gene or protein encoding p300 is a substitution of tyrosine (Y) for aspartic acid (D) at position 1467 (for example, as numbered in SEQ ID NO: 20). The mutation may occur in a sequence of the EP300 gene or protein encoding p300 is a substitution of serine (S) for phenylalanine (F) at position 1289 (for example, as numbered in SEQ ID NO: 20).

In some embodiments, the at least one mutation occurs in a sequence of a CREB binding protein gene or in a sequence encoding CREBB. In some embodiments, the at least one mutation results in a substitution of phosphate (P) for threonine (T) at position 1494 of CREBBP (for example, as numbered in SEQ ID NO: 24). In some embodiments, the at least one mutation results in a substitution of arginine (R) for Leucine (L) at position 1446 of CREBBP (for example, as numbered in SEQ ID NO: 24). In some embodiments, the at least one mutation results in a substitution of Leucine (L) for phosphate (P) at position 1499 of CREBBP (for example, as numbered in SEQ ID NO: 24).

In some embodiments, the subject expresses a wild type EZH2 protein and does not express a mutant EZH2 protein.

In some embodiments, the subject expresses a mutant EZH2 protein. In some embodiments, the mutant EZH2 protein comprises a substitution of any amino acid other than tyrosine (Y) for tyrosine (Y) at position 641 of SEQ ID NO: 1. In some embodiments, the mutant EZH2 protein comprises a substitution of any amino acid other than alanine (A) for alanine (A) at position 682 of SEQ ID NO: 1. In some embodiments, the mutant EZH2 protein comprises a substitution of any amino acid other than alanine (A) for alanine (A) at position 692 of SEQ ID NO: 1.

In some embodiments, the at least one mutation comprises a MYD88, STAT6A, and/or a SOCS1 mutation.

In some embodiments, the subject does not have a MYC and/or a HIST1H1E mutation.

In some embodiments, the subject (a) has a MYD88, STAT6A, and/or a SOCS1 mutation, and (b) does not have a MYC and/or a HIST1H1E mutation.

In some embodiments, the subject has a mutation in a sequence encoding a human histone acetyltransferase (HAT).

In some embodiments, the subject is a human subject. In some embodiments, the subject has cancer.

In some embodiments, the cancer is B-cell lymphoma. In some embodiments, the B-cell lymphoma is an activated B-cell (ABC) type. In some embodiments, the B-cell lymphoma is a germinal B-cell (GBC) type.

In some embodiments, the cancer is follicular lymphoma.

In some embodiments, the at least one mutation associated with a positive response comprise (a) an EZH2 mutation; (b) a histone acetyl transferase (HAT) mutation;(c) a STAT6 mutation;(d) a MYD88 mutation; and/or (e) a SOCS1 mutation.

In some embodiments, the at least one mutation associated with no response or with a negative response comprise (a) a MYC mutation; and/or (b) a HIST1H1E mutation.

In some embodiments, the method comprises detecting the at least one mutation associated with a positive response and/or the at least one mutation associated with no response or a negative response in a sample obtained from the subject.

In some embodiments, the method comprises selecting the subject for treatment with the EZH2 inhibitor based on the subject (a) having at least one of a MYD88 mutation, a STAT6A mutation, and a SOCS1 mutation, and (b) not having at least one of a MYC mutation and/or a HIST1H1E mutation.

In some embodiments, the at least one mutation consists of a single mutation. In some embodiments, the at least one mutation comprises 2 mutations or more. In some embodiments, the at least one mutation comprises 3 mutations or more. In some embodiments, the at least one mutation comprises 4 mutations or more. In some embodiments, the at least one mutation comprises 5 mutations or more.

In some embodiments, the at least one mutation comprises 2 mutations, 3 mutations, 4 mutations, 5 mutations, 6 mutations, 7 mutations, 8 mutations, 9 mutations, 10 mutations, 11 mutations, 12 mutations, 13 mutations, 14 mutations, 15 mutations, 16 mutations, 17 mutations, 18 mutations, 19 mutations, or 20 mutations.

In some embodiments, the at least one mutation comprises at least one positive mutation (e.g., with or without a negative mutation). In some embodiments, the at least one mutation comprises at least one negative mutation (e.g., with or without a positive mutation). In some embodiments, the at least one mutation comprises both positive and negative mutations. The term “positive mutation”, as used herein, refers to a mutation that sensitizes a subject, a cancer, or malignant cell or population of cells, to EZH2 treatment, or, in some embodiments, that renders a subject, cancer, or malignant cell or population of cells, more sensitive to EZH2 treatment. The term “negative mutation”, as used herein, refers to a mutation that desensitizes a subject, a cancer, or malignant cell or population of cells, to EZH2 treatment, or, in some embodiments, that renders a subject, cancer, or malignant cell or population of cells, less sensitive to EZH2 treatment. In some embodiments, the disclosure provides a method of identifying molecular variants in tumor samples harvested from NHL patients treated with a compound of the disclosure. In some embodiments, the disclosure provides a method of identifying molecular variants in cell free circulating tumor DNA (ctDNA) harvested from NHL patients treated with a compound of the disclosure.

In some embodiments, the molecular variants identified therein may correlate with clinical response, minimal residual disease or emergence of resistance.

The summary above is meant to illustrate, in a non-limiting manner, some of the embodiments, advantages, features, and uses of the technology disclosed herein. Other embodiments, advantages, features, and uses of the technology disclosed herein will be apparent from the Detailed Description, the Drawings, the Examples, and the Claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The above and further features will be more clearly appreciated from the following detailed description when taken in conjunction with the accompanying drawings.

FIG. 1 is a schematic diagram showing EZH2 catalyzed chromatin remodeling. EZH2 is the catalytic subunit of the multi-protein PRC2 (polycomb repressive complex 2). PRC2 is the only human protein methyltransferase that can methylate H3K27 Catalyzes mono-, di- and tri-methylation of H3K27. H3K27me3 is a transcriptionally repressive histone mark. H3K27 is the only significant substrate for PRC2. Aberrant trimethylation of H3K27 is oncogenic in a broad spectrum of human cancers, such as B-cell NHL.

FIG. 2 is a schematic diagram depicting how tazemetostat drives apoptosis or differentiation in lymphoma cells independently of EZH2 mutation status.

FIG. 3 is a schematic diagram showing tazemetostat (EPZ-6438) as a potent and highly selective EZH2 inhibitor.

FIG. 4 is a waterfall plot of best response in NHL from the trial described in Table 10.

FIG. 5 is a graph depicting the objective response in NHL from the intended treatment population at RP2D from the trial described in Table 10.

FIG. 6 is a series of photographs and a schematic diagram showing the response in EZH2-mutated DLBCL from the trial described in Table 10.

FIG. 7 a series of photographs, table, and a chart showing tazemetostat dose selection.

FIG. 8 is a graph depicting somatic mutations detected using a 39 gene next generation sequencing (NGS) panel, demonstrating that somatic mutations in histone acetyltransferases may co-segregate with response to tazemetostat.

FIG. 9 is a graph depicting somatic mutations detected using a 39 gene NGS panel.

FIG. 10 is a graph showing the details of baseline tumor mutation profiling.

FIG. 11 is a graph illustrating the duration of therapy and tumor response in a phase 1 clinical trial (all NHL patients, N=21).

FIG. 12 is a scheme illustrating the detection of mutations in cell-free DNA through suppressing NGS errors.

FIG. 13A and FIG. 13B are a pair of graphs showing variant allelic frequencies for a set of 20 validation cases at varying levels of tumor cell line contribution relative to their genomic location, observed in the NHL specific plasma select panel of the disclosure. The individual graphs show the results for the sequence mutation analyses pre-correction (FIG. 13A) and post correction (FIG. 13B). The figure illustrates that the NGS background suppression enables detection of variant alleles down to 0.1% in ctDNA.

FIG. 14 is a graph showing the results of digital karyotyping and personalized analysis of rearranged ends (PARE) to identify structural alterations at varying levels of tumor DNA concentrations. ALK translocations were detected in a cell-free DNA validation test set down to a tumor purity of 0.1%.

FIGS. 15A-D is a series of graphs showing the relative distribution of mutations in the Phase 2 NHL trial with variant allele frequencies of >2% in archive tumors. The bar graphs plot the frequency of appearance of each of the individual gene mutations observed in: (A) all samples, (B) GCB DLCBCL cohorts, (C) Non-GCB DLBCL cohorts, and (D) Follicular Lymphoma cohorts.

FIGS. 16A-D is a series of graphs showing the relative distribution of mutations in the Phase 2 NHL trial with variant allele frequencies of >0.1% in ctDNA. The bar graphs plot the frequency of appearance of each of the individual gene mutations observed in: (A) all samples, (B) GCB DLCBCL cohorts, (C) Non-GCB DLBCL cohorts, and (D) Follicular Lymphoma cohorts.

FIG. 17 is a graph illustrating the duration of therapy and tumor response in phase 2 patients. ctDNA samples were taken at various assessment time points for 16 patients for further ctDNA NGS analysis to monitor for clonal switching, minimum residual disease and emergence of resistance.

FIG. 18A and FIG. 18B are a combination of graphs illustrating mutations of STAT6 observed in the 62 gene NGS panel. The panel covers exons 9-14 (DNA binding domain) of STAT6. FIG. 18A is a scheme of the STAT6 protein domain structure. The approximate location of somatic mutations identified in STAT6 in follicular lymphoma is indicated. FIG. 18B shows a homology model of the STAT6-DNA complex. STAT6 residues undergoing mutation are close to the DNA binding interface and are displayed in ball-and-stick diagrams (see, e.g., Yildiz et al. Blood 2015; 125: 668-679, the content of which is incorporated herein by reference in its entirety). Panel (c) is an enrichment plot of the KEGG_JAK_STAT_signaling_pathway.

FIG. 19A and FIG. 19B show tables summarizing the molecular variants observed in archive tumor in samples from phase 1 patients. Observed molecular variants were frameshift or nonsense mutations, missense mutations, translocations and amplifications. If multiple mutations were found in the same sample only the most damaging alteration are shown. Trends later identified in phase 2 samples also appear in the phase 1 NHL samples (e.g., EZH2, STAT6 and MYC).

FIG. 20A and FIG. 20B show tables summarizing the molecular variants observed in archive tumor tissue from phase 2 Patients. Observed molecular variants were frameshift or nonsense mutations, missense mutations, translocations and amplifications. Variants of interest included, inter alia, EZH2, MYD88 (273P) and MYC. EZH2 mutations were observed in 9 patients, wherein 7 displayed a variant allele frequency of >10%; 2 had variant allele frequencies of ≤10% (10042008, 8%; 10032004, 10%; best response: 4 PR, 3 SD and 2 PD). MYD88 (273P) mutations were observed in 6 patients (best response: 3 CR, 1PR, 1 PD and 1 unknown response); STAT6 mutations were observed in 13 patients (best response: 1 CR, 5 PR, 4 SD and 3 PD). MYC mutations were observed in 7 patients (best response: 5 PD and 2 unknown responses). 2 MYC translocations were associated with lack of response.

FIG. 21A, FIG. 21B and FIG. 21C show tables summarizing the molecular variants with variant allele frequencies of 0.1% observed in ctDNA in phase 2 patients. Observed molecular variants were frameshift or nonsense mutations, missense mutations, translocations and amplifications. Variants of interest included, inter alia, EZH2, MYD88 (273P) and MYC. EZH2 mutations were observed in 11 patients (best response: 5 PR, 2 SD, 3 PD and 1 unknown response). MYD88 (273P) mutations were observed in 6 patients (best response: 2 CR, 1PR, 1 SD and 2 PD); STAT6 mutations were observed in 14 patients (best response: 5 PR, 6 SD and 3 PD). MYC mutations were observed in 18 patients (best response: 2 PR, 3SD, 9 PD and 4 unknown responses). 5 MYC translocations were associated with lack of response.

FIG. 22A, FIG. 22B and FIG. 22C show tables summarizing the molecular variants with variant allele frequencies of 1% observed in ctDNA in phase 2 patients. Observed molecular variants were frameshift or nonsense mutations, missense mutations, translocations and amplifications. Variants of interest included, inter alia, EZH2, MYD88 (273P) and MYC. EZH2 mutations were observed in 8 patients (best response: 4 PR, 1 SD and 3 PD). MYD88 (273P) mutations were observed in 5 patients (best response: 2 CR, 1PR, and 2 PD); STAT6 mutations were observed in 10 patients (best response: 4 PR, 4 SD and 2 PD). MYC mutations were observed in 5 patients (best response: 3 PD and 2 unknown responses). 5 MYC translocations were associated with lack of response.

FIG. 23 is a structure model of partial EZH2 protein based on the A chain of nuclear receptor binding SET domain protein 1 (NSD1). This model corresponds to amino acid residues 533-732 of EZH2 sequence of SEQ ID NO: 1.

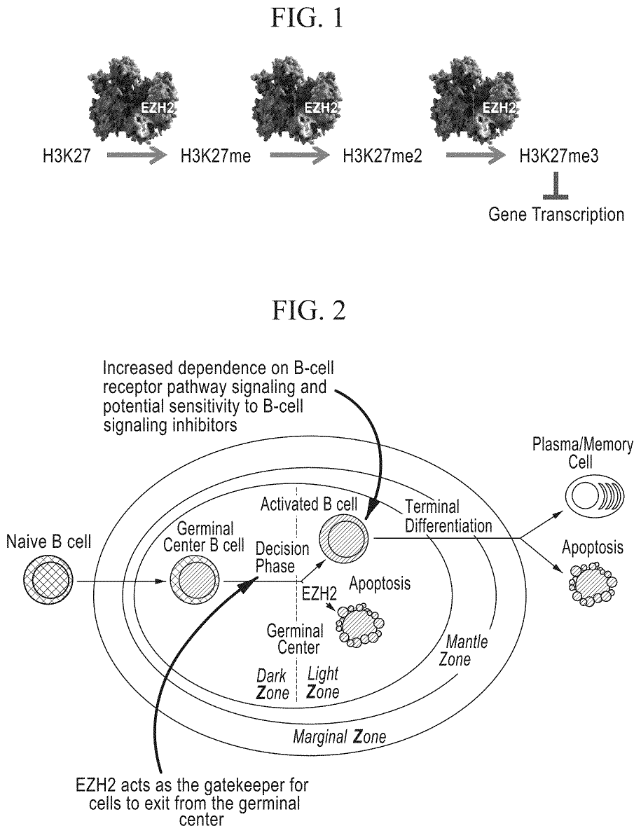

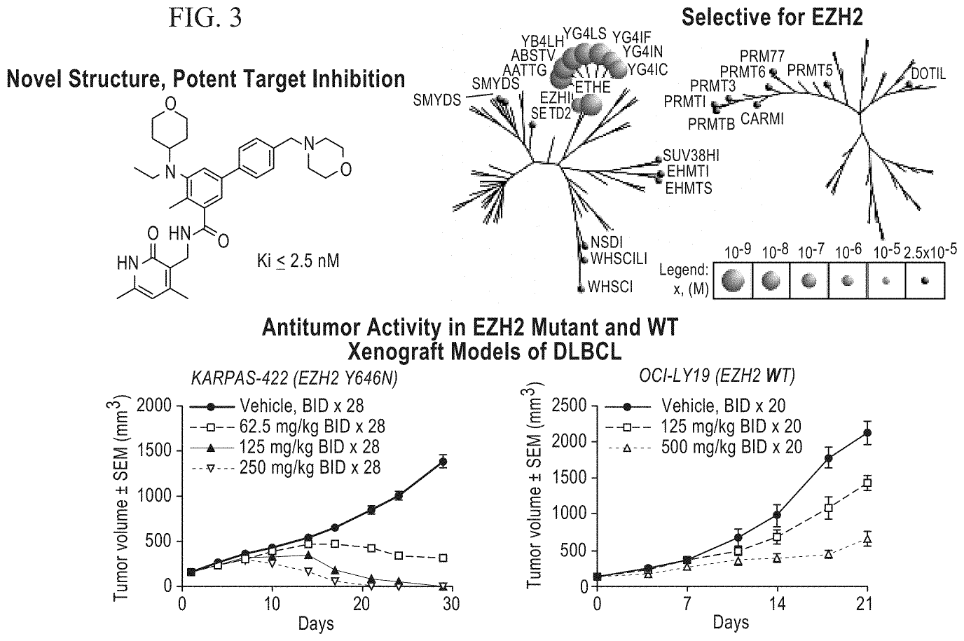

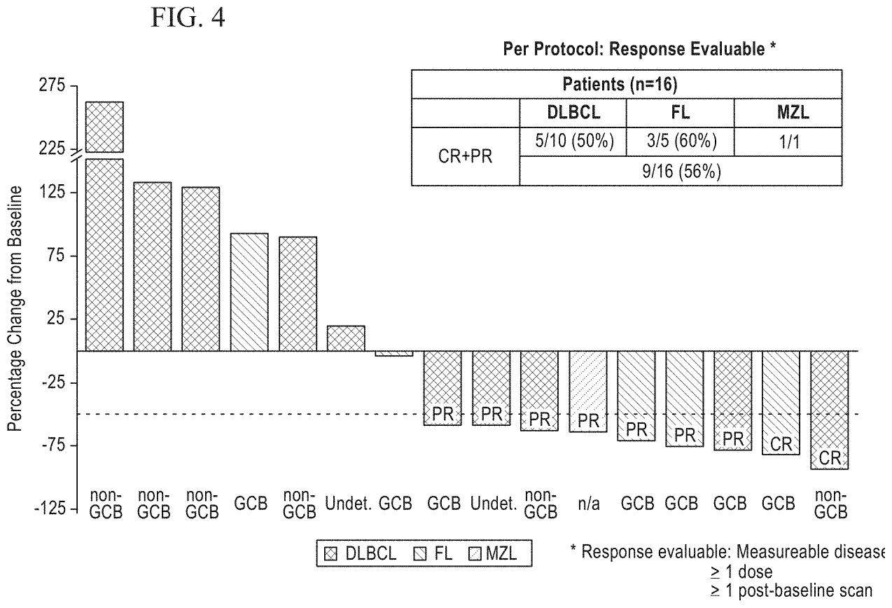

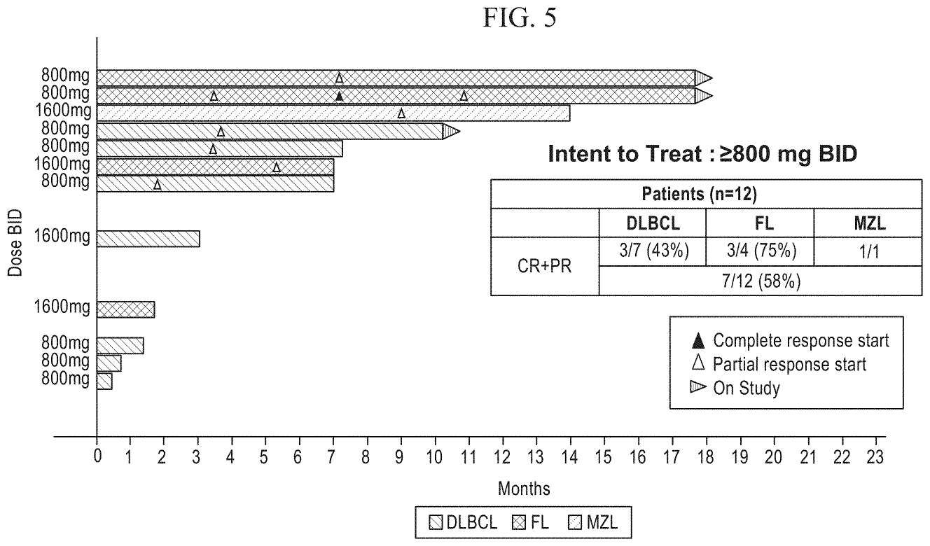

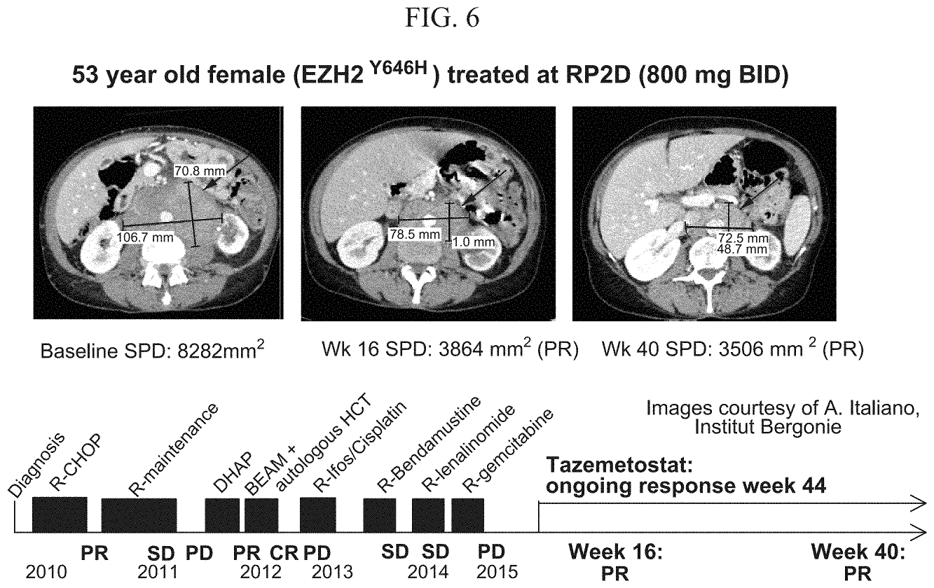

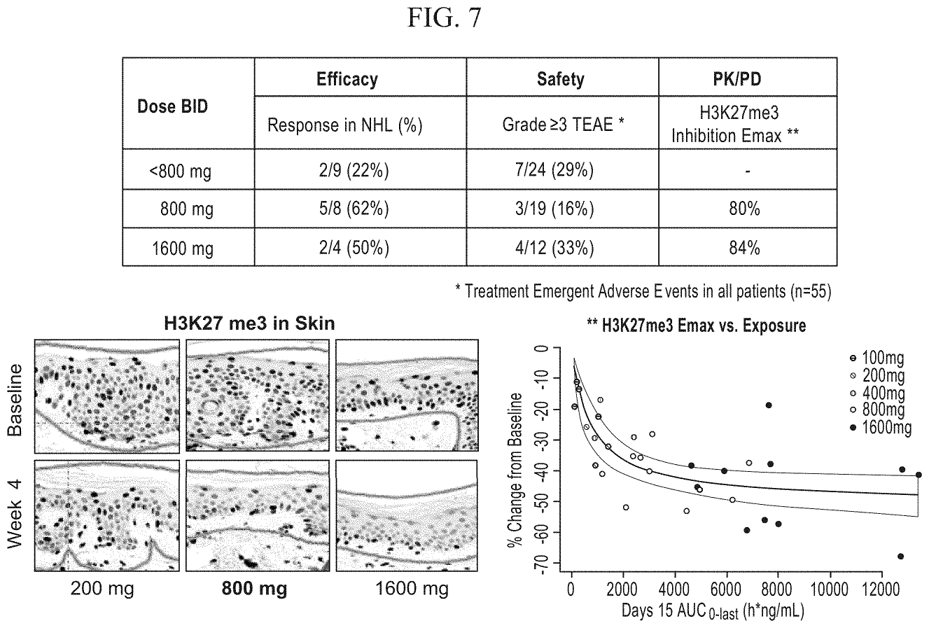

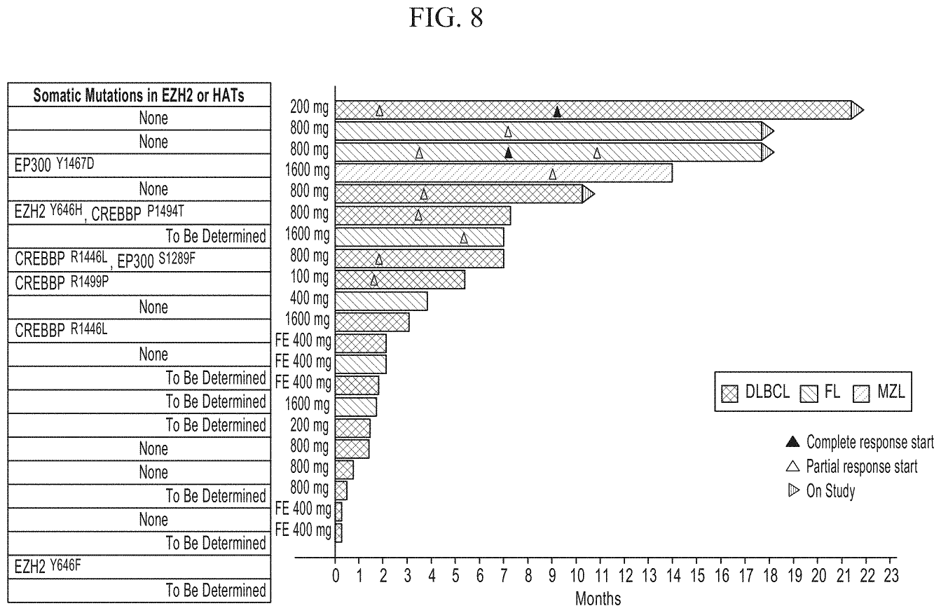

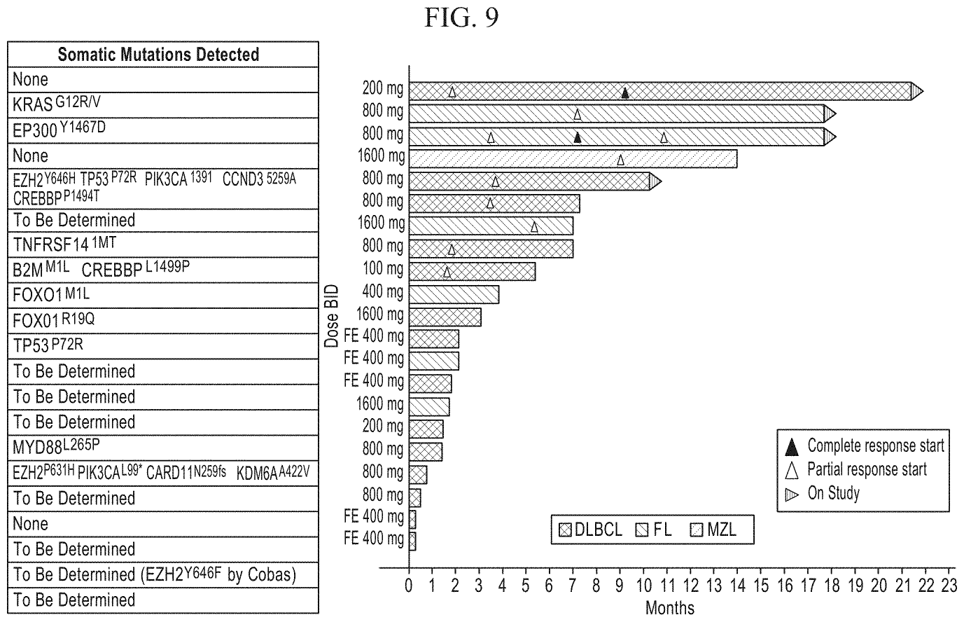

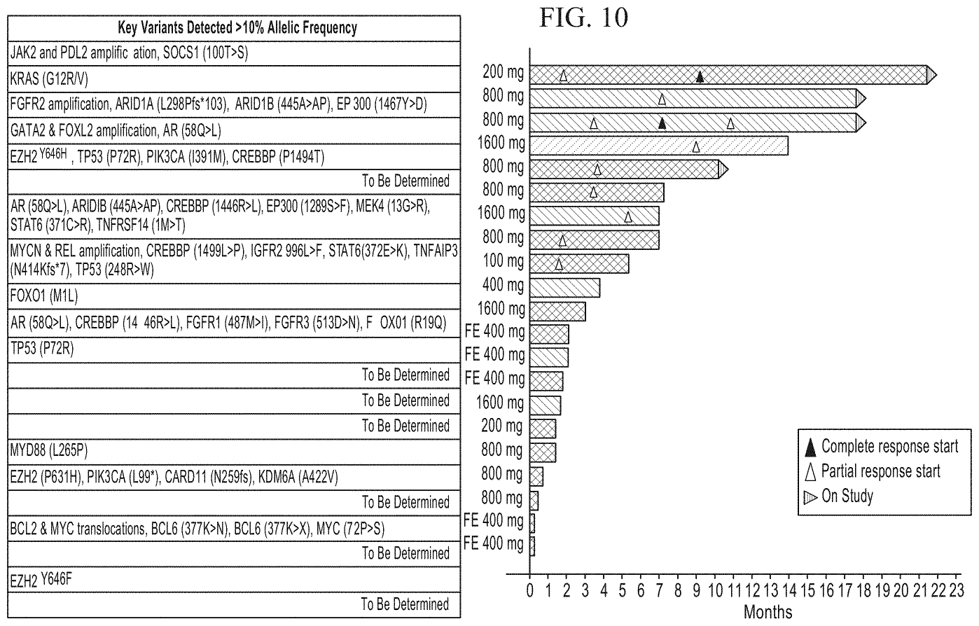

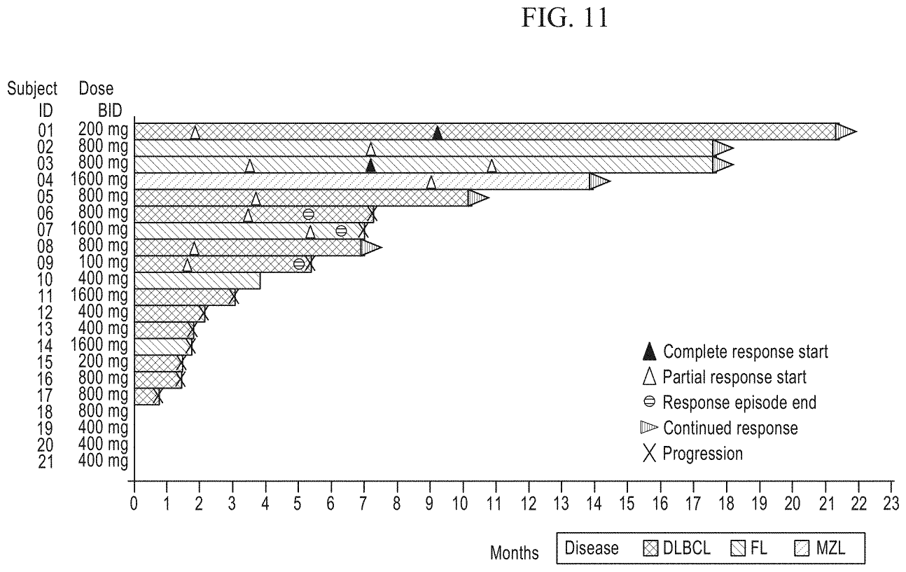

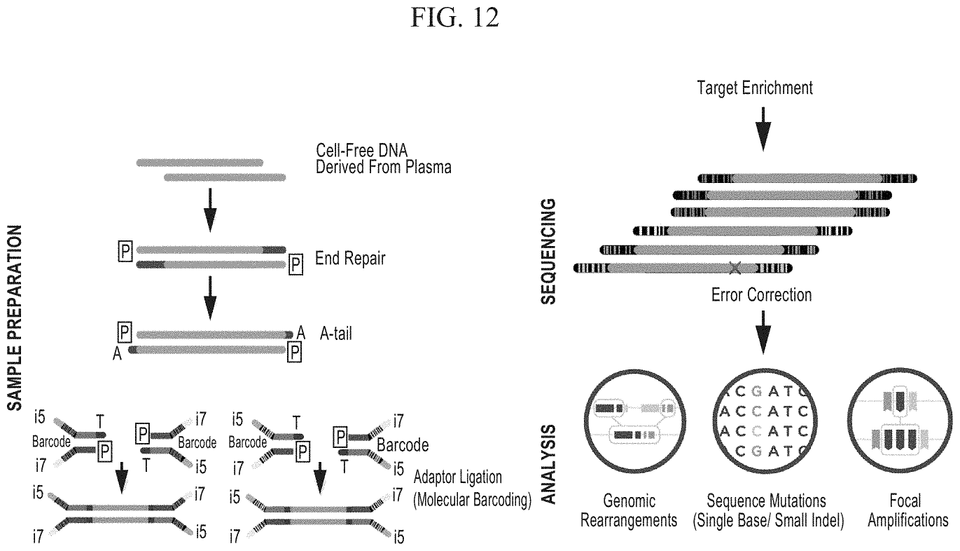

### DETAILED DESCRIPTION

Tazemetostat demonstrates clinical activity as a monotherapy in patients with relapsed or refractory DLBCL (both GCB and non-GCB), follicular lymphoma (FL) and marginal zone lymphomas (MZL). Objective responses in tumors with either wild-type or mutation in EZH2 are durable as patients are ongoing at 7+ to 21+ months. Safety profile as monotherapy continues to be acceptable and favorable for combination development. Recommended phase II dose (RP2D) of 800 mg BID supported by safety, efficacy, PK and PD.

Baseline somatic mutation profiling revealed associations between objective response to tazemetostat and genetic alterations, e.g., mutations in genomic sequences encoding MYD88, STAT6A, SOCS1, MYC, HIST1H1E, and histone acetyltransferases, such as, for example CREBBP and EP300.

### EZH2

EZH2 is a histone methyltransferase that is the catalytic subunit of the PRC2 complex which catalyzes the mono- through tri-methylation of lysine 27 on histone H3 (H3-K27).

Point mutations of the EZH2 gene at a single amino acid residue (e.g., Tyr641, herein referred to as Y641) of EZH2 have been reported to be linked to subsets of human B-cell lymphoma. Morin et al. (2010) Nat Genet 42(2):181-5. In particular, Morin et al. reported that somatic mutations of tyrosine 641 (Y641F, Y641H, Y641N, and Y641S) of EZH2 were associated with follicular lymphoma (FL) and the germinal center B cell-like (GCB) subtype of diffuse large B-cell lymphoma (DLBCL). The mutant allele is always found associated with a wild-type allele (heterozygous) in disease cells, and the mutations were reported to ablate the enzymatic activity of the PRC2 complex for methylating an unmodified peptide substrate.

The mutant EZH2 refers to a mutant EZH2 polypeptide or a nucleic acid sequence encoding a mutant EZH2 polypeptide. Preferably the mutant EZH2 comprises one or more mutations in its substrate pocket domain as defined in SEQ ID NO: 6. For example, the mutation may be a substitution, a point mutation, a nonsense mutation, a missense mutation, a deletion, or an insertion. Exemplary substitution amino acid mutation includes a substitution at amino acid position 677, 687, 674, 685, or 641 of SEQ ID NO: 1, such as, but is not limited to a substitution of glycine (G) for the wild type residue alanine (A) at amino acid position 677 of SEQ ID NO: 1 (A677G); a substitution of valine (V) for the wild type residue alanine (A) at amino acid position 687 of SEQ ID NO: 1 (A687V); a substitution of methionine (M) for the wild type residue valine (V) at amino acid position 674 of SEQ ID NO: 1 (V674M); a substitution of histidine (H) for the wild type residue arginine (R) at amino acid position 685 of SEQ ID NO: 1 (R685H); a substitution of cysteine (C) for the wild type residue arginine (R) at amino acid position 685 of SEQ ID NO: 1 (R685C); a substitution of phenylalanine (F) for the wild type residue tyrosine (Y) at amino acid position 641 of SEQ ID NO: 1 (Y641F); a substitution of histidine (H) for the wild type residue tyrosine (Y) at amino acid position 641 of SEQ ID NO: 1 (Y641H); a substitution of asparagine (N) for the wild type residue tyrosine (Y) at amino acid position 641 of SEQ ID NO: 1 (Y641N); a substitution of serine (S) for the wild type residue tyrosine (Y) at amino acid position 641 of SEQ ID NO: 1 (Y641S); or a substitution of cysteine (C) for the wild type residue tyrosine (Y) at amino acid position 641 of SEQ ID NO: 1 (Y641C).

The mutation may also include a substitution of serine (S) for the wild type residue asparagine (N) at amino acid position 322 of SEQ ID NO: 3 (N322S), a substitution of glutamine (Q) for the wild type residue arginine (R) at amino acid position 288 of SEQ ID NO: 3 (R288Q), a substitution of isoleucine (I) for the wild type residue threonine (T) at amino acid position 573 of SEQ ID NO: 3 (T573I), a substitution of glutamic acid (E) for the wild type residue aspartic acid (D) at amino acid position 664 of SEQ ID NO: 3 (D664E), a substitution of glutamine (Q) for the wild type residue arginine (R) at amino acid position 458 of SEQ ID NO: 5 (R458Q), a substitution of lysine (K) for the wild type residue glutamic acid (E) at amino acid position 249 of SEQ ID NO: 3 (E249K), a substitution of cysteine (C) for the wild type residue arginine (R) at amino acid position 684 of SEQ ID NO: 3 (R684C), a substitution of histidine (H) for the wild type residue arginine (R) at amino acid position 628 of SEQ ID NO: 21 (R628H), a substitution of histidine (H) for the wild type residue glutamine (Q) at amino acid position 501 of SEQ ID NO: 5 (Q501H), a substitution of asparagine (N) for the wild type residue aspartic acid (D) at amino acid position 192 of SEQ ID NO: 3 (D192N), a substitution of valine (V) for the wild type residue aspartic acid (D) at amino acid position 664 of SEQ ID NO: 3 (D664V), a substitution of leucine (L) for the wild type residue valine (V) at amino acid position 704 of SEQ ID NO: 3 (V704L), a substitution of serine (S) for the wild type residue proline (P) at amino acid position 132 of SEQ ID NO: 3 (P132S), a substitution of lysine (K) for the wild type residue glutamic acid (E) at amino acid position 669 of SEQ ID NO: 21 (E669K), a substitution of threonine (T) for the wild type residue alanine (A) at amino acid position 255 of SEQ ID NO: 3 (A255T), a substitution of valine (V) for the wild type residue glutamic acid (E) at amino acid position 726 of SEQ ID NO: 3 (E726V), a substitution of tyrosine (Y) for the wild type residue cysteine (C) at amino acid position 571 of SEQ ID NO: 3 (C571Y), a substitution of cysteine (C) for the wild type residue phenylalanine (F) at amino acid position 145 of SEQ ID NO: 3 (F145C), a substitution of threonine (T) for the wild type residue asparagine (N) at amino acid position 693 of SEQ ID NO: 3 (N693T), a substitution of serine (S) for the wild type residue phenylalanine (F) at amino acid position 145 of SEQ ID NO: 3 (F145S), a substitution of histidine (H) for the wild type residue glutamine (Q) at amino acid position 109 of SEQ ID NO: 21 (Q109H), a substitution of cysteine (C) for the wild type residue phenylalanine (F) at amino acid position 622 of SEQ ID NO: 21 (F622C), a substitution of arginine (R) for the wild type residue glycine (G) at amino acid position 135 of SEQ ID NO: 3 (G135R), a substitution of glutamine (Q) for the wild type residue arginine (R) at amino acid position 168 of SEQ ID NO: 5 (R168Q), a substitution of arginine (R) for the wild type residue glycine (G) at amino acid position 159 of SEQ ID NO: 3 (G159R), a substitution of cysteine (C) for the wild type residue arginine (R) at amino acid position 310 of SEQ ID NO: 5 (R310C), a substitution of histidine (H) for the wild type residue arginine (R) at amino acid position 561 of SEQ ID NO: 3 (R561H), a substitution of histidine (H) for the wild type residue arginine (R) at amino acid position 634 of SEQ ID NO: 21 (R634H), a substitution of arginine (R) for the wild type residue glycine (G) at amino acid position 660 of SEQ ID NO: 3 (G660R), a substitution of cysteine (C) for the wild type residue tyrosine (Y) at amino acid position 181 of SEQ ID NO: 3 (Y181C), a substitution of arginine (R) for the wild type residue histidine (H) at amino acid position 297 of SEQ ID NO: 3 (H297R), a substitution of serine (S) for the wild type residue cysteine (C) at amino acid position 612 of SEQ ID NO: 21 (C612S), a substitution of tyrosine (Y) for the wild type residue histidine (H) at amino acid position 694 of SEQ ID NO: 3 (H694Y), a substitution of alanine (A) for the wild type residue aspartic acid (D) at amino acid position 664 of SEQ ID NO: 3 (D664A), a substitution of threonine (T) for the wild type residue isoleucine (I) at amino acid position 150 of SEQ ID NO: 3 (I150T), a substitution of arginine (R) for the wild type residue isoleucine (I) at amino acid position 264 of SEQ ID NO: 3 (I264R), a substitution of leucine (L) for the wild type residue proline (P) at amino acid position 636 of SEQ ID NO: 3 (P636L), a substitution of threonine (T) for the wild type residue isoleucine (I) at amino acid position 713 of SEQ ID NO: 3 (I713T), a substitution of proline (P) for the wild type residue glutamine (Q) at amino acid position 501 of SEQ ID NO: 5 (Q501P), a substitution of glutamine (Q) for the wild type residue lysine (K) at amino acid position 243 of SEQ ID NO: 3 (K243Q), a substitution of aspartic acid (D) for the wild type residue glutamic acid (E) at amino acid position 130 of SEQ ID NO: 5 (E130D), a substitution of glycine (G) for the wild type residue arginine (R) at amino acid position 509 of SEQ ID NO: 3 (R509G), a substitution of histidine (H) for the wild type residue arginine (R) at amino acid position 566 of SEQ ID NO: 3 (R566H), a substitution of histidine (H) for the wild type residue aspartic acid (D) at amino acid position 677 of SEQ ID NO: 3 (D677H), a substitution of asparagine (N) for the wild type residue lysine (K) at amino acid position 466 of SEQ ID NO: 5 (K466N), a substitution of histidine (H) for the wild type residue arginine (R) at amino acid position 78 of SEQ ID NO: 3 (R78H), a substitution of methionine (M) for the wild type residue lysine (K) at amino acid position 1 of SEQ ID NO: 6 (K6M), a substitution of leucine (L) for the wild type residue serine (S) at amino acid position 538 of SEQ ID NO: 3 (S538L), a substitution of glutamine (Q) for the wild type residue leucine (L) at amino acid position 149 of SEQ ID NO: 3 (L149Q), a substitution of valine (V) for the wild type residue leucine (L) at amino acid position 252 of SEQ ID NO: 3 (L252V), a substitution of valine (V) for the wild type residue leucine (L) at amino acid position 674 of SEQ ID NO: 3 (L674V), a substitution of valine (V) for the wild type residue alanine (A) at amino acid position 656 of SEQ ID NO: 3 (A656V), a substitution of aspartic acid (D) for the wild type residue alanine (A) at amino acid position 731 of SEQ ID NO: 3 (Y731D), a substitution of threonine (T) for the wild type residue alanine (A) at amino acid position 345 of SEQ ID NO: 3 (A345T), a substitution of aspartic acid (D) for the wild type residue alanine (A) at amino acid position 244 of SEQ ID NO: 3 (Y244D), a substitution of tryptophan (W) for the wild type residue cysteine (C) at amino acid position 576 of SEQ ID NO: 3 (C576W), a substitution of lysine (K) for the wild type residue asparagine (N) at amino acid position 640 of SEQ ID NO: 3 (N640K), a substitution of lysine (K) for the wild type residue asparagine (N) at amino acid position 675 of SEQ ID NO: 3 (N675K), a substitution of tyrosine (Y) for the wild type residue aspartic acid (D) at amino acid position 579 of SEQ ID NO: 21 (D579Y), a substitution of isoleucine (I) for the wild type residue asparagine (N) at amino acid position 693 of SEQ ID NO: 3 (N693I), and a substitution of lysine (K) for the wild type residue asparagine (N) at amino acid position 693 of SEQ ID NO: 3 (N693K).

The mutation may be a frameshift at amino acid position 730, 391, 461, 441, 235, 254, 564, 662, 715, 405, 685, 64, 73, 656, 718, 374, 592, 505, 730, or 363 of SEQ ID NO: 3, 5 or 21 or the corresponding nucleotide position of the nucleic acid sequence encoding SEQ ID NO: 3, 5, or 21. The mutation of the EZH2 may also be an insertion of a glutamic acid (E) between amino acid positions 148 and 149 of SEQ ID NO: 3, 5 or 21. Another example of EZH2 mutation is a deletion of glutamic acid (E) and leucine (L) at amino acid positions 148 and 149 of SEQ ID NO: 3, 5 or 21. The mutant EZH2 may further comprise a nonsense mutation at amino acid position 733, 25, 317, 62, 553, 328, 58, 207, 123, 63, 137, or 60 of SEQ ID NO: 3, 5 or 21.

Human EZH2 nucleic acids and polypeptides have previously been described. See, e.g., Chen et al. (1996) Genomics 38:30-7 [746 amino acids]; Swiss-Prot Accession No. Q15910 [746 amino acids]; GenBank Accession Nos. NM_004456 and NP_004447 (isoform a [751 amino acids]); and GenBank Accession Nos. NM_152998 and NP_694543 (isoform b [707 amino acids]), each of which is incorporated herein by reference in its entirety.

Amino acid sequence of human EZH2 (Swiss-Prot Accession No. Q15910)
(SEQ ID NO: 1)
MGQTGKKSEKGPVCWRKRVKSEYMRLRQLKRFRRADEVKSMFSSNRQKILERTEILNQEW
KQRRIQPVHILTSVSSLRGTRECSVTSDLDFPTQVIPLKTLNAVASVPIMYSWSPLQQNF
MVEDETVLHNIPYMGDEVLDQDGTFIEELIKNYDGKVHGDRECGFINDEIFVELVNALGQ
YNDDDDDDDGDDPEEREEKQKDLEDHRDDKESRPPRKFPSDKIFEAISSMFPDKGTAEEL
KEKYKELTEQQLPGALPPECTPNIDGPNAKSVQREQSLHSFHTLFCRRCFKYDCFLHPFH
ATPNTYKRKNTETALDNKPCGPQCYQHLEGAKEFAAALTAERIKTPPKRPGGRRRGRLPN
NSSRPSTPTINVLESKDTDSDREAGTETGGENNDKEEEEKKDETSSSSEANSRCQTPIKM
KPNIEPPENVEWSGAEASMFRVLIGTYYDNFCAIARLIGTKTCRQVYEFRVKESSIIAPA
PAEDVDTPPRKKKRKHRLWAAHCRKIQLKKDGSSNHVYNYQPCDHPRQPCDSSCPCVIAQ
NFCEKFCQCSSECQNRFPGCRCKAQCNTKQCPCYLAVRECDPDLCLTCGAADHWDSKNVS
CKNCSIQRGSKKHLLLAPSDVAGWGIFIKDPVQKNEFISEYCGEIISQDEADRRGKVYDK
YMCSFLFNLNNDFVVDATRKGNKIRFANHSVNPNCYAKVMMVNGDHRIGIFAKRAIQTGE
ELFFDYRYSQADALKYVGIEREMEIP
mRNA sequence of human EZH2, transcript variant 1 (GenBank Accession No.
NM_004456)
(SEQ ID NO: 2)
ggcggcgcttgattgggctgggggggccaaataaaagcgatggcgattgggctgccgcgt
ttggcgctcggtccggtcgcgtccgacacccggtgggactcagaaggcagtggagccccg
gcggcggcggcggcggcgcgcgggggcgacgcgcgggaacaacgcgagtcggcgcgcggg
acgaagaataatcatgggccagactgggaagaaatctgagaagggaccagtttgttggcg
gaagcgtgtaaaatcagagtacatgcgactgagacagctcaagaggttcagacgagctga
tgaagtaaagagtatgtttagttccaatcgtcagaaaattttggaaagaacggaaatctt
aaaccaagaatggaaacagcgaaggatacagcctgtgcacatcctgacttctgtgagctc
attgcgcgggactagggagtgttcggtgaccagtgacttggattttccaacacaagtcat
cccattaaagactctgaatgcagttgcttcagtacccataatgtattcttggtctcccct
acagcagaattttatggtggaagatgaaactgttttacataacattccttatatgggaga
tgaagttttagatcaggatggtactttcattgaagaactaataaaaaattatgatgggaa
agtacacggggatagagaatgtgggtttataaatgatgaaatttttgtggagttggtgaa
tgcccttggtcaatataatgatgatgacgatgatgatgatggagacgatcctgaagaaag
agaagaaaagcagaaagatctggaggatcaccgagatgataaagaaagccgcccacctcg
gaaatttccttctgataaaatttttgaagccatttcctcaatgtttccagataagggcac
agcagaagaactaaaggaaaaatataaagaactcaccgaacagcagctcccaggcgcact
tcctcctgaatgtacccccaacatagatggaccaaatgctaaatctgttcagagagagca
aagcttacactcctttcatacgcttttctgtaggcgatgttttaaatatgactgcttcct
acatcgtaagtgcaattattcttttcatgcaacacccaacacttataagcggaagaacac
agaaacagctctagacaacaaaccttgtggaccacagtgttaccagcatttggagggagc
aaaggagtttgctgctgctctcaccgctgagcggataaagaccccaccaaaacgtccagg
aggccgcagaagaggacggcttcccaataacagtagcaggcccagcacccccaccattaa
tgtgctggaatcaaaggatacagacagtgatagggaagcagggactgaaacggggggaga
gaacaatgataaagaagaagaagagaagaaagatgaaacttcgagctcctctgaagcaaa
ttctcggtgtcaaacaccaataaagatgaagccaaatattgaacctcctgagaatgtgga
gtggagtggtgctgaagcctcaatgtttagagtcctcattggcacttactatgacaattt
ctgtgccattgctaggttaattgggaccaaaacatgtagacaggtgtatgagtttagagt
caaagaatctagcatcatagctccagctcccgctgaggatgtggatactcctccaaggaa
aaagaagaggaaacaccggttgtgggctgcacactgcagaaagatacagctgaaaaagga
cggctcctctaaccatgtttacaactatcaaccctgtgatcatccacggcagccttgtga
cagttcgtgcccttgtgtgatagcacaaaatttttgtgaaaagttttgtcaatgtagttc
agagtgtcaaaaccgctttccgggatgccgctgcaaagcacagtgcaacaccaagcagtg
cccgtgctacctggctgtccgagagtgtgaccctgacctctgtcttacttgtggagccgc
tgaccattgggacagtaaaaatgtgtcctgcaagaactgcagtattcagcggggctccaa
aaagcatctattgctggcaccatctgacgtggcaggctgggggatttttatcaaagatcc
tgtgcagaaaaatgaattcatctcagaatactgtggagagattatttctcaagatgaagc
tgacagaagagggaaagtgtatgataaatacatgtgcagctttctgttcaacttgaacaa
tgattttgtggtggatgcaacccgcaagggtaacaaaattcgttttgcaaatcattcggt
aaatccaaactgctatgcaaaagttatgatggttaacggtgatcacaggataggtatttt
tgccaagagagccatccagactggcgaagagctgttttttgattacagatacagccaggc
tgatgccctgaagtatgtcggcatcgaaagagaaatggaaatcccttgacatctgctacc
tcctcccccctcctctgaaacagctgccttagcttcaggaacctcgagtactgtgggcaa
tttagaaaaagaacatgcagtttgaaattctgaatttgcaaagtactgtaagaataattt
atagtaatgagtttaaaaatcaactttttattgccttctcaccagctgcaaagtgttttg
taccagtgaatttttgcaataatgcagtatggtacatttttcaactttgaataaagaata
cttgaacttgtccttgttgaatc
Full amino acid of EZH2, isoform a (GenBank Accession No. NP_004447)
(SEQ ID NO: 3)
MGQTGKKSEKGPVCWRKRVKSEYMRLRQLKRERRADEVKSMESSNRQKILERTEILNQEW
KQRRIQPVHILTSVSSLRGTRECSVTSDLDFPTQVIPLKTLNAVASVPIMYSWSPLQQNF
MVEDETVLHNIPYMGDEVLDQDGTFIEELIKNYDGKVHGDRECGFINDEIFVELVNALGQ
YNDDDDDDDGDDPEEREEKQKDLEDHRDDKESRPPRKFPSDKIFEATSSMFPDKGTAEEL
KEKYKELTEQQLPGALPPECTPNIDGPNAKSVQREQSLHSFHTLFCRRCFKYDCFLHRKC
NYSFHATPNTYKRKNTETALDNKPCGPQCYQHLEGAKEFAAALTAERIKTPPKRPGGRRR
GRLPNNSSRPSTPTINVLESKDTDSDREAGTETGGENNDKEEEEKKDETSSSSEANSRCQ
TPIKMKPNIEPPENVEWSGAEASMFRVLIGTYYDNFCAIARLIGTKTCRQVYEFRVKESS
IIAPAPAEDVDTPPRKKKRKHRLWAAHCRKIQLKKDGSSNHVYNYQPCDHPRQPCDSSCP
CVIAQNFCEKFCQCSSECQNRFPGCRCKAQCNTKQCPCYLAVRECDPDLCLTCGAADHWD
SKNVSCKNCSIQRGSKKHLLLAPSDVAGWGIFIKDPVQKNEFISEYCGEIISQDEADRRG
KVYDKYMCSFLFNLNNDFVVDATRKGNKIRFANHSVNPNCYAKVMMVNGDHRIGIFAKRA
IQTGEELFFDYRYSQADALKYVGIEREMEIP
mRNA sequence of human EZH2, transcript variant 2 (GenBank Accession No.
NM_152998)
(SEQ ID NO: 4)
ggcggcgcttgattgggctgggggggccaaataaaagcgatggcgattgggctgccgcgt
ttggcgctcggtccggtcgcgtccgacacccggtgggactcagaaggcagtggagccccg
gcggcggcggcggcggcgcgcgggggcgacgcgcgggaacaacgcgagtcggcgcgcggg
acgaagaataatcatgggccagactgggaagaaatctgagaagggaccagtttgttggcg
gaagcgtgtaaaatcagagtacatgcgactgagacagctcaagaggttcagacgagctga
tgaagtaaagagtatgtttagttccaatcgtcagaaaattttggaaagaacggaaatctt
aaaccaagaatggaaacagcgaaggatacagcctgtgcacatcctgacttctgtgagctc
attgcgcgggactagggaggtggaagatgaaactgttttacataacattccttatatggg
agatgaagttttagatcaggatggtactttcattgaagaactaataaaaaattatgatgg
gaaagtacacggggatagagaatgtgggtttataaatgatgaaatttttgtggagttggt
gaatgcccttggtcaatataatgatgatgacgatgatgatgatggagacgatcctgaaga
aagagaagaaaagcagaaagatctggaggatcaccgagatgataaagaaagccgcccacc
tcggaaatttccttctgataaaatttttgaagccatttcctcaatgtttccagataaggg
cacagcagaagaactaaaggaaaaatataaagaactcaccgaacagcagctcccaggcgc
acttcctcctgaatgtacccccaacatagatggaccaaatgctaaatctgttcagagaga
gcaaagcttacactcctttcatacgcttttctgtaggcgatgttttaaatatgactgctt
cctacatccttttcatgcaacacccaacacttataagcggaagaacacagaaacagctct
agacaacaaaccttgtggaccacagtgttaccagcatttggagggagcaaaggagtttgc
tgctgctctcaccgctgagcggataaagaccccaccaaaacgtccaggaggccgcagaag
aggacggcttcccaataacagtagcaggcccagcacccccaccattaatgtgctggaatc
aaaggatacagacagtgatagggaagcagggactgaaacggggggagagaacaatgataa
agaagaagaagagaagaaagatgaaacttcgagctcctctgaagcaaattctcggtgtca
aacaccaataaagatgaagccaaatattgaacctcctgagaatgtggagtggagtggtgc
tgaagcctcaatgtttagagtcctcattggcacttactatgacaatttctgtgccattgc
taggttaattgggaccaaaacatgtagacaggtgtatgagtttagagtcaaagaatctag
catcatagctccagctcccgctgaggatgtggatactcctccaaggaaaaagaagaggaa
acaccggttgtgggctgcacactgcagaaagatacagctgaaaaaggacggctcctctaa
ccatgtttacaactatcaaccctgtgatcatccacggcagccttgtgacagttcgtgccc
ttgtgtgatagcacaaaatttttgtgaaaagttttgtcaatgtagttcagagtgtcaaaa
ccgctttccgggatgccgctgcaaagcacagtgcaacaccaagcagtgcccgtgctacct
ggctgtccgagagtgtgaccctgacctctgtcttacttgtggagccgctgaccattggga
cagtaaaaatgtgtcctgcaagaactgcagtattcagcggggctccaaaaagcatctatt
gctggcaccatctgacgtggcaggctgggggatttttatcaaagatcctgtgcagaaaaa
tgaattcatctcagaatactgtggagagattatttctcaagatgaagctgacagaagagg
gaaagtgtatgataaatacatgtgcagctttctgttcaacttgaacaatgattttgtggt
ggatgcaacccgcaagggtaacaaaattcgttttgcaaatcattcggtaaatccaaactg
ctatgcaaaagttatgatggttaacggtgatcacaggataggtatttttgccaagagagc
catccagactggcgaagagctgttttttgattacagatacagccaggctgatgccctgaa
gtatgtcggcatcgaaagagaaatggaaatcccttgacatctgctacctcctcccccctc
ctctgaaacagctgccttagcttcaggaacctcgagtactgtgggcaatttagaaaaaga
acatgcagtttgaaattctgaatttgcaaagtactgtaagaataatttatagtaatgagt
ttaaaaatcaactttttattgccttctcaccagctgcaaagtgttttgtaccagtgaatt
tttgcaataatgcagtatggtacatttttcaactttgaataaagaatacttgaacttgtc
cttgttgaatc
Full amino acid of EZH2, isoform b (GenBank Accession No. NP_694543)
(SEQ ID NO: 5)
MGQTGKKSEKGPVCWRKRVKSEYMRLRQLKRFRRADEVKSMFSSNRQKIL
ERTEILNQEWKQRRIQPVHILTSVSSLRGTREVEDETVLHNIPYMGDEVL
DQDGTFIEELIKNYDGKVHGDRECGFINDEIFVELVNALGQYNDDDDDDD
GDDPEEREEKQKDLEDHRDDKESRPPRKFPSDKIFEATSSMFPDKGTAEE
LKEKYKELTEQQLPGALPPECTPNIDGPNAKSVQREQSLHSFHTLFCRRC
FKYDCFLHPFHATPNTYKRKNTETALDNKPCGPQCYQHLEGAKEFAAALT
AERIKTPPKRPGGRRRGRLPNNSSRPSTPTINVLESKDTDSDREAGTETG
GENNDKEEEEKKDETSSSSEANSRCQTPIKMKPNIEPPENVEWSGAEASM
FRVLIGTYYDNFCAIARLIGTKTCRQVYEFRVKESSIIAPAPAEDVDTPP
RKKKRKHRLWAAHCRKIQLKKDGSSNHVYNYQPCDHPRQPCDSSCPCVIA
QNFCEKFCQCSSECQNRFPGCRCKAQCNTKQCPCYLAVRECDPDLCLTCG
AADHWDSKNVSCKNCSIQRGSKKHLLLAPSDVAGWGIFIKDPVQKNEFIS
EYCGEIISQDEADRRGKVYDKYMCSFLFNLNNDFVVDATRKGNKIRFANH
SVNPNCYAKVMMVNGDHRIGIFAKRAIQTGEELFFDYRYSQADALKYVGI
EREMEIP
Full amino acid of EZH2, isoform e (GenBank Accession No. NP_001190178.1)
(SEQ ID NO: 21)
MGQTGKKSEKGPVCWRKRVKSEYMRLRQLKRFRRADEVKSMFSSNRQKILERTEILNQEWKQRRIQPVHI
LTSCSVTSDLDFPTQVIPLKTLNAVASVPIMYSWSPLQQNFMVEDETVLHNIPYMGDEVLDQDGTFIEEL
IKNYDGKVHGDRECGFINDEIFVELVNALGQYNDDDDDDDGDDPEEREEKQKDLEDHRDDKESRPPRKFP
SDKIFEAISSMFPDKGTAEELKEKYKELTEQQLPGALPPECTPNIDGPNAKSVQREQSLHSFHTLFCRRC
FKYDCFLHPFHATPNTYKRKNTETALDNKPCGPQCYQHLEGAKEFAAALTAERIKTPPKRPGGRRRGRLP
NNSSRPSTPTINVLESKDTDSDREAGTETGGENNDKEEEEKKDETSSSSEANSRCQTPIKMKPNIEPPEN
VEWSGAEASMFRVLIGTYYDNFCAIARLIGTKTCRQVYEFRVKESSIIAPAPAEDVDTPPRKKKRKHRLW
AAHCRKIQLKKGQNRFPGCRCKAQCNTKQCPCYLAVRECDPDLCLTCGAADHWDSKNVSCKNCSIQRGSK
KHLLLAPSDVAGWGIFIKDPVQKNEFISEYCGEIISQDEADRRGKVYDKYMCSFLFNLNNDFVVDATRKG
NKIRFANHSVNPNCYAKVMMVNGDHRIGIFAKRAIQTGEELFFDYRYSQADALKYVGIEREMEIP
Homo sapiens enhancer of zeste homolog 2 (Drosophila)(EZH2), transcript variant 5,
mRNA (GenBank Accession No. NM_001203249.1)
(SEQ ID NO: 22)
GACGACGTTCGCGGCGGGGAACTCGGAGTAGCTTCGCCTCTGACGTTTCCCCACGACGCACCCCGAAATC
CCCCTGAGCTCCGGCGGTCGCGGGCTGCCCTCGCCGCCTGGTCTGGCTTTATGCTAAGTTTGAGGGAAGA
GTCGAGCTGCTCTGCTCTCTATTGATTGTGTTTCTGGAGGGCGTCCTGTTGAATTCCCACTTCATTGTGT
ACATCCCCTTCCGTTCCCCCCAAAAATCTGTGCCACAGGGTTACTTTTTGAAAGCGGGAGGAATCGAGAA
GCACGATCTTTTGGAAAACTTGGTGAACGCCTAAATAATCATGGGCCAGACTGGGAAGAAATCTGAGAAG
GGACCAGTTTGTTGGCGGAAGCGTGTAAAATCAGAGTACATGCGACTGAGACAGCTCAAGAGGTTCAGAC
GAGCTGATGAAGTAAAGAGTATGTTTAGTTCCAATCGTCAGAAAATTTTGGAAAGAACGGAAATCTTAAA
CCAAGAATGGAAACAGCGAAGGATACAGCCTGTGCACATCCTGACTTCTTGTTCGGTGACCAGTGACTTG
GATTTTCCAACACAAGTCATCCCATTAAAGACTCTGAATGCAGTTGCTTCAGTACCCATAATGTATTCTT
GGTCTCCCCTACAGCAGAATTTTATGGTGGAAGATGAAACTGTTTTACATAACATTCCTTATATGGGAGA
TGAAGTTTTAGATCAGGATGGTACTTTCATTGAAGAACTAATAAAAAATTATGATGGGAAAGTACACGGG
GATAGAGAATGTGGGTTTATAAATGATGAAATTTTTGTGGAGTTGGTGAATGCCCTTGGTCAATATAATG
ATGATGACGATGATGATGATGGAGACGATCCTGAAGAAAGAGAAGAAAAGCAGAAAGATCTGGAGGATCA
CCGAGATGATAAAGAAAGCCGCCCACCTCGGAAATTTCCTTCTGATAAAATTTTTGAAGCCATTTCCTCA
ATGTTTCCAGATAAGGGCACAGCAGAAGAACTAAAGGAAAAATATAAAGAACTCACCGAACAGCAGCTCC
CAGGCGCACTTCCTCCTGAATGTACCCCCAACATAGATGGACCAAATGCTAAATCTGTTCAGAGAGAGCA
AAGCTTACACTCCTTTCATACGCTTTTCTGTAGGCGATGTTTTAAATATGACTGCTTCCTACATCCTTTT
CATGCAACACCCAACACTTATAAGCGGAAGAACACAGAAACAGCTCTAGACAACAAACCTTGTGGACCAC
AGTGTTACCAGCATTTGGAGGGAGCAAAGGAGTTTGCTGCTGCTCTCACCGCTGAGCGGATAAAGACCCC
ACCAAAACGTCCAGGAGGCCGCAGAAGAGGACGGCTTCCCAATAACAGTAGCAGGCCCAGCACCCCCACC
ATTAATGTGCTGGAATCAAAGGATACAGACAGTGATAGGGAAGCAGGGACTGAAACGGGGGGAGAGAACA
ATGATAAAGAAGAAGAAGAGAAGAAAGATGAAACTTCGAGCTCCTCTGAAGCAAATTCTCGGTGTCAAAC
ACCAATAAAGATGAAGCCAAATATTGAACCTCCTGAGAATGTGGAGTGGAGTGGTGCTGAAGCCTCAATG
TTTAGAGTCCTCATTGGCACTTACTATGACAATTTCTGTGCCATTGCTAGGTTAATTGGGACCAAAACAT
GTAGACAGGTGTATGAGTTTAGAGTCAAAGAATCTAGCATCATAGCTCCAGCTCCCGCTGAGGATGTGGA
TACTCCTCCAAGGAAAAAGAAGAGGAAACACCGGTTGTGGGCTGCACACTGCAGAAAGATACAGCTGAAA
AAGGGTCAAAACCGCTTTCCGGGATGCCGCTGCAAAGCACAGTGCAACACCAAGCAGTGCCCGTGCTACC
TGGCTGTCCGAGAGTGTGACCCTGACCTCTGTCTTACTTGTGGAGCCGCTGACCATTGGGACAGTAAAAA
TGTGTCCTGCAAGAACTGCAGTATTCAGCGGGGCTCCAAAAAGCATCTATTGCTGGCACCATCTGACGTG
GCAGGCTGGGGGATTTTTATCAAAGATCCTGTGCAGAAAAATGAATTCATCTCAGAATACTGTGGAGAGA
TTATTTCTCAAGATGAAGCTGACAGAAGAGGGAAAGTGTATGATAAATACATGTGCAGCTTTCTGTTCAA
CTTGAACAATGATTTTGTGGTGGATGCAACCCGCAAGGGTAACAAAATTCGTTTTGCAAATCATTCGGTA
AATCCAAACTGCTATGCAAAAGTTATGATGGTTAACGGTGATCACAGGATAGGTATTTTTGCCAAGAGAG
CCATCCAGACTGGCGAAGAGCTGTTTTTTGATTACAGATACAGCCAGGCTGATGCCCTGAAGTATGTCGG
CATCGAAAGAGAAATGGAAATCCCTTGACATCTGCTACCTCCTCCCCCCTCCTCTGAAACAGCTGCCTTA
GCTTCAGGAACCTCGAGTACTGTGGGCAATTTAGAAAAAGAACATGCAGTTTGAAATTCTGAATTTGCAA
AGTACTGTAAGAATAATTTATAGTAATGAGTTTAAAAATCAACTTTTTATTGCCTTCTCACCAGCTGCAA
AGTGTTTTGTACCAGTGAATTTTTGCAATAATGCAGTATGGTACATTTTTCAACTTTGAATAAAGAATAC
TTGAACTTGTCCTTGTTGAATC

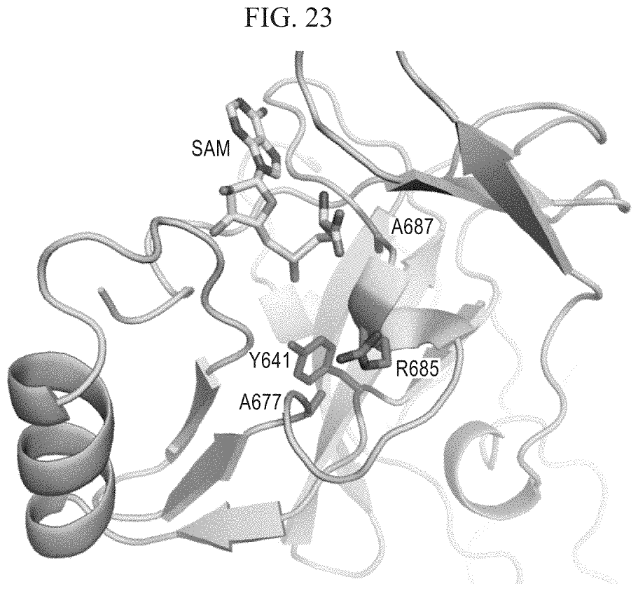

A structure model of partial EZH2 protein based on the A chain of nuclear receptor binding SET domain protein 1 (NSD1) is provided in FIG. 23. This model corresponds to amino acid residues 533-732 of EZH2 sequence of SEQ ID NO: 1.

The corresponding amino acid sequence of this structure model is provided below. The residues in the substrate pocket domain are underlined. The residues in the SET domain are shown italic.

(SEQ ID NO: 6)
SCPCVIAQNFCEKFCQCSSECQNRFPGCRCKAQCNTKQCPCYLAVRECDPDLCLTCG

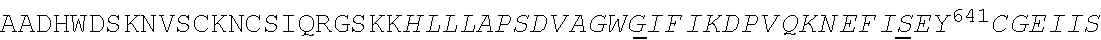

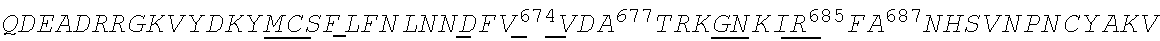

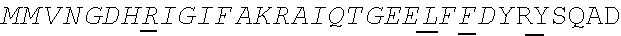

The catalytic site of EZH2 is believed to reside in a conserved domain of the protein known as the SET domain. The amino acid sequence of the SET domain of EZH2 is provided by the following partial sequence spanning amino acid residues 613-726 of Swiss-Prot Accession No. Q15910 (SEQ ID NO: 1):

(SEQ ID NO: 7)
HLLLAPSDVAGWGIFIKDPVQKNEFISEYCGEIISQDEADRRGKVYDKYM
CSFLFNLNNDFVVDATRKGNKIRFANHSVNPNCYAKVMMVNGDHRIGIFA
KRAIQTGEELFFDY.

The tyrosine (Y) residue shown underlined in SEQ ID NO: 7 is Tyr641 (Y641) in Swiss-Prot Accession No. Q15910 (SEQ ID NO: 1).

The SET domain of GenBank Accession No. NP_004447 (SEQ ID NO: 3) spans amino acid residues 618-731 and is identical to SEQ ID NO:6. The tyrosine residue corresponding to Y641 in Swiss-Prot Accession No. Q15910 shown underlined in SEQ ID NO: 7 is Tyr646 (Y646) in GenBank Accession No. NP_004447 (SEQ ID NO: 3).

The SET domain of GenBank Accession No. NP_694543 (SEQ ID NO: 5) spans amino acid residues 574-687 and is identical to SEQ ID NO: 7. The tyrosine residue corresponding to Y641 in Swiss-Prot Accession No. Q15910 shown underlined in SEQ ID NO: 7 is Tyr602 (Y602) in GenBank Accession No. NP_694543 (SEQ ID NO: 5).

The nucleotide sequence encoding the SET domain of GenBank Accession No. NP_004447 is

(SEQ ID NO: 8)
catctattgctggcaccatctgacgtggcaggctgggggatttttatcaa
agatcctgtgcagaaaaatgaattcatctcagaatactgtggagagatta
tttctcaagatgaagctgacagaagagggaaagtgtatgataaatacatg
tgcagctttctgttcaacttgaacaatgattttgtggtggatgcaacccg
caagggtaacaaaattcgttttgcaaatcattcggtaaatccaaactgct
atgcaaaagttatgatggttaacggtgatcacaggataggtatttttgcc
aagagagccatccagactggcgaagagctgttttttgattac,

where the codon encoding Y641 is shown underlined.

For purposes of this application, amino acid residue Y641 of human EZH2 is to be understood to refer to the tyrosine residue that is or corresponds to Y641 in Swiss-Prot Accession No. Q15910.

Full amino acid sequence of Y641 mutant EZH2
(SEQ ID NO: 9)
MGQTGKKSEKGPVCWRKRVKSEYMRLRQLKRERRADEVKSMESSNRQKIL
ERTEILNQEWKQRRIQPVHILTSVSSLRGTRECSVTSDLDFPTQVIPLKT
LNAVASVPIMYSWSPLQQNFMVEDETVLHNIPYMGDEVLDQDGTFIEELI
KNYDGKVHGDRECGFINDEIFVELVNALGQYNDDDDDDDGDDPEEREEKQ
KDLEDHRDDKESRPPRKFPSDKIFEAISSMFPDKGTAEELKEKYKELTEQ
QLPGALPPECTPNIDGPNAKSVQREQSLHSFHTLFCRRCFKYDCFLHPFH
ATPNTYKRKNTETALDNKPCGPQCYQHLEGAKEFAAALTAERIKTPPKRP
GGRRRGRLPNNSSRPSTPTINVLESKDTDSDREAGTETGGENNDKEEEEK
KDETSSSSEANSRCQTPIKMKPNIEPPENVEWSGAEASMFRVLIGTYYDN
FCAIARLIGTKTCRQVYEFRVKESSIIAPAPAEDVDTPPRKKKRKHRLWA
AHCRKIQLKKDGSSNHVYNYQPCDHPRQPCDSSCPCVIAQNFCEKFCQCS
SECQNRFPGCRCKAQCNTKQCPCYLAVRECDPDLCLTCGAADHWDSKNVS
CKNCSIQRGSKKHLLLAPSDVAGWGIFIKDPVQKNEFISEXCGEIISQDE
ADRRGKVYDKYMCSFLENLNNDFVVDATRKGNKIRFANHSVNPNCYAKVM
MVNGDHRIGIFAKRAIQTGEELFFDYRYSQADALKYVGIEREMEIP
Wherein X can be any amino acid residue other than
tyrosine (Y)

A Y641 mutant of human EZH2, and, equivalently, a Y641 mutant of EZH2, is to be understood to refer to a human EZH2 in which the amino acid residue corresponding to Y641 of wild-type human EZH2 is substituted by an amino acid residue other than tyrosine.

In one embodiment the amino acid sequence of a Y641 mutant of EZH2 differs from the amino acid sequence of wild-type human EZH2 only by substitution of a single amino acid residue corresponding to Y641 of wild-type human EZH2 by an amino acid residue other than tyrosine.

In one embodiment the amino acid sequence of a Y641 mutant of EZH2 differs from the amino acid sequence of wild-type human EZH2 only by substitution of phenylalanine (F) for the single amino acid residue corresponding to Y641 of wild-type human EZH2. The Y641 mutant of EZH2 according to this embodiment is referred to herein as a Y641F mutant or, equivalently, Y641F.

Y641F
(SEQ ID NO: 10)
MGQTGKKSEKGPVCWRKRVKSEYMRLRQLKRFRRADEVKSMFSSNRQKIL
ERTEILNQEWKQRRIQPVHILTSVSSLRGTRECSVTSDLDFPTQVIPLKT
LNAVASVPIMYSWSPLQQNFMVEDETVLHNIPYMGDEVLDQDGTFIEELI
KNYDGKVHGDRECGFINDEIFVELVNALGQYNDDDDDDDGDDPEEREEKQ
KDLEDHRDDKESRPPRKFPSDKIFEAISSMFPDKGTAEELKEKYKELTEQ
QLPGALPPECTPNIDGPNAKSVQREQSLHSFHTLFCRRCFKYDCFLHPFH
ATPNTYKRKNTETALDNKPCGPQCYQHLEGAKEFAAALTAERIKTPPKRP
GGRRRGRLPNNSSRPSTPTINVLESKDTDSDREAGTETGGENNDKEEEEK
KDETSSSSEANSRCQTPIKMKPNIEPPENVEWSGAEASMFRVLIGTYYDN
FCAIARLIGTKTCRQVYEFRVKESSIIAPAPAEDVDTPPRKKKRKHRLWA
AHCRKIQLKKDGSSNHVYNYQPCDHPRQPCDSSCPCVIAQNFCEKFCQCS
SECQNRFPGCRCKAQCNTKQCPCYLAVRECDPDLCLTCGAADHWDSKNVS
CKNCSIQRGSKKHLLLAPSDVAGWGIFIKDPVQKNEFISEFCGEIISQDE
ADRRGKVYDKYMCSFLFNLNNDFVVDATRKGNKIRFANHSVNPNCYAKVM
MVNGDHRIGIFAKRAIQTGEELFFDYRYSQADALKYVGIEREMEIP

In one embodiment the amino acid sequence of a Y641 mutant of EZH2 differs from the amino acid sequence of wild-type human EZH2 only by substitution of histidine (H) for the single amino acid residue corresponding to Y641 of wild-type human EZH2. The Y641 mutant of EZH2 according to this embodiment is referred to herein as a Y641H mutant or, equivalently, Y641H.

Y641H
(SEQ ID NO: 11)
MGQTGKKSEKGPVCWRKRVKSEYMRLRQLKRFRRADEVKSMFSSNRQKIL
ERTEILNQEWKQRRIQPVHILTSVSSLRGTRECSVTSDLDFPTQVIPLKT
LNAVASVPIMYSWSPLQQNFMVEDETVLHNIPYMGDEVLDQDGTFIEELI
KNYDGKVHGDRECGFINDEIFVELVNALGQYNDDDDDDDGDDPEEREEKQ
KDLEDHRDDKESRPPRKFPSDKIFEATSSMFPDKGTAEELKEKYKELTEQ
QLPGALPPECTPNIDGPNAKSVQREQSLHSFHTLFCRRCFKYDCFLHPFH
ATPNTYKRKNTETALDNKPCGPQCYQHLEGAKEFAAALTAERIKTPPKRP
GGRRRGRLPNNSSRPSTPTINVLESKDTDSDREAGTETGGENNDKEEEEK
KDETSSSSEANSRCQTPIKMKPNIEPPENVEWSGAEASMFRVLIGTYYDN
FCAIARLIGTKTCRQVYEFRVKESSIIAPAPAEDVDTPPRKKKRKHRLWA
AHCRKIQLKKDGSSNHVYNYQPCDHPRQPCDSSCPCVIAQNFCEKFCQCS
SECQNRFPGCRCKAQCNTKQCPCYLAVRECDPDLCLTCGAADHWDSKNVS
CKNCSIQRGSKKHLLLAPSDVAGWGIFIKDPVQKNEFISEHCGEIISQDE
ADRRGKVYDKYMCSFLFNLNNDFVVDATRKGNKIRFANHSVNPNCYAKVM
MVNGDHRIGIFAKRAIQTGEELFFDYRYSQADALKYVGIEREMEIP

In one embodiment the amino acid sequence of a Y641 mutant of EZH2 differs from the amino acid sequence of wild-type human EZH2 only by substitution of asparagine (N) for the single amino acid residue corresponding to Y641 of wild-type human EZH2. The Y641 mutant of EZH2 according to this embodiment is referred to herein as a Y641N mutant or, equivalently, Y641N.

Y641N
(SEQ ID NO: 12)
MGQTGKKSEKGPVCWRKRVKSEYMRLRQLKRFRRADEVKSMFSSNRQKIL
ERTEILNQEWKQRRIQPVHILTSVSSLRGTRECSVTSDLDFPTQVIPLKT
LNAVASVPIMYSWSPLQQNFMVEDETVLHNIPYMGDEVLDQDGTFIEELI
KNYDGKVHGDRECGFINDEIFVELVNALGQYNDDDDDDDGDDPEEREEKQ
KDLEDHRDDKESRPPRKFPSDKIFEATSSMFPDKGTAEELKEKYKELTEQ
QLPGALPPECTPNIDGPNAKSVQREQSLHSFHTLFCRRCFKYDCFLHPFH
ATPNTYKRKNTETALDNKPCGPQCYQHLEGAKEFAAALTAERIKTPPKRP
GGRRRGRLPNNSSRPSTPTINVLESKDTDSDREAGTETGGENNDKEEEEK
KDETSSSSEANSRCQTPIKMKPNIEPPENVEWSGAEASMFRVLIGTYYDN
FCAIARLIGTKTCRQVYEFRVKESSIIAPAPAEDVDTPPRKKKRKHRLWA
AHCRKIQLKKDGSSNHVYNYQPCDHPRQPCDSSCPCVIAQNFCEKFCQCS
SECQNRFPGCRCKAQCNTKQCPCYLAVRECDPDLCLTCGAADHWDSKNVS
CKNCSIQRGSKKHLLLAPSDVAGWGIFIKDPVQKNEFISENCGEIISQDE
ADRRGKVYDKYMCSFLFNLNNDFVVDATRKGNKIRFANHSVNPNCYAKVM
MVNGDHRIGIFAKRAIQTGEELFFDYRYSQADALKYVGIEREMEIP

In one embodiment the amino acid sequence of a Y641 mutant of EZH2 differs from the amino acid sequence of wild-type human EZH2 only by substitution of serine (S) for the single amino acid residue corresponding to Y641 of wild-type human EZH2. The Y641 mutant of EZH2 according to this embodiment is referred to herein as a Y641S mutant or, equivalently, Y641S.

Y641S
(SEQ ID NO: 13)
MGQTGKKSEKGPVCWRKRVKSEYMRLRQLKRFRRADEVKSMFSSNRQKIL
ERTEILNQEWKQRRIQPVHILTSVSSLRGTRECSVTSDLDFPTQVIPLKT
LNAVASVPIMYSWSPLQQNFMVEDETVLHNIPYMGDEVLDQDGTFIEELI
KNYDGKVHGDRECGFINDEIFVELVNALGQYNDDDDDDDGDDPEEREEKQ
KDLEDHRDDKESRPPRKFPSDKIFEATSSMFPDKGTAEELKEKYKELTEQ
QLPGALPPECTPNIDGPNAKSVQREQSLHSFHTLFCRRCFKYDCFLHPFH
ATPNTYKRKNTETALDNKPCGPQCYQHLEGAKEFAAALTAERIKTPPKRP
GGRRRGRLPNNSSRPSTPTINVLESKDTDSDREAGTETGGENNDKEEEEK
KDETSSSSEANSRCQTPIKMKPNIEPPENVEWSGAEASMFRVLIGTYYDN
FCAIARLIGTKTCRQVYEFRVKESSIIAPAPAEDVDTPPRKKKRKHRLWA
AHCRKIQLKKDGSSNHVYNYQPCDHPRQPCDSSCPCVIAQNFCEKFCQCS
SECQNRFPGCRCKAQCNTKQCPCYLAVRECDPDLCLTCGAADHWDSKNVS
CKNCSIQRGSKKHLLLAPSDVAGWGIFIKDPVQKNEFISESCGEIISQDE
ADRRGKVYDKYMCSFLFNLNNDFVVDATRKGNKIRFANHSVNPNCYAKVM
MVNGDHRIGIFAKRAIQTGEELFFDYRYSQADALKYVGIEREMEIP

In one embodiment the amino acid sequence of a Y641 mutant of EZH2 differs from the amino acid sequence of wild-type human EZH2 only by substitution of cysteine (C) for the single amino acid residue corresponding to Y641 of wild-type human EZH2. The Y641 mutant of EZH2 according to this embodiment is referred to herein as a Y641C mutant or, equivalently, Y641C.

Y641C
(SEQ ID NO: 14)
MGQTGKKSEKGPVCWRKRVKSEYMRLRQLKRFRRADEVKSMFSSNRQKIL
ERTEILNQEWKQRRIQPVHILTSVSSLRGTRECSVTSDLDFPTQVIPLKT
LNAVASVPIMYSWSPLQQNFMVEDETVLHNIPYMGDEVLDQDGTFIEELI
KNYDGKVHGDRECGFINDEIFVELVNALGQYNDDDDDDDGDDPEEREEKQ
KDLEDHRDDKESRPPRKFPSDKIFEAISSMFPDKGTAEELKEKYKELTEQ
QLPGALPPECTPNIDGPNAKSVQREQSLHSFHTLFCRRCFKYDCFLHPFH
ATPNTYKRKNTETALDNKPCGPQCYQHLEGAKEFAAALTAERIKTPPKRP
GGRRRGRLPNNSSRPSTPTINVLESKDTDSDREAGTETGGENNDKEEEEK
KDETSSSSEANSRCQTPIKMKPNIEPPENVEWSGAEASMFRVLIGTYYDN
FCAIARLIGTKTCRQVYEFRVKESSIIAPAPAEDVDTPPRKKKRKHRLWA
AHCRKIQLKKDGSSNHVYNYQPCDHPRQPCDSSCPCVIAQNFCEKFCQCS
SECQNRFPGCRCKAQCNTKQCPCYLAVRECDPDLCLTCGAADHWDSKNVS
CKNCSIQRGSKKHLLLAPSDVAGWGIFIKDPVQKNEFISECCGEIISQDE
ADRRGKVYDKYMCSFLFNLNNDFVVDATRKGNKIRFANHSVNPNCYAKVM
MVNGDHRIGIFAKRAIQTGEELFFDYRYSQADALKYVGIEREMEIP

In one embodiment the amino acid sequence of a A677 mutant of EZH2 differs from the amino acid sequence of wild-type human EZH2 only by substitution of a non-alanine amino acid, preferably glycine (G) for the single amino acid residue corresponding to A677 of wild-type human EZH2. The A677 mutant of EZH2 according to this embodiment is referred to herein as an A677 mutant, and preferably an A677G mutant or, equivalently, A677G.

A677
(SEQ ID NO: 15)
MGQTGKKSEKGPVCWRKRVKSEYMRLRQLKRFRRADEVKSMFSSNRQKIL
ERTEILNQEWKQRRIQPVHILTSVSSLRGTRECSVTSDLDFPTQVIPLKT
LNAVASVPIMYSWSPLQQNFMVEDETVLHNIPYMGDEVLDQDGTFIEELI
KNYDGKVHGDRECGFINDEIFVELVNALGQYNDDDDDDDGDDPEEREEKQ
KDLEDHRDDKESRPPRKFPSDKIFEAISSMFPDKGTAEELKEKYKELTEQ
QLPGALPPECTPNIDGPNAKSVQREQSLHSFHTLFCRRCFKYDCFLHPFH
ATPNTYKRKNTETALDNKPCGPQCYQHLEGAKEFAAALTAERIKTPPKRP
GGRRRGRLPNNSSRPSTPTINVLESKDTDSDREAGTETGGENNDKEEEEK
KDETSSSSEANSRCQTPIKMKPNIEPPENVEWSGAEASMFRVLIGTYYDN
FCAIARLIGTKTCRQVYEFRVKESSIIAPAPAEDVDTPPRKKKRKHRLWA
AHCRKIQLKKDGSSNHVYNYQPCDHPRQPCDSSCPCVIAQNFCEKFCQCS
SECQNRFPGCRCKAQCNTKQCPCYLAVRECDPDLCLTCGAADHWDSKNVS
CKNCSIQRGSKKHLLLAPSDVAGWGIFIKDPVQKNEFISEYCGEIISQDE
ADRRGKVYDKYMCSFLFNLNNDFVVDXTRKGNKIRFANHSVNPNCYAKVM
MVNGDHRIGIFAKRAIQTGEELFFDYRYSQADALKYVGIEREMEIP
Wherein X is preferably a glycine (G).

In one embodiment the amino acid sequence of a A687 mutant of EZH2 differs from the amino acid sequence of wild-type human EZH2 only by substitution of a non-alanine amino acid, preferably valine (V) for the single amino acid residue corresponding to A687 of wild-type human EZH2. The A687 mutant of EZH2 according to this embodiment is referred to herein as an A687 mutant and preferably an A687V mutant or, equivalently, A687V.

A687
(SEQ ID NO: 16)
MGQTGKKSEKGPVCWRKRVKSEYMRLRQLKRERRADEVKSMESSNRQKIL
ERTEILNQEWKQRRIQPVHILTSVSSLRGTRECSVTSDLDFPTQVIPLKT
LNAVASVPIMYSWSPLQQNFMVEDETVLHNIPYMGDEVLDQDGTFIEELI
KNYDGKVHGDRECGFINDEIFVELVNALGQYNDDDDDDDGDDPEEREEKQ
KDLEDHRDDKESRPPRKFPSDKIFEAISSMFPDKGTAEELKEKYKELTEQ
QLPGALPPECTPNIDGPNAKSVQREQSLHSFHTLFCRRCFKYDCFLHPFH
ATPNTYKRKNTETALDNKPCGPQCYQHLEGAKEFAAALTAERIKTPPKRP
GGRRRGRLPNNSSRPSTPTINVLESKDTDSDREAGTETGGENNDKEEEEK
KDETSSSSEANSRCQTPIKMKPNIEPPENVEWSGAEASMFRVLIGTYYDN
FCAIARLIGTKTCRQVYEFRVKESSIIAPAPAEDVDTPPRKKKRKHRLWA
AHCRKIQLKKDGSSNHVYNYQPCDHPRQPCDSSCPCVIAQNFCEKFCQCS
SECQNRFPGCRCKAQCNTKQCPCYLAVRECDPDLCLTCGAADHWDSKNVS
CKNCSIQRGSKKHLLLAPSDVAGWGIFIKDPVQKNEFISEYCGEIISQDE
ADRRGKVYDKYMCSFLENLNNDFVVDATRKGNKIRFXNHSVNPNCYAKVM
MVNGDHRIGIFAKRAIQTGEELFFDYRYSQADALKYVGIEREMEIP
Wherein X is preferably a valine (V).

In one embodiment the amino acid sequence of a R685 mutant of EZH2 differs from the amino acid sequence of wild-type human EZH2 only by substitution of a non-arginine amino acid, preferably histidine (H) or cysteine (C) for the single amino acid residue corresponding to R685 of wild-type human EZH2. The R685 mutant of EZH2 according to this embodiment is referred to herein as an R685 mutant and preferably an R685C mutant or an R685H mutant or, equivalently, R685H or R685C.

A685
(SEQ ID NO: 17)
MGQTGKKSEKGPVCWRKRVKSEYMRLRQLKRFRRADEVKSMFSSNRQKIL
ERTEILNQEWKQRRIQPVHILTSVSSLRGTRECSVTSDLDFPTQVIPLKT
LNAVASVPIMYSWSPLQQNFMVEDETVLHNIPYMGDEVLDQDGTFIEELI
KNYDGKVHGDRECGFINDEIFVELVNALGQYNDDDDDDDGDDPEEREEKQ
KDLEDHRDDKESRPPRKFPSDKIFEAISSMFPDKGTAEELKEKYKELTEQ
QLPGALPPECTPNIDGPNAKSVQREQSLHSFHTLFCRRCFKYDCFLHPFH
ATPNTYKRKNTETALDNKPCGPQCYQHLEGAKEFAAALTAERIKTPPKRP
GGRRRGRLPNNSSRPSTPTINVLESKDTDSDREAGTETGGENNDKEEEEK
KDETSSSSEANSRCQTPIKMKPNIEPPENVEWSGAEASMFRVLIGTYYDN
FCAIARLIGTKTCRQVYEFRVKESSIIAPAPAEDVDTPPRKKKRKHRLWA
AHCRKIQLKKDGSSNHVYNYQPCDHPRQPCDSSCPCVIAQNFCEKFCQCS
SECQNRFPGCRCKAQCNTKQCPCYLAVRECDPDLCLTCGAADHWDSKNVS
CKNCSIQRGSKKHLLLAPSDVAGWGIFIKDPVQKNEFISEYCGEIISQDE
ADRRGKVYDKYMCSFLFNLNNDFVVDATRKGNKIXFANHSVNPNCYAKVM
MVNGDHRIGIFAKRAIQTGEELFFDYRYSQADALKYVGIEREMEIP
Wherein X is preferably a cysteine (C) or a
histidine (H).

In one embodiment the amino acid sequence of a mutant of EZH2 differs from the amino acid sequence of wild-type human EZH2 in one or more amino acid residues in its substrate pocket domain as defined in SEQ ID NO: 6. The mutant of EZH2 according to this embodiment is referred to herein as an EZH2 mutant.

Mutant EZH2 comprising one or more mutations in
the substrate pocket domain
(SEQ ID NO: 18)
MGQTGKKSEKGPVCWRKRVKSEYMRLRQLKRERRADEVKSMESSNRQKIL
ERTEILNQEWKQRRIQPVHILTSVSSLRGTRECSVTSDLDFPTQVIPLKT
LNAVASVPIMYSWSPLQQNFMVEDETVLHNIPYMGDEVLDQDGTFIEELI
KNYDGKVHGDRECGFINDEIFVELVNALGQYNDDDDDDDGDDPEEREEKQ
KDLEDHRDDKESRPPRKFPSDKIFEAISSMFPDKGTAEELKEKYKELTEQ
QLPGALPPECTPNIDGPNAKSVQREQSLHSFHTLFCRRCFKYDCFLHPFH
ATPNTYKRKNTETALDNKPCGPQCYQHLEGAKEFAAALTAERIKTPPKRP
GGRRRGRLPNNSSRPSTPTINVLESKDTDSDREAGTETGGENNDKEEEEK
KDETSSSSEANSRCQTPIKMKPNIEPPENVEWSGAEASMFRVLIGTYYDN
FCAIARLIGTKTCRQVYEFRVKESSIIAPAPAEDVDTPPRKKKRKHRLWA
AHCRKIQLKKDGSSNHVYNYQPCDHPRQPCDSSCPCVIAQNFCEKFCQCS
SECQNRFPGCRCKAQCNTKQCPCYLAVRECDPDLCLTCGAADHWDSKNVS
CKNCSIQRGSKKHLLLAPSDVAGWGIFIKDPVQKNEFISEXCGEIISQDE
ADRRGKVYDKYMXXXLXNLNNDFXXDXTRKGNKXXXXHSVNPNCYAKVMM
VNGDHRXGIFAKRAIQTGEELFXDXRYSXADALKYVGIEREMEIP
Wherein X can be any amino acid except the
corresponding wild type residue.

### Histone Acetyltransferases

Histone acetyltransferase (HAT) enzymes of the disclosure activate gene transcription by transferring an acetyl group from acetyl CoA to form ε-N-acetyllysine, which serves to modify histones and increase transcription by, for example, generating or exposing binding sites for protein-protein interaction domains.

HAT enzymes of the disclosure include, but are not limited to, those enzymes of the p300/CBP family.

In certain embodiments, a mutation of the disclosure may occur in a sequence encoding the p300 HAT, including the nucleotide sequence of the EP300 gene, encoding p300 (below, corresponding to GenBank Accession No. NM_001429.3, defined as Homo sapiens E1A binding protein p300 (EP300), mRNA; and identified as SEQ ID NO: 19).

| | |
|---|---|
| 1 | GCCGAGGAGG AAGAGGTTGA TGGCGGCGGC GGAGCTCCGA GAGACCTCGG CTGGGCAGGG |
| 61 | GCCGGCCGTG GCGGGCCGGG GACTGCGCCT CTAGAGCCGC GAGTTCTCGG GAATTCGCCG |
| 121 | CAGCGGACGC GCTCGGCGAA TTTGTGCTCT TGTGCCCTCC TCCGGGCTTG GGCCCAGGCC |
| 181 | CGGCCCCTCG CACTTGCCCT TACCTTTTCT ATCGAGTCCG CATCCCTCTC CAGCCACTGC |
| 241 | GACCCGGCGA AGAGAAAAAG GAACTTCCCC CACCCCCTCG GGTGCCGTCG GAGCCCCCCA |
| 301 | GCCCACCCCT GGGTGCGGCG CGGGGACCCC GGGCCGAAGA AGAGATTTCC TGAGGATTCT |
| 361 | GGTTTTCCTC GCTTGTATCT CCGAAAGAAT TAAAAATGGC CGAGAATGTG GTGGAACCGG |
| 421 | GGCCGCCTTC AGCCAAGCGG CCTAAACTCT CATCTCCGGC CCTCTCGGCG TCCGCCAGCG |
| 481 | ATGGCACAGA TTTTGGCTCT CTATTTGACT TGGAGCACGA CTTACCAGAT GAATTAATCA |
| 541 | ACTCTACAGA ATTGGGACTA ACCAATGGTG GTGATATTAA TCAGCTTCAG ACAAGTCTTG |
| 601 | GCATGGTACA AGATGCAGCT TCTAAACATA AACAGCTGTC AGAATTGCTG CGATCTGGTA |
| 661 | GTTCCCCTAA CCTCAATATG GGAGTTGGTG GCCCAGGTCA AGTCATGGCC AGCCAGGCCC |
| 721 | AACAGAGCAG TCCTGGATTA GGTTTGATAA ATAGCATGGT CAAAAGCCCA ATGACACAGG |
| 781 | CAGGCTTGAC TTCTCCCAAC ATGGGGATGG GCACTAGTGG ACCAAATCAG GGTCCTACGC |
| 841 | AGTCAACAGG TATGATGAAC AGTCCAGTAA ATCAGCCTGC CATGGGAATG AACACAGGGA |
| 901 | TGAATGCGGG CATGAATCCT GGAATGTTGG CTGCAGGCAA TGGACAAGGG ATAATGCCTA |
| 961 | ATCAAGTCAT GAACGGTTCA ATTGGAGCAG GCCGAGGGCG ACAGAATATG CAGTACCCAA |
| 1021 | ACCCAGGCAT GGGAAGTGCT GGCAACTTAC TGACTGAGCC TCTTCAGCAG GGCTCTCCCC |
| 1081 | AGATGGGAGG ACAAACAGGA TTGAGAGGCC CCCAGCCTCT TAAGATGGGA ATGATGAACA |
| 1141 | ACCCCAATCC TTATGGTTCA CCATATACTC AGAATCCTGG ACAGCAGATT GGAGCCAGTG |
| 1201 | GCCTTGGTCT CCAGATTCAG ACAAAAACTG TACTATCAAA TAACTTATCT CCATTTGCTA |
| 1261 | TGGACAAAAA GGCAGTTCCT GGTGGAGGAA TGCCCAACAT GGGTCAACAG CCAGCCCCGC |
| 1321 | AGGTCCAGCA GCCAGGCCTG GTGACTCCAG TTGCCCAAGG GATGGGTTCT GGAGCACATA |
| 1381 | CAGCTGATCC AGAGAAGCGC AAGCTCATCC AGCAGCAGCT TGTTCTCCTT TTGCATGCTC |
| 1441 | ACAAGTGCCA GCGCCGGGAA CAGGCCAATG GGGAAGTGAG GCAGTGCAAC CTTCCCCACT |
| 1501 | GTCGCACAAT GAAGAATGTC CTAAACCACA TGACACACTG CCAGTCAGGC AAGTCTTGCC |
| 1561 | AAGTGGCACA CTGTGCATCT TCTCGACAAA TCATTTCACA CTGGAAGAAT TGTACAAGAC |
| 1621 | ATGATTGTCC TGTGTGTCTC CCCCTCAAAA ATGCTGGTGA TAAGAGAAAT CAACAGCCAA |
| 1681 | TTTTGACTGG AGCACCCGTT GGACTTGGAA ATCCTAGCTC TCTAGGGGTG GGTCAACAGT |
| 1741 | CTGCCCCCAA CCTAAGCACT GTTAGTCAGA TTGATCCCAG CTCCATAGAA AGAGCCTATG |
| 1801 | CAGCTCTTGG ACTACCCTAT CAAGTAAATC AGATGCCGAC ACAACCCCAG GTGCAAGCAA |
| 1861 | AGAACCAGCA GAATCAGCAG CCTGGGCAGT CTCCCCAAGG CATGCGGCCC ATGAGCAACA |
| 1921 | TGAGTGCTAG TCCTATGGGA GTAAATGGAG GTGTAGGAGT TCAAACGCCG AGTCTTCTTT |
| 1981 | CTGACTCAAT GTTGCATTCA GCCATAAATT CTCAAAACCC AATGATGAGT GAAAATGCCA |
| 2041 | GTGTGCCCTC CCTGGGTCCT ATGCCAACAG CAGCTCAACC ATCCACTACT GGAATTCGGA |
| 2101 | AACAGTGGCA CGAAGATATT ACTCAGGATC TTCGAAATCA TCTTGTTCAC AAACTCGTCC |
| 2161 | AAGCCATATT TCCTACGCCG GATCCTGCTG CTTTAAAAGA CAGACGGATG GAAAACCTAG |
| 2221 | TTGCATATGC TCGGAAAGTT GAAGGGGACA TGTATGAATC TGCAAACAAT CGAGCGGAAT |
| 2281 | ACTACCACCT TCTAGCTGAG AAAATCTATA AGATCCAGAA AGAACTAGAA GAAAAACGAA |
| 2341 | GGACCAGACT ACAGAAGCAG AACATGCTAC CAAATGCTGC AGGCATGGTT CCAGTTTCCA |
| 2401 | TGAATCCAGG GCCTAACATG GGACAGCCGC AACCAGGAAT GACTTCTAAT GGCCCTCTAC |
| 2461 | CTGACCCAAG TATGATCCGT GGCAGTGTGC CAAACCAGAT GATGCCTCGA ATAACTCCAC |
| 2521 | AATCTGGTTT GAATCAATTT GGCCAGATGA GCATGGCCCA GCCCCCTATT GTACCCCGGC |
| 2581 | AAACCCCTCC TCTTCAGCAC CATGGACAGT TGGCTCAACC TGGAGCTCTC AACCCGCCTA |
| 2641 | TGGGCTATGG GCCTCGTATG CAACAGCCTT CCAACCAGGG CCAGTTCCTT CCTCAGACTC |
| 2701 | AGTTCCCATC ACAGGGAATG AATGTAACAA ATATCCCTTT GGCTCCGTCC AGCGGTCAAG |
| 2761 | CTCCAGTGTC TCAAGCACAA ATGTCTAGTT CTTCCTGCCC GGTGAACTCT CCTATAATGC |
| 2821 | CTCCAGGGTC TCAGGGGAGC CACATTCACT GTCCCCAGCT TCCTCAACCA GCTCTTCATC |
| 2881 | AGAATTCACC CTCGCCTGTA CCTAGTCGTA CCCCCACCCC TCACCATACT CCCCCAAGCA |
| 2941 | TAGGGGCTCA GCAGCCACCA GCAACAACAA TTCCAGCCCC TGTTCCTACA CCTCCTGCCA |
| 3001 | TGCCACCTGG GCCACAGTCC CAGGCTCTAC ATCCCCCTCC AAGGCAGACA CCTACACCAC |
| 3061 | CAACAACACA ACTTCCCCAA CAAGTGCAGC CTTCACTTCC TGCTGCACCT TCTGCTGACC |
| 3121 | AGCCCCAGCA GCAGCCTCGC TCACAGCAGA GCACAGCAGC GTCTGTTCCT ACCCCAACAG |
| 3181 | CACCGCTGCT TCCTCCGCAG CCTGCAACTC CACTTTCCCA GCCAGCTGTA AGCATTGAAG |
| 3241 | GACAGGTATC AAATCCTCCA TCTACTAGTA GCACAGAAGT GAATTCTCAG GCCATTGCTG |
| 3301 | AGAAGCAGCC TTCCCAGGAA GTGAAGATGG AGGCCAAAAT GGAAGTGGAT CAACCAGAAC |
| 3361 | CAGCAGATAC TCAGCCGGAG GATATTTCAG AGTCTAAAGT GGAAGACTGT AAAATGGAAT |
| 3421 | CTACCGAAAC AGAAGAGAGA AGCACTGAGT TAAAAACTGA AATAAAAGAG GAGGAAGACC |
| 3481 | AGCCAAGTAC TTCAGCTACC CAGTCATCTC CGGCTCCAGG ACAGTCAAAG AAAAAGATTT |
| 3541 | TCAAACCAGA AGAACTACGA CAGGCACTGA TGCCAACTTT GGAGGCACTT TACCGTCAGG |
| 3601 | ATCCAGAATC CCTTCCCTTT CGTCAACCTG TGGACCCTCA GCTTTTAGGA ATCCCTGATT |
| 3661 | ACTTTGATAT TGTGAAGAGC CCCATGGATC TTTCTACCAT TAAGAGGAAG TTAGACACTG |
| 3721 | GACAGTATCA GGAGCCCTGG CAGTATGTCG ATGATATTTG GCTTATGTTC AATAATGCCT |
| 3781 | GGTTATATAA CCGGAAAACA TCACGGGTAT ACAAATACTG CTCCAAGCTC TCTGAGGTCT |
| 3841 | TTGAACAAGA AATTGACCCA GTGATGCAAA GCCTTGGATA CTGTTGTGGC AGAAAGTTGG |
| 3901 | AGTTCTCTCC ACAGACACTG TGTTGCTACG GCAAACAGTT GTGCACAATA CCTCGTGATG |
| 3961 | CCACTTATTA CAGTTACCAG AACAGGTATC ATTTCTGTGA GAAGTGTTTC AATGAGATCC |
| 4021 | AAGGGGAGAG CGTTTCTTTG GGGGATGACC CTTCCCAGCC TCAAACTACA ATAAATAAAG |
| 4081 | AACAATTTTC CAAGAGAAAA AATGACACAC TGGATCCTGA ACTGTTTGTT GAATGTACAG |
| 4141 | AGTGCGGAAG AAAGATGCAT CAGATCTGTG TCCTTCACCA TGAGATCATC TGGCCTGCTG |
| 4201 | GATTCGTCTG TGATGGCTGT TTAAAGAAAA GTGCACGAAC TAGGAAAGAA AATAAGTTTT |
| 4261 | CTGCTAAAAG GTTGCCATCT ACCAGACTTG GCACCTTTCT AGAGAATCGT GTGAATGACT |
| 4321 | TTCTGAGGCG ACAGAATCAC CCTGAGTCAG GAGAGGTCAC TGTTAGAGTA GTTCATGCTT |
| 4381 | CTGACAAAAC CGTGGAAGTA AAACCAGGCA TGAAAGCAAG GTTTGTGGAC AGTGGAGAGA |
| 4441 | TGGCAGAATC CTTTCCATAC CGAACCAAAG CCCTCTTTGC CTTTGAAGAA ATTGATGGTG |
| 4501 | TTGACCTGTG CTTCTTTGGC ATGCATGTTC AAGAGTATGG CTCTGACTGC CCTCCACCCA |
| 4561 | ACCAGAGGAG AGTATACATA TCTTACCTCG ATAGTGTTCA TTTCTTCCGT CCTAAATGCT |
| 4621 | TGAGGACTGC AGTCTATCAT GAAATCCTAA TTGGATATTT AGAATATGTC AAGAAATTAG |
| 4681 | GTTACACAAC AGGGCATATT TGGGCATGTC CACCAAGTGA GGGAGATGAT TATATCTTCC |
| 4741 | ATTGCCATCC TCCTGACCAG AAGATACCCA AGCCCAAGCG ACTGCAGGAA TGGTACAAAA |
| 4801 | AAATGCTTGA CAAGGCTGTA TCAGAGCGTA TTGTCCATGA CTACAAGGAT ATTTTTAAAC |
| 4861 | AAGCTACTGA AGATAGATTA ACAAGTGCAA AGGAATTGCC TTATTTCGAG GGTGATTTCT |
| 4921 | GGCCCAATGT TCTGGAAGAA AGCATTAAGG AACTGGAACA GGAGGAAGAA GAGAGAAAAC |
| 4981 | GAGAGGAAAA CACCAGCAAT GAAAGCACAG ATGTGACCAA GGGAGACAGC AAAAATGCTA |
| 5041 | AAAAGAAGAA TAATAAGAAA ACCAGCAAAA ATAAGAGCAG CCTGAGTAGG GGCAACAAGA |
| 5101 | AGAAACCCGG GATGCCCAAT GTATCTAACG ACCTCTCACA GAAACTATAT GCCACCATGG |
| 5161 | AGAAGCATAA AGAGGTCTTC TTTGTGATCC GCCTCATTGC TGGCCCTGCT GCCAACTCCC |
| 5221 | TGCCTCCCAT TGTTGATCCT GATCCTCTCA TCCCCTGCGA TCTGATGGAT GGTCGGGATG |
| 5281 | CGTTTCTCAC GCTGGCAAGG GACAAGCACC TGGAGTTCTC TTCACTCCGA AGAGCCCAGT |
| 5341 | GGTCCACCAT GTGCATGCTG GTGGAGCTGC ACACGCAGAG CCAGGACCGC TTTGTCTACA |
| 5401 | CCTGCAATGA ATGCAAGCAC CATGTGGAGA CACGCTGGCA CTGTACTGTC TGTGAGGATT |
| 5461 | ATGACTTGTG TATCACCTGC TATAACACTA AAAACCATGA CCACAAAATG GAGAAACTAG |
| 5521 | GCCTTGGCTT AGATGATGAG AGCAACAACC AGCAGGCTGC AGCCACCCAG AGCCCAGGCG |
| 5581 | ATTCTCGCCG CCTGAGTATC CAGCGCTGCA TCCAGTCTCT GGTCCATGCT TGCCAGTGTC |
| 5641 | GGAATGCCAA TTGCTCACTG CCATCCTGCC AGAAGATGAA GCGGGTTGTG CAGCATACCA |
| 5701 | AGGGTTGCAA ACGGAAAACC AATGGCGGGT GCCCCATCTG CAAGCAGCTC ATTGCCCTCT |
| 5761 | GCTGCTACCA TGCCAAGCAC TGCCAGGAGA ACAAATGCCC GGTGCCGTTC TGCCTAAACA |
| 5821 | TCAAGCAGAA GCTCCGGCAG CAACAGCTGC AGCACCGACT ACAGCAGGCC CAAATGCTTC |
| 5881 | GCAGGAGGAT GGCCAGCATG CAGCGGACTG GTGTGGTTGG GCAGCAACAG GGCCTCCCTT |
| 5941 | CCCCCACTCC TGCCACTCCA ACGACACCAA CTGGCCAACA GCCAACCACC CCGCAGACGC |
| 6001 | CCCAGCCCAC TTCTCAGCCT CAGCCTACCC CTCCCAATAG CATGCCACCC TACTTGCCCA |
| 6061 | GGACTCAAGC TGCTGGCCCT GTGTCCCAGG GTAAGGCAGC AGGCCAGGTG ACCCCTCCAA |
| 6121 | CCCCTCCTCA GACTGCTCAG CCACCCCTTC CAGGGCCCCC ACCTGCAGCA GTGGAAATGG |
| 6181 | CAATGCAGAT TCAGAGAGCA GCGGAGACGC AGCGCCAGAT GGCCCACGTG CAAATTTTTC |
| 6241 | AAAGGCCAAT CCAACACCAG ATGCCCCCGA TGACTCCCAT GGCCCCCATG GGTATGAACC |
| 6301 | CACCTCCCAT GACCAGAGGT CCCAGTGGGC ATTTGGAGCC AGGGATGGGA CCGACAGGGA |
| 6361 | TGCAGCAACA GCCACCCTGG AGCCAAGGAG GATTGCCTCA GCCCCAGCAA CTACAGTCTG |
| 6421 | GGATGCCAAG GCCAGCCATG ATGTCAGTGG CCCAGCATGG TCAACCTTTG AACATGGCTC |
| 6481 | CACAACCAGG ATTGGGCCAG GTAGGTATCA GCCCACTCAA ACCAGGCACT GTGTCTCAAC |
| 6541 | AAGCCTTACA AAACCTTTTG CGGACTCTCA GGTCTCCCAG CTCTCCCCTG CAGCAGCAAC |
| 6601 | AGGTGCTTAG TATCCTTCAC GCCAACCCCC AGCTGTTGGC TGCATTCATC AAGCAGCGGG |
| 6661 | CTGCCAAGTA TGCCAACTCT AATCCACAAC CCATCCCTGG GCAGCCTGGC ATGCCCCAGG |
| 6721 | GGCAGCCAGG GCTACAGCCA CCTACCATGC CAGGTCAGCA GGGGGTCCAC TCCAATCCAG |
| 6781 | CCATGCAGAA CATGAATCCA ATGCAGGCGG GCGTTCAGAG GGCTGGCCTG CCCCAGCAGC |
| 6841 | AACCACAGCA GCAACTCCAG CCACCCATGG GAGGGATGAG CCCCCAGGCT CAGCAGATGA |
| 6901 | ACATGAACCA CAACACCATG CCTTCACAAT TCCGAGACAT CTTGAGACGA CAGCAAATGA |
| 6961 | TGCAACAGCA GCAGCAACAG GGAGCAGGGC CAGGAATAGG CCCTGGAATG GCCAACCATA |
| 7021 | ACCAGTTCCA GCAACCCCAA GGAGTTGGCT ACCCACCACA GCAGCAGCAG CGGATGCAGC |
| 7081 | ATCACATGCA ACAGATGCAA CAAGGAAATA TGGGACAGAT AGGCCAGCTT CCCCAGGCCT |
| 7141 | TGGGAGCAGA GGCAGGTGCC AGTCTACAGG CCTATCAGCA GCGACTCCTT CAGCAACAGA |
| 7201 | TGGGGTCCCC TGTTCAGCCC AACCCCATGA GCCCCCAGCA GCATATGCTC CCAAATCAGG |
| 7261 | CCCAGTCCCC ACACCTACAA GGCCAGCAGA TCCCTAATTC TCTCTCCAAT CAAGTGCGCT |
| 7321 | CTCCCCAGCC TGTCCCTTCT CCACGGCCAC AGTCCCAGCC CCCCCACTCC AGTCCTTCCC |
| 7381 | CAAGGATGCA GCCTCAGCCT TCTCCACACC ACGTTTCCCC ACAGACAAGT TCCCCACATC |
| 7441 | CTGGACTGGT AGCTGCCCAG GCCAACCCCA TGGAACAAGG GCATTTTGCC AGCCCGGACC |
| 7501 | AGAATTCAAT GCTTTCTCAG CTTGCTAGCA ATCCAGGCAT GGCAAACCTC CATGGTGCAA |
| 7561 | GCGCCACGGA CCTGGGACTC AGCACCGATA ACTCAGACTT GAATTCAAAC CTCTCACAGA |
| 7621 | GTACACTAGA CATACACTAG AGACACCTTG TAGTATTTTG GGAGCAAAAA AATTATTTTC |
| 7681 | TCTTAACAAG ACTTTTTGTA CTGAAAACAA TTTTTTTGAA TCTTTCGTAG CCTAAAAGAC |
| 7741 | AATTTTCCTT GGAACACATA AGAACTGTGC AGTAGCCGTT TGTGGTTTAA AGCAAACATG |
| 7801 | CAAGATGAAC CTGAGGGATG ATAGAATACA AAGAATATAT TTTTGTTATG GCTGGTTACC |
| 7861 | ACCAGCCTTT CTTCCCCTTT GTGTGTGTGG TTCAAGTGTG CACTGGGAGG AGGCTGAGGC |
| 7921 | CTGTGAAGCC AAACAATATG CTCCTGCCTT GCACCTCCAA TAGGTTTTAT TATTTTTTTT |
| 7981 | AAATTAATGA ACATATGTAA TATTAATAGT TATTATTTAC TGGTGCAGAT GGTTGACATT |
| 8041 | TTTCCCTATT TTCCTCACTT TATGGAAGAG TTAAAACATT TCTAAACCAG AGGACAAAAG |
| 8101 | GGGTTAATGT TACTTTAAAA TTACATTCTA TATATATATA AATATATATA AATATATATT |
| 8161 | AAAATACCAG TTTTTTTTCT CTGGGTGCAA AGATGTTCAT TCTTTTAAAA AATGTTTAAA |
| 8221 | AAAAAAAAAA AACTGCCTTT CTTCCCCTCA AGTCAACTTT TGTGCTCCAG AAAATTTTCT |
| 8281 | ATTCTGTAAG TCTGAGCGTA AAACTTCAAG TATTAAAATA ATTTGTACAT GTAGAGAGAA |
| 8341 | AAATGACTTT TTCAAAAATA TACAGGGGCA GCTGCCAAAT TGATGTATTA TATATTGTGG |
| 8401 | TTTCTGTTTC TTGAAAGAAT TTTTTTCGTT ATTTTTACAT CTAACAAAGT AAAAAAATTA |
| 8461 | AAAAGAGGGT AAGAAACGAT TCCGGTGGGA TGATTTTAAC ATGCAAAATG TCCCTGGGGG |
| 8521 | TTTCTTCTTT GCTTGCTTTC TTCCTCCTTA CCCTACCCCC CACTCACACA CACACACACA |
| 8581 | CACACACACA CACACACACA CACACACTTT CTATAAAACT TGAAAATAGC AAAAACCCTC |
| 8641 | AACTGTTGTA AATCATGCAA TTAAAGTTGA TTACTTATAA ATATGAACTT TGGATCACTG |
| 8701 | TATAGACTGT TAAATTTGAT TTCTTATTAC CTATTGTTAA ATAAACTGTG TGAGACAGAC |
| 8761 | A |

In certain embodiments, a mutation of the disclosure may occur in a sequence encoding the p300 HAT, including the amino acid sequence of the p300 protein (below, corresponding to GenBank Accession No. NP 001420.2, defined as Homo sapiens E1A-binding protein, 300 kD; E1A-associated protein p300; p300 HAT; and identified as SEQ ID NO: 20).

| | |
|---|---|
| 1 | MAENVVEPGP PSAKRPKLSS PALSASASDG TDFGSLFDLE HDLPDELINS TELGLTNGGD |
| 61 | INQLQTSLGM VQDAASKHKQ LSELLRSGSS PNLNMGVGGP GQVMASQAQQ SSPGLGLINS |
| 121 | MVKSPMTQAG LTSPNMGMGT SGPNQGPTQS TGMMNSPVNQ PAMGMNTGMN AGMNPGMLAA |
| 181 | GNGQGIMPNQ VMNGSIGAGR GRQNMQYPNP GMGSAGNLLT EPLQQGSPQM GGQTGLRGPQ |
| 241 | PLKMGMMNNP NPYGSPYTQN PGQQIGASGL GLQIQTKTVL SNNLSPFAMD KKAVPGGGMP |
| 301 | NMGQQPAPQV QQPGLVTPVA QGMGSGAHTA DPEKRKLIQQ QLVLLLHAHK CQRREQANGE |
| 361 | VRQCNLPHCR TMKNVLNHMT HCQSGKSCQV AHCASSRQII SHWKNCTRHD CPVCLPLKNA |
| 421 | GDKRNQQPIL TGAPVGLGNP SSLGVGQQSA PNLSTVSQID PSSIERAYAA LGLPYQVNQM |
| 481 | PTQPQVQAKN QQNQQPGQSP QGMRPMSNMS ASPMGVNGGV GVQTPSLLSD SMLHSAINSQ |
| 541 | NPMMSENASV PSLGPMPTAA QPSTTGIRKQ WHEDITQDLR NHLVHKLVQA IFPTPDPAAL |
| 601 | KDRRMENLVA YARKVEGDMY ESANNRAEYY HLLAEKIYKI QKELEEKRRT RLQKQNMLPN |
| 661 | AAGMVPVSMN PGPNMGQPQP GMTSNGPLPD PSMIRGSVPN QMMPRITPQS GLNQFGQMSM |
| 721 | AQPPIVPRQT PPLQHHGQLA QPGALNPPMG YGPRMQQPSN QGQFLPQTQF PSQGMNVTNI |
| 781 | PLAPSSGQAP VSQAQMSSSS CPVNSPIMPP GSQGSHIHCP QLPQPALHQN SPSPVPSRTP |
| 841 | TPHHTPPSIG AQQPPATTIP APVPTPPAMP PGPQSQALHP PPRQTPTPPT TQLPQQVQPS |
| 901 | LPAAPSADQP QQQPRSQQST AASVPTPTAP LLPPQPATPL SQPAVSIEGQ VSNPPSTSST |
| 961 | EVNSQAIAEK QPSQEVKMEA KMEVDQPEPA DTQPEDISES KVEDCKMEST ETEERSTELK |
| 1021 | TEIKEEEDQP STSATQSSPA PGQSKKKIFK PEELRQALMP TLEALYRQDP ESLPFRQPVD |
| 1081 | PQLLGIPDYF DIVKSPMDLS TIKRKLDTGQ YQEPWQYVDD IWLMFNNAWL YNRKTSRVYK |
| 1141 | YCSKLSEVFE QEIDPVMQSL GYCCGRKLEF SPQTLCCYGK QLCTIPRDAT YYSYQNRYHF |
| 1201 | CEKCFNEIQG ESVSLGDDPS QPQTTINKEQ FSKRKNDTLD PELFVECTEC GRKMHQICVL |
| 1261 | HHEIIWPAGF VCDGCLKKSA RTRKENKFSA KRLPSTRLGT FLENRVNDFL RRQNHPESGE |
| 1321 | VTVRVVHASD KTVEVKPGMK ARFVDSGEMA ESFPYRTKAL FAFEEIDGVD LCFFGMHVQE |
| 1381 | YGSDCPPPNQ RRVYISYLDS VHFFRPKCLR TAVYHEILIG YLEYVKKLGY TTGHIWACPP |
| 1441 | SEGDDYIFHC HPPDQKIPKP KRLQEWYKKM LDKAVSERIV HDYKDIFKQA TEDRLTSAKE |
| 1501 | LPYFEGDFWP NVLEESIKEL EQEEEERKRE ENTSNESTDV TKGDSKNAKK KNNKKTSKNK |
| 1561 | SSLSRGNKKK PGMPNVSNDL SQKLYATMEK HKEVFFVIRL IAGPAANSLP PIVDPDPLIP |
| 1621 | CDLMDGRDAF LTLARDKHLE FSSLRRAQWS TMCMLVELHT QSQDRFVYTC NECKHHVETR |
| 1681 | WHCTVCEDYD LCITCYNTKN HDHKMEKLGL GLDDESNNQQ AAATQSPGDS RRLSIQRCIQ |
| 1741 | SLVHACQCRN ANCSLPSCQK MKRVVQHTKG CKRKTNGGCP ICKQLIALCC YHAKHCQENK |
| 1801 | CPVPFCLNIK QKLRQQQLQH RLQQAQMLRR RMASMQRTGV VGQQQGLPSP TPATPTTPTG |
| 1861 | QQPTTPQTPQ PTSQPQPTPP NSMPPYLPRT QAAGPVSQGK AAGQVTPPTP PQTAQPPLPG |
| 1921 | PPPAAVEMAM QIQRAAETQR QMAHVQIFQR PIQHQMPPMT PMAPMGMNPP PMTRGPSGHL |
| 1981 | EPGMGPTGMQ QQPPWSQGGL PQPQQLQSGM PRPAMMSVAQ HGQPLNMAPQ PGLGQVGISP |
| 2041 | LKPGTVSQQA LQNLLRTLRS PSSPLQQQQV LSILHANPQL LAAFIKQRAA KYANSNPQPI |
| 2101 | PGQPGMPQGQ PGLQPPTMPG QQGVHSNPAM QNMNPMQAGV QRAGLPQQQP QQQLQPPMGG |
| 2161 | MSPQAQQMNM NHNTMPSQFR DILRRQQMMQ QQQQQGAGPG IGPGMANHNQ FQQPQGVGYP |
| 2221 | PQQQQRMQHH MQQMQQGNMG QIGQLPQALG AEAGASLQAY QQRLLQQQMG SPVQPNPMSP |
| 2281 | QQHMLPNQAQ SPHLQGQQIP NSLSNQVRSP QPVPSPRPQS QPPHSSPSPR MQPQPSPHHV |
| 2341 | SPQTSSPHPG LVAAQANPME QGHFASPDQN SMLSQLASNP GMANLHGASA TDLGLSTDNS |
| 2401 | DLNSNLSQST LDIH |

In certain embodiments, a mutation of the disclosure may occur in a sequence encoding the CREB Binding Protein (CREBBP) HAT, including the nucleotide sequence encoding CREBBP (below, corresponding to GenBank Accession No. NM_004380, defined as Homo sapiens CREB binding protein (CREBBP), transcript variant 1, mRNA; and identified as SEQ ID NO: 23).

| | |
|---|---|
| 1 | CTGCGGGGCG CTGTTGCTGT GGCTGAGATT TGGCCGCCGC CTCCCCCACC CGGCCTGCGC |
| 61 | CCTCCCTCTC CCTCGGCGCC CGCCCGCCCG CTCGCGGCCC GCGCTCGCTC CTCTCCCTCG |
| 121 | CAGCCGGCAG GGCCCCCGAC CCCCGTCCGG GCCCTCGCCG GCCCGGCCGC CCGTGCCCGG |
| 181 | GGCTGTTTTC GCGAGCAGGT GAAAATGGCT GAGAACTTGC TGGACGGACC GCCCAACCCC |
| 241 | AAAAGAGCCA AACTCAGCTC GCCCGGTTTC TCGGCGAATG ACAGCACAGA TTTTGGATCA |
| 301 | TTGTTTGACT TGGAAAATGA TCTTCCTGAT GAGCTGATAC CCAATGGAGG AGAATTAGGC |
| 361 | CTTTTAAACA GTGGGAACCT TGTTCCAGAT GCTGCTTCCA AACATAAACA ACTGTCGGAG |
| 421 | CTTCTACGAG GAGGCAGCGG CTCTAGTATC AACCCAGGAA TAGGAAATGT GAGCGCCAGC |
| 481 | AGCCCCGTGC AGCAGGGCCT GGGTGGCCAG GCTCAAGGGC AGCCGAACAG TGCTAACATG |
| 541 | GCCAGCCTCA GTGCCATGGG CAAGAGCCCT CTGAGCCAGG GAGATTCTTC AGCCCCCAGC |
| 601 | CTGCCTAAAC AGGCAGCCAG CACCTCTGGG CCCACCCCCG CTGCCTCCCA AGCACTGAAT |
| 661 | CCGCAAGCAC AAAAGCAAGT GGGGCTGGCG ACTAGCAGCC CTGCCACGTC ACAGACTGGA |
| 721 | CCTGGTATCT GCATGAATGC TAACTTTAAC CAGACCCACC CAGGCCTCCT CAATAGTAAC |
| 781 | TCTGGCCATA GCTTAATTAA TCAGGCTTCA CAAGGGCAGG CGCAAGTCAT GAATGGATCT |
| 841 | CTTGGGGCTG CTGGCAGAGG AAGGGGAGCT GGAATGCCGT ACCCTACTCC AGCCATGCAG |
| 901 | GGCGCCTCGA GCAGCGTGCT GGCTGAGACC CTAACGCAGG TTTCCCCGCA AATGACTGGT |
| 961 | CACGCGGGAC TGAACACCGC ACAGGCAGGA GGCATGGCCA AGATGGGAAT AACTGGGAAC |
| 1021 | ACAAGTCCAT TTGGACAGCC CTTTAGTCAA GCTGGAGGGC AGCCAATGGG AGCCACTGGA |
| 1081 | GTGAACCCCC AGTTAGCCAG CAAACAGAGC ATGGTCAACA GTTTGCCCAC CTTCCCTACA |
| 1141 | GATATCAAGA ATACTTCAGT CACCAACGTG CCAAATATGT CTCAGATGCA AACATCAGTG |
| 1201 | GGAATTGTAC CCACACAAGC AATTGCAACA GGCCCCACTG CAGATCCTGA AAAACGCAAA |
| 1261 | CTGATACAGC AGCAGCTGGT TCTACTGCTT CATGCTCATA AGTGTCAGAG ACGAGAGCAA |
| 1321 | GCAAACGGAG AGGTTCGGGC CTGCTCGCTC CCGCATTGTC GAACCATGAA AAACGTTTTG |
| 1381 | AATCACATGA CGCATTGTCA GGCTGGGAAA GCCTGCCAAG TTGCCCATTG TGCATCTTCA |
| 1441 | CGACAAATCA TCTCTCATTG GAAGAACTGC ACACGACATG ACTGTCCTGT TTGCCTCCCT |
| 1501 | TTGAAAAATG CCAGTGACAA GCGAAACCAA CAAACCATCC TGGGGTCTCC AGCTAGTGGA |
| 1561 | ATTCAAAACA CAATTGGTTC TGTTGGCACA GGGCAACAGA ATGCCACTTC TTTAAGTAAC |
| 1621 | CCAAATCCCA TAGACCCCAG CTCCATGCAG CGAGCCTATG CTGCTCTCGG ACTCCCCTAC |
| 1681 | ATGAACCAGC CCCAGACGCA GCTGCAGCCT CAGGTTCCTG GCCAGCAACC AGCACAGCCT |
| 1741 | CAAACCCACC AGCAGATGAG GACTCTCAAC CCCCTGGGAA ATAATCCAAT GAACATTCCA |
| 1801 | GCAGGAGGAA TAACAACAGA TCAGCAGCCC CCAAACTTGA TTTCAGAATC AGCTCTTCCG |
| 1861 | ACTTCCCTGG GGGCCACAAA CCCACTGATG AACGATGGCT CCAACTCTGG TAACATTGGA |
| 1921 | ACCCTCAGCA CTATACCAAC AGCAGCTCCT CCTTCTAGCA CCGGTGTAAG GAAAGGCTGG |
| 1981 | CACGAACATG TCACTCAGGA CCTGCGGAGC CATCTAGTGC ATAAACTCGT CCAAGCCATC |
| 2041 | TTCCCAACAC CTGATCCCGC AGCTCTAAAG GATCGCCGCA TGGAAAACCT GGTAGCCTAT |
| 2101 | GCTAAGAAAG TGGAAGGGGA CATGTACGAG TCTGCCAACA GCAGGGATGA ATATTATCAC |
| 2161 | TTATTAGCAG AGAAAATCTA CAAGATACAA AAAGAACTAG AAGAAAAACG GAGGTCGCGT |
| 2221 | TTACATAAAC AAGGCATCTT GGGGAACCAG CCAGCCTTAC CAGCCCCGGG GGCTCAGCCC |
| 2281 | CCTGTGATTC CACAGGCACA ACCTGTGAGA CCTCCAAATG GACCCCTGTC CCTGCCAGTG |
| 2341 | AATCGCATGC AAGTTTCTCA AGGGATGAAT TCATTTAACC CCATGTCCTT GGGGAACGTC |
| 2401 | CAGTTGCCAC AAGCACCCAT GGGACCTCGT GCAGCCTCCC CAATGAACCA CTCTGTCCAG |
| 2461 | ATGAACAGCA TGGGCTCAGT GCCAGGGATG GCCATTTCTC CTTCCCGAAT GCCTCAGCCT |
| 2521 | CCGAACATGA TGGGTGCACA CACCAACAAC ATGATGGCCC AGGCGCCCGC TCAGAGCCAG |
| 2581 | TTTCTGCCAC AGAACCAGTT CCCGTCATCC AGCGGGGCGA TGAGTGTGGG CATGGGGCAG |
| 2641 | CCGCCAGCCC AAACAGGCGT GTCACAGGGA CAGGTGCCTG GTGCTGCTCT TCCTAACCCT |
| 2701 | CTCAACATGC TGGGGCCTCA GGCCAGCCAG CTACCTTGCC CTCCAGTGAC ACAGTCACCA |
| 2761 | CTGCACCCAA CACCGCCTCC TGCTTCCACG GCTGCTGGCA TGCCATCTCT CCAGCACACG |
| 2821 | ACACCACCTG GGATGACTCC TCCCCAGCCA GCAGCTCCCA CTCAGCCATC AACTCCTGTG |
| 2881 | TCGTCTTCCG GGCAGACTCC CACCCCGACT CCTGGCTCAG TGCCCAGTGC TACCCAAACC |
| 2941 | CAGAGCACCC CTACAGTCCA GGCAGCAGCC CAGGCCCAGG TGACCCCGCA GCCTCAAACC |
| 3001 | CCAGTTCAGC CCCCGTCTGT GGCTACCCCT CAGTCATCGC AGCAACAGCC GACGCCTGTG |
| 3061 | CACGCCCAGC CTCCTGGCAC ACCGCTTTCC CAGGCAGCAG CCAGCATTGA TAACAGAGTC |
| 3121 | CCTACCCCCT CCTCGGTGGC CAGCGCAGAA ACCAATTCCC AGCAGCCAGG ACCTGACGTA |
| 3181 | CCTGTGCTGG AAATGAAGAC GGAGACCCAA GCAGAGGACA CTGAGCCCGA TCCTGGTGAA |
| 3241 | TCCAAAGGGG AGCCCAGGTC TGAGATGATG GAGGAGGATT TGCAAGGAGC TTCCCAAGTT |
| 3301 | AAAGAAGAAA CAGACATAGC AGAGCAGAAA TCAGAACCAA TGGAAGTGGA TGAAAAGAAA |
| 3361 | CCTGAAGTGA AAGTAGAAGT TAAAGAGGAA GAAGAGAGTA GCAGTAACGG CACAGCCTCT |
| 3421 | CAGTCAACAT CTCCTTCGCA GCCGCGCAAA AAAATCTTTA AACCAGAGGA GTTACGCCAG |
| 3481 | GCCCTCATGC CAACCCTAGA AGCACTGTAT CGACAGGACC CAGAGTCATT ACCTTTCCGG |
| 3541 | CAGCCTGTAG ATCCCCAGCT CCTCGGAATT CCAGACTATT TTGACATCGT AAAGAATCCC |
| 3601 | ATGGACCTCT CCACCATCAA GCGGAAGCTG GACACAGGGC AATACCAAGA GCCCTGGCAG |
| 3661 | TACGTGGACG ACGTCTGGCT CATGTTCAAC AATGCCTGGC TCTATAATCG CAAGACATCC |
| 3721 | CGAGTCTATA AGTTTTGCAG TAAGCTTGCA GAGGTCTTTG AGCAGGAAAT TGACCCTGTC |
| 3781 | ATGCAGTCCC TTGGATATTG CTGTGGACGC AAGTATGAGT TTTCCCCACA GACTTTGTGC |
| 3841 | TGCTATGGGA AGCAGCTGTG TACCATTCCT CGCGATGCTG CCTACTACAG CTATCAGAAT |
| 3901 | AGGTATCATT TCTGTGAGAA GTGTTTCACA GAGATCCAGG GCGAGAATGT GACCCTGGGT |
| 3961 | GACGACCCTT CACAGCCCCA GACGACAATT TCAAAGGATC AGTTTGAAAA GAAGAAAAAT |
| 4021 | GATACCTTAG ACCCCGAACC TTTCGTTGAT TGCAAGGAGT GTGGCCGGAA GATGCATCAG |
| 4081 | ATTTGCGTTC TGCACTATGA CATCATTTGG CCTTCAGGTT TTGTGTGCGA CAACTGCTTG |
| 4141 | AAGAAAACTG GCAGACCTCG AAAAGAAAAC AAATTCAGTG CTAAGAGGCT GCAGACCACA |
| 4201 | AGACTGGGAA ACCACTTGGA AGACCGAGTG AACAAATTTT TGCGGCGCCA GAATCACCCT |
| 4261 | GAAGCCGGGG AGGTTTTTGT CCGAGTGGTG GCCAGCTCAG ACAAGACGGT GGAGGTCAAG |
| 4321 | CCCGGGATGA AGTCACGGTT TGTGGATTCT GGGGAAATGT CTGAATCTTT CCCATATCGA |
| 4381 | ACCAAAGCTC TGTTTGCTTT TGAGGAAATT GACGGCGTGG ATGTCTGCTT TTTTGGAATG |
| 4441 | CACGTCCAAG AATACGGCTC TGATTGCCCC CCTCCAAACA CGAGGCGTGT GTACATTTCT |
| 4501 | TATCTGGATA GTATTCATTT CTTCCGGCCA CGTTGCCTCC GCACAGCCGT TTACCATGAG |
| 4561 | ATCCTTATTG GATATTTAGA GTATGTGAAG AAATTAGGGT ATGTGACAGG GCACATCTGG |
| 4621 | GCCTGTCCTC CAAGTGAAGG AGATGATTAC ATCTTCCATT GCCACCCACC TGATCAAAAA |
| 4681 | ATACCCAAGC CAAAACGACT GCAGGAGTGG TACAAAAAGA TGCTGGACAA GGCGTTTGCA |
| 4741 | GAGCGGATCA TCCATGACTA CAAGGATATT TTCAAACAAG CAACTGAAGA CAGGCTCACC |
| 4801 | AGTGCCAAGG AACTGCCCTA TTTTGAAGGT GATTTCTGGC CCAATGTGTT AGAAGAGAGC |
| 4861 | ATTAAGGAAC TAGAACAAGA AGAAGAGGAG AGGAAAAAGG AAGAGAGCAC TGCAGCCAGT |
| 4921 | GAAACCACTG AGGGCAGTCA GGGCGACAGC AAGAATGCCA AGAAGAAGAA CAACAAGAAA |
| 4981 | ACCAACAAGA ACAAAAGCAG CATCAGCCGC GCCAACAAGA AGAAGCCCAG CATGCCCAAC |
| 5041 | GTGTCCAATG ACCTGTCCCA GAAGCTGTAT GCCACCATGG AGAAGCACAA GGAGGTCTTC |
| 5101 | TTCGTGATCC ACCTGCACGC TGGGCCTGTC ATCAACACCC TGCCCCCCAT CGTCGACCCC |
| 5161 | GACCCCCTGC TCAGCTGTGA CCTCATGGAT GGGCGCGACG CCTTCCTCAC CCTCGCCAGA |
| 5221 | GACAAGCACT GGGAGTTCTC CTCCTTGCGC CGCTCCAAGT GGTCCACGCT CTGCATGCTG |
| 5281 | GTGGAGCTGC ACACCCAGGG CCAGGACCGC TTTGTCTACA CCTGCAACGA GTGCAAGCAC |
| 5341 | CACGTGGAGA CGCGCTGGCA CTGCACTGTG TGCGAGGACT ACGACCTCTG CATCAACTGC |
| 5401 | TATAACACGA AGAGCCATGC CCATAAGATG GTGAAGTGGG GGCTGGGCCT GGATGACGAG |
| 5461 | GGCAGCAGCC AGGGCGAGCC ACAGTCAAAG AGCCCCCAGG AGTCACGCCG GCTGAGCATC |
| 5521 | CAGCGCTGCA TCCAGTCGCT GGTGCACGCG TGCCAGTGCC GCAACGCCAA CTGCTCGCTG |
| 5581 | CCATCCTGCC AGAAGATGAA GCGGGTGGTG CAGCACACCA AGGGCTGCAA ACGCAAGACC |
| 5641 | AACGGGGGCT GCCCGGTGTG CAAGCAGCTC ATCGCCCTCT GCTGCTACCA CGCCAAGCAC |
| 5701 | TGCCAAGAAA ACAAATGCCC CGTGCCCTTC TGCCTCAACA TCAAACACAA GCTCCGCCAG |
| 5761 | CAGCAGATCC AGCACCGCCT GCAGCAGGCC CAGCTCATGC GCCGGCGGAT GGCCACCATG |
| 5821 | AACACCCGCA ACGTGCCTCA GCAGAGTCTG CCTTCTCCTA CCTCAGCACC GCCCGGGACC |
| 5881 | CCCACACAGC AGCCCAGCAC ACCCCAGACG CCGCAGCCCC CTGCCCAGCC CCAACCCTCA |
| 5941 | CCCGTGAGCA TGTCACCAGC TGGCTTCCCC AGCGTGGCCC GGACTCAGCC CCCCACCACG |
| 6001 | GTGTCCACAG GGAAGCCTAC CAGCCAGGTG CCGGCCCCCC CACCCCCGGC CCAGCCCCCT |
| 6061 | CCTGCAGCGG TGGAAGCGGC TCGGCAGATC GAGCGTGAGG CCCAGCAGCA GCAGCACCTG |
| 6121 | TACCGGGTGA ACATCAACAA CAGCATGCCC CCAGGACGCA CGGGCATGGG GACCCCGGGG |
| 6181 | AGCCAGATGG CCCCCGTGAG CCTGAATGTG CCCCGACCCA ACCAGGTGAG CGGGCCCGTC |
| 6241 | ATGCCCAGCA TGCCTCCCGG GCAGTGGCAG CAGGCGCCCC TTCCCCAGCA GCAGCCCATG |
| 6301 | CCAGGCTTGC CCAGGCCTGT GATATCCATG CAGGCCCAGG CGGCCGTGGC TGGGCCCCGG |
| 6361 | ATGCCCAGCG TGCAGCCACC CAGGAGCATC TCACCCAGCG CTCTGCAAGA CCTGCTGCGG |
| 6421 | ACCCTGAAGT CGCCCAGCTC CCCTCAGCAG CAACAGCAGG TGCTGAACAT TCTCAAATCA |
| 6481 | AACCCGCAGC TAATGGCAGC TTTCATCAAA CAGCGCACAG CCAAGTACGT GGCCAATCAG |
| 6541 | CCCGGCATGC AGCCCCAGCC TGGCCTCCAG TCCCAGCCCG GCATGCAACC CCAGCCTGGC |
| 6601 | ATGCACCAGC AGCCCAGCCT GCAGAACCTG AATGCCATGC AGGCTGGCGT GCCGCGGCCC |
| 6661 | GGTGTGCCTC CACAGCAGCA GGCGATGGGA GGCCTGAACC CCCAGGGCCA GGCCTTGAAC |
| 6721 | ATCATGAACC CAGGACACAA CCCCAACATG GCGAGTATGA ATCCACAGTA CCGAGAAATG |
| 6781 | TTACGGAGGC AGCTGCTGCA GCAGCAGCAG CAACAGCAGC AGCAACAACA GCAGCAACAG |
| 6841 | CAGCAGCAGC AAGGGAGTGC CGGCATGGCT GGGGGCATGG CGGGGCACGG CCAGTTCCAG |
| 6901 | CAGCCTCAAG GACCCGGAGG CTACCCACCG GCCATGCAGC AGCAGCAGCG CATGCAGCAG |
| 6961 | CATCTCCCCC TCCAGGGCAG CTCCATGGGC CAGATGGCGG CTCAGATGGG ACAGCTTGGC |
| 7021 | CAGATGGGGC AGCCGGGGCT GGGGGCAGAC AGCACCCCCA ACATCCAGCA AGCCCTGCAG |
| 7081 | CAGCGGATTC TGCAGCAACA GCAGATGAAG CAGCAGATTG GGTCCCCAGG CCAGCCGAAC |
| 7141 | CCCATGAGCC CCCAGCAACA CATGCTCTCA GGACAGCCAC AGGCCTCGCA TCTCCCTGGC |
| 7201 | CAGCAGATCG CCACGTCCCT TAGTAACCAG GTGCGGTCTC CAGCCCCTGT CCAGTCTCCA |
| 7261 | CGGCCCCAGT CCCAGCCTCC ACATTCCAGC CCGTCACCAC GGATACAGCC CCAGCCTTCG |
| 7321 | CCACACCACG TCTCACCCCA GACTGGTTCC CCCCACCCCG GACTCGCAGT CACCATGGCC |
| 7381 | AGCTCCATAG ATCAGGGACA CTTGGGGAAC CCCGAACAGA GTGCAATGCT CCCCCAGCTG |
| 7441 | AACACCCCCA GCAGGAGTGC GCTGTCCAGC GAACTGTCCC TGGTCGGGGA CACCACGGGG |
| 7501 | GACACGCTAG AGAAGTTTGT GGAGGGCTTG TAGCATTGTG AGAGCATCAC CTTTTCCCTT |
| 7561 | TCATGTTCTT GGACCTTTTG TACTGAAAAT CCAGGCATCT AGGTTCTTTT TATTCCTAGA |
| 7621 | TGGAACTGCG ACTTCCGAGC CATGGAAGGG TGGATTGATG TTTAAAGAAA CAATACAAAG |
| 7681 | AATATATTTT TTTGTTAAAA ACCAGTTGAT TTAAATATCT GGTCTCTCTC TTTGGTTTTT |
| 7741 | TTTTGGCGGG GGGGTGGGGG GGGTTCTTTT TTTTCCGTTT TGTTTTTGTT TGGGGGGAGG |
| 7801 | GGGGTTTTGT TTGGATTCTT TTTGTCGTCA TTGCTGGTGA CTCATGCCTT TTTTTAACGG |
| 7861 | GAAAAACAAG TTCATTATAT TCATATTTTT TATTTGTATT TTCAAGACTT TAAACATTTA |
| 7921 | TGTTTAAAAG TAAGAAGAAA AATAATATTC AGAACTGATT CCTGAAATAA TGCAAGCTTA |
| 7981 | TAATGTATCC CGATAACTTT GTGATGTTTC GGGAAGATTT TTTTCTATAG TGAACTCTGT |
| 8041 | GGGCGTCTCC CAGTATTACC CTGGATGATA GGAATTGACT CCGGCGTGCA CACACGTACA |
| 8101 | CACCCACACA CATCTATCTA TACATAATGG CTGAAGCCAA ACTTGTCTTG CAGATGTAGA |
| 8161 | AATTGTTGCT TTGTTTCTCT GATAAAACTG GTTTTAGACA AAAAATAGGG ATGATCACTC |
| 8221 | TTAGACCATG CTAATGTTAC TAGAGAAGAA GCCTTCTTTT CTTTCTTCTA TGTGAAACTT |
| 8281 | GAAATGAGGA AAAGCAATTC TAGTGTAAAT CATGCAAGCG CTCTAATTCC TATAAATACG |
| 8341 | AAACTCGAGA AGATTCAATC ACTGTATAGA ATGGTAAAAT ACCAACTCAT TTCTTATATC |
| 8401 | ATATTGTTAA ATAAACTGTG TGCAACAGAC AAAAAGGGTG GTCCTTCTTG AATTCATGTA |
| 8461 | CATGGTATTA ACACTTAGTG TTCGGGGTTT TTTGTTATGA AAATGCTGTT TTCAACATTG |
| 8521 | TATTTGGACT ATGCATGTGT TTTTTCCCCA TTGTATATAA AGTACCGCTT AAAATTGATA |
| 8581 | TAAATTACTG AGGTTTTTAA CATGTATTCT GTTCTTTAAG ATCCCTGTAA GAATGTTTAA |
| 8641 | GGTTTTTATT TATTTATATA TATTTTTTGA GTCTGTTCTT TGTAAGACAT GGTTCTGGTT |
| 8701 | GTTCGCTCAT AGCGGAGAGG CTGGGGCTGC GGTTGTGGTT GTGGCGGCGT GGGTGGTGGC |
| 8761 | TGGGAACTGT GGCCCAGGCT TAGCGGCCGC CCGGAGGCTT TTCTTCCCGG AGACTGAGGT |
| 8821 | GGGCGACTGA GGTGGGCGGC TCAGCGTTGG CCCCACACAT TCGAGGCTCA CAGGTGATTG |
| 8881 | TCGCTCACAC AGTTAGGGTC GTCAGTTGGT CTGAAACTGC ATTTGGCCCA CTCCTCCATC |
| 8941 | CTCCCTGTCC GTCGTAGCTG CCACCCCCAG AGGCGGCGCT TCTTCCCGTG TTCAGGCGGC |
| 9001 | TCCCCCCCCC CGTACACGAC TCCCAGAATC TGAGGCAGAG AGTGCTCCAG GCTCGCGAGG |
| 9061 | TGCTTTCTGA CTTCCCCCCA AATCCTGCCG CTGCCGCGCA GCATGTCCCG TGTGGCGTTT |
| 9121 | GAGGAAATGC TGAGGGACAG ACACCTTGGA GCACCAGCTC CGGTCCCTGT TACAGTGAGA |
| 9181 | AAGGTCCCCC ACTTCGGGGG ATACTTGCAC TTAGCCACAT GGTCCTGCCT CCCTTGGAGT |
| 9241 | CCAGTTCCAG GCTCCCTTAC TGAGTGGGTG AGACAAGTTC ACAAAAACCG TAAAACTGAG |
| 9301 | AGGAGGACCA TGGGCAGGGG AGCTGAAGTT CATCCCCTAA GTCTACCACC CCCAGCACCC |
| 9361 | AGAGAACCCA CTTTATCCCT AGTCCCCCAA CAAAGGCTGG TCTAGGTGGG GGTGATGGTA |
| 9421 | ATTTTAGAAA TCACGCCCCA AATAGCTTCC GTTTGGGCCC TTACATTCAC AGATAGGTTT |
| 9481 | TAAATAGCTG AATACTTGGT TTGGGAATCT GAATTCGAGG AACCTTTCTA AGAAGTTGGA |
| 9541 | AAGGTCCGAT CTAGTTTTAG CACAGAGCTT TGAACCTTGA GTTATAAAAT GCAGAATAAT |
| 9601 | TCAAGTAAAA ATAAGACCAC CATCTGGCAC CCCTGACCAG CCCCCATTCA CCCCATCCCA |
| 9661 | GGAGGGGAAG CACAGGCCGG GCCTCCGGTG GAGATTGCTG CCACTGCTCG GCCTGCTGGG |
| 9721 | TTCTTAACCT CCAGTGTCCT CTTCATCTTT TCCACCCGTA GGGAAACCTT GAGCCATGTG |
| 9781 | TTCAAACAAG AAGTGGGGCT AGAGCCCGAG AGCAGCAGCT CTAAGCCCAC ACTCAGAAAG |
| 9841 | TGGCGCCCTC CTGGTTGTGC AGCCTTTTAA TGTGGGCAGT GGAGGGGCCT CTGTTTCAGG |
| 9901 | TTATCCTGGA ATTCAAAACG TTATGTACCA ACCTCATCCT CTTTGGAGTC TGCATCCTGT |
| 9961 | GCAACCGTCT TGGGCAATCC AGATGTCGAA GGATGTGACC GAGAGCATGG TCTGTGGATG |
| 10021 | CTAACCCTAA GTTTGTCGTA AGGAAATTTC TGTAAGAAAC CTGGAAAGCC CCAACGCTGT |
| 10081 | GTCTCATGCT GTATACTTAA GAGGAGAAGA AAAAGTCCTA TATTTGTGAT CAAAAAGAGG |
| 10141 | AAACTTGAAA TGTGATGGTG TTTATAATAA AAGATGGTAA AACTACTTGG ATTCAAA |

In certain embodiments, a mutation of the disclosure may occur in a sequence encoding the CREB Binding Protein (CREBBP) HAT, including the amino acid sequence encoding CREBBP (below, corresponding to GenBank Accession No. NP 004371, defined as Homo sapiens CREB-binding protein isoform a; and identified as SEQ ID NO: 24).

| | |
|---|---|
| 1 | MAENLLDGPP NPKRAKLSSP GFSANDSTDF GSLFDLENDL |
| | PDELIPNGGE LGLLNSGNLV |
| 61 | PDAASKHKQL SELLRGGSGS SINPGIGNVS ASSPVQQGLG |
| | GQAQGQPNSA NMASLSAMGK |
| 121 | SPLSQGDSSA PSLPKQAAST SGPTPAASQA LNPQAQKQVG |
| | LATSSPATSQ TGPGICMNAN |
| 181 | FNQTHPGLLN SNSGHSLINQ ASQGQAQVMN GSLGAAGRGR |
| | GAGMPYPTPA MQGASSSVLA |
| 241 | ETLTQVSPQM TGHAGLNTAQ AGGMAKMGIT GNTSPFGQPF |
| | SQAGGQPMGA TGVNPQLASK |
| 301 | QSMVNSLPTF PTDIKNTSVT NVPNMSQMQT SVGIVPTQAI |
| | ATGPTADPEK RKLIQQQLVL |
| 361 | LLHAHKCQRR EQANGEVRAC SLPHCRTMKN VLNHMTHCQA |
| | GKACQVAHCA SSRQIISHWK |
| 421 | NCTRHDCPVC LPLKNASDKR NQQTILGSPA SGIQNTIGSV |
| | GTGQQNATSL SNPNPIDPSS |
| 481 | MQRAYAALGL PYMNQPQTQL QPQVPGQQPA QPQTHQQMRT |
| | LNPLGNNPMN IPAGGITTDQ |
| 541 | QPPNLISESA LPTSLGATNP LMNDGSNSGN IGTLSTIPTA |
| | APPSSTGVRK GWHEHVTQDL |
| 601 | RSHLVHKLVQ AIFPTPDPAA LKDRRMENLV AYAKKVEGDM |
| | YESANSRDEY YHLLAEKIYK |
| 661 | IQKELEEKRR SRLHKQGILG NQPALPAPGA QPPVIPQAQP |
| | VRPPNGPLSL PVNRMQVSQG |
| 721 | MNSFNPMSLG NVQLPQAPMG PRAASPMNHS VQMNSMGSVP |
| | GMAISPSRMP QPPNMMGAHT |
| 781 | NNMMAQAPAQ SQFLPQNQFP SSSGAMSVGM GQPPAQTGVS |
| | QGQVPGAALP NPLNMLGPQA |
| 841 | SQLPCPPVTQ SPLHPTPPPA STAAGMPSLQ HTTPPGMTPP |
| | QPAAPTQPST PVSSSGQTPT |
| 901 | PTPGSVPSAT QTQSTPTVQA AAQAQVTPQP QTPVQPPSVA |
| | TPQSSQQQPT PVHAQPPGTP |
| 961 | LSQAAASIDN RVPTPSSVAS AETNSQQPGP DVPVLEMKTE |
| | TQAEDTEPDP GESKGEPRSE |
| 1021 | MMEEDLQGAS QVKEETDIAE QKSEPMEVDE KKPEVKVEVK |
| | EEEESSSNGT ASQSTSPSQP |
| 1081 | RKKIFKPEEL RQALMPTLEA LYRQDPESLP FRQPVDPQLL |
| | GIPDYFDIVK NPMDLSTIKR |
| 1141 | KLDTGQYQEP WQYVDDVWLM FNNAWLYNRK TSRVYKFCSK |
| | LAEVFEQEID PVMQSLGYCC |
| 1201 | GRKYEFSPQT LCCYGKQLCT IPRDAAYYSY QNRYHFCEKC |
| | FTEIQGENVT LGDDPSQPQT |
| 1261 | TISKDQFEKK KNDTLDPEPF VDCKECGRKM HQICVLHYDI |
| | IWPSGFVCDN CLKKTGRPRK |
| 1321 | ENKFSAKRLQ TTRLGNHLED RVNKFLRRQN HPEAGEVFVR |
| | VVASSDKTVE VKPGMKSRFV |
| 1381 | DSGEMSESFP YRTKALFAFE EIDGVDVCFF GMHVQEYGSD |
| | CPPPNTRRVY ISYLDSIHFF |
| 1441 | RPRCLRTAVY HEILIGYLEY VKKLGYVTGH IWACPPSEGD |
| | DYIFHCHPPD QKIPKPKRLQ |
| 1501 | EWYKKMLDKA FAERIIHDYK DIFKQATEDR LTSAKELPYF |
| | EGDFWPNVLE ESIKELEQEE |
| 1561 | EERKKEESTA ASETTEGSQG DSKNAKKKNN KKTNKNKSSI |
| | SRANKKKPSM PNVSNDLSQK |
| 1621 | LYATMEKHKE VFFVIHLHAG PVINTLPPIV DPDPLLSCDL |
| | MDGRDAFLTL ARDKHWEFSS |
| 1681 | LRRSKWSTLC MLVELHTQGQ DRFVYTCNEC KHHVETRWHC |
| | TVCEDYDLCI NCYNTKSHAH |
| 1741 | KMVKWGLGLD DEGSSQGEPQ SKSPQESRRL SIQRCIQSLV |
| | HACQCRNANC SLPSCQKMKR |
| 1801 | VVQHTKGCKR KTNGGCPVCK QLIALCCYHA KHCQENKCPV |
| | PFCLNIKHKL RQQQIQHRLQ |
| 1861 | QAQLMRRRMA TMNTRNVPQQ SLPSPTSAPP GTPTQQPSTP |
| | QTPQPPAQPQ PSPVSMSPAG |
| 1921 | FPSVARTQPP TTVSTGKPTS QVPAPPPPAQ PPPAAVEAAR |
| | QIEREAQQQQ HLYRVNINNS |
| 1981 | MPPGRTGMGT PGSQMAPVSL NVPRPNQVSG PVMPSMPPGQ |
| | WQQAPLPQQQ PMPGLPRPVI |
| 2041 | SMQAQAAVAG PRMPSVQPPR SISPSALQDL LRTLKSPSSP |
| | QQQQQVLNIL KSNPQLMAAF |
| 2101 | IKQRTAKYVA NQPGMQPQPG LQSQPGMQPQ PGMHQQPSLQ |
| | NLNAMQAGVP RPGVPPQQQA |
| 2161 | MGGLNPQGQA LNIMNPGHNP NMASMNPQYR EMLRRQLLQQ |
| | QQQQQQQQQQ QQQQQQGSAG |
| 2221 | MAGGMAGHGQ FQQPQGPGGY PPAMQQQQRM QQHLPLQGSS |
| | MGQMAAQMGQ LGQMGQPGLG |
| 2281 | ADSTPNIQQA LQQRILQQQQ MKQQIGSPGQ PNPMSPQQHM |
| | LSGQPQASHL PGQQIATSLS |
| 2341 | NQVRSPAPVQ SPRPQSQPPH SSPSPRIQPQ PSPHHVSPQT |
| | GSPHPGLAVT MASSIDQGHL |
| 2401 | GNPEQSAMLP QLNTPSRSAL SSELSLVGDT TGDTLEKFVE |
| | GL |

In certain embodiments, a mutation of the disclosure may occur in a sequence encoding the CREB Binding Protein (CREBBP) HAT, including the nucleotide sequence encoding CREBBP (below, corresponding to GenBank Accession No. NM_001079846, defined as Homo sapiens CREB binding protein (CREBBP), transcript variant 2, mRNA; and identified as SEQ ID NO: 25).

| | |
|---|---|
| 1 | CTGCGGGGCG CTGTTGCTGT GGCTGAGATT TGGCCGCCGC CTCCCCCACC CGGCCTGCGC |
| 61 | CCTCCCTCTC CCTCGGCGCC CGCCCGCCCG CTCGCGGCCC GCGCTCGCTC CTCTCCCTCG |
| 121 | CAGCCGGCAG GGCCCCCGAC CCCCGTCCGG GCCCTCGCCG GCCCGGCCGC CCGTGCCCGG |
| 181 | GGCTGTTTTC GCGAGCAGGT GAAAATGGCT GAGAACTTGC TGGACGGACC GCCCAACCCC |
| 241 | AAAAGAGCCA AACTCAGCTC GCCCGGTTTC TCGGCGAATG ACAGCACAGA TTTTGGATCA |
| 301 | TTGTTTGACT TGGAAAATGA TCTTCCTGAT GAGCTGATAC CCAATGGAGG AGAATTAGGC |
| 361 | CTTTTAAACA GTGGGAACCT TGTTCCAGAT GCTGCTTCCA AACATAAACA ACTGTCGGAG |
| 421 | CTTCTACGAG GAGGCAGCGG CTCTAGTATC AACCCAGGAA TAGGAAATGT GAGCGCCAGC |
| 481 | AGCCCCGTGC AGCAGGGCCT GGGTGGCCAG GCTCAAGGGC AGCCGAACAG TGCTAACATG |
| 541 | GCCAGCCTCA GTGCCATGGG CAAGAGCCCT CTGAGCCAGG GAGATTCTTC AGCCCCCAGC |
| 601 | CTGCCTAAAC AGGCAGCCAG CACCTCTGGG CCCACCCCCG CTGCCTCCCA AGCACTGAAT |
| 661 | CCGCAAGCAC AAAAGCAAGT GGGGCTGGCG ACTAGCAGCC CTGCCACGTC ACAGACTGGA |
| 721 | CCTGGTATCT GCATGAATGC TAACTTTAAC CAGACCCACC CAGGCCTCCT CAATAGTAAC |
| 781 | TCTGGCCATA GCTTAATTAA TCAGGCTTCA CAAGGGCAGG CGCAAGTCAT GAATGGATCT |
| 841 | CTTGGGGCTG CTGGCAGAGG AAGGGGAGCT GGAATGCCGT ACCCTACTCC AGCCATGCAG |
| 901 | GGCGCCTCGA GCAGCGTGCT GGCTGAGACC CTAACGCAGG TTTCCCCGCA AATGACTGGT |
| 961 | CACGCGGGAC TGAACACCGC ACAGGCAGGA GGCATGGCCA AGATGGGAAT AACTGGGAAC |
| 1021 | ACAAGTCCAT TTGGACAGCC CTTTAGTCAA GCTGGAGGGC AGCCAATGGG AGCCACTGGA |
| 1081 | GTGAACCCCC AGTTAGCCAG CAAACAGAGC ATGGTCAACA GTTTGCCCAC CTTCCCTACA |
| 1141 | GATATCAAGA ATACTTCAGT CACCAACGTG CCAAATATGT CTCAGATGCA AACATCAGTG |
| 1201 | GGAATTGTAC CCACACAAGC AATTGCAACA GGCCCCACTG CAGATCCTGA AAAACGCAAA |
| 1261 | CTGATACAGC AGCAGCTGGT TCTACTGCTT CATGCTCATA AGTGTCAGAG ACGAGAGCAA |
| 1321 | GCAAACGGAG AGGTTCGGGC CTGCTCGCTC CCGCATTGTC GAACCATGAA AAACGTTTTG |
| 1381 | AATCACATGA CGCATTGTCA GGCTGGGAAA GCCTGCCAAG CCATCCTGGG GTCTCCAGCT |
| 1441 | AGTGGAATTC AAAACACAAT TGGTTCTGTT GGCACAGGGC AACAGAATGC CACTTCTTTA |
| 1501 | AGTAACCCAA ATCCCATAGA CCCCAGCTCC ATGCAGCGAG CCTATGCTGC TCTCGGACTC |
| 1561 | CCCTACATGA ACCAGCCCCA GACGCAGCTG CAGCCTCAGG TTCCTGGCCA GCAACCAGCA |
| 1621 | CAGCCTCAAA CCCACCAGCA GATGAGGACT CTCAACCCCC TGGGAAATAA TCCAATGAAC |
| 1681 | ATTCCAGCAG GAGGAATAAC AACAGATCAG CAGCCCCCAA ACTTGATTTC AGAATCAGCT |
| 1741 | CTTCCGACTT CCCTGGGGGC CACAAACCCA CTGATGAACG ATGGCTCCAA CTCTGGTAAC |
| 1801 | ATTGGAACCC TCAGCACTAT ACCAACAGCA GCTCCTCCTT CTAGCACCGG TGTAAGGAAA |
| 1861 | GGCTGGCACG AACATGTCAC TCAGGACCTG CGGAGCCATC TAGTGCATAA ACTCGTCCAA |
| 1921 | GCCATCTTCC CAACACCTGA TCCCGCAGCT CTAAAGGATC GCCGCATGGA AAACCTGGTA |
| 1981 | GCCTATGCTA AGAAAGTGGA AGGGGACATG TACGAGTCTG CCAACAGCAG GGATGAATAT |
| 2041 | TATCACTTAT TAGCAGAGAA AATCTACAAG ATACAAAAAG AACTAGAAGA AAAACGGAGG |
| 2101 | TCGCGTTTAC ATAAACAAGG CATCTTGGGG AACCAGCCAG CCTTACCAGC CCCGGGGGCT |
| 2161 | CAGCCCCCTG TGATTCCACA GGCACAACCT GTGAGACCTC CAAATGGACC CCTGTCCCTG |
| 2221 | CCAGTGAATC GCATGCAAGT TTCTCAAGGG ATGAATTCAT TTAACCCCAT GTCCTTGGGG |
| 2281 | AACGTCCAGT TGCCACAAGC ACCCATGGGA CCTCGTGCAG CCTCCCCAAT GAACCACTCT |
| 2341 | GTCCAGATGA ACAGCATGGG CTCAGTGCCA GGGATGGCCA TTTCTCCTTC CCGAATGCCT |
| 2401 | CAGCCTCCGA ACATGATGGG TGCACACACC AACAACATGA TGGCCCAGGC GCCCGCTCAG |
| 2461 | AGCCAGTTTC TGCCACAGAA CCAGTTCCCG TCATCCAGCG GGGCGATGAG TGTGGGCATG |
| 2521 | GGGCAGCCGC CAGCCCAAAC AGGCGTGTCA CAGGGACAGG TGCCTGGTGC TGCTCTTCCT |
| 2581 | AACCCTCTCA ACATGCTGGG GCCTCAGGCC AGCCAGCTAC CTTGCCCTCC AGTGACACAG |
| 2641 | TCACCACTGC ACCCAACACC GCCTCCTGCT TCCACGGCTG CTGGCATGCC ATCTCTCCAG |
| 2701 | CACACGACAC CACCTGGGAT GACTCCTCCC CAGCCAGCAG CTCCCACTCA GCCATCAACT |
| 2761 | CCTGTGTCGT CTTCCGGGCA GACTCCCACC CCGACTCCTG GCTCAGTGCC CAGTGCTACC |
| 2821 | CAAACCCAGA GCACCCCTAC AGTCCAGGCA GCAGCCCAGG CCCAGGTGAC CCCGCAGCCT |
| 2881 | CAAACCCCAG TTCAGCCCCC GTCTGTGGCT ACCCCTCAGT CATCGCAGCA ACAGCCGACG |
| 2941 | CCTGTGCACG CCCAGCCTCC TGGCACACCG CTTTCCCAGG CAGCAGCCAG CATTGATAAC |
| 3001 | AGAGTCCCTA CCCCCTCCTC GGTGGCCAGC GCAGAAACCA ATTCCCAGCA GCCAGGACCT |
| 3061 | GACGTACCTG TGCTGGAAAT GAAGACGGAG ACCCAAGCAG AGGACACTGA GCCCGATCCT |
| 3121 | GGTGAATCCA AAGGGGAGCC CAGGTCTGAG ATGATGGAGG AGGATTTGCA AGGAGCTTCC |
| 3181 | CAAGTTAAAG AAGAAACAGA CATAGCAGAG CAGAAATCAG AACCAATGGA AGTGGATGAA |
| 3241 | AAGAAACCTG AAGTGAAAGT AGAAGTTAAA GAGGAAGAAG AGAGTAGCAG TAACGGCACA |
| 3301 | GCCTCTCAGT CAACATCTCC TTCGCAGCCG CGCAAAAAAA TCTTTAAACC AGAGGAGTTA |
| 3361 | CGCCAGGCCC TCATGCCAAC CCTAGAAGCA CTGTATCGAC AGGACCCAGA GTCATTACCT |
| 3421 | TTCCGGCAGC CTGTAGATCC CCAGCTCCTC GGAATTCCAG ACTATTTTGA CATCGTAAAG |
| 3481 | AATCCCATGG ACCTCTCCAC CATCAAGCGG AAGCTGGACA CAGGGCAATA CCAAGAGCCC |
| 3541 | TGGCAGTACG TGGACGACGT CTGGCTCATG TTCAACAATG CCTGGCTCTA TAATCGCAAG |
| 3601 | ACATCCCGAG TCTATAAGTT TTGCAGTAAG CTTGCAGAGG TCTTTGAGCA GGAAATTGAC |
| 3661 | CCTGTCATGC AGTCCCTTGG ATATTGCTGT GGACGCAAGT ATGAGTTTTC CCCACAGACT |
| 3721 | TTGTGCTGCT ATGGGAAGCA GCTGTGTACC ATTCCTCGCG ATGCTGCCTA CTACAGCTAT |
| 3781 | CAGAATAGGT ATCATTTCTG TGAGAAGTGT TTCACAGAGA TCCAGGGCGA GAATGTGACC |
| 3841 | CTGGGTGACG ACCCTTCACA GCCCCAGACG ACAATTTCAA AGGATCAGTT TGAAAAGAAG |
| 3901 | AAAAATGATA CCTTAGACCC CGAACCTTTC GTTGATTGCA AGGAGTGTGG CCGGAAGATG |
| 3961 | CATCAGATTT GCGTTCTGCA CTATGACATC ATTTGGCCTT CAGGTTTTGT GTGCGACAAC |
| 4021 | TGCTTGAAGA AAACTGGCAG ACCTCGAAAA GAAAACAAAT TCAGTGCTAA GAGGCTGCAG |
| 4081 | ACCACAAGAC TGGGAAACCA CTTGGAAGAC CGAGTGAACA AATTTTTGCG GCGCCAGAAT |
| 4141 | CACCCTGAAG CCGGGGAGGT TTTTGTCCGA GTGGTGGCCA GCTCAGACAA GACGGTGGAG |
| 4201 | GTCAAGCCCG GGATGAAGTC ACGGTTTGTG GATTCTGGGG AAATGTCTGA ATCTTTCCCA |
| 4261 | TATCGAACCA AAGCTCTGTT TGCTTTTGAG GAAATTGACG GCGTGGATGT CTGCTTTTTT |
| 4321 | GGAATGCACG TCCAAGAATA CGGCTCTGAT TGCCCCCCTC CAAACACGAG GCGTGTGTAC |
| 4381 | ATTTCTTATC TGGATAGTAT TCATTTCTTC CGGCCACGTT GCCTCCGCAC AGCCGTTTAC |
| 4441 | CATGAGATCC TTATTGGATA TTTAGAGTAT GTGAAGAAAT TAGGGTATGT GACAGGGCAC |
| 4501 | ATCTGGGCCT GTCCTCCAAG TGAAGGAGAT GATTACATCT TCCATTGCCA CCCACCTGAT |
| 4561 | CAAAAAATAC CCAAGCCAAA ACGACTGCAG GAGTGGTACA AAAAGATGCT GGACAAGGCG |
| 4621 | TTTGCAGAGC GGATCATCCA TGACTACAAG GATATTTTCA AACAAGCAAC TGAAGACAGG |
| 4681 | CTCACCAGTG CCAAGGAACT GCCCTATTTT GAAGGTGATT TCTGGCCCAA TGTGTTAGAA |
| 4741 | GAGAGCATTA AGGAACTAGA ACAAGAAGAA GAGGAGAGGA AAAAGGAAGA GAGCACTGCA |
| 4801 | GCCAGTGAAA CCACTGAGGG CAGTCAGGGC GACAGCAAGA ATGCCAAGAA GAAGAACAAC |
| 4861 | AAGAAAACCA ACAAGAACAA AAGCAGCATC AGCCGCGCCA ACAAGAAGAA GCCCAGCATG |
| 4921 | CCCAACGTGT CCAATGACCT GTCCCAGAAG CTGTATGCCA CCATGGAGAA GCACAAGGAG |
| 4981 | GTCTTCTTCG TGATCCACCT GCACGCTGGG CCTGTCATCA ACACCCTGCC CCCCATCGTC |
| 5041 | GACCCCGACC CCCTGCTCAG CTGTGACCTC ATGGATGGGC GCGACGCCTT CCTCACCCTC |
| 5101 | GCCAGAGACA AGCACTGGGA GTTCTCCTCC TTGCGCCGCT CCAAGTGGTC CACGCTCTGC |
| 5161 | ATGCTGGTGG AGCTGCACAC CCAGGGCCAG GACCGCTTTG TCTACACCTG CAACGAGTGC |
| 5221 | AAGCACCACG TGGAGACGCG CTGGCACTGC ACTGTGTGCG AGGACTACGA CCTCTGCATC |
| 5281 | AACTGCTATA ACACGAAGAG CCATGCCCAT AAGATGGTGA AGTGGGGGCT GGGCCTGGAT |
| 5341 | GACGAGGGCA GCAGCCAGGG CGAGCCACAG TCAAAGAGCC CCCAGGAGTC ACGCCGGCTG |
| 5401 | AGCATCCAGC GCTGCATCCA GTCGCTGGTG CACGCGTGCC AGTGCCGCAA CGCCAACTGC |
| 5461 | TCGCTGCCAT CCTGCCAGAA GATGAAGCGG GTGGTGCAGC ACACCAAGGG CTGCAAACGC |
| 5521 | AAGACCAACG GGGGCTGCCC GGTGTGCAAG CAGCTCATCG CCCTCTGCTG CTACCACGCC |
| 5581 | AAGCACTGCC AAGAAAACAA ATGCCCCGTG CCCTTCTGCC TCAACATCAA ACACAAGCTC |
| 5641 | CGCCAGCAGC AGATCCAGCA CCGCCTGCAG CAGGCCCAGC TCATGCGCCG GCGGATGGCC |
| 5701 | ACCATGAACA CCCGCAACGT GCCTCAGCAG AGTCTGCCTT CTCCTACCTC AGCACCGCCC |
| 5761 | GGGACCCCCA CACAGCAGCC CAGCACACCC CAGACGCCGC AGCCCCCTGC CCAGCCCCAA |
| 5821 | CCCTCACCCG TGAGCATGTC ACCAGCTGGC TTCCCCAGCG TGGCCCGGAC TCAGCCCCCC |
| 5881 | ACCACGGTGT CCACAGGGAA GCCTACCAGC CAGGTGCCGG CCCCCCCACC CCCGGCCCAG |
| 5941 | CCCCCTCCTG CAGCGGTGGA AGCGGCTCGG CAGATCGAGC GTGAGGCCCA GCAGCAGCAG |
| 6001 | CACCTGTACC GGGTGAACAT CAACAACAGC ATGCCCCCAG GACGCACGGG CATGGGGACC |
| 6061 | CCGGGGAGCC AGATGGCCCC CGTGAGCCTG AATGTGCCCC GACCCAACCA GGTGAGCGGG |
| 6121 | CCCGTCATGC CCAGCATGCC TCCCGGGCAG TGGCAGCAGG CGCCCCTTCC CCAGCAGCAG |
| 6181 | CCCATGCCAG GCTTGCCCAG GCCTGTGATA TCCATGCAGG CCCAGGCGGC CGTGGCTGGG |
| 6241 | CCCCGGATGC CCAGCGTGCA GCCACCCAGG AGCATCTCAC CCAGCGCTCT GCAAGACCTG |
| 6301 | CTGCGGACCC TGAAGTCGCC CAGCTCCCCT CAGCAGCAAC AGCAGGTGCT GAACATTCTC |
| 6361 | AAATCAAACC CGCAGCTAAT GGCAGCTTTC ATCAAACAGC GCACAGCCAA GTACGTGGCC |
| 6421 | AATCAGCCCG GCATGCAGCC CCAGCCTGGC CTCCAGTCCC AGCCCGGCAT GCAACCCCAG |
| 6481 | CCTGGCATGC ACCAGCAGCC CAGCCTGCAG AACCTGAATG CCATGCAGGC TGGCGTGCCG |
| 6541 | CGGCCCGGTG TGCCTCCACA GCAGCAGGCG ATGGGAGGCC TGAACCCCCA GGGCCAGGCC |
| 6601 | TTGAACATCA TGAACCCAGG ACACAACCCC AACATGGCGA GTATGAATCC ACAGTACCGA |
| 6661 | GAAATGTTAC GGAGGCAGCT GCTGCAGCAG CAGCAGCAAC AGCAGCAGCA ACAACAGCAG |
| 6721 | CAACAGCAGC AGCAGCAAGG GAGTGCCGGC ATGGCTGGGG GCATGGCGGG GCACGGCCAG |
| 6781 | TTCCAGCAGC CTCAAGGACC CGGAGGCTAC CCACCGGCCA TGCAGCAGCA GCAGCGCATG |
| 6841 | CAGCAGCATC TCCCCCTCCA GGGCAGCTCC ATGGGCCAGA TGGCGGCTCA GATGGGACAG |
| 6901 | CTTGGCCAGA TGGGGCAGCC GGGGCTGGGG GCAGACAGCA CCCCCAACAT CCAGCAAGCC |
| 6961 | CTGCAGCAGC GGATTCTGCA GCAACAGCAG ATGAAGCAGC AGATTGGGTC CCCAGGCCAG |
| 7021 | CCGAACCCCA TGAGCCCCCA GCAACACATG CTCTCAGGAC AGCCACAGGC CTCGCATCTC |
| 7081 | CCTGGCCAGC AGATCGCCAC GTCCCTTAGT AACCAGGTGC GGTCTCCAGC CCCTGTCCAG |
| 7141 | TCTCCACGGC CCCAGTCCCA GCCTCCACAT TCCAGCCCGT CACCACGGAT ACAGCCCCAG |
| 7201 | CCTTCGCCAC ACCACGTCTC ACCCCAGACT GGTTCCCCCC ACCCCGGACT CGCAGTCACC |
| 7261 | ATGGCCAGCT CCATAGATCA GGGACACTTG GGGAACCCCG AACAGAGTGC AATGCTCCCC |
| 7321 | CAGCTGAACA CCCCCAGCAG GAGTGCGCTG TCCAGCGAAC TGTCCCTGGT CGGGGACACC |
| 7381 | ACGGGGGACA CGCTAGAGAA GTTTGTGGAG GGCTTGTAGC ATTGTGAGAG CATCACCTTT |
| 7441 | TCCCTTTCAT GTTCTTGGAC CTTTTGTACT GAAAATCCAG GCATCTAGGT TCTTTTTATT |
| 7501 | CCTAGATGGA ACTGCGACTT CCGAGCCATG GAAGGGTGGA TTGATGTTTA AAGAAACAAT |
| 7561 | ACAAAGAATA TATTTTTTTG TTAAAAACCA GTTGATTTAA ATATCTGGTC TCTCTCTTTG |
| 7621 | GTTTTTTTTT GGCGGGGGGG TGGGGGGGGT TCTTTTTTTT CCGTTTTGTT TTTGTTTGGG |
| 7681 | GGGAGGGGGG TTTTGTTTGG ATTCTTTTTG TCGTCATTGC TGGTGACTCA TGCCTTTTTT |
| 7741 | TAACGGGAAA AACAAGTTCA TTATATTCAT ATTTTTTATT TGTATTTTCA AGACTTTAAA |
| 7801 | CATTTATGTT TAAAAGTAAG AAGAAAAATA ATATTCAGAA CTGATTCCTG AAATAATGCA |
| 7861 | AGCTTATAAT GTATCCCGAT AACTTTGTGA TGTTTCGGGA AGATTTTTTT CTATAGTGAA |
| 7921 | CTCTGTGGGC GTCTCCCAGT ATTACCCTGG ATGATAGGAA TTGACTCCGG CGTGCACACA |
| 7981 | CGTACACACC CACACACATC TATCTATACA TAATGGCTGA AGCCAAACTT GTCTTGCAGA |
| 8041 | TGTAGAAATT GTTGCTTTGT TTCTCTGATA AAACTGGTTT TAGACAAAAA ATAGGGATGA |
| 8101 | TCACTCTTAG ACCATGCTAA TGTTACTAGA GAAGAAGCCT TCTTTTCTTT CTTCTATGTG |
| 8161 | AAACTTGAAA TGAGGAAAAG CAATTCTAGT GTAAATCATG CAAGCGCTCT AATTCCTATA |
| 8221 | AATACGAAAC TCGAGAAGAT TCAATCACTG TATAGAATGG TAAAATACCA ACTCATTTCT |
| 8281 | TATATCATAT TGTTAAATAA ACTGTGTGCA ACAGACAAAA AGGGTGGTCC TTCTTGAATT |
| 8341 | CATGTACATG GTATTAACAC TTAGTGTTCG GGGTTTTTTG TTATGAAAAT GCTGTTTTCA |
| 8401 | ACATTGTATT TGGACTATGC ATGTGTTTTT TCCCCATTGT ATATAAAGTA CCGCTTAAAA |
| 8461 | TTGATATAAA TTACTGAGGT TTTTAACATG TATTCTGTTC TTTAAGATCC CTGTAAGAAT |
| 8521 | GTTTAAGGTT TTTATTTATT TATATATATT TTTTGAGTCT GTTCTTTGTA AGACATGGTT |
| 8581 | CTGGTTGTTC GCTCATAGCG GAGAGGCTGG GGCTGCGGTT GTGGTTGTGG CGGCGTGGGT |
| 8641 | GGTGGCTGGG AACTGTGGCC CAGGCTTAGC GGCCGCCCGG AGGCTTTTCT TCCCGGAGAC |
| 8701 | TGAGGTGGGC GACTGAGGTG GGCGGCTCAG CGTTGGCCCC ACACATTCGA GGCTCACAGG |
| 8761 | TGATTGTCGC TCACACAGTT AGGGTCGTCA GTTGGTCTGA AACTGCATTT GGCCCACTCC |
| 8821 | TCCATCCTCC CTGTCCGTCG TAGCTGCCAC CCCCAGAGGC GGCGCTTCTT CCCGTGTTCA |
| 8881 | GGCGGCTCCC CCCCCCCGTA CACGACTCCC AGAATCTGAG GCAGAGAGTG CTCCAGGCTC |
| 8941 | GCGAGGTGCT TTCTGACTTC CCCCCAAATC CTGCCGCTGC CGCGCAGCAT GTCCCGTGTG |
| 9001 | GCGTTTGAGG AAATGCTGAG GGACAGACAC CTTGGAGCAC CAGCTCCGGT CCCTGTTACA |
| 9061 | GTGAGAAAGG TCCCCCACTT CGGGGGATAC TTGCACTTAG CCACATGGTC CTGCCTCCCT |
| 9121 | TGGAGTCCAG TTCCAGGCTC CCTTACTGAG TGGGTGAGAC AAGTTCACAA AAACCGTAAA |
| 9181 | ACTGAGAGGA GGACCATGGG CAGGGGAGCT GAAGTTCATC CCCTAAGTCT ACCACCCCCA |
| 9241 | GCACCCAGAG AACCCACTTT ATCCCTAGTC CCCCAACAAA GGCTGGTCTA GGTGGGGGTG |
| 9301 | ATGGTAATTT TAGAAATCAC GCCCCAAATA GCTTCCGTTT GGGCCCTTAC ATTCACAGAT |
| 9361 | AGGTTTTAAA TAGCTGAATA CTTGGTTTGG GAATCTGAAT TCGAGGAACC TTTCTAAGAA |
| 9421 | GTTGGAAAGG TCCGATCTAG TTTTAGCACA GAGCTTTGAA CCTTGAGTTA TAAAATGCAG |
| 9481 | AATAATTCAA GTAAAAATAA GACCACCATC TGGCACCCCT GACCAGCCCC CATTCACCCC |
| 9541 | ATCCCAGGAG GGGAAGCACA GGCCGGGCCT CCGGTGGAGA TTGCTGCCAC TGCTCGGCCT |
| 9601 | GCTGGGTTCT TAACCTCCAG TGTCCTCTTC ATCTTTTCCA CCCGTAGGGA AACCTTGAGC |
| 9661 | CATGTGTTCA AACAAGAAGT GGGGCTAGAG CCCGAGAGCA GCAGCTCTAA GCCCACACTC |
| 9721 | AGAAAGTGGC GCCCTCCTGG TTGTGCAGCC TTTTAATGTG GGCAGTGGAG GGGCCTCTGT |
| 9781 | TTCAGGTTAT CCTGGAATTC AAAACGTTAT GTACCAACCT CATCCTCTTT GGAGTCTGCA |
| 9841 | TCCTGTGCAA CCGTCTTGGG CAATCCAGAT GTCGAAGGAT GTGACCGAGA GCATGGTCTG |
| 9901 | TGGATGCTAA CCCTAAGTTT GTCGTAAGGA AATTTCTGTA AGAAACCTGG AAAGCCCCAA |
| 9961 | CGCTGTGTCT CATGCTGTAT ACTTAAGAGG AGAAGAAAAA GTCCTATATT TGTGATCAAA |
| 10021 | AAGAGGAAAC TTGAAATGTG ATGGTGTTTA TAATAAAAGA TGGTAAAACT ACTTGGATTC |
| 10081 | AAA |

In certain embodiments, a mutation of the disclosure may occur in a sequence encoding the CREB Binding Protein (CREBBP) HAT, including the amino acid sequence encoding CREBBP (below, corresponding to GenBank Accession No. NP_001073315.1, defined as Homo sapiens CREB-binding protein isoform b; and identified as SEQ ID NO: 26).

MAENLLDGPPNPKRAKLSSPGFSANDSTDFGSLFDLENDLPDELIPNGGE
LGLLNSGNLVPDAASKHKQLSELLRGGSGSSINPGIGNVSASSPVQQGLG
GQAQGQPNSANMASLSAMGKSPLSQGDSSAPSLPKQAASTSGPTPAASQA
LNPQAQKQVGLATSSPATSQTGPGICMNANFNQTHPGLLNSNSGHSLINQ
ASQGQAQVMNGSLGAAGRGRGAGMPYPTPAMQGASSSVLAETLTQVSPQM
TGHAGLNTAQAGGMAKMGITGNTSPFGQPFSQAGGQPMGATGVNPQLASK
QSMVNSLPTFPTDIKNTSVTNVPNMSQMQTSVGIVPTQAIATGPTADPEK
RKLIQQQLVLLLHAHKCQRREQANGEVRACSLPHCRTMKNVLNHMTHCQA
GKACQAILGSPASGIQNTIGSVGTGQQNATSLSNPNPIDPSSMQRAYAAL
GLPYMNQPQTQLQPQVPGQQPAQPQTHQQMRTLNPLGNNPMNIPAGGITT
DQQPPNLISESALPTSLGATNPLMNDGSNSGNIGTLSTIPTAAPPSSTGV
RKGWHEHVTQDLRSHLVHKLVQAIFPTPDPAALKDRRMENLVAYAKKVEG
DMYESANSRDEYYHLLAEKIYKIQKELEEKRRSRLHKQGILGNQPALPAP
GAQPPVIPQAQPVRPPNGPLSLPVNRMQVSQGMNSFNPMSLGNVQLPQAP
MGPRAASPMNHSVQMNSMGSVPGMAISPSRMPQPPNMMGAHTNNMMAQAP
AQSQFLPQNQFPSSSGAMSVGMGQPPAQTGVSQGQVPGAALPNPLNMLGP
QASQLPCPPVTQSPLHPTPPPASTAAGMPSLQHTTPPGMTPPQPAAPTQP
STPVSSSGQTPTPTPGSVPSATQTQSTPTVQAAAQAQVTPQPQTPVQPPS
VATPQSSQQQPTPVHAQPPGTPLSQAAASIDNRVPTPSSVASAETNSQQP
GPDVPVLEMKTETQAEDTEPDPGESKGEPRSEMMEEDLQGASQVKEETDI
AEQKSEPMEVDEKKPEVKVEVKEEEESSSNGTASQSTSPSQPRKKIFKPE
ELRQALMPTLEALYRQDPESLPFRQPVDPQLLGIPDYFDIVKNPMDLSTI
KRKLDTGQYQEPWQYVDDVWLMFNNAWLYNRKTSRVYKFCSKLAEVFEQE
IDPVMQSLGYCCGRKYEFSPQTLCCYGKQLCTIPRDAAYYSYQNRYHFCE
KCFTEIQGENVTLGDDPSQPQTTISKDQFEKKKNDTLDPEPFVDCKECGR
KMHQICVLHYDIIWPSGFVCDNCLKKTGRPRKENKFSAKRLQTTRLGNHL
EDRVNKFLRRQNHPEAGEVFVRVVASSDKTVEVKPGMKSRFVDSGEMSES
FPYRTKALFAFEEIDGVDVCFFGMHVQEYGSDCPPPNTRRVYISYLDSIH
FFRPRCLRTAVYHEILIGYLEYVKKLGYVTGHIWACPPSEGDDYIFHCHP
PDQKIPKPKRLQEWYKKMLDKAFAERIIHDYKDIFKQATEDRLTSAKELP
YFEGDFWPNVLEESIKELEQEEEERKKEESTAASETTEGSQGDSKNAKKK
NNKKTNKNKSSISRANKKKPSMPNVSNDLSQKLYATMEKHKEVFFVIHLH
AGPVINTLPPIVDPDPLLSCDLMDGRDAFLTLARDKHWEFSSLRRSKWST
LCMLVELHTQGQDRFVYTCNECKHHVETRWHCTVCEDYDLCINCYNTKSH
AHKMVKWGLGLDDEGSSQGEPQSKSPQESRRLSIQRCIQSLVHACQCRNA
NCSLPSCQKMKRVVQHTKGCKRKTNGGCPVCKQLIALCCYHAKHCQENKC
PVPFCLNIKHKLRQQQIQHRLQQAQLMRRRMATMNTRNVPQQSLPSPTSA
PPGTPTQQPSTPQTPQPPAQPQPSPVSMSPAGFPSVARTQPPTTVSTGKP
TSQVPAPPPPAQPPPAAVEAARQIEREAQQQQHLYRVNINNSMPPGRTGM
GTPGSQMAPVSLNVPRPNQVSGPVMPSMPPGQWQQAPLPQQQPMPGLPRP
VISMQAQAAVAGPRMPSVQPPRSISPSALQDLLRTLKSPSSPQQQQQVLN
ILKSNPQLMAAFIKQRTAKYVANQPGMQPQPGLQSQPGMQPQPGMHQQPS
LQNLNAMQAGVPRPGVPPQQQAMGGLNPQGQALNIMNPGHNPNMASMNPQ
YREMLRRQLLQQQQQQQQQQQQQQQQQQGSAGMAGGMAGHGQFQQPQGPG
GYPPAMQQQQRMQQHLPLQGSSMGQMAAQMGQLGQMGQPGLGADSTPNIQ
QALQQRILQQQQMKQQIGSPGQPNPMSPQQHMLSGQPQASHLPGQQIATS
LSNQVRSPAPVQSPRPQSQPPHSSPSPRIQPQPSPHHVSPQTGSPHPGLA
VTMASSIDQGHLGNPEQSAMLPQLNTPSRSALSSELSLVGDTTGDTLEKF
VEGL

### Next Generation Sequencing

The compounds of the disclosure are inhibitors of the histone methyltransferase EZH2 for use in the treatment of patients with non-Hodgkin lymphoma (NHL), and in patients with certain genetically defined solid tumors. Activating EZH2 mutations present in NHL patients has been implicated to predict response to EZH2 inhibition (Knutson et al., Nat. Chem. Biol. 2012; 8: 890-896, the content of which is incorporated herein by reference in its entirety). Furthermore, a phase 1 clinical trial of tazemetostat demonstrated clinical responses in both EZH2 mutant and wild type patients (ClinicalTrials.gov identifier: NCT01897571). However, the impact of somatic mutations other than EZH2 on likelihood of response to tazemetostat in NHL patients is currently unknown. In some aspects, the present disclosure provides a multi-gene NHL targeted next generation sequencing (NGS) panel (e.g., a 39-gene panel or a 62-gene panel, or a panel combining a plurality of genes or gene products referred to herein) capable of analyzing samples from malignant cells, tissues, or body fluids, e.g., archive tissue or cell-free circulating tumor DNA (ctDNA) isolated from plasma. In some aspects, the NGS panel is capable of identifying molecular variants, including specific somatic sequence mutations (single base and insertion/deletion, e.g., EZH2), amplifications (e.g., BLC2) and translocations (e.g., BCL2 and MYC) in the tumor and ctDNA samples down to variant allele frequencies of 2% and 0.1% for archive and ctDNA respectively. For example, molecular variants associated with positive (e.g., EZH2, STAT6, MYD88, and SOCS1 mutations) and negative (e.g., MYC and HIST1H1E mutations) clinical responses to tazemetostat treatment were identified. Furthermore, sequencing of phase 1 NHL patients utilizing a 62 gene NHL NGS panel revealed a complex genetic landscape with epigenetic modifiers CREBBP and KMT2D representing the most frequently mutated genes in this sample set. Further aspects of the disclosure provide for an NGS panel with the ability to determine molecular profiles using ctDNA that enables patient characterization where archive tumor tissue or DNA is absent or limiting. Additionally, profiling ctDNA enables longitudinal monitoring of a patient's mutation burden without the need for tumor biopsies.

Without wishing to be bound by theory, mutations identified by the NGS panel disclosed herein, may be used for patient stratification. Accordingly, in some embodiments, the disclosure provides a method of selecting a patient for cancer treatment if the patient has one or more mutations disclosed herein. In some embodiments, the patient selected for the cancer treatment has two or more (e.g., two, three, four, five, six, seven, eight, or more) mutations disclosed herein.

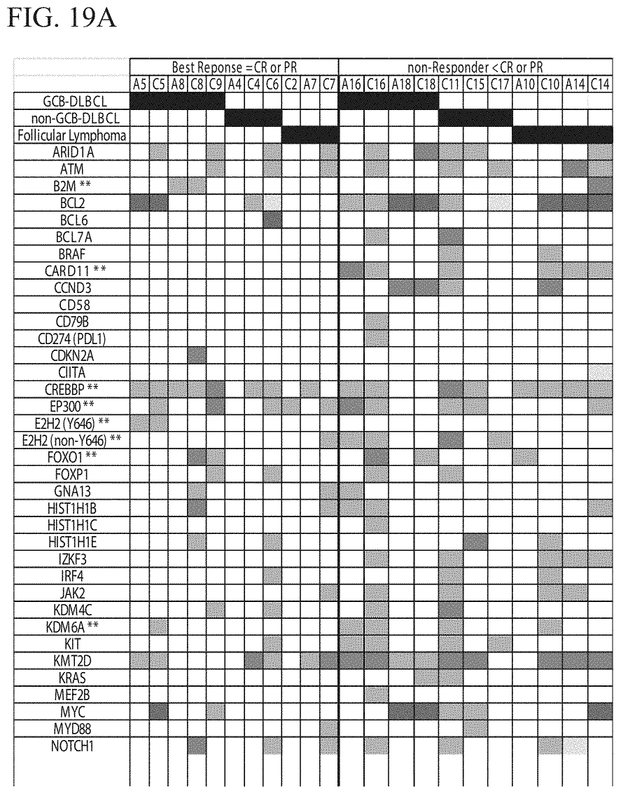

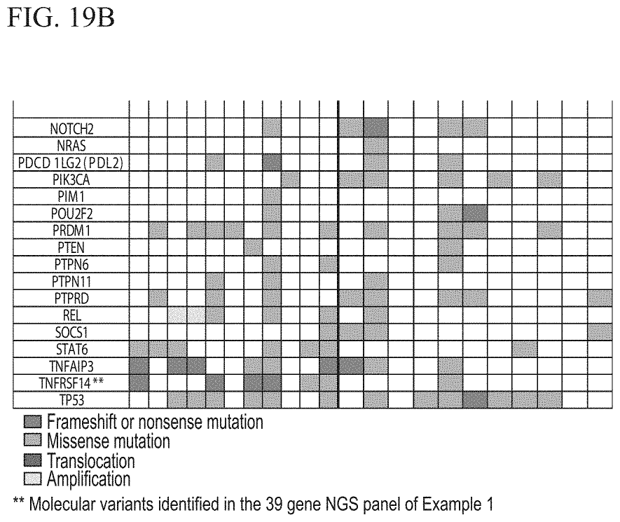

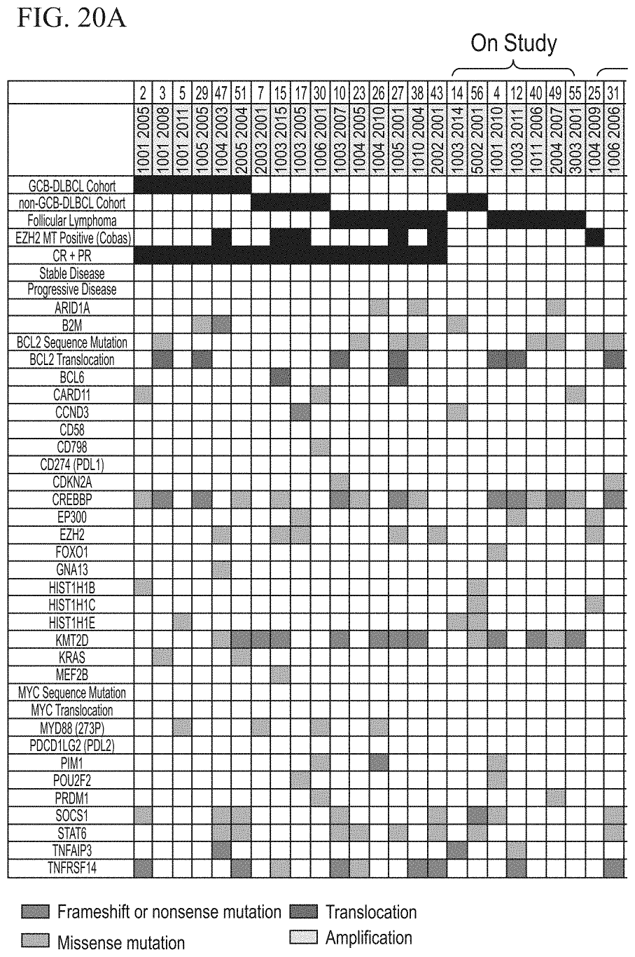

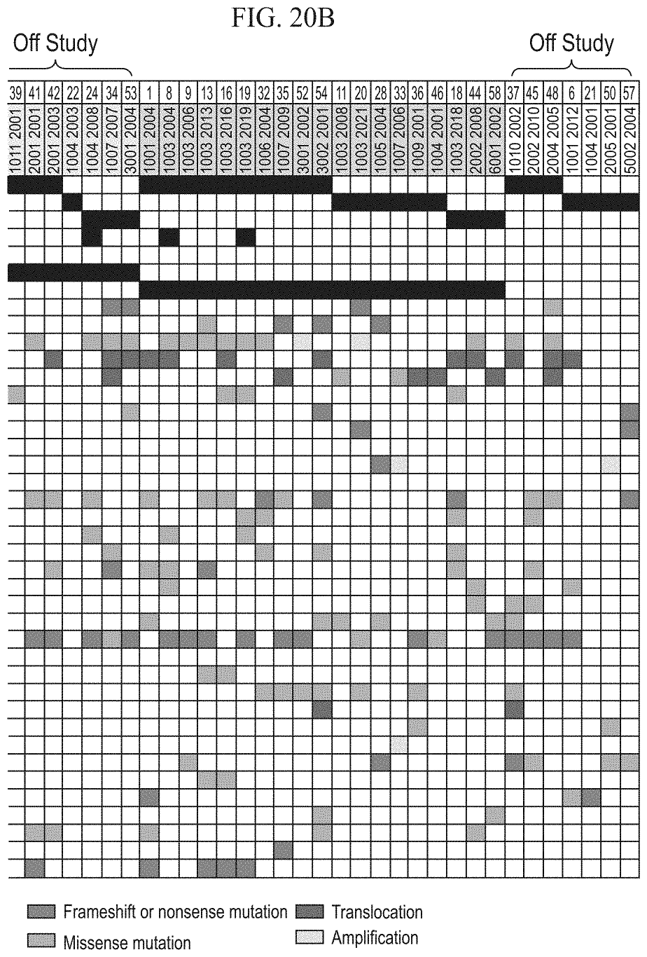

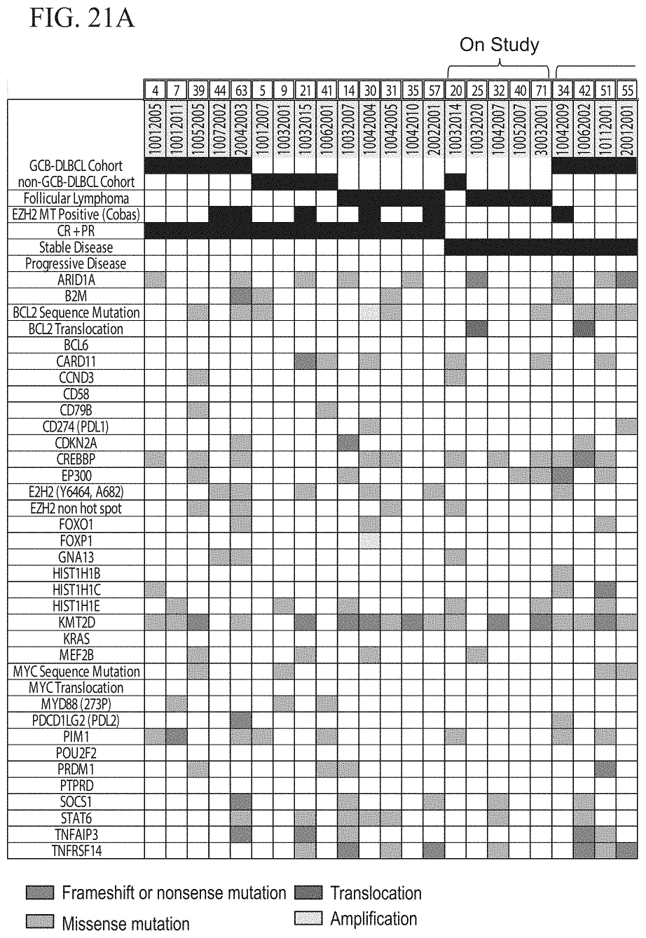

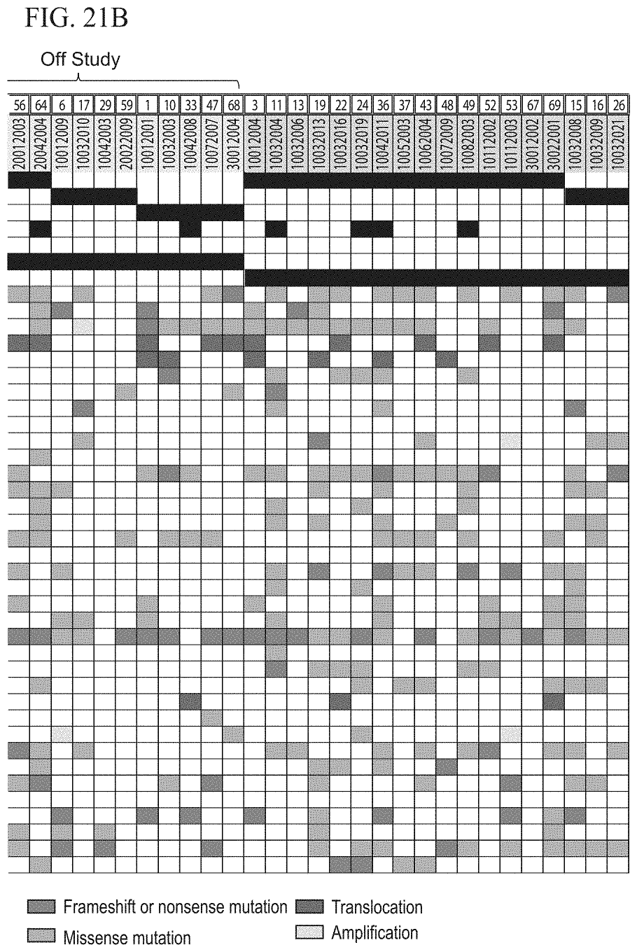

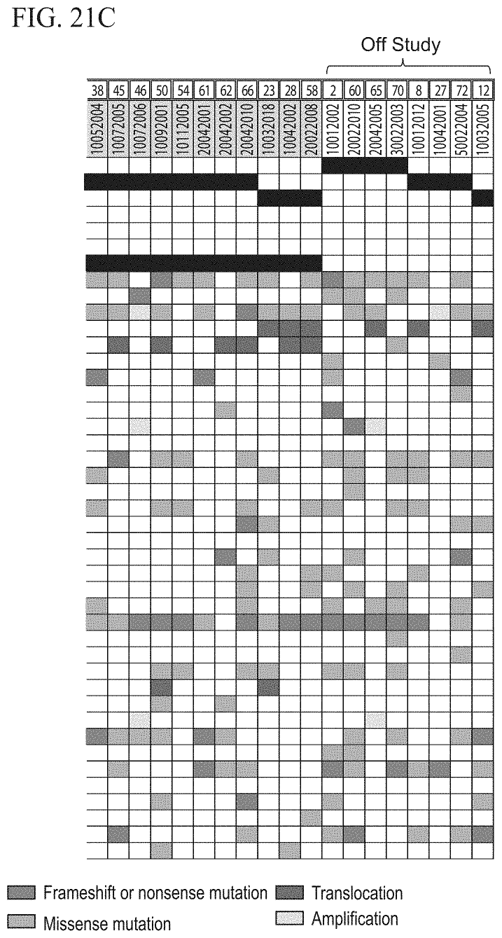

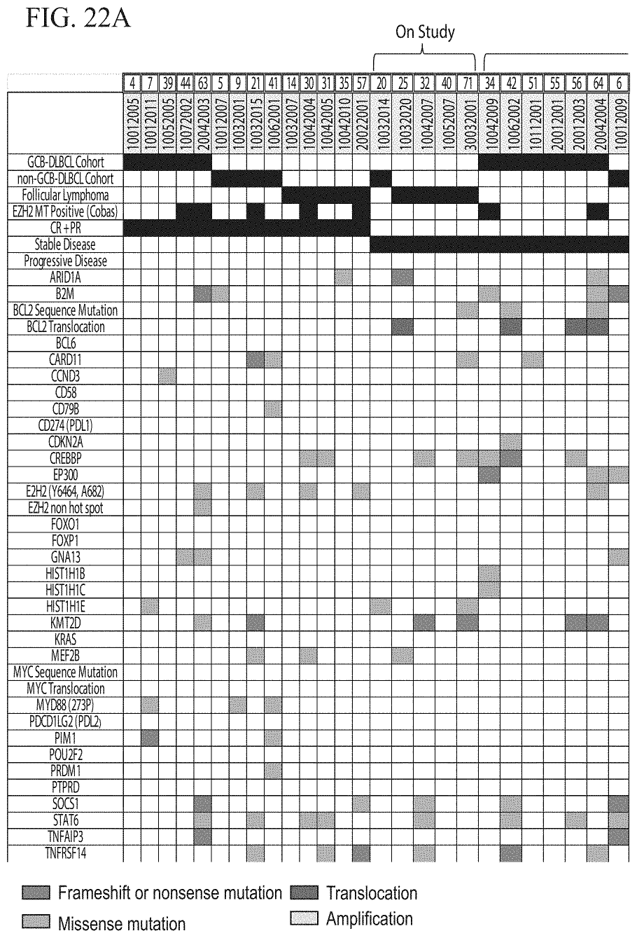

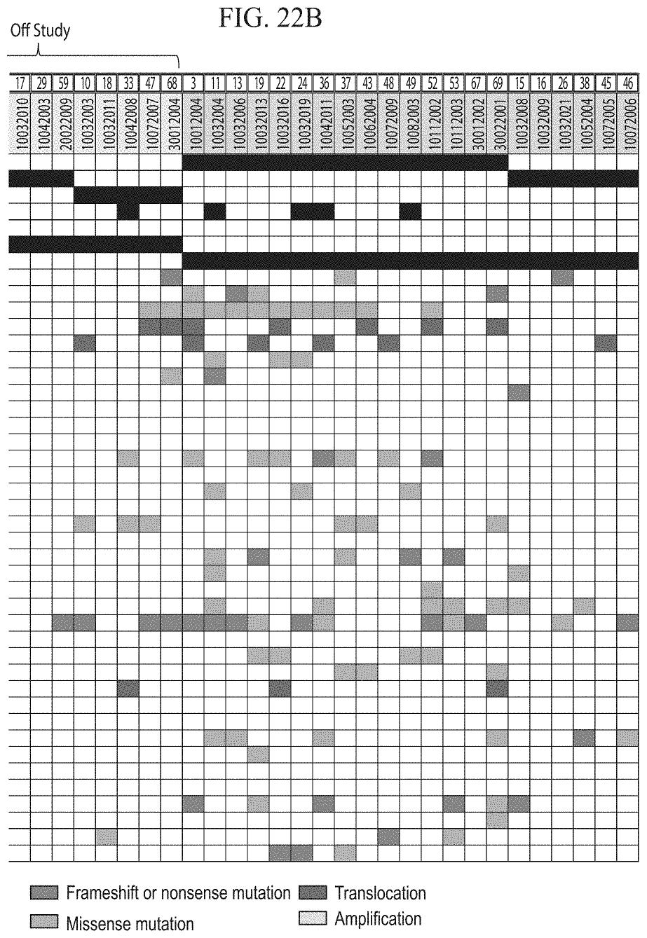

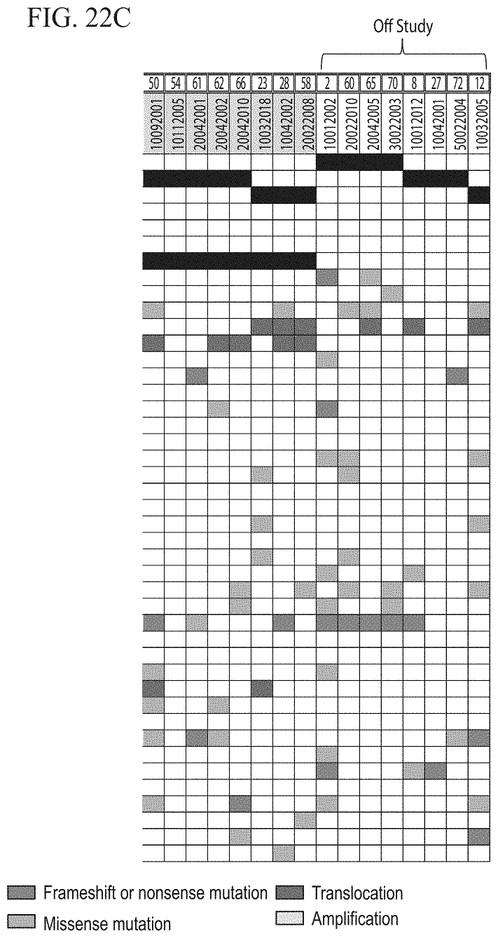

In some embodiments, a method is provided in which a subject having cancer is selected for treatment with an EZH2 inhibitor, e.g., an EZH2 inhibitor disclosed herein, based on the presence of one or more mutations associated with a positive response to such treatment in the subject, e.g., as determined by ctDNA analysis. In some embodiments, a mutation (or a combination of two or more mutations) associated with a positive response is a mutation (or a combination of mutations) that is present only in patients who responded with complete or partial response or, in some embodiments, with stable disease in any of the studies presented herein, e.g., those summarized in FIGS. 19A-22C. In some embodiments, a mutation (or a combination of two or more mutations) associated with a positive response is a mutation (or a combination of mutations) that is not randomly distributed within the patient population examined, but is overrepresented in those patients who responded with a complete or partial response or, in some embodiments, stable disease, in any of the studies presented herein, e.g., those summarized in FIGS. 19A-22C. In some embodiments, a mutation (or combination of mutations) associated with a positive response is a mutation (or combination of mutations) that is overrepresented in the responding (CR, PR, or, in some embodiments, SD) patient population at least 2-fold, at least 3-fold, at least 4-fold, at least 5-fold, or at least 10-fold, as compared to the patient population that did not respond or responded with progressive disease (PD).

In some embodiments, a method is provided in which a subject having cancer is selected for treatment with an EZH2 inhibitor, e.g., an EZH2 inhibitor disclosed herein, based on the absence of one or more mutations associated with a negative response to such treatment in the subject, e.g., as determined by ctDNA analysis. In some embodiments, a mutation (or a combination of two or more mutations) associated with a negative response is a mutation (or a combination of mutations) that is present only in patients who did not respond or responded with progressive disease (PD) in any of the studies presented herein, e.g., those summarized in FIGS. 19A-22C. In some embodiments, a mutation (or a combination of two or more mutations) associated with a negative response is a mutation (or a combination of mutations) that is not randomly distributed within the patient population examined, but is overrepresented in those patients who did not respond or responded with progressive disease in any of the studies presented herein, e.g., those summarized in FIGS. 19A-22C. In some embodiments, a mutation (or combination of mutations) associated with a negative response is a mutation (or combination of mutations) that is overrepresented in the non-responding or progressive disease (PD) patient population at least 2-fold, at least 3-fold, at least 4-fold, at least 5-fold, or at least 10-fold, as compared to the patient population that responded with CR, PR, or, in some embodiments, SD.

In some embodiments, a subject having cancer is selected for treatment with an EZH2 inhibitor, e.g., an EZH2 inhibitor disclosed herein, based on the presence of two or more (e.g., two, three, four, five, six, seven, eight, or more) mutations in the subject that match the mutations observed in a profile of a patient who exhibited a complete or partial response in any of the studies described herein (e.g., those summarized in FIGS. 19A-22C). In some embodiments, a subject having cancer is selected for treatment with an EZH2 inhibitor, e.g., an EZH2 inhibitor disclosed herein, based on the presence of a mutation profile (e.g., of two or more (e.g., two, three, four, five, six, seven, eight, or more)) mutations in the subject that match the mutation profile of a patient who exhibited a complete or partial response in any of the studies described herein (e.g., those summarized in FIGS. 19A-22C). Typically, a mutation in a gene or gene product (e.g., in a transcript, mRNA, or protein) is detected by comparing a given sequence with a reference sequence, e.g., a human reference genome sequence (e.g., human reference genome hg19), and identifying a mismatch in the sequence at hand as compared to the reference sequence.

In some embodiments, a subject having cancer is selected for treatment with an EZH2 inhibitor, e.g., an EZH2 inhibitor disclosed herein, based on the presence of two or more (e.g., two, three, four, five, six, seven, eight, or more) mutations in the subject that match the mutations observed in a profile of a patient who exhibited stable disease in any of the studies described herein (e.g., those summarized in FIGS. 19A-22C). In some embodiments, a subject having cancer is selected for treatment with an EZH2 inhibitor, e.g., an EZH2 inhibitor disclosed herein, based on the presence of a mutation profile (e.g., two or more (e.g., two, three, four, five, six, seven, eight, or more)) mutations in the subject that match the mutation profile of a patient who exhibited stable disease in any of the studies described herein (e.g., those summarized in FIGS. 19A-22C).

In some embodiments, methods of treating cancer is provided that comprises administering a therapeutically effective amount of an inhibitor of EZH2 to a subject in need thereof, wherein the subject has at least one mutation in one or more sequences encoding a gene or a gene product (e.g., a transcript, mRNA, or protein) listed in Tables 1-9, Tables 17-19, and/or FIGS. 19A-22C. In some embodiments, the subject has at least one mutation in in one or more sequences encoding: MYD88, STAT6A, SOCS1, MYC, HIST1H1E, ABL1, ACVR1, AKT1, AKT2, ALK, APC, AR, ARID1A, ARID1B, ASXL1, ATM, ATRX, AURKA, AXIN2, BAP1, BCL2, BCR, BLM, BMPR1A, BRAF, BRCA1, BRCA2, BRIP1, BTK, BUB1B, CALR, CBL, CCND1, CCNE1, CDC73, CDH1, CDK4, CDK6, CDKN1B, CDKN2A, CDKN2B, CDKN2C, CEBPA, CHEK2, CIC, CREBBP, CSF1R, CTNNB1, CYLD, DAXX, DDB2, DDR2, DICER1, DNMT3A, EGFR, EP300, ERBB2, ERBB3, ERBB4, ERCC1, ERCC2, ERCC3, ERCC4, ERCC5, ESR1, ETV1, ETV5, EWSR1, EXT1, EXT2, FANCA, FANCB, FANCC, FANCD2, FANCE, FANCF, FANCG, FANCI, FANCL, FANCM, FBXW7, FGFR1, FGFR2, FGFR3, FGFR4, FH, FLCN, FLT3, FLT4, FOXL2, GATA1, GATA2, GNA11, GNAQ, GNAS, GPC3, H3F3A, H3F3B, HNF1A, HRAS, IDH1, IDH2, IGF1R, IGF2R, IKZF1, JAK1, JAK2, JAK3, KDR, KIT, KRAS, MAML1, MAP2K1, MAP2K4, MDM2, MDM4, MED12, MEN1, MET, MLH1, MLL, MPL, MSH2, MSH6, MTOR, MUTYH, MYCL1, MYCN, NBN, NCOA3, NF1, NF2, NKX2-1, NOTCH1, NOTCH2, NOTCH3, NOTCH4, NPM1, NRAS, NTRK1, PALB2, PAX5, PBRM1, PDGFRA, PHOX2B, PIK3CA, PIK3R1, PMS1, PMS2, POLD1, POLE, POLH, POT1, PRKAR1A, PRSS1, PTCH1, PTEN, PTPN11, RAD51C, RAF1, RB1, RECQL4, RET, RNF43, ROS1, RUNX1, SBDS, SDHAF2, SDHB, SDHC, SDHD, SF3B1, SMAD2, SMAD3, SMAD4, SMARCB1, SMO, SRC, STAG2, STK11, SUFU, TERT, TET2, TGFBR2, TNFAIP3, TOP1, TP53, TSC1, TSC2, TSHR, VHL, WAS, WRN, WT1, XPA, XPC, and/or XRCC1. In some embodiments, the subject has at least one mutation in one or more sequences encoding ABL1, ACVR1, AKT1, AKT2, ALK, APC, AR, ARID1A, ARID1B, ASXL1, ATM, ATRX, AURKA, AXIN2, BAP1, BCL2, BCR, BLM, BMPR1A, BRAF, BRCA1, BRCA2, BRIP1, BTK, BUB1B, CALR, CBL, CCND1, CCNE1, CDC73, CDH1, CDK4, CDK6, CDKN1B, CDKN2A, CDKN2B, CDKN2C, CEBPA, CHEK2, CIC, CREBBP, CSF1R, CTNNB1, CYLD, DAXX, DDB2, DDR2, DICER1, DNMT3A, EGFR, EP300, ERBB2, ERBB3, ERBB4, ERCC1, ERCC2, ERCC3, ERCC4, ERCC5, ESR1, ETV1, ETV5, EWSR1, EXT1, EXT2, EZH2, FANCA, FANCB, FANCC, FANCD2, FANCE, FANCF, FANCG, FANCI, FANCL, FANCM, FBXW7, FGFR1, FGFR2, FGFR3, FGFR4, FH, FLCN, FLT3, FLT4, FOXL2, GATA1, GATA2, GNA11, GNAQ, GNAS, GPC3, H3F3A, H3F3B, HNF1A, HRAS, IDH1, IDH2, IGF1R, IGF2R, IKZF1, JAK1, JAK2, JAK3, KDR, KIT, KRAS, MAML1, MAP2K1, MAP2K4, MDM2, MDM4, MED12, MEN1, MET, MLH1, MLL, MPL, MSH2, MSH6, MTOR, MUTYH, MYCL1, MYCN, NBN, NCOA3, NF1, NF2, NKX2-1, NOTCH1, NOTCH2, NOTCH3, NOTCH4, NPM1, NRAS, NTRK1, PALB2, PAX5, PBRM1, PDGFRA, PHOX2B, PIK3CA, PIK3R1, PMS1, PMS2, POLD1, POLE, POLH, POT1, PRKAR1A, PRSS1, PTCH1, PTEN, PTPN11, RAD51C, RAF1, RB1, RECQL4, RET, RNF43, ROS1, RUNX1, SBDS, SDHAF2, SDHB, SDHC, SDHD, SF3B1, SMAD2, SMAD3, SMAD4, SMARCB1, SMO, SRC, STAG2, STK11, SUFU, TERT, TET2, TGFBR2, TNFAIP3, TOP1, TP53, TSC1, TSC2, TSHR, VHL, WAS, WRN, WT1, XPA, XPC, and/or XRCC1. In some embodiments, the subject has at least one mutation in one or more sequences encoding ARID1A, ATM, B2M, BCL2, BCL6, BCL7A, BRAF, BTG1, CARD11, CCND3, CD58, CD79B, CDKN2A, CREBBP, EP300, EZH2, FOXO1, GNA13, HIST1H1B, HIST1H1C, HIST1H1E, IKZF3, IRF4, ITPKB, KDM6A, KIT, KMT2D, KRAS, MEF2B, MYC, MYD88, NOTCH1, NOTCH2, NRAS, PIK3CA, PIM1, POU2F2, PRDM1, PTEN, PTPN1, PTPN11, PTPN6, PTPRD, RB1, S1PR2, SGK1, SMARCB1, SOCS1, STAT6, TBL1XR1, TNFAIP3, TNFRSF14, TP53, XPO1. In some embodiments, the subject has at least one mutation in one or more sequences encoding AKT1, ALK, ARID1A, ATM, B2M, BCL2, BCL6, BCL7A, BTG2, CARD11, CCND3, CD79B, CDKN2A, CREBBP, EP300, EZH2, FBXW7, FOXO1, HLA-C, HRAS, IKZF3, IRF4, KDM6A, KRAS, MEF2B, MYD88, NOTCH1, NPM1, NRAS, PIK3CA, PIM1, PRDM1, PTEN, RB1, RBBP4, SMARCB1, SUZ12, TNFRSF14, and/or TP53. In some embodiments, the subject has at least one mutation in one or more sequences encoding ALK, EWSR1, ROS1, BCL2, MLL, TMPRSS2, BCR, MYC, FGFR3, BRAF, NTRK1, TACC3, DNAJB1, PDGFRA, EGFR, PDGFRB, ETV1, PRKACA, ETV4, RAF1, ETV5, RARA, ETV6, RET. In some embodiments, the subject has at least one mutation in one or more sequences encoding ALK (Intron 19), BCL2 (MBR breakpoint region), BCL2 (MCR breakpoint region), BCL6, CD274, CIITA, MYC (entire Gene+40 kbp upstream), and/or PDCD1LG2. In some embodiments, the subject has at least one mutation in one or more sequences encoding BCL2, CD274 (PDL1), FOXP1, JAK2, KDM4C, PDCD1LG2 (PDL2), and/or REL. In some embodiments, the subject has at least one mutation in one or more sequences encoding ARID1A, ATM, B2M, BCL2, BCL6, BCL7A, BRAF, CARD11, CCND3, CD274 (PDL1), CD58, CD79B, CDKN2A, CIITA, CREBBP, EZH2 (non-Y646), EZH2 (Y646), EP300, FOXO1, FOXP1, GNA13, HIST1H1B, HIST1H1C, HIST1H1E, IRF4, IZKF3, JAK2, KDM4C, KDM6A, KIT, KMT2D, KRAS, MEF2B, MYC, MYD88, NOTCH1, NOTCH2, NRAS, PDCD1LG2 (PDL2), PIK3CA, PIM1, POU2F2, PRDM1, PTEN, PTPN11, PTPN6, PTPRD, REL, SOCS1, STAT6, TNFAIP3, TNFRSF14, and/or TP53. In some embodiments, the subject has at least one mutation in one or more sequences encoding ARID1A, B2M, BCL2, BCL6, CARD11, CCND3, CD274 (PDL1), CD58, CD79B, CDKN2A, CREBBP, EZH2, EP300, FOXO1, GNA13, HIST1H1B, HIST1H1C, HIST1H1E, KMT2D, KRAS, MEF2B, MYC, MYD88 (273P), PDCD1LG2 (PDL2), PIM1, POU2F2, PRDM1, SOCS1, STAT6, TNFAIP3, and/or TNFRSF14. In some embodiments, the subject has at least one mutation in in one or more sequences encoding: EZH2, MYD88, STAT6A, SOCS1, MYC, and/or HIST1H1E,

In some embodiments, the subject has at least one mutation that decreases or abolishes the function of a gene product (e.g., a transcript, mRNA, or protein) encoded by the mutated sequence as compared to the function of the respective gene product encoded by the wild-type sequence. Such mutations are also sometimes referred to as loss-of-function mutations. Many loss-of-function mutations for the genes and gene products referred to herein that are suitable for some embodiments of this disclosure will be known to the skilled artisan. For example, in some exemplary embodiments, the subject has a loss-of-function mutation in SOCS1. In some embodiments, the subject has at least one mutation that increases the function of a gene product (e.g., a transcript, mRNA, or protein) encoded by the mutated sequence as compared to the function of the respective gene product encoded by the wild-type sequence. Such mutations are also sometimes referred to as gain-of-function mutations or activating mutations. Many gain-of-function mutations for the genes and gene products referred to herein that are suitable for some embodiments of this disclosure will be known to the skilled artisan. For example, in some embodiments, the subject has a gain-of-function mutation in a sequence encoding EZH2, MYD88, STAT6, or MYC. In some embodiments, the subject has at least one loss-of-function and at least one gain-of function mutation. For example, in some embodiments, the subject has at least one gain-of-function mutation in a sequence encoding EZH2 or STAT6, and at least one loss-of-function mutation in a sequence encoding SOCS1. In some embodiments, the subject does not have a specific mutation, e.g., a gain-of-function in a sequence encoding MYC or a loss-of-function mutation in SOCS1.

In some embodiments, the subject expresses a mutant EZH2 protein. In some embodiments, the mutant EZH2 protein comprises a substitution of any amino acid other than tyrosine (Y) for tyrosine (Y) at position 641 of SEQ ID NO: 1, a substitution of any amino acid other than alanine (A) for alanine (A) at position 682 of SEQ ID NO: 1, and/or a substitution of any amino acid other than alanine (A) for alanine (A) at position 692 of SEQ ID NO: 1. In some embodiments, the subject expresses at least one mutant MYD88, STAT6, and/or a SOCS1 protein, either in addition to the mutant EZH2 protein or in the absence of a mutant EZH2 protein. In some embodiments, the subject does not express a mutant MYC and/or a mutant HIST1H1E protein. In some embodiments, the mutant EZH2 protein, the mutant MYD88 protein, the mutant STAT6 protein, and/or the mutant MYC protein exhibits an increase in activity as compared to the respective wild-type protein. In some embodiments, the mutant SOCS1 protein exhibits a decreased activity as compared to the respective wild-type SOCS1 protein.

In some embodiments, the methods provided herein further comprise detecting the at least one mutation in the subject. Such detecting may, in some embodiments, comprise subjecting a sample obtained from the subject to a suitable sequence analysis assay, e.g., to a next generation sequencing assay. Suitable sequencing assays are provided herein or otherwise known to those of skill in the art, and the disclosure is not limited in this respect.

Some aspects of this disclosure provide methods comprising selecting a subject having cancer for treatment with an EZH2 inhibitor based on the presence of at least one mutation associated with a positive response to such treatment in the subject and/or based on the absence of at least one mutation associated with no response or with a negative response to such treatment in the subject. In some embodiments, the at least one mutation associated with a positive response comprises (a) an EZH2 mutation (e.g., a gain-of-function EZH2 mutation); (b) a histone acetyl transferase (HAT) mutation; (c) a STAT6 mutation (e.g., a gain-of-function STAT6 mutation); (d) a MYD88 mutation (e.g., a gain-of-function MYD88 mutation); and/or (e) a SOCS1 mutation (e.g., a loss-of-function SOCS1 mutation). In some embodiments, the at least one mutation associated with no response or with a negative response comprises (a) a MYC mutation (e.g., a gain-of-function MYC mutation); and/or (b) a HIST1H1E mutation. In some embodiments, the method comprises detecting the at least one mutation associated with a positive response and/or the at least one mutation associated with no response or a negative response in a sample obtained from the subject by subjecting the sample to a suitable sequence analysis assay. In some embodiments, the method comprises selecting the subject for treatment with the EZH2 inhibitor based on the subject (a) having at least one of a MYD88 mutation, a STAT6A mutation, and a SOCS1 mutation, and/or (b) not having at least one of a MYC mutation and/or a HIST1H1E mutation. In some embodiments, the method comprises selecting the subject for treatment with the EZH2 inhibitor based on the subject (a) having at least one of a MYD88 mutation, a STAT6A mutation, and a SOCS1 mutation, and (b) not having a MYC mutation and a HIST1H1E mutation.

Some aspects of this disclosure provide methods for selecting a subject having cancer for treatment with an EZH2 inhibitor based on the presence of a mutation profile in the subject that matches a mutation profile (e.g., at least 2, at least 3, at least 4, or at least 5, or more mutations, or, in some embodiments, all mutations), of a patient exhibiting a complete or partial response or stable disease as described in any of FIGS. 19A-22C.

### Definitions

According to the methods of the disclosure, a “normal” cell may be used as a basis of comparison for one or more characteristics of a cancer cell, including the presence of one or more mutations in a histone acetyltransferase that result in a decreased activity of the enzyme. For example, the one or more mutations in a histone acetyltransferase may result in a decreased acetylation activity or efficacy of the enzyme, and, consequently, a reduced or decreased level of acetylation of at least one lysine on Histone 3 (H3). In certain embodiments, the one or more mutations in a histone acetyltransferase may result in a decreased acetylation activity or efficacy of the enzyme, and, consequently, a reduced or decreased level of acetylation of lysine 27 on Histone 3 (H3) (H3K27). As used herein, a “normal cell” is a cell that cannot be classified as part of a “cell proliferative disorder”. A normal cell lacks unregulated or abnormal growth, or both, that can lead to the development of an unwanted condition or disease. Preferably, a normal cell expresses a comparable amount of EZH2 as a cancer cell. Preferably a normal cell contains a wild type sequence for all histone acetyltransferases, expresses a histone acetyltransferase transcript without mutations, and expresses a histone acetyltransferase protein without mutations that retains all functions a normal activity levels.

As used herein, “contacting a cell” refers to a condition in which a compound or other composition of matter is in direct contact with a cell, or is close enough to induce a desired biological effect in a cell.

As used herein, “treating” or “treat” describes the management and care of a subject for the purpose of combating a disease, condition, or disorder and includes the administration of an EZH2 inhibitor of the disclosure, or a pharmaceutically acceptable salt, prodrug, metabolite, polymorph or solvate thereof, to alleviate the symptoms or complications of cancer or to eliminate the cancer.

As used herein, the term “alleviate” is meant to describe a process by which the severity of a sign or symptom of cancer is decreased. Importantly, a sign or symptom can be alleviated without being eliminated. In a preferred embodiment, the administration of pharmaceutical compositions of the disclosure leads to the elimination of a sign or symptom, however, elimination is not required. Effective dosages are expected to decrease the severity of a sign or symptom. For instance, a sign or symptom of a disorder such as cancer, which can occur in multiple locations, is alleviated if the severity of the cancer is decreased within at least one of multiple locations.

As used herein, the term “severity” is meant to describe the potential of cancer to transform from a precancerous, or benign, state into a malignant state. Alternatively, or in addition, severity is meant to describe a cancer stage, for example, according to the TNM system (accepted by the International Union Against Cancer (UICC) and the American Joint Committee on Cancer (AJCC)) or by other art-recognized methods. Cancer stage refers to the extent or severity of the cancer, based on factors such as the location of the primary tumor, tumor size, number of tumors, and lymph node involvement (spread of cancer into lymph nodes). Alternatively, or in addition, severity is meant to describe the tumor grade by art-recognized methods (see, National Cancer Institute, www.cancer.gov). Tumor grade is a system used to classify cancer cells in terms of how abnormal they look under a microscope and how quickly the tumor is likely to grow and spread. Many factors are considered when determining tumor grade, including the structure and growth pattern of the cells. The specific factors used to determine tumor grade vary with each type of cancer. Severity also describes a histologic grade, also called differentiation, which refers to how much the tumor cells resemble normal cells of the same tissue type (see, National Cancer Institute, www.cancer.gov). Furthermore, severity describes a nuclear grade, which refers to the size and shape of the nucleus in tumor cells and the percentage of tumor cells that are dividing (see, National Cancer Institute, www.cancer.gov).

In another aspect of the disclosure, severity describes the degree to which a tumor has secreted growth factors, degraded the extracellular matrix, become vascularized, lost adhesion to juxtaposed tissues, or metastasized. Moreover, severity describes the number of locations to which a primary tumor has metastasized. Finally, severity includes the difficulty of treating tumors of varying types and locations. For example, inoperable tumors, those cancers which have greater access to multiple body systems (hematological and immunological tumors), and those which are the most resistant to traditional treatments are considered most severe. In these situations, prolonging the life expectancy of the subject and/or reducing pain, decreasing the proportion of cancerous cells or restricting cells to one system, and improving cancer stage/tumor grade/histological grade/nuclear grade are considered alleviating a sign or symptom of the cancer.

As used herein the term “symptom” is defined as an indication of disease, illness, injury, or that something is not right in the body. Symptoms are felt or noticed by the individual experiencing the symptom, but may not easily be noticed by others. Others are defined as non-health-care professionals.

As used herein the term “sign” is also defined as an indication that something is not right in the body. But signs are defined as things that can be seen by a doctor, nurse, or other health care professional.

Cancer is a group of diseases that may cause almost any sign or symptom. The signs and symptoms will depend on where the cancer is, the size of the cancer, and how much it affects the nearby organs or structures. If a cancer spreads (metastasizes), then symptoms may appear in different parts of the body.

As a cancer grows, it begins to push on nearby organs, blood vessels, and nerves. This pressure creates some of the signs and symptoms of cancer. Cancers may form in places where it does not cause any symptoms until the cancer has grown quite large.

Cancer may also cause symptoms such as fever, fatigue, or weight loss. This may be because cancer cells use up much of the body's energy supply or release substances that change the body's metabolism. Or the cancer may cause the immune system to react in ways that produce these symptoms. While the signs and symptoms listed above are the more common ones seen with cancer, there are many others that are less common and are not listed here. However, all art-recognized signs and symptoms of cancer are contemplated and encompassed by the disclosure.

Treating cancer may result in a reduction in size of a tumor. A reduction in size of a tumor may also be referred to as “tumor regression”. Preferably, after treatment according to the methods of the disclosure, tumor size is reduced by 5% or greater relative to its size prior to treatment; more preferably, tumor size is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Size of a tumor may be measured by any reproducible means of measurement. The size of a tumor may be measured as a diameter of the tumor.

Treating cancer may result in a reduction in tumor volume. Preferably, after treatment according to the methods of the disclosure, tumor volume is reduced by 5% or greater relative to its size prior to treatment; more preferably, tumor volume is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Tumor volume may be measured by any reproducible means of measurement.

Treating cancer may result in a decrease in number of tumors. Preferably, after treatment, tumor number is reduced by 5% or greater relative to number prior to treatment; more preferably, tumor number is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. Number of tumors may be measured by any reproducible means of measurement. The number of tumors may be measured by counting tumors visible to the naked eye or at a specified magnification. Preferably, the specified magnification is 2×, 3×, 4×, 5×, 10×, or 50×.

Treating cancer may result in a decrease in number of metastatic lesions in other tissues or organs distant from the primary tumor site. Preferably, after treatment according to the methods of the disclosure, the number of metastatic lesions is reduced by 5% or greater relative to number prior to treatment; more preferably, the number of metastatic lesions is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. The number of metastatic lesions may be measured by any reproducible means of measurement. The number of metastatic lesions may be measured by counting metastatic lesions visible to the naked eye or at a specified magnification. Preferably, the specified magnification is 2×, 3×, 4×, 5×, 10×, or 50×.

An effective amount of an EZH2 inhibitor of the disclosure, or a pharmaceutically acceptable salt, prodrug, metabolite, polymorph or solvate thereof, is not significantly cytotoxic to normal cells. For example, a therapeutically effective amount of an EZH2 inhibitor of the disclosure is not significantly cytotoxic to normal cells if administration of the EZH2 inhibitor of the disclosure in a therapeutically effective amount does not induce cell death in greater than 10% of normal cells. A therapeutically effective amount of an EZH2 inhibitor of the disclosure does not significantly affect the viability of normal cells if administration of the compound in a therapeutically effective amount does not induce cell death in greater than 10% of normal cells.

Contacting a cell with an EZH2 inhibitor of the disclosure, or a pharmaceutically acceptable salt, prodrug, metabolite, polymorph or solvate thereof, can inhibit EZH2 activity selectively in cancer cells. Administering to a subject in need thereof an EZH2 inhibitor of the disclosure, or a pharmaceutically acceptable salt, prodrug, metabolite, polymorph or solvate thereof, can inhibit EZH2 activity selectively in cancer cells.

### EZH2 Inhibitors

EZH2 inhibitors of the disclosure comprise tazemetostat (EPZ-6438):

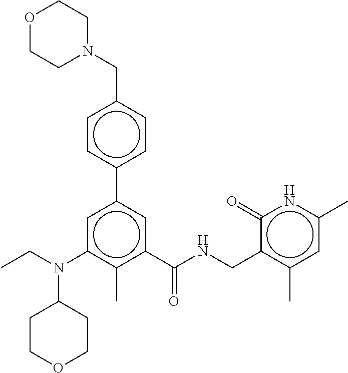

or a pharmaceutically acceptable salt thereof.

Tazemetostat is also described in U.S. Pat. Nos. 8,410,088, 8,765,732, and 9,090,562 (the contents of which are each incorporated herein in their entireties).

Tazemetostat or a pharmaceutically acceptable salt thereof, as described herein, is potent in targeting both WT and mutant EZH2. Tazemetostat is orally bioavailable and has high selectivity to EZH2 compared with other histone methyltransferases (i.e., >20,000 fold selectivity by Ki). Importantly, tazemetostat has targeted methyl mark inhibition that results in the killing of genetically defined cancer cells in vitro. Animal models have also shown sustained in vivo efficacy following inhibition of the target methyl mark. Clinical trial results described herein also demonstrate the safety and efficacy of tazemetostat.

In some embodiments, tazemetostat or a pharmaceutically acceptable salt thereof is administered to the subject at a dose of approximately 100 mg to approximately 3200 mg daily, such as about 100 mg BID to about 1600 mg BID (e.g., 100 mg BID, 200 mg BID, 400 mg BID, 800 mg BID, or 1600 mg BID), for treating a NHL. On one embodiment the dose is 800 mg BID.

EZH2 inhibitors of the disclosure may comprise, consist essentially of or consist of:

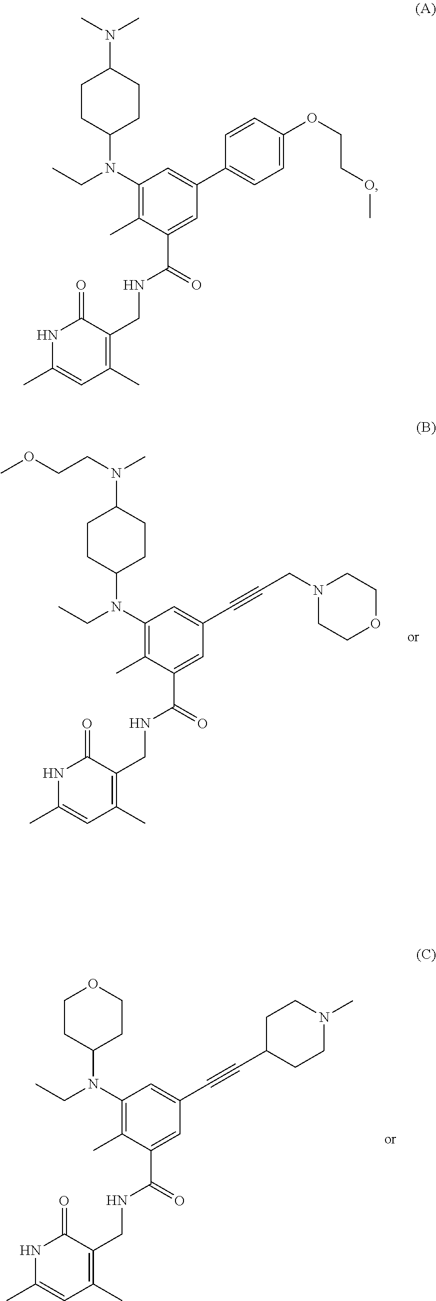

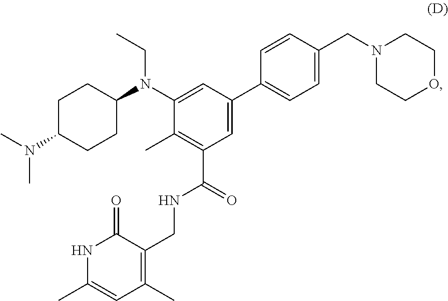 [SMILES: CCN(c1cc(-c2ccc(CN3CCOCC3)cc2)cc(C(=O)NCc2c(C)cc(C)[nH]c2=O)c1C)[C@H]1CC[C@H](N(C)C)CC1]

or stereoisomers thereof or pharmaceutically acceptable salts and solvates thereof.

EZH2 inhibitors of the disclosure may comprise, consist essentially of or consist of Compound E:

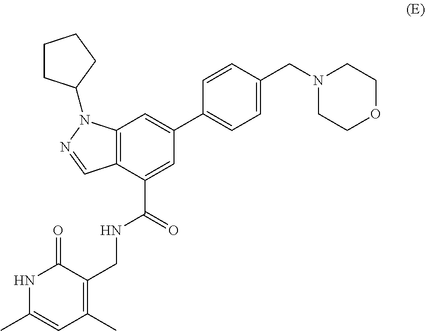 [SMILES: Cc1cc(C)c(CNC(=O)c2cc(-c3ccc(CN4CCOCC4)cc3)cc3c2cnn3C2CCCC2)c(=O)[nH]1]

or pharmaceutically acceptable salts thereof.

EZH2 inhibitors of the disclosure may comprise, consist essentially of or consist of GSK-126, having the following formula:

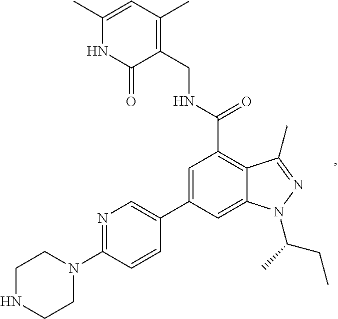 [SMILES: CC[C@H](C)n1nc(C)c2c(C(=O)NCc3c(C)cc(C)[nH]c3=O)cc(-c3ccc(N4CCNCC4)nc3)cc21]

stereoisomers thereof, or pharmaceutically acceptable salts or solvates thereof.

EZH2 inhibitors of the disclosure may comprise, consist essentially of or consist of Compound F:

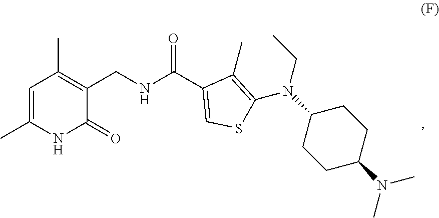

or stereoisomers thereof or pharmaceutically acceptable salts and solvates thereof.

EZH2 inhibitors of the disclosure may comprise, consist essentially of or consist of any one of Compounds Ga-Gc:

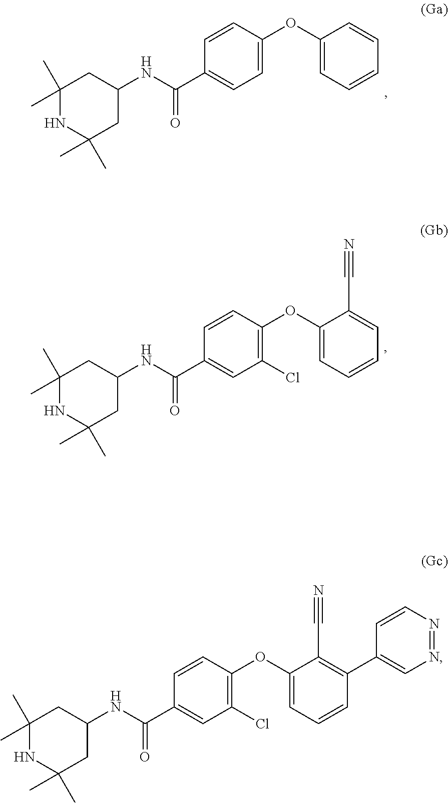 [SMILES: CC1(C)CC(NC(=O)c2ccc(Oc3ccccc3)cc2)CC(C)(C)N1 ; CC1(C)CC(NC(=O)c2ccc(Oc3ccccc3C#N)c(Cl)c2)CC(C)(C)N1 ; CC1(C)CC(NC(=O)c2ccc(Oc3cccc(-c4ccnnc4)c3C#N)c(Cl)c2)CC(C)(C)N1]

or a stereoisomer, pharmaceutically acceptable salt or solvate thereof.

EZH2 inhibitors of the disclosure may comprise, consist essentially of or consist of CPI-1205 or GSK343.

Additional suitable EZH2 inhibitors will be apparent to those skilled in the art. In some embodiments of the strategies, treatment modalities, methods, combinations, and compositions provided herein, the EZH2 inhibitor is an EZH2 inhibitor described in U.S. Pat. No. 8,536,179 (describing GSK-126 among other compounds and corresponding to WO 2011/140324), the entire contents of each of which are incorporated herein by reference.

In some embodiments of the strategies, treatment modalities, methods, combinations, and compositions provided herein, the EZH2 inhibitor is an EZH2 inhibitor described in PCT/US2014/015706, published as WO 2014/124418, in PCT/US2013/025639, published as WO 2013/120104, and in U.S. Ser. No. 14/839,273, published as US 2015/0368229, the entire contents of each of which are incorporated herein by reference.

In some embodiments, the compound disclosed herein is the compound itself, i.e., the free base or “naked” molecule. In some embodiments, the compound is a salt thereof, e.g., a mono-HCl or tri-HCl salt, mono-HBr or tri-HBr salt of the naked molecule.

Compounds disclosed herein that contain nitrogens can be converted to N-oxides by treatment with an oxidizing agent (e.g., 3-chloroperoxybenzoic acid (mCPBA) and/or hydrogen peroxides) to afford other compounds suitable for any methods disclosed herein. Thus, all shown and claimed nitrogen-containing compounds are considered, when allowed by valency and structure, to include both the compound as shown and its N-oxide derivative (which can be designated as N□O or N⁺—O⁻). Furthermore, in other instances, the nitrogens in the compounds disclosed herein can be converted to N-hydroxy or N-alkoxy compounds. For example, N-hydroxy compounds can be prepared by oxidation of the parent amine by an oxidizing agent such as m-CPBA. All shown and claimed nitrogen-containing compounds are also considered, when allowed by valency and structure, to cover both the compound as shown and its N-hydroxy (i.e., N—OH) and N-alkoxy (i.e., N—OR, wherein R is substituted or unsubstituted C₁-C₆ alkyl, C₁-C₆ alkenyl, C₁-C₆ alkynyl, 3-14-membered carbocycle or 3-14-membered heterocycle) derivatives.

“Isomerism” means compounds that have identical molecular formulae but differ in the sequence of bonding of their atoms or in the arrangement of their atoms in space. Isomers that differ in the arrangement of their atoms in space are termed “stereoisomers.” Stereoisomers that are not mirror images of one another are termed “diastereoisomers,” and stereoisomers that are non-superimposable mirror images of each other are termed “enantiomers” or sometimes optical isomers. A mixture containing equal amounts of individual enantiomeric forms of opposite chirality is termed a “racemic mixture.”

A carbon atom bonded to four nonidentical substituents is termed a “chiral center.”

“Chiral isomer” means a compound with at least one chiral center. Compounds with more than one chiral center may exist either as an individual diastereomer or as a mixture of diastereomers, termed “diastereomeric mixture.” When one chiral center is present, a stereoisomer may be characterized by the absolute configuration (R or S) of that chiral center. Absolute configuration refers to the arrangement in space of the substituents attached to the chiral center. The substituents attached to the chiral center under consideration are ranked in accordance with the Sequence Rule of Cahn, Ingold and Prelog. (Cahn et al., Angew. Chem. Inter. Edit. 1966, 5, 385; errata 511; Cahn et al., Angew. Chem. 1966, 78, 413; Cahn and Ingold, J. Chem. Soc. 1951 (London), 612; Cahn et al., Experientia 1956, 12, 81; Cahn, J. Chem. Educ. 1964, 41, 116).

“Geometric isomer” means the diastereomers that owe their existence to hindered rotation about double bonds or a cycloalkyl linker (e.g., 1,3-cylcobutyl). These configurations are differentiated in their names by the prefixes cis and trans, or Z and E, which indicate that the groups are on the same or opposite side of the double bond in the molecule according to the Cahn-Ingold-Prelog rules.

It is to be understood that the compounds disclosed herein may be depicted as different chiral isomers or geometric isomers. It should also be understood that when compounds have chiral isomeric or geometric isomeric forms, all isomeric forms are intended to be included in the scope of the disclosure, and the naming of the compounds does not exclude any isomeric forms.

Furthermore, the structures and other compounds discussed in this disclosure include all atropic isomers thereof “Atropic isomers” are a type of stereoisomer in which the atoms of two isomers are arranged differently in space. Atropic isomers owe their existence to a restricted rotation caused by hindrance of rotation of large groups about a central bond. Such atropic isomers typically exist as a mixture, however as a result of recent advances in chromatography techniques; it has been possible to separate mixtures of two atropic isomers in select cases.

“Tautomer” is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solutions where tautomerization is possible, a chemical equilibrium of the tautomers will be reached. The exact ratio of the tautomers depends on several factors, including temperature, solvent and pH. The concept of tautomers that are interconvertible by tautomerization is called tautomerism.

Of the various types of tautomerism that are possible, two are commonly observed. In keto-enol tautomerism a simultaneous shift of electrons and a hydrogen atom occurs. Ring-chain tautomerism arises as a result of the aldehyde group (—CHO) in a sugar chain molecule reacting with one of the hydroxy groups (—OH) in the same molecule to give it a cyclic (ring-shaped) form as exhibited by glucose.

Common tautomeric pairs are: ketone-enol, amide-nitrile, lactam-lactim, amide-imidic acid tautomerism in heterocyclic rings (e.g., in nucleobases such as guanine, thymine and cytosine), imine-enamine and enamine-enamine. An example of keto-enol equilibria is between pyridin-2(1H)-ones and the corresponding pyridin-2-ols, as shown below.

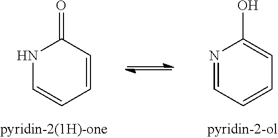 [SMILES: O=c1cccc[nH]1 ; Oc1ccccn1]

It is to be understood that the compounds disclosed herein may be depicted as different tautomers. It should also be understood that when compounds have tautomeric forms, all tautomeric forms are intended to be included in the scope of the disclosure, and the naming of the compounds does not exclude any tautomer form.

The compounds disclosed herein include the compounds themselves, as well as their salts and their solvates, if applicable. A salt, for example, can be formed between an anion and a positively charged group (e.g., amino) on an aryl- or heteroaryl-substituted benzene compound. Suitable anions include chloride, bromide, iodide, sulfate, bisulfate, sulfamate, nitrate, phosphate, citrate, methanesulfonate, trifluoroacetate, glutamate, glucuronate, glutarate, malate, maleate, succinate, fumarate, tartrate, tosylate, salicylate, lactate, naphthalenesulfonate, and acetate (e.g., trifluoroacetate). The term “pharmaceutically acceptable anion” refers to an anion suitable for forming a pharmaceutically acceptable salt. Likewise, a salt can also be formed between a cation and a negatively charged group (e.g., carboxylate) on an aryl- or heteroaryl-substituted benzene compound. Suitable cations include sodium ion, potassium ion, magnesium ion, calcium ion, and an ammonium cation such as tetramethylammonium ion. The aryl- or heteroaryl-substituted benzene compounds also include those salts containing quaternary nitrogen atoms. In the salt form, it is understood that the ratio of the compound to the cation or anion of the salt can be 1:1, or any ration other than 1:1, e.g., 3:1, 2:1, 1:2, or 1:3.

Additionally, the compounds disclosed herein, for example, the salts of the compounds, can exist in either hydrated or unhydrated (the anhydrous) form or as solvates with other solvent molecules. Nonlimiting examples of hydrates include monohydrates, dihydrates, etc. Nonlimiting examples of solvates include ethanol solvates, acetone solvates, etc.

“Solvate” means solvent addition forms that contain either stoichiometric or non-stoichiometric amounts of solvent. Some compounds have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O.

As used herein, the term “analog” refers to a chemical compound that is structurally similar to another but differs slightly in composition (as in the replacement of one atom by an atom of a different element or in the presence of a particular functional group, or the replacement of one functional group by another functional group). Thus, an analog is a compound that is similar or comparable in function and appearance, but not in structure or origin to the reference compound.

As defined herein, the term “derivative” refers to compounds that have a common core structure, and are substituted with various groups as described herein. For example, all of the compounds represented by Formula (I) are aryl- or heteroaryl-substituted benzene compounds, and have Formula (I) as a common core.

The term “bioisostere” refers to a compound resulting from the exchange of an atom or of a group of atoms with another, broadly similar, atom or group of atoms. The objective of a bioisosteric replacement is to create a new compound with similar biological properties to the parent compound. The bioisosteric replacement may be physicochemically or topologically based. Examples of carboxylic acid bioisosteres include, but are not limited to, acyl sulfonimides, tetrazoles, sulfonates and phosphonates. See, e.g., Patani and LaVoie, Chem. Rev. 96, 3147-3176, 1996.

The present disclosure is intended to include all isotopes of atoms occurring in the present compounds. Isotopes include those atoms having the same atomic number but different mass numbers. By way of general example and without limitation, isotopes of hydrogen include tritium and deuterium, and isotopes of carbon include C-13 and C-14.

### Pharmaceutical Formulations

The present disclosure also provides pharmaceutical compositions comprising at least one EZH2 inhibitor described herein in combination with at least one pharmaceutically acceptable excipient or carrier.

A “pharmaceutical composition” is a formulation containing the EZH2 inhibitors of the present disclosure in a form suitable for administration to a subject. In some embodiments, the pharmaceutical composition is in bulk or in unit dosage form. The unit dosage form is any of a variety of forms, including, for example, a capsule, an IV bag, a tablet, a single pump on an aerosol inhaler or a vial. The quantity of active ingredient (e.g., a formulation of the disclosed compound or salt, hydrate, solvate or isomer thereof) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a compound of this disclosure include powders, sprays, ointments, pastes, creams, lotions, gels, solutions, patches and inhalants. In some embodiments, the active compound is mixed under sterile conditions with a pharmaceutically acceptable carrier, and with any preservatives, buffers or propellants that are required.

As used herein, the phrase “pharmaceutically acceptable” refers to those compounds, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the disclosure includes both one and more than one such excipient.

A pharmaceutical composition of the disclosure is formulated to be compatible with its intended route of administration. Examples of routes of administration include parenteral, e.g., intravenous, intradermal, subcutaneous, oral (e.g., inhalation), transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

A compound or pharmaceutical composition of the disclosure can be administered to a subject in many of the well-known methods currently used for chemotherapeutic treatment. For example, for treatment of cancers, a compound of the disclosure may be injected directly into tumors, injected into the blood stream or body cavities or taken orally or applied through the skin with patches. The dose chosen should be sufficient to constitute effective treatment but not as high as to cause unacceptable side effects. The state of the disease condition (e.g., cancer, precancer, and the like) and the health of the patient should preferably be closely monitored during and for a reasonable period after treatment.

The term “therapeutically effective amount”, as used herein, refers to an amount of an EZH2 inhibitor, composition, or pharmaceutical composition thereof effective to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician. In a preferred aspect, the disease or condition to be treated is cancer, including but not limited to, B cell lymphoma, including activated B-cell (ABC) and germinal B-cell (GBC) subtypes.

For any EZH2 inhibitor of the disclosure, the therapeutically effective amount can be estimated initially either in cell culture assays, e.g., of neoplastic cells, or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LD50 (the dose lethal to 50% of the population). The dose ratio between toxic and therapeutic effects is the therapeutic index, and it can be expressed as the ratio, LD₅₀/ED₅₀. Pharmaceutical compositions that exhibit large therapeutic indices are preferred. The dosage may vary within this range depending upon the dosage form employed, sensitivity of the patient, and the route of administration.

Dosage and administration are adjusted to provide sufficient levels of the active agent(s) or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

The pharmaceutical compositions containing an EZH2 inhibitor of the present disclosure may be manufactured in a manner that is generally known, e.g., by means of conventional mixing, dissolving, granulating, dragee-making, levigating, emulsifying, encapsulating, entrapping, or lyophilizing processes. Pharmaceutical compositions may be formulated in a conventional manner using one or more pharmaceutically acceptable carriers comprising excipients and/or auxiliaries that facilitate processing of the active compounds into preparations that can be used pharmaceutically. Of course, the appropriate formulation is dependent upon the route of administration chosen.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser, which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The active compounds (e.g., EZH2 inhibitors of the disclosure) can be prepared with pharmaceutically acceptable carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the particular therapeutic effect to be achieved.

In therapeutic applications, the dosages of the pharmaceutical compositions used in accordance with the disclosure vary depending on the agent, the age, weight, and clinical condition of the recipient patient, and the experience and judgment of the clinician or practitioner administering the therapy, among other factors affecting the selected dosage. Generally, the dose should be sufficient to result in slowing, and preferably regressing, the growth of the tumors and also preferably causing complete regression of the cancer. An effective amount of a pharmaceutical agent is that which provides an objectively identifiable improvement as noted by the clinician or other qualified observer. For example, regression of a tumor in a patient may be measured with reference to the diameter of a tumor. Decrease in the diameter of a tumor indicates regression. Regression is also indicated by failure of tumors to reoccur after treatment has stopped. As used herein, the term “dosage effective manner” refers to amount of an active compound to produce the desired biological effect in a subject or cell.

The pharmaceutical compositions can be included in a container, pack, or dispenser together with instructions for administration.

The compounds of the present disclosure are capable of further forming salts. All of these forms are also contemplated within the scope of the claimed disclosure.

As used herein, “pharmaceutically acceptable salts” refer to derivatives of the compounds of the present disclosure wherein the parent compound is modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicyclic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc.

Other examples of pharmaceutically acceptable salts include hexanoic acid, cyclopentane propionic acid, pyruvic acid, malonic acid, 3-(4-hydroxybenzoyl)benzoic acid, cinnamic acid, 4-chlorobenzenesulfonic acid, 2-naphthalenesulfonic acid, 4-toluenesulfonic acid, camphorsulfonic acid, 4-methylbicyclo-[2.2.2]-oct-2-ene-1-carboxylic acid, 3-phenylpropionic acid, trimethylacetic acid, tertiary butylacetic acid, muconic acid, and the like. The present disclosure also encompasses salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

It should be understood that all references to pharmaceutically acceptable salts include solvent addition forms (solvates) or crystal forms (polymorphs) as defined herein, of the same salt.

The EZH2 inhibitors of the present disclosure can also be prepared as esters, for example, pharmaceutically acceptable esters. For example, a carboxylic acid function group in a compound can be converted to its corresponding ester, e.g., a methyl, ethyl or other ester. Also, an alcohol group in a compound can be converted to its corresponding ester, e.g., an acetate, propionate or other ester.

The EZH2 inhibitors of the present disclosure can also be prepared as prodrugs, for example, pharmaceutically acceptable prodrugs. The terms “pro-drug” and “prodrug” are used interchangeably herein and refer to any compound which releases an active parent drug in vivo. Since prodrugs are known to enhance numerous desirable qualities of pharmaceuticals (e.g., solubility, bioavailability, manufacturing, etc.), the compounds of the present disclosure can be delivered in prodrug form. Thus, the present disclosure is intended to cover prodrugs of the presently claimed compounds, methods of delivering the same and compositions containing the same. “Prodrugs” are intended to include any covalently bonded carriers that release an active parent drug of the present disclosure in vivo when such prodrug is administered to a subject. Prodrugs in the present disclosure are prepared by modifying functional groups present in the compound in such a way that the modifications are cleaved, either in routine manipulation or in vivo, to the parent compound. Prodrugs include compounds of the present disclosure wherein a hydroxy, amino, sulfhydryl, carboxy or carbonyl group is bonded to any group that may be cleaved in vivo to form a free hydroxyl, free amino, free sulfhydryl, free carboxy or free carbonyl group, respectively.

Examples of prodrugs include, but are not limited to, esters (e.g., acetate, dialkylaminoacetates, formates, phosphates, sulfates and benzoate derivatives) and carbamates (e.g., N,N-dimethylaminocarbonyl) of hydroxy functional groups, esters (e.g., ethyl esters, morpholinoethanol esters) of carboxyl functional groups, N-acyl derivatives (e.g., N-acetyl) N-Mannich bases, Schiff bases and enaminones of amino functional groups, oximes, acetals, ketals and enol esters of ketone and aldehyde functional groups in compounds of the disclosure, and the like, See Bundegaard, H., Design of Prodrugs, p 1-92, Elesevier, New York-Oxford (1985).

The EZH2 inhibitors, or pharmaceutically acceptable salts, esters or prodrugs thereof, are administered orally, nasally, transdermally, pulmonary, inhalationally, buccally, sublingually, intraperintoneally, subcutaneously, intramuscularly, intravenously, rectally, intrapleurally, intrathecally and parenterally. In some embodiments, the compound is administered orally. One skilled in the art will recognize the advantages of certain routes of administration.

The dosage regimen utilizing the compounds is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter or arrest the progress of the condition.

The dosage regimen can be daily administration (e.g., every 24 hours) of a compound of the present disclosure. The dosage regimen can be daily administration for consecutive days, for example, at least two, at least three, at least four, at least five, at least six or at least seven consecutive days. Dosing can be more than one time daily, for example, twice, three times or four times daily (per a 24 hour period). The dosing regimen can be a daily administration followed by at least one day, at least two days, at least three days, at least four days, at least five days, or at least six days, without administration.

Techniques for formulation and administration of the disclosed compounds of the disclosure can be found in Remington: the Science and Practice of Pharmacy, 19^{th} edition, Mack Publishing Co., Easton, Pa. (1995). In some embodiments, the compounds described herein, and the pharmaceutically acceptable salts thereof, are used in pharmaceutical preparations in combination with a pharmaceutically acceptable carrier or diluent. Suitable pharmaceutically acceptable carriers include inert solid fillers or diluents and sterile aqueous or organic solutions. The compounds will be present in such pharmaceutical compositions in amounts sufficient to provide the desired dosage amount in the range described herein.

Methods of the disclosure for treating cancer including treating a B cell lymphoma, including the activated B-cell (ABC) and germinal B-cell (GBC) subtypes. In preferred embodiments, methods of the disclosure are used to treat a subject having a B cell lymphoma. In certain embodiments, the B cell lymphoma cell and/or the subject are characterized as having one or more mutations in a sequence that encodes a histone acetyltransferase (HAT). B cell lymphoma cells may contain a mutation in a gene that encodes a HAT, a corresponding HAT transcript (or cDNA copy thereof), or a HAT protein that decreases/inhibits an activity of a HAT protein. In preferred embodiments, the mutation in a gene that encodes a HAT, a corresponding HAT transcript (or cDNA copy thereof), or a HAT protein that decreases/inhibits an activity of a HAT protein, decreases or inhibits an acetylation activity or efficacy of the enzyme, resulting in a decreased level of acetylation of one or more lysines of histone 3 (H3) (e.g., H3K27). The presence of the HAT mutation resulting in a decreased level of acetylation of one or more lysines of histone 3 (H3) (e.g., H3K27) in a cell renders that cell sensitive to oncogenic transformation and treatment with an EZH2 inhibitor.

Methods of the disclosure may be used to treat a subject who has one or more mutations in a HAT that decrease/inhibit the ability of the HAT to acetylate one or more lysines of histone 3 (H3) (e.g., H3K27) or who has one or more cells with one or more mutations in a HAT that decrease/inhibit the ability of the HAT to acetylate one or more lysines of histone 3 (H3) (e.g., H3K27). HAT expression and/or HAT function may be evaluated by fluorescent and non-fluorescent immunohistochemistry (IHC) methods, including well known to one of ordinary skill in the art. In a certain embodiment the method comprises: (a) obtaining a biological sample from the subject; (b) contacting the biological sample or a portion thereof with an antibody that specifically binds HAT; and (c) detecting an amount of the antibody that is bound to HAT. Alternatively, or in addition, HAT expression and/or HAT function may be evaluated by a method comprising: (a) obtaining a biological sample from the subject; (b) sequencing at least one DNA sequence encoding a HAT protein from the biological sample or a portion thereof; and (c) determining if the at least one DNA sequence encoding a HAT protein contains a mutation affecting the expression and/or function of the HAT protein. HAT expression or a function of HAT may be evaluated by detecting an amount of the antibody that is bound to HAT and by sequencing at least one DNA sequence encoding a HAT protein, optionally, using the same biological sample from the subject.

All percentages and ratios used herein, unless otherwise indicated, are by weight.

Other features and advantages of the present disclosure are apparent from the different examples. The provided examples illustrate different components and methodology useful in practicing the present disclosure. The examples do not limit the claimed disclosure. Based on the present disclosure the skilled artisan can identify and employ other components and methodology useful for practicing the present disclosure.

### EXAMPLES

In order that the invention disclosed herein may be more efficiently understood, examples are provided below. It should be understood that these examples are for illustrative purposes only and are not to be construed as limiting the disclosure in any manner.

### Example 1: Identification of One or More Mutant Histone Acetyltransferase from 39 Gene Panel

Analysis of somatic sequence mutations (including single base and insertion/deletions) for 39 genes (Table 1 below) was performed on DNA from archival tumor tissue isolated and embedded in paraffin blocks prior to the treatment with EZH2 inhibitor Tazemetostat. DNA was extracted from up to four 10-micron slides sectioned from a formalin fixed paraffin embedded tumor sample. Samples were macrodissected if tumor content was determined to be less than 80% by a trained pathologist. Amplicon based library prep using custom Ampli-Seq primers (ThermoFisher) was performed using 10 ng of DNA as input. Quantitation of the library was completed using emulsion PCR and then sequenced using the Ion Torrent Personal Genome Machine (ThermoFisher) to an average depth of 500×. Base calling, mapping and mutation calling was performed by Torrent Suite 3.6.2 or later and Variant caller plug-in 3.6.63335 or later. Mutation calls were reported only for mutations with greater than 500X coverage and supported by at least 10% allelic frequency.

TABLE 1
Custom 39 gene sequencing panel.

| Gene | # of Amplicons |
|---|---|
| AKT1 | 2 |
| ALK | 2 |
| ARID1A | 6 |
| ATM | 17 |
| B2M | 1 |
| BCL2 | 1 |
| BCL6 | 1 |
| BCL7A | 1 |
| BTG2 | 1 |
| CARD11 | 3 |
| CCND3 | 1 |
| CD79B | 1 |
| CDKN2A | 2 |
| CREBBP | 1 |
| EP300 | 1 |
| EZH2* | 35 |
| FBXW7 | 5 |
| FOXO1 | 1 |
| HLA-C | 1 |
| HRAS | 2 |
| IKZF3 | 1 |
| IRF4 | 1 |
| KDM6A* | 63 |
| KRAS | 3 |
| MEF2B | 3 |
| MYD88 | 3 |
| NOTCH1 | 3 |
| NPM1 | 1 |
| NRAS | 3 |
| PIK3CA | 11 |
| PIM1 | 2 |
| PRDM1 | 2 |
| PTEN | 9 |
| RB1 | 7 |
| RBBP4 | 1 |
| SMARCB1 | 5 |
| SUZ12 | 1 |
| TNFRSF14 | 1 |
| TP53 | 11 |

*EZH2 & KDM6A covered the entire Coding Region

### Example 2: Identification of One or More Mutant Histone Acetyltransferase from 62 Gene Panel from Non-Hodgkin's Lymphoma (NHL) Tissue

A panel of 62 NHL specific and 203 well-characterized cancer genes was designed to selectively analyze regions of the genome previously identified as somatically altered (Tables 2 through 6). The panel was designed to capture somatic sequence mutations (single base and small insertions/deletions), amplifications, translocations, and microsatellite instability (MSI). DNA was extracted from up to five, 5-micron slides sectioned from a formalin fixed paraffin embedded tumor sample that was prepared prior to the start of Tazemetostat treatment. Targeted genomic capture was performed using 100 ng of input DNA and then sequenced to an average depth of 1500-fold using the Illumina HiSeq2500 platform with 100 bp paired-end reads. Bioinformatics was performed by aligning the filtered data to the hg19 reference genome allowing for the identification of tumor specific sequence alterations (single base and small insertion/deletion alterations). Further analysis for identification of copy number alterations and translocations was performed using digital karyotyping and PARE analyses respectively. The validation of the panel was completed through the analyses of cell line specimens with an experimental tumor purity of 20-100% using 50-100 ng of DNA yielded sensitivity and specificity of 100% for detection of 358 previously characterized sequence mutations and structural variants.

TABLE 2
Custom Lymphoma CancerSelect ™ Sequence Mutation Gene List (in
addition to the CancerSelect-R ™ 203 Gene Panel).

| | Sequence | | Sequence |
|---|---|---|---|
| Gene | Region(s) | Gene | Region(s) |
| Name | Included | Name | Included |
| PRDM1 | Full Coding Sequence | KIT | Specific Exon(s) |
| EZH2 | Full Coding Sequence | KRAS | Specific Exon(s) |
| KDM6A | Full Coding Sequence | MEF2B | Specific Exon(s) |
| KMT2D | Full Coding Sequence | MYC | Specific Exon(s) |
| ARID1A | Specific Exon(s) | MYD88 | Specific Exon(s) |
| ATM | Specific Exon(s) | NOTCH1 | Specific Exon(s) |
| B2M | Specific Exon(s) | NOTCH2 | Specific Exon(s) |
| BCL2 | Specific Exon(s) | NRAS | Specific Exon(s) |
| BCL6 | Specific Exon(s) | PIK3CA | Specific Exon(s) |
| BCL7A | Specific Exon(s) | PIM1 | Specific Exon(s) |
| BRAF | Specific Exon(s) | POU2F2 | Specific Exon(s) |
| BTG1 | Specific Exon(s) | PTEN | Specific Exon(s) |
| CARD11 | Specific Exon(s) | PTPN1 | Specific Exon(s) |
| CCND3 | Specific Exon(s) | PTPN11 | Specific Exon(s) |
| CD58 | Specific Exon(s) | PTPN6 | Specific Exon(s) |
| CD79B | Specific Exon(s) | PTPRD | Specific Exon(s) |
| CDKN2A | Specific Exon(s) | RB1 | Specific Exon(s) |
| CREBBP | Specific Exon(s) | S1PR2 | Specific Exon(s) |
| EP300 | Specific Exon(s) | SGK1 | Specific Exon(s) |
| FOXO1 | Specific Exon(s) | SMARCB1 | Specific Exon(s) |
| GNA13 | Specific Exon(s) | SOCS1 | Specific Exon(s) |
| HIST1H1B | Specific Exon(s) | STAT6 | Specific Exon(s) |
| HIST1H1C | Specific Exon(s) | TBL1XR1 | Specific Exon(s) |
| HIST1H1E | Specific Exon(s) | TNFAIP3 | Specific Exon(s) |
| IKZF3 | Specific Exon(s) | TNFRSF14 | Specific Exon(s) |
| IRF4 | Specific Exon(s) | TP53 | Specific Exon(s) |
| ITPKB | Specific Exon(s) | XPO1 | Specific Exon(s) |

*Specific exons were chosen based on those regions which were mutated recurrently in COSMIC

TABLE 3
Custom Lymphoma CancerSelect ™ Translocation
Analyses Gene List (in addition to the CancerSelect-
R ™ 203 Gene Panel).

| | Sequence | | Sequence |
|---|---|---|---|
| Gene | Region(s) | Gene | Region(s) |
| Name | Included | Name | Included |
| ALK | ALK_NM_004304_Intron19 | CIITA | Entire Gene |
| BCL2 | BCL2_MCR_Break- | MYC | Entire Gene + |
| | point_Region | | 40 kbp |
| | | | upstream |
| BCL2 | BCL2_MBR_Break- | CD274 | Entire Gene |
| | point_Region | (PDL1) | |
| BCL6 | Entire Gene | PDCD1LG2 | Entire Gene |
| | | (PDL2) | |

TABLE 4
Custom Lymphoma CancerSelect ™ Amplification
Analyses Gene List (in addition to the CancerSelect-
R ™ 203 Gene Panel).

| Gene Name | Gene Name |
|---|---|
| BCL2 | JAK2 |
| CD274 (PDL1) | KDM4C |
| FOXP1 | PDCD1LG2 (PDL2) |
| | REL |

TABLE 5
CancerSelect-R ™ 203 Gene Panel (Sequence and
copy number* analyses for the full coding sequence of
195 well-characterized cancer genes).

| Gene Name | Gene Name | Gene Name | Gene Name | Gene Name |
|---|---|---|---|---|
| ABL1* | CBL* | ERBB3* | FGFR2* | KDR* |
| ACVR1 | CCND1* | ERBB4* | FGFR3* | KIT* |
| AKT1* | CCNE1* | ERCC1 | FGFR4* | KRAS* |
| AKT2* | CDC73 | ERCC2 | FH | MAML1* |
| ALK* | CDH1 | ERCC3 | FLCN | MAP2K1* |
| APC | CDK4* | ERCC4 | FLT3* | MAP2K4 |
| AR* | CDK6* | ERCC5 | FLT4 | MDM2* |
| ARID1A | CDKN1B | ESR1 | FOXL2* | MDM4* |
| ARID1B | CDKN2A | ETV1 | GATA1 | MED12* |
| ASXL1 | CDKN2B | ETV5 | GATA2* | MEN1 |
| ATM | CDKN2C | EWSR1 | GNA11* | MET* |
| ATRX | CEBPA | EXT1 | GNAQ* | MLH1 |
| AURKA | CHEK2 | EXT2 | GNAS* | MLL* |
| AXIN2 | CIC | EZH2* | GPC3 | MPL* |
| BAP1 | CREBBP | FANCA | H3F3A* | MSH2 |
| BCL2* | CSF1R* | FANCB | H3F3B | MSH6 |
| BCR | CTNNB1* | FANCC | HNF1A | MTOR |
| BLM | CYLD | FANCD2 | HRAS* | MUTYH |
| BMPR1A | DAXX | FANCE | IDH1* | MYC* |
| BRAF* | DDB2 | FANCF | IDH2* | MYCL1* |
| BRCA1 | DDR2 | FANCG | IGF1R* | MYCN* |
| BRCA2 | DICER1 | FANCI | IGF2R* | MYD88* |
| BRIP1 | DNMT3A* | FANCL | IKZF1 | NBN |
| BTK | EGFR* | FANCM | JAK1* | NCOA3* |
| BUB1B | EP300 | FBXW7 | JAK2* | NF1 |
| CALR | ERBB2* | FGFR1 | JAK3* | NF2 |
| NKX2-1* | PIK3CA* | RAD51C | SF3B1* | TNFAIP3 |
| NOTCH1* | PIK3R1 | RAF1 | SMAD2 | TOP1 |
| NOTCH2* | PMS1 | RB1 | SMAD3 | TP53 |
| NOTCH3* | PMS2 | RECQL4 | SMAD4 | TSC1 |
| NOTCH4* | POLD1 | RET* | SMARCB1 | TSC2 |
| NPM1 | POLE | RNF43 | SMO* | TSHR* |
| NRAS* | POLH | ROS1 | SRC | VHL |
| NTRK1 | POT1 | RUNX1* | STAG2 | WAS |
| PALB2 | PRKAR1A | SBDS | STK11 | WRN |
| PAX5* | PRSS1 | SDHAF2 | SUFU | WT1 |
| PBRM1 | PTCH1 | SDHB | TERT | XPA |
| PDGFRA* | PTEN | SDHC | TET2 | XPC |
| PHOX2B | PTPN11* | SDHD | TGFBR2 | XRCC1 |

TABLE 6
CancerSelect-R ™ 203 Gene Panel (Rearrangement analyses
for selected regions of 24 well-characterized genes.

| Gene Name | Gene Name | Gene Name |
|---|---|---|
| ALK | EWSR1 | ROS1 |
| BCL2 | MLL | TMPRSS2 |
| BCR | MYC | FGFR3 |
| BRAF | NTRK1 | TACC3 |
| DNAJB1 | PDGFRA | |
| EGFR | PDGFRB | |
| ETV1 | PRKACA | |
| ETV4 | RAF1 | |
| ETV5 | RARA | |
| ETV6 | RET | |

### Example 3: Non-Hodgkin's Lymphoma Circulating DNA Panel

A panel of 62 NHL specific genes was designed to selectively analyze regions of the genome previously identified as somatically altered (Table 7) with high specificity down to an allelic frequency of 0.1%. The panel was designed to capture somatic sequence mutations (single base and small insertions/deletions), amplifications, translocations, and microsatellite instability (MSI). DNA was extracted from plasma derived from up to 20 mLs of peripheral blood. Blood was collected prior to treatment and at defined time points during the course of Tazemetostat treatment. Targeted genomic capture was performed using 150 ng of input DNA and then sequenced using the Illumina HiSeq2500 platform with 100 bp paired-end reads. The average depth of sequencing coverage was approximately 20,000-fold for sequence mutations and 5,000-fold for structural alterations. Bioinformatic analyses were accomplished by aligning the filtered data to the hg19 reference genome allowing for the identification of tumor specific sequence alterations (single base and small insertion/deletion alterations). Further analyses for identification of copy number alterations and translocations was performed by digital karyotyping and PARE analyses respectively. The validation of the panel was completed using analyses of fragmented cell line and plasma derived DNA with an experimental tumor purity of 0.10%-25.0% using 9-167 ng of DNA yielded a sensitivity of 100% for detection of over 100 genetic variants.

TABLE 7
Custom Lymphoma CancerSelect ™ Sequence
Mutation Gene List.

| | Sequence | | Sequence |
|---|---|---|---|
| Gene | Region(s) | Gene | Region(s) |
| Name | Included | Name | Included |
| PRDM1 | Full Coding Sequence | KIT | Specific Exon(s) |
| EZH2 | Full Coding Sequence | KRAS | Specific Exon(s) |
| KDM6A | Full Coding Sequence | MEF2B | Specific Exon(s) |
| KMT2D | Full Coding Sequence | MYC | Specific Exon(s) |
| ARID1A | Specific Exon(s) | MYD88 | Specific Exon(s) |
| ATM | Specific Exon(s) | NOTCH1 | Specific Exon(s) |
| B2M | Specific Exon(s) | NOTCH2 | Specific Exon(s) |
| BCL2 | Specific Exon(s) | NRAS | Specific Exon(s) |
| BCL6 | Specific Exon(s) | PIK3CA | Specific Exon(s) |
| BCL7A | Specific Exon(s) | PIM1 | Specific Exon(s) |
| BRAF | Specific Exon(s) | POU2F2 | Specific Exon(s) |
| BTG1 | Specific Exon(s) | PTEN | Specific Exon(s) |
| CARD11 | Specific Exon(s) | PTPN1 | Specific Exon(s) |
| CCND3 | Specific Exon(s) | PTPN11 | Specific Exon(s) |
| CD58 | Specific Exon(s) | PTPN6 | Specific Exon(s) |
| CD79B | Specific Exon(s) | PTPRD | Specific Exon(s) |
| CDKN2A | Specific Exon(s) | RB1 | Specific Exon(s) |
| CREBBP | Specific Exon(s) | S1PR2 | Specific Exon(s) |
| EP300 | Specific Exon(s) | SGK1 | Specific Exon(s) |
| FOXO1 | Specific Exon(s) | SMARCB1 | Specific Exon(s) |
| GNA13 | Specific Exon(s) | SOCS1 | Specific Exon(s) |
| HIST1H1B | Specific Exon(s) | STAT6 | Specific Exon(s) |
| HIST1H1C | Specific Exon(s) | TBL1XR1 | Specific Exon(s) |
| HIST1H1E | Specific Exon(s) | TNFAIP3 | Specific Exon(s) |
| IKZF3 | Specific Exon(s) | TNFRSF14 | Specific Exon(s) |
| IRF4 | Specific Exon(s) | TP53 | Specific Exon(s) |
| ITPKB | Specific Exon(s) | XPO1 | Specific Exon(s) |

*Specific exons were chosen based on those regions which were mutated recurrently in COSMIC

TABLE 8
Custom Lymphoma CancerSelect ™
Translocation Analyses Gene List.

| | Sequence | | Sequence |
|---|---|---|---|
| Gene | Region(s) | Gene | Region(s) |
| Name | Included | Name | Included |
| ALK | ALK_NM_004304_Intron19 | CIITA | Entire Gene |
| BCL2 | BCL2_MCR_Break- | MYC | Entire Gene + |
| | point_Region | | 40 kbp |
| | | | upstream |
| BCL2 | BCL2_MBR_Break- | CD274 | Entire Gene |
| | point_Region | (PDL1) | |
| BCL6 | Entire Gene | PDCD1LG2 | Entire Gene |
| | | (PDL2) | |

TABLE 9
Custom Lymphoma CancerSelect ™
Amplification Analyses Gene List.

| Gene Name | Gene Name |
|---|---|
| BCL2 | JAK2 |
| CD274 (PDL1) | KDM4C |
| FOXP1 | PDCD1LG2 (PDL2) |
| | REL |

Table 10 describes a Phase 1 clinical trial design (sponsor protocol no.: E7438-G000-001, ClinicalTrials.gov identifier: NCT01897571). The study population included subjects with relapsed or refractory solid tumors or B-cell lymphoma. Subjects received a 3+3 dose escalation in expansion cohorts receiving 800 mg BID and 1600 mg BID, respectively, or a cohort for ascertaining the effect of food on dosing at 400 mg BID. The primary endpoint was a determination of recommended phase II dose (RP2D)/maximum tolerated dose (MTD). Secondary endpoints included safety, pharmacokinetics (PK), pharmacodynamics (PD) and tumor response, assessed every 8 wks.

TABLE 10

| Dose | Patients | Solid tumors | B-cell NHL |
|---|---|---|---|
| (mg BID) | (n = 58) | (1 = 37)** | (n = 21) |
| 100* | 6 | 5 | 1 |
| 200 | 3 | 1 | 2 |
| 400 | 3 | 2 | 1 |
| 800 | 14 | 6 | 8 |
| 1600 | 12 | 8 | 4 |
| Food Effect | 13 | 8 | 5 |
| Drug-Drug | 7 | 7 | 0 |

*2 formulations

Table 11 provides patient tumor type data from the trial described in Table 10.

TABLE 11

| Relapsed or refractory NHL | | n = 21 |
|---|---|---|
| Diffuse Large B cell | GCB | 5 |
| Lymphoma (DLBCL)* | Non GCB | 6 |
| | undetermined | 3 |

| | |
|---|---|
| Follicular lymphoma (FL)* | 6 |
| Marginal Zone lymphoma (MZL) | 1 |

| Relapsed or refractory solid tumors | | n = 37 |
|---|---|---|
| INI1-deficient or | Malignant rhabdoid tumor | 5 |
| negative | Epithelioid sarcoma | 3 |
| | Synovial sarcoma | 4 |

| | |
|---|---|
| SMARCA4-negative tumors | 3 |
| Other solid tumors | 22 |

2/17 NHL patients tested to date are EZH2 mutant by Cobas® test (Roche Molecular Systems, Inc.)

Table 12 summarizes solid tumor patient demographics from the trial described in Table 10.

TABLE 12

| | Characteristic | | n = 21 (%) | |
|---|---|---|---|---|
| Median age, years [range] | | | 63 | [24-84] |
| Sex (M/F) | | | 15/6 | |
| | # of prior therapeutic | 1 | 2 | (10) |
| | systemic regimens | 2 | 1 | (5) |
| | | 3 | 8 | (38) |
| | | 4 | 3 | (14) |
| | | ≥5 | 7 | (33) |

| | | |
|---|---|---|
| Prior autologous hematopoietic cell | 8 | (38) |
| Prior radiotherapy | 17 | (57) |

Table 13 describes a safety profile in NHL (non-Hodgkin's lymphoma) and solid tumor patients (n-51)

TABLE 13

| | All Events | | All Treatment-Related | |
|---|---|---|---|---|
| | All Grades * | Grade ≥3 | All Grades | Grade ≥3 ** |
| Asthenia | 23 | 0 | 13 | 0 |
| Decreased appetite | 9 | 1 | 4 | 0 |
| Thrombocytopenia | 8 | 2 | 7 | 1 |
| Nausea | 8 | 0 | 8 | 0 |
| Constipation | 7 | 0 | 2 | 0 |
| Diarrhea | 6 | 0 | 4 | 0 |
| Vomiting | 6 | 0 | 5 | 0 |
| Anemia | 5 | 0 | 3 | 0 |
| Dry skin | 5 | 0 | 4 | 0 |
| Dysgeusia | 5 | 0 | 5 | 0 |
| Dyspnea | 5 | 0 | 0 | 0 |
| Muscle spasms | 5 | 0 | 3 | 0 |
| Abdominal pain | 4 | 1 | 1 | 0 |
| Hypophosphatemia | 4 | 0 | 1 | 0 |
| Anxiety | 3 | 0 | 1 | 0 |
| Depression | 3 | 2 | 1 | 0 |
| Hypertension | 3 | 1 | 2 | 1 |
| Insomnia | 3 | 0 | 0 | 0 |
| Neutropenia | 3 | 1 | 3 | 1 |
| Night sweats | 3 | 0 | 3 | 0 |
| Peripheral edema | 3 | 0 | 2 | 0 |
| Hepatocellular | 2 | 1 | 1 | 1 |
| injury | | | | |

* All AEs with frequency >5% regardless of attribution shown
** All grade ≥3 treatment-related events shown

Table 14 describes a panel of biomarkers for tumor somatic profiling the 39 gene NGS of the disclosure (Example 1). Somatic mutations were determined in archived tumor tissue from 13 Phase 1 patients. Somatic mutations were identified when 1) variant allele frequency was greater than or equal to 10%, 2) sequence coverage was greater than or equal to 1000, and 3) the variant was not identified in dbSNP.

TABLE 14

| | # of genes | | Average |
|---|---|---|---|
| | assessed | DNA Sequencing Modality | Coverage |
| Panel 1 | 39 | 37 genes specific exons only | 1000x |
| | | All coding exons = EZH2, KDM6A | |

### Example 4: Detection of Mutation in ct-DNA through Suppressing NGS Errors

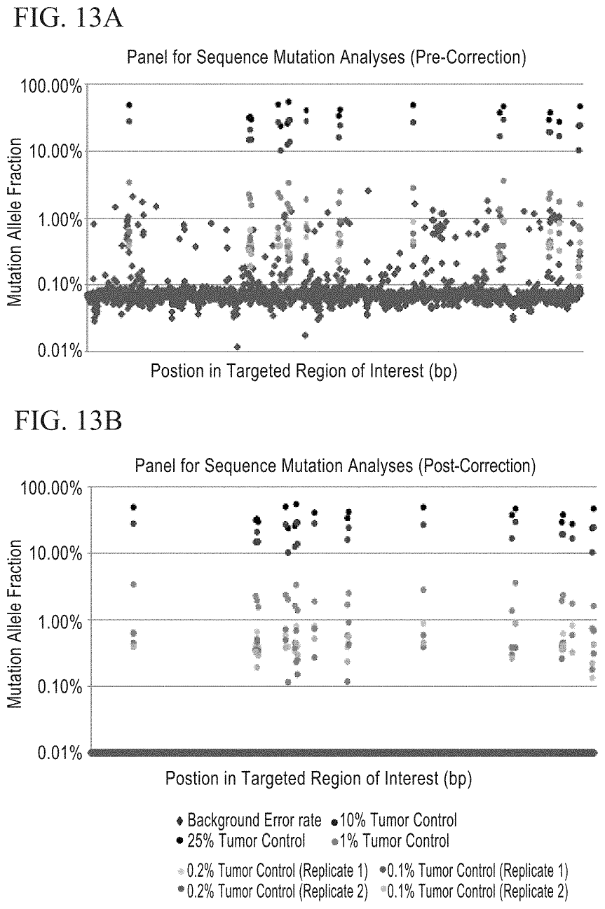

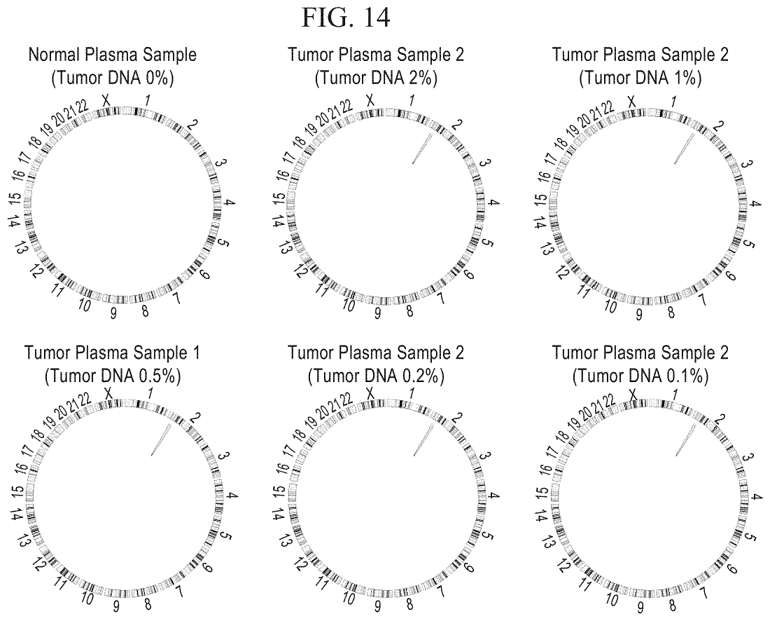

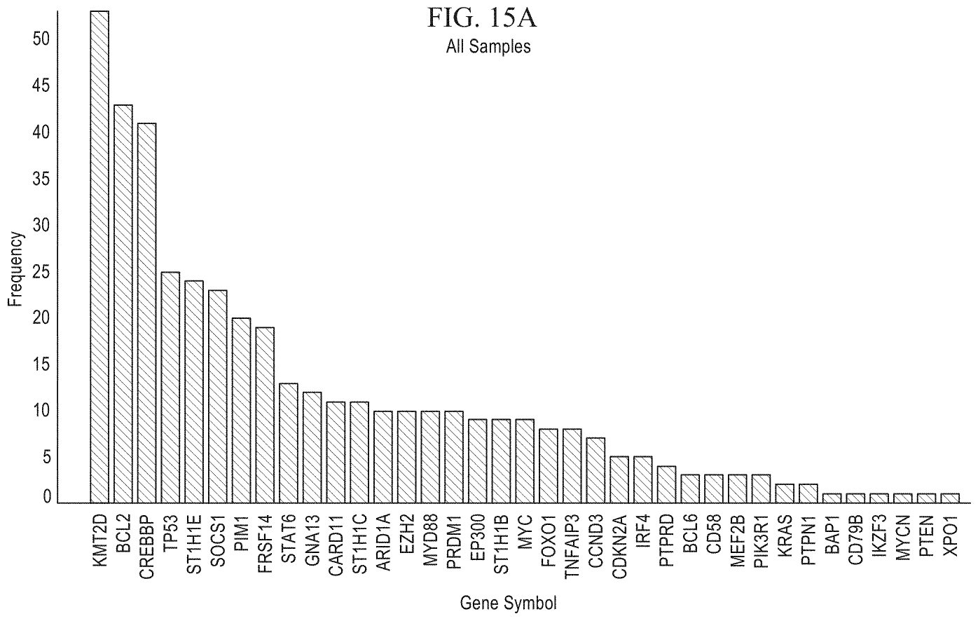

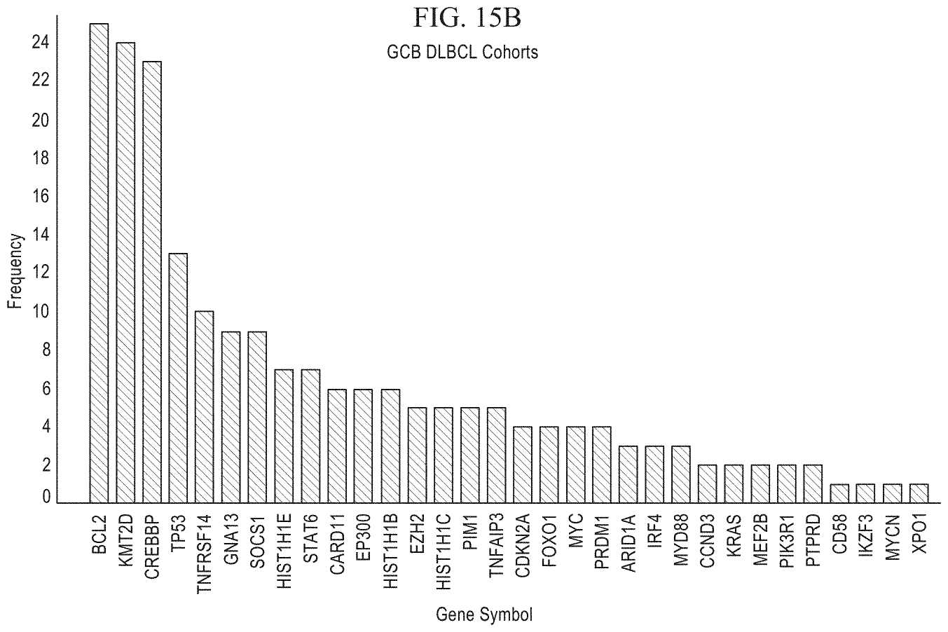

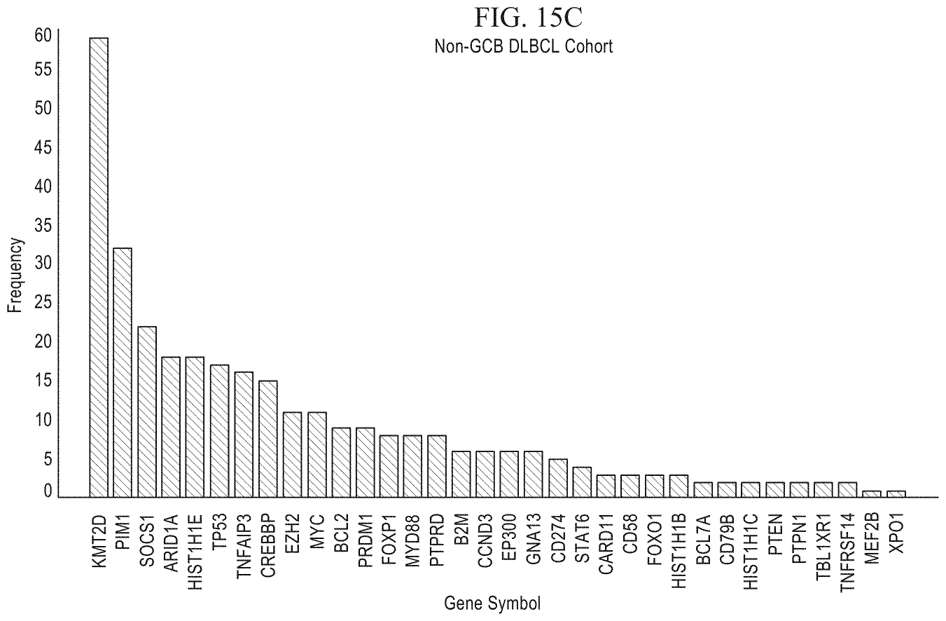

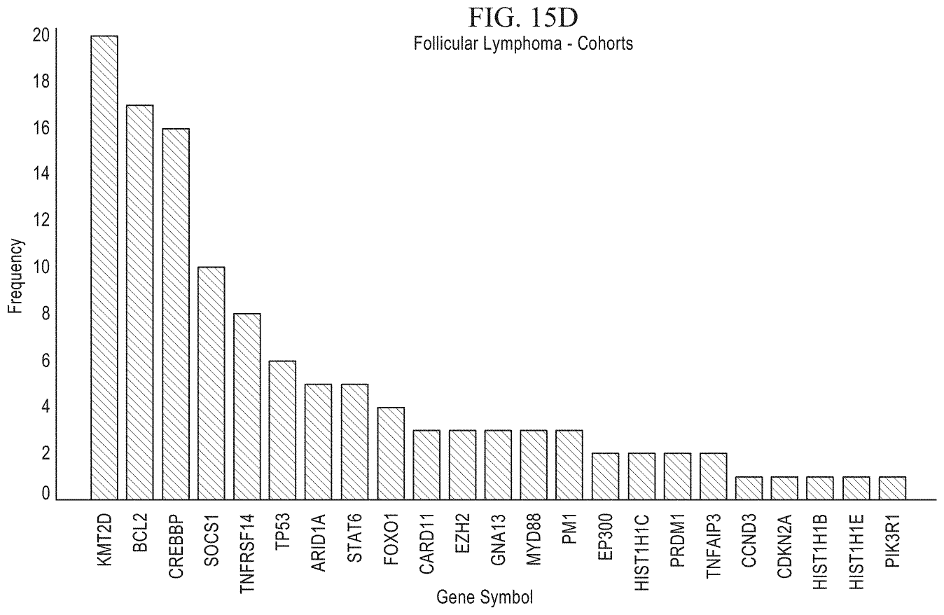

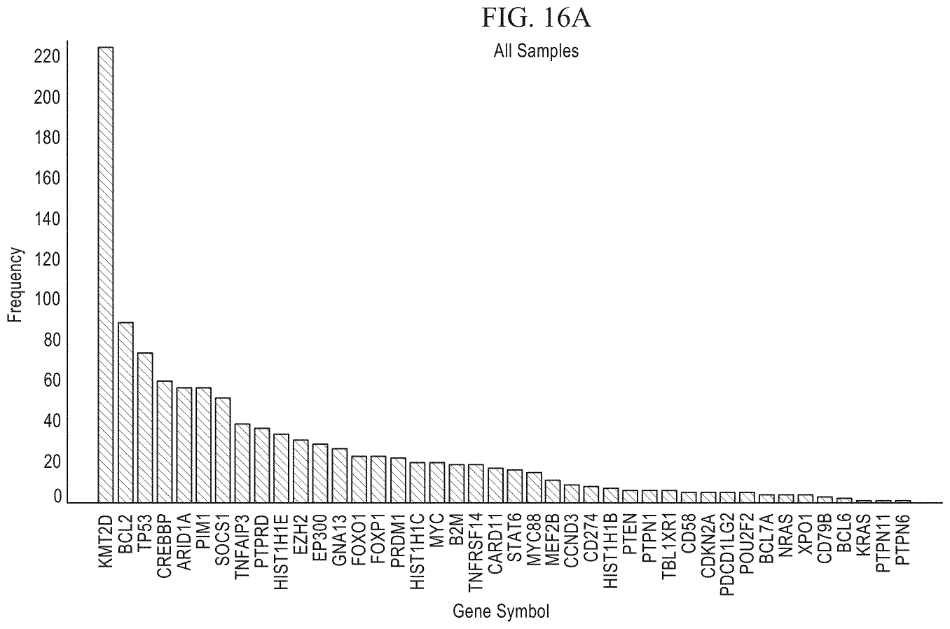

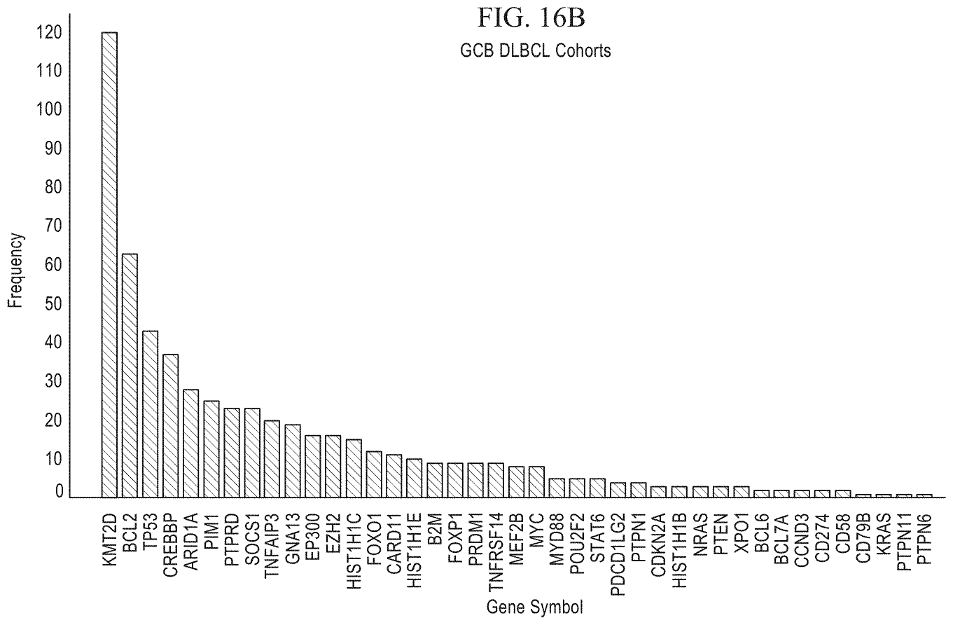

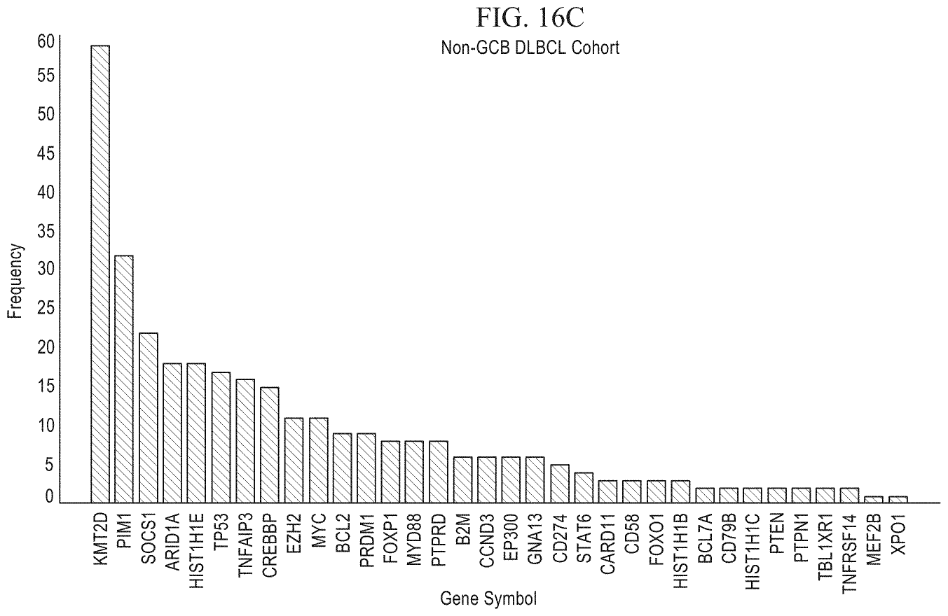

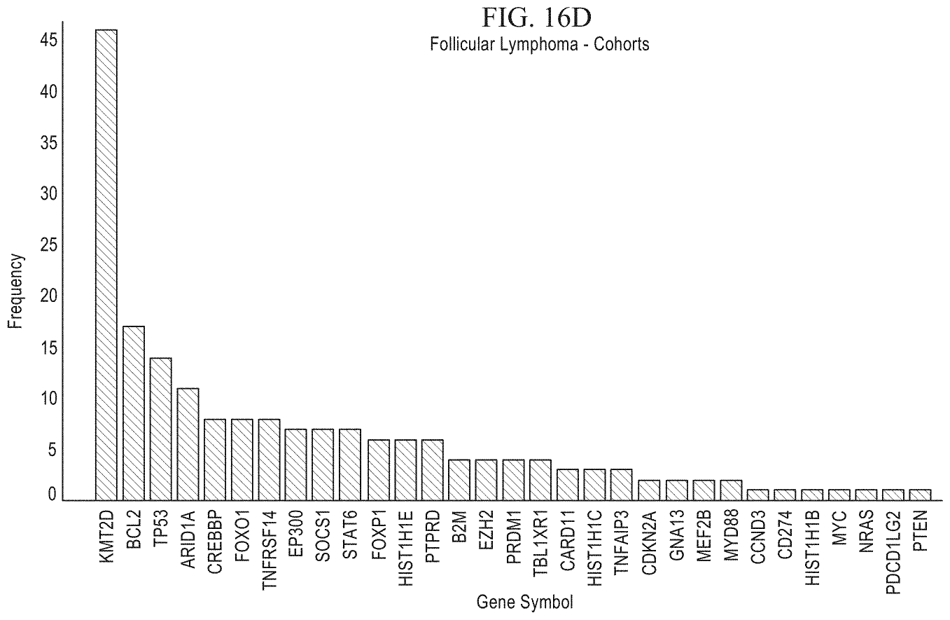

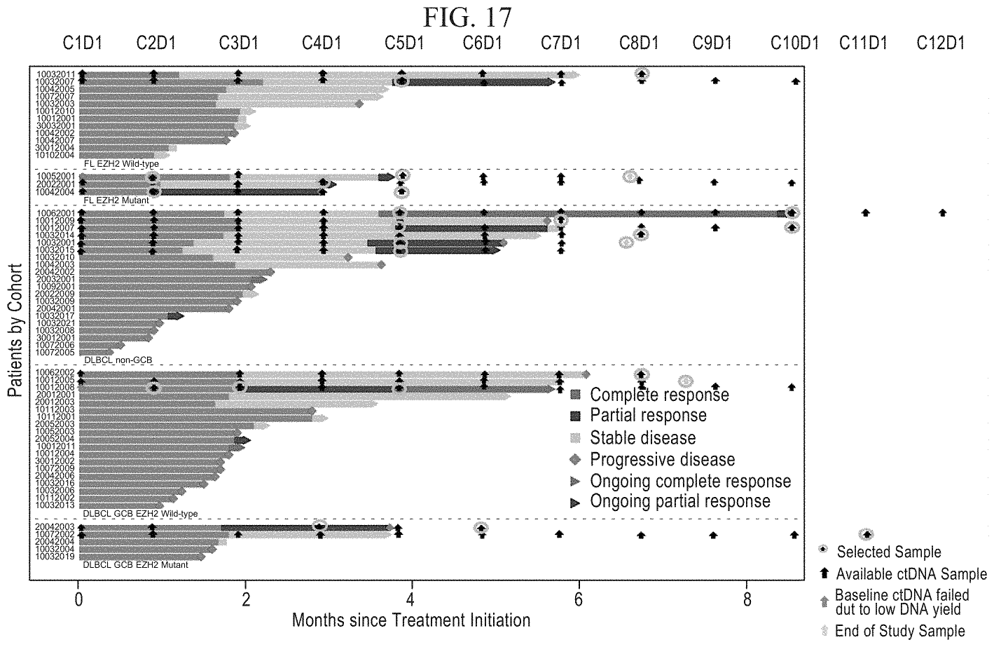

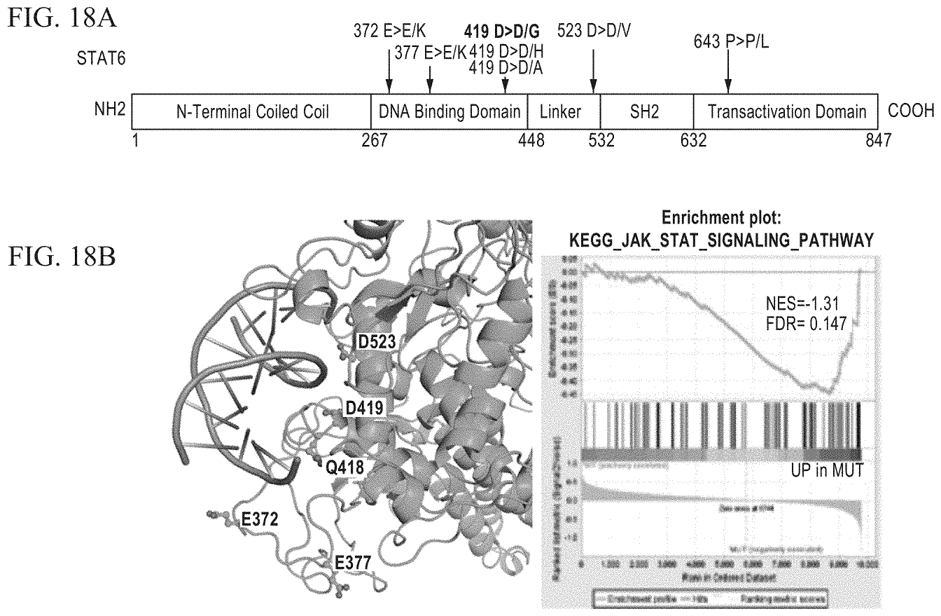

Archive and cell-free tumor DNA collected from relapsed refractory NHL patients phase I and II trials, were tested in the NGS panel as described in Examples 1 and 2. The content of the panel included molecular variants occurring in NHL at ≥5% frequency. (Tables 15 and 17-19, FIGS. 19A-22C). Redundant sequencing and molecular barcoding was found to suppress NGS error rates such as to enable the identification of mutations in archive tumor DNA down to 2% allelic frequency. Through correction of the background error by molecular bar coding the NHL specific plasma select panel was able to accurately detect mutations down to 0.1% allelic frequency (FIG. 13A and FIG. 13B). Translocations of ALK were detected in a cell-free DNA validation test set with samples from the phase I patients at a tumor purity of as low as 0.1% (FIG. 14). Sequencing of phase 1 NHL patients utilizing the 62 gene NHL NGS panel was completed for 10 archive tumor samples and 15 ctDNA samples (Table 15, FIG. 19A and FIG. 19B). In addition, microsatellite instability was monitored through the analysis of 5 distinct markers (BAT-25, BAT-26, MONO-27, NR-21 and NR-24), leading to one patient in the phase I trial being identified as microsatellite unstable based on the five tested markers (Table 15 and FIG. 19A and FIG. 19B, columns A16 and C16). Sequencing and an initial analysis of samples from patients in a phase 2 trial was completed with 58 archive tumor and 72 ctDNA baseline patient samples, wherein 48 of the archive tumor patients and 68 of the ctDNA patients were sequenced with reported response data.

Table 15 summarizes the molecular variants observed in archive tumor in samples from phase 1 patients. Observed molecular variants were frameshift or nonsense mutations, missense mutations, translocations and amplifications. If multiple mutations were found in the same sample only the most damaging alteration are shown. Trends later identified in phase 2 samples also appear in the phase 1 NHL samples (e.g., EZH2, STAT6 and MYC).

TABLE 15

| | Best Reponse = CR or PR | | | | | | | | | | |
|---|---|---|---|---|---|---|---|---|---|---|---|
| | A5 | C5 | A8 | C8 | C9 | A4 | C4 | C6 | C2 | A7 | C7 |
| GCB-DLBCL | N/A | N/A | N/A | N/A | N/A | | | | | | |
| non-GCB-DLBCL | | | | | | N/A | N/A | N/A | | | |
| [symbol] Lymphoma | | | | | | | | | N/A | N/A | N/A |
| ARID [symbol] A | | M | | | M | | | M | | | M |
| ATM | | | | | M | | | M | | | M |
| B2M ** | | | M | M | | | | | | | |
| BCL2 | T | T | | | | | M | A | | | |
| BCL5 | | | | | | | | T | | | |
| BCL7A | | | | | | | | | | | |
| BRAF | | | | | | | | | | | |
| CARD11 ** | | | | | | | | | | | |
| CCN [symbol] 3 | | | | | | | | | | | |
| CD5B | | | | | | | | | | | |
| CD [symbol] | | | | | | | | | | | |
| CD274 (P [symbol] L1) | | | | | | | | | | | |
| CD [symbol] N2A | | | | F | | | | | | | |
| CIITA | | | | | | | | | | | |
| CREBBP ** | M | M | M | M | F | | M | M | | M | |
| EP300 ** | | M | | | F | | | M | M | | M |
| E2H2 (Y646) ** | M | M | | | | | | | | | |
| E2H2 (non-Y646) ** | | | | | | | | | | | M |
| FOXO1 | | | | F | M | | | | | | |
| FOXP1 | | | | | M | | | M | | | |
| GNA13 | | | | M | | | | | | | M |
| HIST [symbol] H [symbol] B | | | | F | | | | | | | M |
| HIST [symbol] H [symbol] C | | | | | | | | | | | |
| HIST [symbol] H [symbol] E | | | | M | | | | M | | | |
| IZ [symbol] | | | | | | | | | | | |
| IRF4 | | | | | | | | M | | | |
| JA [symbol] 2 | | | | | | | | | | | M |
| KDM4C | | | | | M | | | M | | | |
| KDM6A ** | | M | | | | | | | | | |
| KIT | | | | | | | | M | | | |
| KMT2D | M | M | | | | | F | M | | M | F |
| [symbol] RAS | | | | | | | | | | | |
| MEF2B | | | | | | | | | | | |
| MYC | | T | | | M | | | | | | |
| MYD88 | | | | | | | | | | | M |
| NOTCH1 | | | | F | | | | M | | | M |
| NOTCH2 | | | | | | | | M | | | |
| NRAS | | | | | | | | | | | |
| P [symbol] ( [symbol] 2) | | | | | M | | | F | | | |
| PIK3CA | | | | | | | | | M | | |
| PIM1 | | | | | | | | M | | | |
| POU2F2 | | | | | | | | M | | | |
| PRDM1 | | M | | M | M | M | | M | | | M |
| PTEN | | | | | | | M | | | | |
| PTPN6 | | | | | | | | M | | | M |
| PTPN11 | | | | | M | | | M | | | |
| PTPRD | | M | | | M | | | M | | | |
| [symbol] | | | A | A | M | | | M | | | M |
| SOC [symbol] 1 | | | | | | | | | | | M |
| STAT6 | M | M | M | | | | | M | | M | M |
| TNFAIP3 | F | | F | F | | | M | M | | | F |
| TNFRSF14 ** | F | | | | F | | F | F | | M | M |
| TP53 | | | M | M | M | | M | M | | | M |
| | non-Responder < CR or PR | | | | | | | | | | |
| | A16 | C16 | A18 | C18 | C11 | C15 | C17 | A10 | C10 | A14 | C14 |
| GCB-DLBCL | N/A | N/A | N/A | N/A | | | | | | | |
| non-GCB-DLBCL | | | | | N/A | N/A | N/A | | | | |
| [symbol] Lymphoma | | | | | | | | N/A | N/A | N/A | N/A |
| ARID [symbol] A | | M | | F | M | M | M | | | | M |
| ATM | | M | | | M | | | | | F | M |
| B2M ** | | | | | | | | | | | F |
| BCL2 | M | M | T | T | M | | A | | T | T | T |
| BCL5 | | | | | | | | | | | |
| BCL7A | | M | | | F | | | | | | |
| BRAF | | | | | M | | | | M | | |
| CARD11 ** | F | M | | | M | | | | M | M | M |
| CCN [symbol] 3 | | | F | F | M | | | | F | | |
| CD5B | | | | | | | | | | | |
| CD [symbol] | | M | | | | | | | | | |
| CD274 (P [symbol] L1) | | M | | | | | | | | | |
| CD [symbol] N2A | | | | | | | | | | | |
| CIITA | | | | | | | | | | | A |
| CREBBP ** | M | M | | | F | M | | M | M | M | M |
| EP300 ** | F | M | | | M | M | | | | | M |
| E2H2 (Y646) ** | | | | | | | | | | | |
| E2H2 (non-Y646) ** | M | M | | | F | | M | | | | |
| FOXO1 | | F | | M | | | | M | | | |
| FOXP1 | | M | | | M | | | | | | |
| GNA13 | M | | | | | | | | | | |
| HIST [symbol] H [symbol] B | M | M | | | | | | | | | M |
| HIST [symbol] H [symbol] C | | M | | | | | | | | | |
| HIST [symbol] H [symbol] E | | | | | | F | | | M | | |
| IZ [symbol] | | M | | | M | | | | M | M | M |
| IRF4 | | | | | M | | | | M | | |
| JA [symbol] 2 | | M | | | M | | | | M | M | |
| KDM4C | | M | | | F | | | | | | |
| KDM6A ** | M | M | | | M | | | | M | | |
| KIT | M | M | | | M | | M | | | | |
| KMT2D | F | F | M | M | F | F | | | F | F | F |
| [symbol] RAS | | | | M | M | | | | | | |
| MEF2B | | M | | | | | | | | | |
| MYC | | | T | T | M | M | | | | | T |
| MYD88 | | | | | | M | | | | | |
| NOTCH1 | | M | | | M | | | | M | A | |
| NOTCH2 | M | F | | | M | M | | | | | |
| NRAS | | M | | | | | | | | | |
| P [symbol] ( [symbol] 2) | | M | | | M | | | | | | |
| PIK3CA | M | M | | | M | | M | | M | | |
| PIM1 | | | | | | | | | | | |
| POU2F2 | | | | | M | F | | | | | |
| PRDM1 | | M | | | M | M | | | M | | |
| PTEN | | | | | M | | | | | | |
| PTPN6 | | | | | M | | | | | | |
| PTPN11 | | M | | | | | | | | | |
| PTPRD | M | M | | | M | M | | | | | M |
| [symbol] | | M | | | | | | | | | |
| SOC [symbol] 1 | M | M | | | | | | | | | M |
| STAT6 | | | | | | | | M | | | |
| TNFAIP3 | F | M | | | M | | | | | | |
| TNFRSF14 ** | | | | | M | | | | | | |
| TP53 | | M | | M | M | F | M | M | M | | |

“F” = Frameshift or nonsense mutation;
“M” = Missense mutation;
“T” = Translocation
“A” = Amplification
** Molecular variants identified in the 39 gene NGS panel of Example 1. [symbol]
indicates data missing or illegible when filed

Table 16 shows a comparison between a Cobas® test (Roche Molecular Systems, Inc.) and the 62 gene NGS Panel of the disclosure in the of detection of EZH2 hot spot mutations.

Table 17 summarizes the molecular variants observed in archive tumor in phase 2 Patients. Observed molecular variants were frameshift or nonsense mutations, missense mutations, translocations and amplifications. Variants of interest included, inter alia, EZH2, MYD88 (273P) and MYC. EZH2 mutations were observed in 9 patients, wherein 7 displayed a variant allele frequency of >10%; 2 had variant allele frequencies of ≤10% (10042008, 8%; 10032004, 10%; best response: 4 PR, 3 SD and 2 PD). MYD88 (273P) mutations were observed in 6 patients (best response: 3 CR, 1PR, 1 PD and 1 unknown response); STAT6 mutations were observed in 13 patients (best response: 1 CR, 5 PR, 4 SD and 3 PD). MYC mutations were observed in 7 patients (best response: 5 PD and 2 unknown responses). 2 MYC translocations were associated with lack of response.

Table 18 summarizes the molecular variants with variant allele frequencies of 0.1% observed in ctDNA in phase 2 patients. Observed molecular variants were frameshift or nonsense mutations, missense mutations, translocations and amplifications. Variants of interest included, inter alia, EZH2, MYD88 (273P) and MYC. EZH2 mutations were observed in 11 patients (best response: 5 PR, 2 SD, 3 PD and 1 unknown response). MYD88 (273P) mutations were observed in 6 patients (best response: 2 CR, 1PR, 1 SD and 2 PD); STAT6 mutations were observed in 14 patients (best response: 5 PR, 6 SD and 3 PD). MYC mutations were observed in 18 patients (best response: 2 PR, 3SD, 9 PD and 4 unknown responses). 5 MYC translocations were associated with lack of response.

Table 19 summarizes the molecular variants with variant allele frequencies of 1% observed in ctDNA in phase 2 patients. Observed molecular variants were frameshift or nonsense mutations, missense mutations, translocations and amplifications. Variants of interest included, inter alia, EZH2, MYD88 (273P) and MYC. EZH2 mutations were observed in 8 patients (best response: 4 PR, 1 SD and 3 PD). MYD88 (273P) mutations were observed in 5 patients (best response: 2 CR, 1PR, and 2 PD); STAT6 mutations were observed in 10 patients (best response: 4 PR, 4 SD and 2 PD). MYC mutations were observed in 5 patients (best response: 3 PD and 2 unknown responses). 5 MYC translocations were associated with lack of response.

TABLE 16

| | | | EZH2 | | | | Clonal or |
|---|---|---|---|---|---|---|---|
| | | Cell of Origin | Cobas ® | Tumor Content for | Archive Tumor NGS | ctDNA | Subclonal EZH2 |
| Patient ID ² | Cohort Designation | (Nanostring) | Result | Cobas ® Assay | Result (vaf) | NGS Result (vaf) | mutation ¹ |
| 1003-2004 | GCB-DLBCL EZH2 | GCB DLBCL | Y646F | 100% | EZH2 Y646F (10%) | EZH2 Y646F | Subclonal |
| | MT | | | | | (1.3%) | |
| 1003-2015 | Non-GCB DLBCL | GCB DLBCL | Y646X | 20% | EZH2 Y646H (19%) | EZH2 Y646H | Clonal |
| | | | | | | (12.7%) | |
| 1003-2019 | GCB-DLBCL EZH2 | GCB DLBCL | Y646F | 100% | EZH2 Y646F (38%) | EZH2 Y646F | Clonal |
| | MT | | | | | (8.94%) | |
| 1004-2004 | FL EZH2 mutant | N/A | Y646N | 100% | Not sequenced | EZH2 Y646N | Unknown |
| | | | | | (failed library) | (34.9%) | |
| 1004-2008 | FL EZH2 mutant | N/A | Y646F | 100% | EZH2 Y646F (8%) | Not detected | Subclonal |
| 1004-2009 | GCB-DLBCL EZH2 | Not performed | A682G | 95% | EZH2 A682G (34%) | EZH2 A682G | Clonal |
| | MT | | | | | (0.9%) | |
| 1004-2011 | GCB-DLBCL EZH2 | GCB DLBCL | WT | 100% | Low DNA Yield | Not detected | Unknown |
| | MT | | | | | | |
| 1005-2001 | FL EZH2 mutant | N/A | Y646N | 90% | EZH2 Y646N (22%) | Low DNA yield | Clonal |
| 1007-2002 | GCB-DLBCL EZH2 | GCB DLBCL | Y646N | 70% | Not sequenced | EZH2 Y646F | Unknown |
| | MT | | | | (failed library) | (0.36%) | |
| 1008-2003 | GCB-DLBCL EZH2 | Not performed | Y646N | 70% | Not sequenced | EZH2 Y646N | Unknown |
| | MT | | | | (failed library) | (3.18%) | |
| 2002-2001 | FL EZH2 mutant | N/A | Y646X | 100% | EZH2 Y646S (22%) | EZH2 Y646S | Clonal |
| | | | | | | (6.6%) | |
| 2002-2010 | GCB-DLBCL EZH2 | GCB DLBCL | WT | 100% | Not detected | EZH2 Y646C | Unknown |
| | WT | | | | | (0.33%) | |
| 2004-2003 | GCB-DLBCL EZH2 | GCB DLBCL | Y646X | Unknown | EZH2 Y646H (25%) | EZH2 Y646H | Unknown |
| | MT | | | | | (28%) | |
| 2004-2004 | GCB-DLBCL EZH2 | GCB DLBCL | Y646N | 20% | Not sequenced | EZH2 Y646N | Unknown |
| | MT | | | | (failed library) | (39.2%) | |

¹ Patients determined to have EZH2 mutant tumor DNA copies ≥20% were considered clonal
² All EZH2 mutant patients enrolled before May 1^{st,} 2016 are represented in this table.

TABLE 17

| | 2 | 3 | 5 | 29 | 47 | 51 | 7 | 15 | 17 | 30 |
|---|---|---|---|---|---|---|---|---|---|---|
| | [symbol] | [symbol] | [symbol] | [symbol] | [symbol] | [symbol] | [symbol] | [symbol] | [symbol] | [symbol] |
| GCB-DLBCL Cohort | N/A | N/A | N/A | N/A | N/A | N/A | | | | |
| non-GCB-DLBCL Cohort | | | | | | | N/A | N/A | N/A | N/A |
| Follicular Lymphoma | | | | | | | | | | |
| [symbol] Positive (Cobas) | | | | | N/A | | | N/A | N/A | |
| CR + PR | N/A | N/A | N/A | N/A | N/A | N/A | N/A | N/A | N/A | N/A |
| Stable Disease | | | | | | | | | | |
| Progressive Disease | | | | | | | | | | |
| ARID [symbol] | | | | | | | | | | |
| B [symbol] | | | | M | F | | | | | |
| BCL2 [symbol] Mutation | | M | | | | | | | | |
| BCL2 [symbol] | | T | | T | | | | | | |
| BCL6 | | | | | | | | T | | |
| C [symbol] | M | | | | | | | | | M |
| CC [symbol] D3 | | | | | | | | | F | |
| CD [symbol] | | | | | | | | | | |
| CD [symbol] | | | | | | | | | | M |
| [symbol] ( [symbol] ) | | | | | | | | | | |
| C [symbol] | | | | | | | | | | |
| C [symbol] | M | F | | F | | M | | M | | |
| E [symbol] | | | | | | | | | M | |
| E [symbol] | | | | | M | | | M | M | |
| FOXO1 | | | | | | | | | | |
| G [symbol] 3 | | | | | M | | | | | |
| HIST [symbol] | M | | | | | | | | | |
| HIST [symbol] | | | | | | | | | | |
| HIST [symbol] | | | M | | | | | | | |
| [symbol] | | | | | M | F | F | F | | |
| [symbol] | | M | | | | M | | | | |
| [symbol] | | | | | | | | M | | |
| MYC [symbol] | | | | | | | | | | |
| M [symbol] C [symbol] | | | | | | | | | | |
| M [symbol] (273P) | | | M | | | | M | | | M |
| [symbol] (POL2) | | | | | | | | | | |
| P [symbol] | | | | | | | | | | M |
| [symbol] 2 | | | | | | | | | M | |
| [symbol] 1 | | | | | | | | | | M |
| [symbol] 1 | M | | | | M | M | | | | |
| STAT [symbol] | | | | | M | M | | | | |
| TN [symbol] | | | | | F | | | | | |
| TN [symbol] | F | | | | | F | | M | | |
| | 10 | 23 | 25 | 27 | 38 | 43 | 14 | 56 | 4 | 12 |
| | [symbol] | [symbol] | [symbol] | [symbol] | [symbol] | [symbol] | [symbol] | [symbol] | [symbol] | [symbol] |
| GCB-DLBCL Cohort | | | | | | | | | | |
| non-GCB-DLBCL Cohort | | | | | | | N/A | N/A | | |
| Follicular Lymphoma | N/A | N/A | N/A | N/A | N/A | N/A | | | N/A | N/A |
| [symbol] Positive (Cobas) | | | | N/A | | N/A | | | | |
| CR + PR | N/A | N/A | N/A | N/A | N/A | N/A | | | | |
| Stable Disease | | | | | | | N/A | N/A | N/A | N/A |
| Progressive Disease | | | | | | | | | | |
| ARID [symbol] | | | M | | M | | | | | |
| B [symbol] | | | | | | | M | | | |
| BCL2 [symbol] Mutation | | M | | M | M | | | | | |
| BCL2 [symbol] | T | | | T | | | | | T | T |
| BCL6 | | | | T | | | | | | |
| C [symbol] | | | | | | | | | | |
| CC [symbol] D3 | | | | | | | M | | | |
| CD [symbol] | | | | | | | | | | |
| CD [symbol] | | | | | | | | | | |
| [symbol] ( [symbol] ) | | | | | | | | | | |
| C [symbol] | M | | | | | | | | | |
| C [symbol] | F | M | | F | M | | | | F | F |
| E [symbol] | | | | | | | | | | M |
| E [symbol] | | | | M | | M | | | | |
| FOXO1 | | | | | | | | | M | |
| G [symbol] 3 | | | | | | | | | | |
| HIST [symbol] | | | | | | | | M | | |
| HIST [symbol] | | | | | | | | M | | |
| HIST [symbol] | | | | | | | M | M | | |
| [symbol] | F | | F | F | F | | | M | F | |
| [symbol] | | | | | | | | | | |
| [symbol] | | | | | | | | | | |
| MYC [symbol] | | | | | | | | | | |
| M [symbol] C [symbol] | | | | | | | | | | |
| M [symbol] (273P) | | | M | | | | | | | |
| [symbol] (POL2) | | | | | | | | | | |
| P [symbol] | | | F | | | | | | M | |
| [symbol] 2 | | | | | | | | | M | |
| [symbol] 1 | | | | | | | | | | |
| [symbol] 1 | M | | | | | M | | F | M | |
| STAT [symbol] | M | M | | M | | M | | M | | |
| TN [symbol] | | | | | | | F | | | M |
| TN [symbol] | F | M | | | F | F | | | | F |
| | 40 | 49 | 55 | 25 | 31 | 39 | 41 | 42 | 22 | 24 |
| | [symbol] | [symbol] | [symbol] | [symbol] | [symbol] | [symbol] | [symbol] | [symbol] | [symbol] | [symbol] |
| GCB-DLBCL Cohort | | | | N/A | N/A | N/A | N/A | N/A | | |
| non-GCB-DLBCL Cohort | | | | | | | | | N/A | |
| Follicular Lymphoma | N/A | N/A | N/A | | | | | | | N/A |
| [symbol] Positive (Cobas) | | | | N/A | | | | | | N/A |
| CR + PR | | | | | | | | | | |
| Stable Disease | N/A | N/A | N/A | N/A | N/A | N/A | N/A | N/A | N/A | N/A |
| Progressive Disease | | | | | | | | | | |
| ARID [symbol] | | M | | | | | | | | |
| B [symbol] | | | | | | | | | | |
| BCL2 [symbol] Mutation | M | M | | M | M | | M | | | M |
| BCL2 [symbol] | | | | | T | | | T | | |
| BCL6 | | | | | | | | | | |
| C [symbol] | | | M | | | M | | | | |
| CC [symbol] D3 | | | | | | | | | | |
| CD [symbol] | | | | | | | | | | |
| CD [symbol] | | | | | | | | | | |
| [symbol] ( [symbol] ) | | | | | | | | | | |
| C [symbol] | | | | | M | | | | | |
| C [symbol] | M | F | M | | F | | M | M | | M |
| E [symbol] | | | | M | | | | | | |
| E [symbol] | | | | M | | | | | | M |
| FOXO1 | | | | | | | | | | |
| G [symbol] 3 | | | | | | | | M | | |
| HIST [symbol] | | | | | | | | | | |
| HIST [symbol] | | | | M | | | | | | |
| HIST [symbol] | | | | | | | | | | |
| [symbol] | F | M | F | | | | F | F | | F |
| [symbol] | | | | | | | | | | |
| [symbol] | | | | | | | | | | |
| MYC [symbol] | | | | | | | | | | |
| M [symbol] C [symbol] | | | | | | | | | | |
| M [symbol] (273P) | | | | | | | | | | |
| [symbol] (POL2) | | | | | | | | | | |
| P [symbol] | | | | | | | | | | |
| [symbol] 2 | | | | | | | | | | |
| [symbol] 1 | | M | | | | | | | | |
| [symbol] 1 | | | | | M | | | | | |
| STAT [symbol] | | | | | M | | M | M | | |
| TN [symbol] | | | | | | | | | | |
| TN [symbol] | | | | | F | | F | | | |
| | 34 | 53 | 1 | 8 | 9 | 13 | 15 | 19 | 32 | 35 |
| | [symbol] | [symbol] | [symbol] | [symbol] | [symbol] | [symbol] | [symbol] | [symbol] | [symbol] | [symbol] |
| GCB-DLBCL Cohort | | | N/A | N/A | N/A | N/A | N/A | N/A | N/A | N/A |
| non-GCB-DLBCL Cohort | | | | | | | | | | |
| Follicular Lymphoma | N/A | N/A | | | | | | | | |
| [symbol] Positive (Cobas) | | | | N/A | | | | N/A | | |
| CR + PR | | | | | | | | | | |
| Stable Disease | N/A | N/A | | | | | | | | |
| Progressive Disease | | | N/A | N/A | N/A | N/A | N/A | N/A | N/A | N/A |
| ARID [symbol] | F | F | | | | | | | | |
| B [symbol] | | | | | | M | | | | F |
| BCL2 [symbol] Mutation | M | M | | M | M | M | M | M | M | |
| BCL2 [symbol] | T | T | T | T | | | T | | | |
| BCL6 | T | | | | | | | | | T |
| C [symbol] | | | | | | | M | M | | |
| CC [symbol] D3 | | M | | | | | | | | |
| CD [symbol] | | | | | | | | | | |
| CD [symbol] | | | | | | | | | | |
| [symbol] ( [symbol] ) | | | | | | | | | | |
| C [symbol] | | | | | | | | | | |
| C [symbol] | | | M | | | M | M | | F | M |
| E [symbol] | | | | | | | | M | M | |
| E [symbol] | | | | M | | | | M | | |
| FOXO1 | M | | | | | | | | M | |
| G [symbol] 3 | F | | M | M | | F | | | | |
| HIST [symbol] | | | | M | | | | | | |
| HIST [symbol] | | | | | | | | | | |
| HIST [symbol] | | | M | | | | | | | |
| [symbol] | M | F | | F | F | F | | F | | F |
| [symbol] | | | | | | | | | | |
| [symbol] | | | | | | M | M | | | |
| MYC [symbol] | | | | | | | | | M | M |
| M [symbol] C [symbol] | | | | | | | | | | |
| M [symbol] (273P) | | | | | | | | | | |
| [symbol] (POL2) | | | | | | | | | | |
| P [symbol] | | | | | M | | | | | |
| [symbol] 2 | | | | | | M | M | | | |
| [symbol] 1 | | | F | | | | | | | |
| [symbol] 1 | | | | | | | | | | |
| STAT [symbol] | | | M | | | | | | | |
| TN [symbol] | | | | | | | | | | F |
| TN [symbol] | | | F | | | F | F | F | | |
| | 52 | 54 | 11 | 20 | 28 | 33 | 36 | 46 | 18 | 44 |
| | [symbol] | [symbol] | [symbol] | [symbol] | [symbol] | [symbol] | [symbol] | [symbol] | [symbol] | [symbol] |
| GCB-DLBCL Cohort | N/A | N/A | | | | | | | | |
| non-GCB-DLBCL Cohort | | | N/A | N/A | N/A | N/A | N/A | N/A | | |
| Follicular Lymphoma | | | | | | | | | N/A | N/A |
| [symbol] Positive (Cobas) | | | | | | | | | | |
| CR + PR | | | | | | | | | | |
| Stable Disease | | | | | | | | | | |
| Progressive Disease | N/A | N/A | N/A | N/A | N/A | N/A | N/A | N/A | N/A | N/A |
| ARID [symbol] | | | | F | | | | | | |
| B [symbol] | | F | | | F | | | | | |
| BCL2 [symbol] Mutation | A | | | A | | | | | | M |
| BCL2 [symbol] | | T | | | | | | | T | T |
| BCL6 | | | M | | | M | T | T | | |
| C [symbol] | | | | | | | | | M | |
| CC [symbol] D3 | | F | | | | | | | | |
| CD [symbol] | | | | F | | | | | | |
| CD [symbol] | | | | | | | | | | |
| [symbol] ( [symbol] ) | | | | | F | A | | | | |
| C [symbol] | | | | | | | | | | |
| C [symbol] | | F | | | | | | | F | |
| E [symbol] | | | | | | | | | M | |
| E [symbol] | | | | | | | | | | |
| FOXO1 | | M | | | | | | | M | |
| G [symbol] 3 | | | | | | | | | M | |
| HIST [symbol] | | | | | | | | | | M |
| HIST [symbol] | | | | | | | | | | M |
| HIST [symbol] | | M | M | | M | | | | | |
| [symbol] | F | | | M | | | F | M | | |
| [symbol] | | | | | | | | | | |
| [symbol] | | | | | | | | | | |
| MYC [symbol] | M | M | | M | | | M | | | |
| M [symbol] C [symbol] | | T | | | | | | | | |
| M [symbol] (273P) | | | | | | | M | | | |
| [symbol] (POL2) | | | | | | A | | | | |
| P [symbol] | | | | | F | | | | | |
| [symbol] 2 | | | | | | | | | | |
| [symbol] 1 | | | | | | | | | | |
| [symbol] 1 | | M | | | | | | | | |
| STAT [symbol] | | M | | | | | | | | M |
| TN [symbol] | | | | | | | | | | |
| TN [symbol] | | | | | | | | | | |

| | 58 | 37 | 45 | 48 | 6 | 21 | 50 | 57 |
|---|---|---|---|---|---|---|---|---|
| | [symbol] | [symbol] | [symbol] | [symbol] | [symbol] | [symbol] | [symbol] | [symbol] |
| GCB-DLBCL Cohort | | N/A | N/A | N/A | | | | |
| non-GCB-DLBCL Cohort | | | | | N/A | N/A | N/A | N/A |
| Follicular Lymphoma | N/A | | | | | | | |
| [symbol] Positive (Cobas) | | | | | | | | |
| CR + PR | | | | | | | | |
| Stable Disease | | | | | | | | |
| Progressive Disease | N/A | | | | | | | |
| ARID [symbol] | | | | M | | | | |
| B [symbol] | | | | | | | | |
| BCL2 [symbol] Mutation | | M | | M | | | | |
| BCL2 [symbol] | | T | | T | T | | | |
| BCL6 | T | | | T | | | | |
| C [symbol] | | | | | | | | |
| CC [symbol] D3 | | | | | | | | F |
| CD [symbol] | | | | | | | | F |
| CD [symbol] | | | | | | | | |
| [symbol] ( [symbol] ) | | | | | | | A | |
| C [symbol] | | | | | | | | |
| C [symbol] | | | M | M | | | | F |
| E [symbol] | | | M | | | | | |
| E [symbol] | | | | | | | | |
| FOXO1 | | M | | | | | | |
| G [symbol] 3 | | | M | | | | | |
| HIST [symbol] | | | | | M | | | |
| HIST [symbol] | | M | M | | | | | |
| HIST [symbol] | M | M | | | | | | |
| [symbol] | F | F | F | F | F | | | |
| [symbol] | | | | | | | | |
| [symbol] | | | | | | | | |
| MYC [symbol] | | M | | | | | | |
| M [symbol] C [symbol] | | T | | | | | | |
| M [symbol] (273P) | | | | | | | M | |
| [symbol] (POL2) | | | | | | | | |
| P [symbol] | | F | M | | | | M | M |
| [symbol] 2 | | | | | | | | |
| [symbol] 1 | | | | | M | F | | |
| [symbol] 1 | M | | | | | | | |
| STAT [symbol] | | | | | | | | |
| TN [symbol] | | | | | | | | |
| TN [symbol] | | | | | | | | |

“F” = Frameshift or nonsense mutation;
“M” = Missense mutation;
“T” = Translocation
“A” = Amplification [symbol]
indicates data missing or illegible when filed

TABLE 18

| | | | | | | | | | | | | | | | On Study | | |
|---|---|---|---|---|---|---|---|---|---|---|---|---|---|---|---|---|---|
| | 4 | 7 | [symbol] | [symbol] | [symbol] | 5 | [symbol] | 21 | 41 | [symbol] | [symbol] | 31 | [symbol] | 57 | 20 | 25 | 32 |
| | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] - | N/A | N/A | N/A | N/A | N/A | | | | | | | | | | | | |
| DLBCL | | | | | | | | | | | | | | | | | |
| Cohort | | | | | | | | | | | | | | | | | |
| non- [symbol] - | | | | | | N/A | N/A | N/A | N/A | | | | | | N/A | | |
| DLBCL | | | | | | | | | | | | | | | | | |
| Cohort | | | | | | | | | | | | | | | | | |
| [symbol] | | | | | | | | | | N/A | N/A | N/A | N/A | N/A | | N/A | N/A |
| [symbol] ( [symbol] ) | | | | N/A | N/A | | | N/A | | | N/A | | | N/A | | | |
| CR + PR | N/A | N/A | N/A | N/A | N/A | N/A | N/A | N/A | N/A | N/A | N/A | N/A | N/A | N/A | | | |
| [symbol] | | | | | | | | | | | | | | | N/A | N/A | N/A |
| [symbol] D [symbol] | | | | | | | | | | | | | | | | | |
| [symbol] | M | | | | M | | | M | | M | | | M | | | [symbol] | |
| [symbol] | | | | | [symbol] | M | | | | | | M | | | | | |
| [symbol] | | | M | | M | M | | | | | A | M | | | | | |
| [symbol] | | | | | | | | | | | | | | | | T | |
| [symbol] | | | | | | | | | | | | | | | | | |
| [symbol] | | | | | | | | [symbol] | M | | M | | | | M | | |
| [symbol] | | | M | | | | | | | | | | | | M | | |
| [symbol] | | | | | | | | | | | | | | | | | |
| [symbol] | | | M | | | | | | M | | | | | | | | |
| [symbol] ( [symbol] ) | | | | | | | | | | | M | | | | | | |
| [symbol] | | | | | M | | | | | [symbol] | | | | | | | |
| [symbol] | M | | M | | M | | | | | | M | M | | | M | | M |
| [symbol] | | | M | | | | | | | M | | | | | | | |
| [symbol] ( [symbol] ) | | | | M | M | | | M | | | M | | | M | | | |
| [symbol] | | | M | | M | | | | | | | M | | | M | | |
| [symbol] | | | | | M | | | | | | M | | | | | | |
| [symbol] | | | | | | | | | | | A | | | | | | |
| [symbol] | | | | M | M | | | | | | | | | | M | | |
| [symbol] | | | | | | | | | | | | | | | | | |
| [symbol] | M | | | | | | | | | | | | | | | | |
| [symbol] | | M | | | | | M | | | M | | | | | M | | |
| [symbol] | M | M | [symbol] | | M | | | [symbol] | | [symbol] | [symbol] | M | [symbol] | M | M | | [symbol] |
| [symbol] | | | | | | | | | | | | | | | | | |
| [symbol] | | | M | | | | | M | | | M | | | | | M | |
| [symbol] | | | M | | | | M | | | | | | | | | | |
| [symbol] | | | | | | | | | | | | | | | | | |
| [symbol] ( [symbol] ) | | M | | | | | M | | M | | | | | | | | |
| [symbol] ( [symbol] ) | | | | | [symbol] | | | | | | | | | | | | |
| [symbol] | M | [symbol] | | | M | M | | | M | | | | | | M | | |
| [symbol] | | | | | | | | | | | | | | | | | |
| [symbol] | | | M | | | | | | M | M | | | | | | | |
| [symbol] | | | | | | | | | | | | | | | | | |
| [symbol] | | | | | [symbol] | | | | | M | | | | M | | | M |
| [symbol] | | | | | M | | | M | | M | M | M | | | | | M |
| [symbol] | | | | | [symbol] | | | [symbol] | | M | | | | | | | |
| [symbol] | | | | | | | | M | | [symbol] | | M | | [symbol] | | | M |
| | On Study | | | Off Study | | | | | | | | | | | | | |
| | [symbol] | 71 | [symbol] | 42 | 51 | [symbol] | [symbol] | [symbol] | [symbol] | 1 [symbol] | 2 [symbol] | [symbol] | 1 | 1 [symbol] | 33 | 4 [symbol] | 68 |
| | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] - | | | N/A | N/A | N/A | N/A | N/A | N/A | | | | | | | | | |
| DLBCL | | | | | | | | | | | | | | | | | |
| Cohort | | | | | | | | | | | | | | | | | |
| non- [symbol] - | | | | | | | | | N/A | N/A | N/A | N/A | | | | | |
| DLBCL | | | | | | | | | | | | | | | | | |
| Cohort | | | | | | | | | | | | | | | | | |
| [symbol] | N/A | N/A | | | | | | | | | | | N/A | N/A | N/A | N/A | N/A |
| [symbol] ( [symbol] ) | | | N/A | | | | | N/A | | | | | | | N/A | | |
| CR + PR | | | | | | | | | | | | | | | | | |
| [symbol] | N/A | N/A | N/A | N/A | N/A | N/A | N/A | N/A | N/A | N/A | N/A | N/A | N/A | N/A | N/A | N/A | N/A |
| [symbol] D [symbol] | | | | | | | | | | | | | | | | | |
| [symbol] | | | M | | M | [symbol] | M | M | | M | | | | | | M | [symbol] |
| [symbol] | | | M | | | | | M | [symbol] | | | | [symbol] | | | | |
| [symbol] | | M | | M | M | M | | M | | A | | | [symbol] | M | M | M | M |
| [symbol] | | | | T | | | T | T | | | | | T | | | T | T |
| [symbol] | | | | | | | | | | | | | T | T | | | |
| [symbol] | | M | | | M | | | | | | | | | [symbol] | | | |
| [symbol] | | | | | | | | | | | | M | | | | | M |
| [symbol] | | | | | | | | | | [symbol] | | | | | | | |
| [symbol] | | | | | | | | | | | | | | | | | |
| [symbol] ( [symbol] ) | | | | | | M | | | | M | | | | | | | |
| [symbol] | | | | M | | | | M | | | | | | | | | |
| [symbol] | | M | M | [symbol] | M | | M | | | | | | M | [symbol] | M | | |
| [symbol] | M | M | [symbol] | | M | | M | M | M | | | | | | | | |
| [symbol] ( [symbol] ) | | | M | | | | | M | | | | | | | | | |
| [symbol] | | | | | | | | M | | | | | | | | | |
| [symbol] | | | | | M | | M | M | | | | M | | M | M | M | |
| [symbol] | | | | | | | | | | | | | | | | | |
| [symbol] | | | | | | | M | | M | | | | | | | | |
| [symbol] | | | M | | | | | | | | | | | | | | |
| [symbol] | | | M | | [symbol] | | M | | | | | | M | | | | |
| [symbol] | | M | | | M | | | | M | M | | | M | | | | |
| [symbol] | | [symbol] | M | M | [symbol] | M | [symbol] | [symbol] | M | M | | [symbol] | [symbol] | [symbol] | | [symbol] | [symbol] |
| [symbol] | | | | | | | | | | | | | | | | | |
| [symbol] | | | | | | | | | | | | | | | | | |
| [symbol] | | | | | M | [symbol] | | M | | | | | | | | | |
| [symbol] | | | | | | | | | | | | | | | T | | |
| [symbol] ( [symbol] ) | | | | | | | | | | | | | | | | M | |
| [symbol] ( [symbol] ) | | | M | | | | | | A | | | | | | | | M |
| [symbol] | | | M | | M | | [symbol] | M | | M | | | | | | | |
| [symbol] | | | | | | | | M | | | | | | | | | |
| [symbol] | | | | | [symbol] | | M | [symbol] | | | | | | M | | [symbol] | |
| [symbol] | | | | | | | | | | | | | | | | | |
| [symbol] | | | | M | | | | | [symbol] | | | | [symbol] | | [symbol] | | |
| [symbol] | | | | M | | | M | | M | | M | | | | | | |
| [symbol] | | | | [symbol] | M | | M | | [symbol] | | [symbol] | | | | | [symbol] | |
| [symbol] | | | | [symbol] | M | [symbol] | | M | | | | | | | | | |

| | 2 | 1 [symbol] | 13 | 1 [symbol] | 22 | 24 | 2 [symbol] | [symbol] | [symbol] | [symbol] | [symbol] | 52 | [symbol] | [symbol] | [symbol] | 15 | 16 | 26 | [symbol] |
|---|---|---|---|---|---|---|---|---|---|---|---|---|---|---|---|---|---|---|---|
| | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] - | N/A | N/A | N/A | N/A | N/A | N/A | N/A | N/A | N/A | N/A | N/A | N/A | N/A | N/A | N/A | | | | |
| DLBCL | | | | | | | | | | | | | | | | | | | |
| Cohort | | | | | | | | | | | | | | | | | | | |
| non- [symbol] - | | | | | | | | | | | | | | | | N/A | N/A | N/A | N/A |
| DLBCL | | | | | | | | | | | | | | | | | | | |
| Cohort | | | | | | | | | | | | | | | | | | | |
| [symbol] | | | | | | | | | | | | | | | | | | | |
| [symbol] ( [symbol] ) | | N/A | | | | N/A | N/A | | | | N/A | | | | | | | | |
| CR + PR | | | | | | | | | | | | | | | | | | | |
| [symbol] | | | | | | | | | | | | | | | | | | | |
| [symbol] D [symbol] | N/A | N/A | N/A | N/A | N/A | N/A | N/A | N/A | N/A | N/A | N/A | N/A | N/A | N/A | N/A | N/A | N/A | N/A | N/A |
| [symbol] | | M | | M | M | | M | M | M | | M | | M | | M | M | | [symbol] | M |
| [symbol] | M | | [symbol] | M | | | | | | | | | | | [symbol] | | | | |
| [symbol] | M | M | M | M | M | M | M | M | M | | | M | | | M | M | | | M |
| [symbol] | T | | | | T | | | | T | | | T | | | T | | | | |
| [symbol] | T | | | T | | | T | | | T | | | | | | | | | |
| [symbol] | | M | | | M | M | M | | | | M | | | | | | | | |
| [symbol] | | [symbol] | | | | | | | | | | | | | | | | | [symbol] |
| [symbol] | | M | | | | | M | | | | | | | | | [symbol] | | | |
| [symbol] | | | | | | | | | | | | | | | | | | | |
| [symbol] ( [symbol] ) | | | | [symbol] | | | | | M | | | | A | | | | M | M | |
| [symbol] | | | | | | | | | | | | | | | | | | | |
| [symbol] | M | M | | M | M | M | [symbol] | M | M | M | M | [symbol] | | | | M | | [symbol] | |
| [symbol] | | | | M | | | M | | | | M | | | | | M | M | | M |
| [symbol] ( [symbol] ) | | M | | | | M | | | | | M | | | | | | | | |
| [symbol] | | M | | M | | | M | | | M | | | | | | M | M | | M |
| [symbol] | | | | | | | M | M | M | | M | | | | M | | M | | |
| [symbol] | | | | | | | | | | | | | | | | | | | |
| [symbol] | | M | | [symbol] | | | [symbol] | M | M | | [symbol] | | [symbol] | | M | M | | | |
| [symbol] | | M | | | | M | | | | | | | | | | M | | | |
| [symbol] | M | | | | | | M | | | | | M | | | M | M | | | |
| [symbol] | | M | | | | | M | | | | | M | M | | M | M | | | M |
| [symbol] | [symbol] | [symbol] | [symbol] | M | M | [symbol] | M | | [symbol] | | M | [symbol] | M | [symbol] | M | [symbol] | M | M | M |
| [symbol] | | M | | | | | | | | | | | | | | | | | |
| [symbol] | | [symbol] | | M | M | M | | | | | M | M | | | | | | | |
| [symbol] | | | | | | M | | M | M | | | | | | M | M | M | | |
| [symbol] | | | | | T | | | | | | | | | | T | | | | |
| [symbol] ( [symbol] ) | | | | | | | | | | | | | | | | | | | |
| [symbol] ( [symbol] ) | | | | | | M | | | | | | | A | | | | | | |
| [symbol] | | M | M | | | | M | | M | | M | [symbol] | | | M | M | | M | [symbol] |
| [symbol] | | | | M | M | | M | | | [symbol] | | | | | | | | | |
| [symbol] | | | | M | | | | | M | | | | [symbol] | | | M | M | | |
| [symbol] | | | | | | | | | | | | | | | | | | | |
| [symbol] | [symbol] | | | M | | | [symbol] | | | | | | [symbol] | | M | [symbol] | | | |
| [symbol] | | | | M | | | | | | | | | | | M | | | | |
| [symbol] | | | | M | | M | M | | | [symbol] | M | | M | | M | M | M | M | |
| [symbol] | | | | | [symbol] | [symbol] | | M | M | | | | | | | | | | |

| | | | | | | | | | | | Off Study | | | | | | | |
|---|---|---|---|---|---|---|---|---|---|---|---|---|---|---|---|---|---|---|
| | [symbol] | [symbol] | [symbol] | [symbol] | 61 | 62 | [symbol] | 23 | 28 | [symbol] | 2 | [symbol] | [symbol] | 70 | [symbol] | [symbol] | 72 | 12 |
| | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] - | | | | | | | | | | | N/A | N/A | N/A | N/A | | | | |
| DLBCL | | | | | | | | | | | | | | | | | | |
| Cohort | | | | | | | | | | | | | | | | | | |
| non- [symbol] - | N/A | N/A | N/A | N/A | N/A | N/A | N/A | | | | | | | | N/A | N/A | N/A | |
| DLBCL | | | | | | | | | | | | | | | | | | |
| Cohort | | | | | | | | | | | | | | | | | | |
| [symbol] | | | | | | | | N/A | N/A | N/A | | | | | | | | N/A |
| [symbol] ( [symbol] ) | | | | | | | | | | | | | | | | | | |
| CR + PR | | | | | | | | | | | | | | | | | | |
| [symbol] | | | | | | | | | | | | | | | | | | |
| [symbol] D [symbol] | N/A | N/A | N/A | N/A | N/A | N/A | N/A | N/A | N/A | N/A | | | | | | | | |
| [symbol] | M | | [symbol] | M | M | | M | M | | M | [symbol] | M | M | M | M | | M | |
| [symbol] | | [symbol] | | | | | | | | | M | M | | M | | | | |
| [symbol] | M | A | M | | M | | [symbol] | M | M | M | | M | M | | | A | M | M |
| [symbol] | | | | | | | | T | T | T | | | T | | T | | | T |
| [symbol] | T | | T | | | T | T | | T | T | | | | M | | | | |
| [symbol] | | | | | | | | | | | M | | | | | M | | |
| [symbol] | | | | | [symbol] | | | | | | M | | | | | | [symbol] | |
| [symbol] | | | | | | | | | | | | | | | | | M | |
| [symbol] | | | | | | M | | | | | [symbol] | | | | | | | |
| [symbol] ( [symbol] ) | | A | | | | | | | | | | [symbol] | A | | | | | |
| [symbol] | | | | | | | | | | | | | | | | | | |
| [symbol] | [symbol] | | M | M | | | M | | | | M | M | | M | M | | M | M |
| [symbol] | | | | | | | | M | | | | M | | M | M | | | |
| [symbol] ( [symbol] ) | | | | | | | | | | | | M | | | | | | |
| [symbol] | | | M | M | | | M | | | M | M | | | M | M | | | |
| [symbol] | | | | | | | [symbol] | M | | | | | | | | | M | M |
| [symbol] | | | | | | | | | | | | | | | | | | |
| [symbol] | | | | | | [symbol] | | M | | | | M | | | | | [symbol] | |
| [symbol] | | | | | | | M | | | M | M | | | | M | | | |
| [symbol] | | | | | | | M | | | M | | M | | M | | | | M |
| [symbol] | | | | | | | M | | | | M | | M | M | | | M | |
| [symbol] | M | [symbol] | [symbol] | [symbol] | M | | [symbol] | M | [symbol] | [symbol] | [symbol] | [symbol] | [symbol] | [symbol] | [symbol] | | M | |
| [symbol] | | | | | | | | | | | | | | M | | | | |
| [symbol] | | | | | | | | | | | | | | | | | M | |
| [symbol] | | | M | M | | | M | M | | | M | M | | M | | | | |
| [symbol] | | | T | | | | | [symbol] | | | | | | | | | | |
| [symbol] ( [symbol] ) | | | M | | | M | | | | | | | | | | | | |
| [symbol] ( [symbol] ) | | A | | | | | | | | | | | [symbol] | | | | | |
| [symbol] | M | M | M | | [symbol] | M | M | | | | | M | | M | | | M | [symbol] |
| [symbol] | | | | | | | | | | | M | M | | | | | | |
| [symbol] | M | | | | [symbol] | M | [symbol] | | | | [symbol] | M | | [symbol] | M | [symbol] | | M |
| [symbol] | | | | | | | | | | | | | | | | | | |
| [symbol] | | | M | | | | M | | | | M | | | | | | | |
| [symbol] | | | | | | | | | | M | | | | | | | | |
| [symbol] | [symbol] | | | | | | | | | | M | [symbol] | | | M | | M | [symbol] |
| [symbol] | | | M | | | | | | M | | | | | | | | | |

“F” = Frameshift or nonsense mutation;
“M” = Missense mutation;
“T” = Translocation
“A” = Amplification [symbol]
indicates data missing or illegible when filed

TABLE 19

| | | | | | | | | | | | | | | | On Study | | | |
|---|---|---|---|---|---|---|---|---|---|---|---|---|---|---|---|---|---|---|
| | 4 | 7 | [symbol] | [symbol] | [symbol] | [symbol] | [symbol] | 21 | [symbol] | 14 | [symbol] | 31 | [symbol] | [symbol] | [symbol] | [symbol] | [symbol] | 40 |
| | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| GCB- | N/A | N/A | N/A | N/A | N/A | | | | | | | | | | | | | |
| DLBCL | | | | | | | | | | | | | | | | | | |
| Cohort | | | | | | | | | | | | | | | | | | |
| non-GCB- | | | | | | N/A | N/A | N/A | N/A | | | | | | N/A | | | |
| DLBCL | | | | | | | | | | | | | | | | | | |
| Cohort | | | | | | | | | | | | | | | | | | |
| Follicular | | | | | | | | | | N/A | N/A | N/A | N/A | N/A | | N/A | N/A | N/A |
| Lymphoma | | | | | | | | | | | | | | | | | | |
| EZH2 MT | | | | N/A | N/A | | | N/A | | | N/A | | | N/A | | | | |
| Positive | | | | | | | | | | | | | | | | | | |
| (Cobas) | | | | | | | | | | | | | | | | | | |
| CR + PR | N/A | N/A | N/A | N/A | N/A | N/A | N/A | N/A | N/A | N/A | N/A | N/A | N/A | N/A | | | | |
| Stable | | | | | | | | | | | | | | | N/A | N/A | N/A | N/A |
| Disease | | | | | | | | | | | | | | | | | | |
| Progressive | | | | | | | | | | | | | | | | | | |
| Disease | | | | | | | | | | | | | | | | | | |
| ARID1A | | | | | | | | | | | | | M | | | [symbol] | | |
| B2M | | | | | [symbol] | M | | | | | | | | | | | | |
| BCL2 | | | | | | | | | | | | | | | | | | |
| Sequence | | | | | | | | | | | | | | | | | | |
| Mutation | | | | | | | | | | | | | | | | | | |
| BCL2 Trans- | | | | | | | | | | | | | | | | T | | |
| location | | | | | | | | | | | | | | | | | | |
| BCL6 | | | | | | | | | | | | | | | | | | |
| CARD11 | | | | | | | | [symbol] | M | | | | | | | | | |
| CC [symbol] D3 | | | M | | | | | | | | | | | | | | | |
| CD58 | | | | | | | | | | | | | | | | | | |
| CD79 [symbol] | | | | | | | | | M | | | | | | | | | |
| CD274 | | | | | | | | | | | | | | | | | | |
| (PDL1) | | | | | | | | | | | | | | | | | | |
| CD [symbol] 2A | | | | | | | | | | | | | | | | | | |
| CRE [symbol] BP | | | | | | | | | | | M | M | | | | | M | |
| EP300 | | | | | | | | | | | | | | | | | | |
| E2H2 | | | | | M | | | M | | | M | | | M | | | | |
| (Y [symbol] , | | | | | | | | | | | | | | | | | | |
| A [symbol] ) | | | | | | | | | | | | | | | | | | |
| E2H2 non | | | | | M | | | | | | | | | | | | | |
| [symbol] | | | | | | | | | | | | | | | | | | |
| FOXO1 | | | | | | | | | | | | | | | | | | |
| FOX [symbol] | | | | | | | | | | | | | | | | | | |
| GMA13 | | | | M | M | | | | | | | | | | | | | |
| HIST [symbol] | | | | | | | | | | | | | | | | | | |
| HIST [symbol] | | | | | | | | | | | | | | | | | | |
| HIST [symbol] | | M | | | | | | | | | | | | | M | | | |
| KMT2D | | | | | M | | | [symbol] | | | | | | | | | [symbol] | |
| KRAS | | | | | | | | | | | | | | | | | | |
| MEF2B | | | | | | | | M | | | M | | | | | M | | |
| MYC | | | | | | | | | | | | | | | | | | |
| Sequence | | | | | | | | | | | | | | | | | | |
| Mutation | | | | | | | | | | | | | | | | | | |
| MYC Trans- | | | | | | | | | | | | | | | | | | |
| location | | | | | | | | | | | | | | | | | | |
| MYDB8 | | M | | | | | M | | M | | | | | | | | | |
| (273P) | | | | | | | | | | | | | | | | | | |
| PDCD1LG2 | | | | | | | | | | | | | | | | | | |
| (POL2) | | | | | | | | | | | | | | | | | | |
| [symbol] | | [symbol] | | | | | | | M | | | | | | | | | |
| [symbol] | | | | | | | | | | | | | | | | | | |
| PRDM1 | | | | | | | | | M | | | | | | | | | |
| P [symbol] | | | | | | | | | | | | | | | | | | |
| SOCS1 | | | | | [symbol] | | | | | | | | | M | | | M | |
| STAT6 | | | | | M | | | M | | | M | M | | | | | M | |
| TNFAIP3 | | | | | [symbol] | | | | | | | | | | | | | |
| TNFRSF14 | | | | | | | | M | | | | M | | [symbol] | | | M | |

| | On Study | Off Study | | | | | | | | | | | | | | |
|---|---|---|---|---|---|---|---|---|---|---|---|---|---|---|---|---|
| | 71 | 34 | [symbol] | [symbol] | [symbol] | [symbol] | [symbol] | [symbol] | 17 | [symbol] | [symbol] | 10 | 18 | [symbol] | [symbol] | [symbol] |
| | [symbol] | [symbol] | [symbol] [symbol] | [symbol] | [symbol] | [symbol] | [symbol] | [symbol] | [symbol] | [symbol] | [symbol] | [symbol] | [symbol] | [symbol] | [symbol] | [symbol] |
| GCB- | | N/A | N/A | N/A | N/A | N/A | N/A | | | | | | | | | |
| DLBCL | | | | | | | | | | | | | | | | |
| Cohort | | | | | | | | | | | | | | | | |
| non-GCB- | | | | | | | | N/A | N/A | N/A | N/A | | | | | |
| DLBCL | | | | | | | | | | | | | | | | |
| Cohort | | | | | | | | | | | | | | | | |
| Follicular | N/A | | | | | | | | | | | N/A | N/A | N/A | N/A | N/A |
| Lymphoma | | | | | | | | | | | | | | | | |
| EZH2 MT | | N/A | | | | | N/A | | | | | | | N/A | | |
| Positive | | | | | | | | | | | | | | | | |
| (Cobas) | | | | | | | | | | | | | | | | |
| CR + PR | | | | | | | | | | | | | | | | |
| Stable | N/A | N/A | N/A | N/A | N/A | N/A | N/A | N/A | N/A | N/A | N/A | N/A | N/A | N/A | N/A | N/A |
| Disease | | | | | | | | | | | | | | | | |
| Progressive | | | | | | | | | | | | | | | | |
| Disease | | | | | | | | | | | | | | | | |
| ARID1A | | | | | | | M | | | | | | | | | [symbol] |
| B2M | | M | | | | | M | [symbol] | | | | | | | | |
| BCL2 | M | | M | | | | M | | | | | | | | M | M |
| Sequence | | | | | | | | | | | | | | | | |
| Mutation | | | | | | | | | | | | | | | | |
| BCL2 Trans- | | | T | | | T | T | | | | | | | | T | T |
| location | | | | | | | | | | | | | | | | |
| BCL6 | | | | | | | | | | | | T | | | | |
| CARD11 | M | | | M | | | | | | | | | | | | |
| CC [symbol] D3 | | | | | | | | | | | | | | | | M |
| CD58 | | | | | | | | | | | | | | | | |
| CD79 [symbol] | | | | | | | | | | | | | | | | |
| CD274 | | | | | | | | | | | | | | | | |
| (PDL1) | | | | | | | | | | | | | | | | |
| CD [symbol] 2A | | | M | | | | | | | | | | | | | |
| CRE [symbol] BP | M | M | [symbol] | | | M | | | | | | | | M | | |
| EP300 | | [symbol] | | | | | M | M | | | | | | | | |
| E2H2 | | | | | | | M | | | | | | | | | |
| (Y [symbol] , | | | | | | | | | | | | | | | | |
| A [symbol] ) | | | | | | | | | | | | | | | | |
| E2H2 non | | | | | | | | | | | | | | | | |
| [symbol] | | | | | | | | | | | | | | | | |
| FOXO1 | | | | | | | | | | | | M | | M | M | |
| FOX [symbol] | | | | | | | | | | | | | | | | |
| GMA13 | | | | | | | | M | | | | | | | | |
| HIST [symbol] | | M | | | | | | | | | | | | | | |
| HIST [symbol] | | M | | | | | | | | | | | | | | |
| HIST [symbol] | M | | | | | | | | | | | | | | | |
| KMT2D | [symbol] | | | | | [symbol] | [symbol] | | | | [symbol] | [symbol] | | | [symbol] | [symbol] |
| KRAS | | | | | | | | | | | | | | | | |
| MEF2B | | | | | | | | | | | | | | | | |
| MYC | | | | | | | | | | | | | | | | |
| Sequence | | | | | | | | | | | | | | | | |
| Mutation | | | | | | | | | | | | | | | | |
| MYC Trans- | | | | | | | | | | | | | | T | | |
| location | | | | | | | | | | | | | | | | |
| MYDB8 | | | | | | | | | | | | | | | | |
| (273P) | | | | | | | | | | | | | | | | |
| PDCD1LG2 | | | | | | | | | | | | | | | | |
| (POL2) | | | | | | | | | | | | | | | | |
| [symbol] | | | | | | | | | | | | | | | | |
| [symbol] | | | | | | | | | | | | | | | | |
| PRDM1 | | | | | | | | | | | | | | | | |
| P [symbol] | | | | | | | | | | | | | | | | |
| SOCS1 | | | M | | | | | [symbol] | | | | | | | | |
| STAT6 | | | M | | | M | | M | | | | | | | | |
| TNFAIP3 | | | | | | | | [symbol] | | | | | M | | | |
| TNFRSF14 | | | [symbol] | | | | M | | | | | | | | | |

| | 3 | 11 | 13 | 19 | 22 | 24 | 36 | 37 | 43 | 48 | 49 | 52 | 53 | 67 | 69 | 15 | 16 | 26 | 38 |
|---|---|---|---|---|---|---|---|---|---|---|---|---|---|---|---|---|---|---|---|
| | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| GCB- | N/A | N/A | N/A | N/A | N/A | N/A | N/A | N/A | N/A | N/A | N/A | N/A | N/A | N/A | N/A | | | | |
| DLBCL | | | | | | | | | | | | | | | | | | | |
| Cohort | | | | | | | | | | | | | | | | | | | |
| non-GCB- | | | | | | | | | | | | | | | | N/A | N/A | N/A | N/A |
| DLBCL | | | | | | | | | | | | | | | | | | | |
| Cohort | | | | | | | | | | | | | | | | | | | |
| Follicular | | | | | | | | | | | | | | | | | | | |
| Lymphoma | | | | | | | | | | | | | | | | | | | |
| EZH2 MT | | N/A | | | | N/A | N/A | | | | N/A | | | | | | | | |
| Positive | | | | | | | | | | | | | | | | | | | |
| (Cobas) | | | | | | | | | | | | | | | | | | | |
| CR + PR | | | | | | | | | | | | | | | | | | | |
| Stable | | | | | | | | | | | | | | | | | | | |
| Disease | | | | | | | | | | | | | | | | | | | |
| Progressive | N/A | N/A | N/A | N/A | N/A | N/A | N/A | N/A | N/A | N/A | N/A | N/A | N/A | N/A | N/A | N/A | N/A | N/A | N/A |
| Disease | | | | | | | | | | | | | | | | | | | |
| ARID1A | | | | | | | | M | | | | | | | | | | [symbol] | |
| B2M | M | | [symbol] | M | | | | | | | | | | | [symbol] | | | | |
| BCL2 | M | M | M | M | M | M | M | M | M | | | M | | | | | | | |
| Sequence | | | | | | | | | | | | | | | | | | | |
| Mutation | | | | | | | | | | | | | | | | | | | |
| BCL2 Trans- | T | | | | T | | | | T | | | T | | | T | | | | |
| location | | | | | | | | | | | | | | | | | | | |
| BCL6 | T | | | T | | | T | | | T | | | | | | | | | |
| CARD11 | | M | | | M | M | | | | | | | | | | | | | |
| CC [symbol] D3 | | [symbol] | | | | | | | | | | | | | | | | | |
| CD58 | | | | | | | | | | | | | | | | [symbol] | | | |
| CD79 [symbol] | | | | | | | | | | | | | | | | | | | |
| CD274 | | | | | | | | | | | | | | | | | | | |
| (PDL1) | | | | | | | | | | | | | | | | | | | |
| CD [symbol] 2A | | | | | | | | | | | | | | | | | | | |
| CRE [symbol] BP | M | | | M | M | | [symbol] | M | | M | | [symbol] | | | | | | | |
| EP300 | | | | | | | | | | | | | | | | | | | |
| E2H2 | | M | | | | M | | | | | M | | | | | | | | |
| (Y [symbol] , | | | | | | | | | | | | | | | | | | | |
| A [symbol] ) | | | | | | | | | | | | | | | | | | | |
| E2H2 non | | | | | | | | | | | | | | | | | | | |
| [symbol] | | | | | | | | | | | | | | | | | | | |
| FOXO1 | | | | | | | | M | M | | | | | | M | | | | |
| FOX [symbol] | | | | | | | | | | | | | | | | | | | |
| GMA13 | | M | | [symbol] | | | | M | | | [symbol] | | [symbol] | | | | | | |
| HIST [symbol] | | M | | | | | | | | | | | | | | M | | | |
| HIST [symbol] | | | | | | | | | | | | M | | | | | | | |
| HIST [symbol] | | M | | | | | M | | | | | M | M | | M | M | | | M |
| KMT2D | [symbol] | [symbol] | [symbol] | M | | [symbol] | M | | | | | M [symbol] | M | [symbol] | | | | M | |
| KRAS | | | | | | | | | | | | | | | | | | | |
| MEF2B | | | | M | M | | | | | | M | M | | | | | | | |
| MYC | | | | | | | | M | M | | | | | | M | | | | |
| Sequence | | | | | | | | | | | | | | | | | | | |
| Mutation | | | | | | | | | | | | | | | | | | | |
| MYC Trans- | | | | | [symbol] | | | | | | | | | | [symbol] | | | | |
| location | | | | | | | | | | | | | | | | | | | |
| MYDB8 | | | | | | | | | | | | | | | | | | | |
| (273P) | | | | | | | | | | | | | | | | | | | |
| PDCD1LG2 | | | | | | | | | | | | | | | | | | | |
| (POL2) | | | | | | | | | | | | | | | | | | | |
| [symbol] | | M | M | | | | M | | | | | | | | M | | | | [symbol] |
| [symbol] | | | | M | | | | | | | | | | | | | | | |
| PRDM1 | | | | | | | | | | | | | | | | | | | |
| P [symbol] | | | | | | | | | | | | | | | | | | | |
| SOCS1 | [symbol] | | | M | | | [symbol] | | | | | | [symbol] | | M | [symbol] | | | |
| STAT6 | | | | | | | | | | | | | | | M | | | | |
| TNFAIP3 | | | | | | | | | | [symbol] | | | M | | | | | | |
| TNFRSF14 | | | | | [symbol] | [symbol] | | M | | | | | | | | | | | |

| | | | | | | | | | | | Off Study | | | | | | | |
|---|---|---|---|---|---|---|---|---|---|---|---|---|---|---|---|---|---|---|
| | [symbol] | [symbol] | [symbol] | [symbol] | 61 | 62 | 66 | 23 | 28 | [symbol] | 2 | 60 | [symbol] | 7 [symbol] | [symbol] | 27 | 72 | 12 |
| | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| GCB- | | | | | | | | | | | N/A | N/A | N/A | N/A | | | | |
| DLBCL | | | | | | | | | | | | | | | | | | |
| Cohort | | | | | | | | | | | | | | | | | | |
| non-GCB- | N/A | N/A | N/A | N/A | N/A | N/A | N/A | | | | | | | | N/A | N/A | N/A | |
| DLBCL | | | | | | | | | | | | | | | | | | |
| Cohort | | | | | | | | | | | | | | | | | | |
| Follicular | | | | | | | | N/A | N/A | N/A | | | | | | | | N/A |
| Lymphoma | | | | | | | | | | | | | | | | | | |
| EZH2 MT | | | | | | | | | | | | | | | | | | |
| Positive | | | | | | | | | | | | | | | | | | |
| (Cobas) | | | | | | | | | | | | | | | | | | |
| CR + PR | | | | | | | | | | | | | | | | | | |
| Stable | | | | | | | | | | | | | | | | | | |
| Disease | | | | | | | | | | | | | | | | | | |
| Progressive | N/A | N/A | N/A | N/A | N/A | N/A | N/A | N/A | N/A | N/A | | | | | | | | |
| Disease | | | | | | | | | | | | | | | | | | |
| ARID1A | | | | | | | | | | | [symbol] | | M | | | | | |
| B2M | | | | | | | | | | | | | | M | | | | |
| BCL2 | | | M | | | | | | M | | | M | M | | | | | M |
| Sequence | | | | | | | | | | | | | | | | | | |
| Mutation | | | | | | | | | | | | | | | | | | |
| BCL2 Trans- | | | | | | | | T | T | T | | | T | | T | | | T |
| location | | | | | | | | | | | | | | | | | | |
| BCL6 | T | | T | | | T | T | | T | T | | | | | | | | |
| CARD11 | | | | | | | | | | | M | | | | | | | |
| CC [symbol] D3 | | | | | [symbol] | | | | | | | | | | | | [symbol] | |
| CD58 | | | | | | | | | | | | | | | | | | |
| CD79 [symbol] | | | | | | M | | | | | [symbol] | | | | | | | |
| CD274 | | | | | | | | | | | | | | | | | | |
| (PDL1) | | | | | | | | | | | | | | | | | | |
| CD [symbol] 2A | | | | | | | | | | | | | | | | | | |
| CRE [symbol] BP | | | | | | | | | | | M | M | | | | | | M |
| EP300 | | | | | | | | M | | | | M | | | | | | |
| E2H2 | | | | | | | | | | | | | | | | | | |
| (Y [symbol] , | | | | | | | | | | | | | | | | | | |
| A [symbol] ) | | | | | | | | | | | | | | | | | | |
| E2H2 non | | | | | | | | | | | | | | | | | | |
| [symbol] | | | | | | | | | | | | | | | | | | |
| FOXO1 | | | | | | | | M | | | | | | | | | | M |
| FOX [symbol] | | | | | | | | | | | | | | | | | | |
| GMA13 | | | | | | | | M | | | | M | | | | | | |
| HIST [symbol] | | | | | | | | | | | M | | | | M | | | |
| HIST [symbol] | | | | | | | M | | | M | | M | | M | | | | M |
| HIST [symbol] | | | | | | | M | | | | M | | | M | | | | |
| KMT2D | | [symbol] | [symbol] | | M | | | | [symbol] | | [symbol] | [symbol] | [symbol] | [symbol] | [symbol] | | | |
| KRAS | | | | | | | | | | | | | | | | | | |
| MEF2B | | | | | | | | | | | | | | | | | | |
| MYC | | | M | | | | | | | | M | | | | | | | |
| Sequence | | | | | | | | | | | | | | | | | | |
| Mutation | | | | | | | | | | | | | | | | | | |
| MYC Trans- | | | [symbol] | | | | | [symbol] | | | | | | | | | | |
| location | | | | | | | | | | | | | | | | | | |
| MYDB8 | | | M | | | M | | | | | | | | | | | | |
| (273P) | | | | | | | | | | | | | | | | | | |
| PDCD1LG2 | | | | | | | | | | | | | | | | | | |
| (POL2) | | | | | | | | | | | | | | | | | | |
| [symbol] | | M | M | | [symbol] | M | | | | | | | | | | | M | [symbol] |
| [symbol] | | | | | | | | | | | M | | | | | | | |
| PRDM1 | | | | | | | | | | | [symbol] | | | | M | [symbol] | | |
| P [symbol] | | | | | | | | | | | | | | | | | | |
| SOCS1 | | | M | | | | [symbol] | | | | M | | | | | | | M |
| STAT6 | | | | | | | | | | M | | | | | | | | |
| TNFAIP3 | | | | | | | M | | | | | | | | | | | [symbol] |
| TNFRSF14 | | | | | | | | | M | | | | | | | | | |

“F” = Frameshift or nonsense mutation;
“M” = Missense mutation;
“T” = Translocation
“A” = Amplification [symbol]
indicates data missing or illegible when filed

Table 20 summarizes specific variants of STAT6, and their variant allele frequencies, observed in patients of different patient cohorts (DLBCL GCB EZH2 wild type, FL EZH2 wild type, FL EZH2 mutant and DLBCL non-GCB).

TABLE 20

| Sample ID | Variant | vaf | Response | Cohort |
|---|---|---|---|---|
| 10012004 | 419D > G | 42% | Progressive Disease | DLBCL GCB EZH2 Wild-type |
| 10032007 | 419D > G | 36% | Partial Response | FL EZH2 Wild-type |
| 10042005 | 419D > G | 19% | Partial Response | FL EZH2 Wild-type |
| 10052001 | 419D > G | 24% | Partial Response | FL EZH2 Mutant |
| 10062002 | 419D > G | 29% | Stable Disease | DLBCL GCB EZH2 Wild-type |
| 20012001 | 286Q > R | 24% | Stable Disease | DLBCL GCB EZH2 Wild-type |
| 20012003 | 417N > S | 27% | Stable Disease | DLBCL GCB EZH2 Wild-type |
| 20022001 | 377E > K | 33% | Partial Response | FL EZH2 Mutant |
| 20022008 | 371C > R | 35% | Progressive Disease | FL EZH2 Wild-type |
| 20042003 | 419D > A | 39% | Partial Response | DLBCL GCB EZH2 Mutant |
| 20052004 | 419D > A | 30% | Complete Response | DLBCL GCB EZH2 Wild-type |
| 30022001 | 419D > H | 42% | Progressive Disease | DLBCL GCB EZH2 Wild-type |
| 50022001 | 419D > Y | 39% | Stable Disease | DLBCL non-GCB |

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples below are for purposes of illustration and not limitation of the claims that follow. Where names of cell lines or genes are used, abbreviations and names conform to the nomenclature of the American Type Culture Collection (ATCC) or the National Center for Biotechnology Information (NCBI), unless otherwise noted or evident from the context.

The invention can be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

1. A method of treating cancer comprising administering a an inhibitor of Enhancer to Zeste Homolog 2 (EZH2) to a subject in need thereof, wherein the subject has at least one mutation in one or more sequences encoding STAT6
wherein the at least one mutation results in:
a substitution of glycine (G), alanine (A), histidine (H) or tyrosine (Y) for
aspartate (D) at position 419 (D419G/A/H/Y);
a substitution of serine (S) for asparagine (N) at position 417 (N417S);
a substitution of arginine (R) for cysteine (C) at position 371 (C371R); or
a substitution of lysine (K) for glutamate (E) at position 377 (E377K),
wherein the inhibitor of EZH2 is 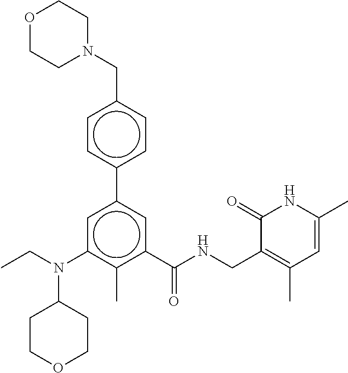
or a pharmaceutically-acceptable salt thereof.

2.-10. (canceled)

11. The method of claim 1, wherein the at least one mutation decreases the function of a protein encoded by the mutated sequence as compared to the function of the protein encoded by the wild-type sequence.

12. The method of claim 1, wherein the at least one mutation is a loss-of-function mutation.

13.-15. (canceled)

16. The method of claim 1, wherein the inhibitor of EZH2 is administered orally.

17. The method of claim 16, wherein the inhibitor of EZH2 is formulated as a tablet.

18. The method of claim 1, wherein the amount of the inhibitor of EZH2 is between 100 mg and 3200 mg per day.

19. The method of claim 18, wherein the amount of the inhibitor of EZH2 is 100 mg, 200 mg, 400 mg, 600 mg, 800 mg, 1000 mg, 1200 mg, 1400 mg, 1600 mg or 3200 mg per day.

20. The method of claim 19, wherein the amount of the inhibitor of EZH2 is 1600 mg per day.

21. The method of claim 1, wherein the amount of the inhibitor of EZH2 is administered at 800 mg twice per day (BID).

22. The method of claim 1, wherein the at least one mutation decreases a level of acetylation of a lysine (K) on histone (3) compared to a level of acetylation of the same lysine by a wild type HAT.

23. The method of claim 22, wherein the lysine (K) on histone (3) is at position 27 (H3K27).

24.-30. (canceled)

31. The method of claim 1, wherein the subject expresses a wild type EZH2 protein and does not express a mutant EZH2 protein.

32. The method of claim 1, wherein the subject expresses a mutant EZH2 protein.

33.-36. (canceled)

37. The method of claim 1, wherein the subject does not have a MYC and/or a HIST1H1E mutation.

38.-41. (canceled)

42. The method of claim 1, wherein the cancer is B-cell lymphoma.

43. The method of claim 42, wherein the B-cell lymphoma is an activated B-cell (ABC) type.

44. The method of claim 42, wherein the B-cell lymphoma is a germinal B-cell (GBC) type.

45. The method of claim 1, wherein the cancer is follicular lymphoma.

46.-56. (canceled)

57. A method of selecting a subject having cancer for treatment with an inhibitor of Enhancer to Zeste Homolog 2 (EZH2), the method comprising:
a) detecting the presence or absence of at least one mutation in one or more sequences encoding STAT6 in a sample obtained from the subject,
wherein the at least one mutation results in:
a substitution of glycine (G), alanine (A), histidine (H) or tyrosine (Y) for aspartate (D) at position 419 (D419G/A/H/Y);
a substitution of serine (S) for asparagine (N) at position 417 (N417S);
a substitution of arginine (R) for cysteine (C) at position 371 (C371R); or
a substitution of lysine (K) for glutamate (E) at position 377 (E377K);
b) selecting the subject for treatment with the inhibitor of EZH2 when the presence of the at least one mutation in one or more sequence encoding STAT6 is detected, wherein the inhibitor of EZH2 is 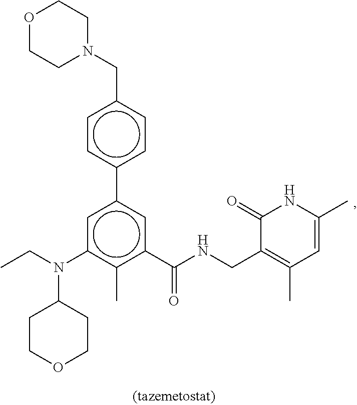
or a pharmaceutically-acceptable salt thereof.

58. A method of treating a subject having cancer comprising:
a) detecting the presence of at least one mutation in one or more sequences encoding STATE in a sample obtained from the subject,
wherein the at least one mutation results in:
a substitution of glycine (G), alanine (A), histidine (H) or tyrosine (Y) for
aspartate (D) at position 419 (D419G/A/H/Y);
a substitution of serine (S) for asparagine (N) at position 417 (N417S);
a substitution of arginine (R) for cysteine (C) at position 371 (C371R); or
a substitution of lysine (K) for glutamate (E) at position 377 (E377K);
b) administering to the subject an inhibitor of Enhancer to Zeste Homolog 2 (EZH2), wherein the inhibitor of EZH2 is 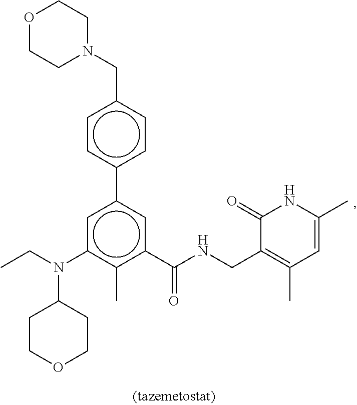
or a pharmaceutically-acceptable salt thereof.
